SEQUENCE LISTING (108 sequences)

SEQ ID NO: 1 - DNA, 627 bp, Artificial Sequence
  Description of Artificial Sequence Synthetic polynucleotide
atggccgccatgcccaagcccgctgcgttctggaacgaccgctttgccaacgaagaatacgtgtacggcgaagcccccaaccgtttcgtcgcgagcgccgcccgtacgtggctgccggaagccggtgaagttctcctgctcggggcgggcgaagggcgtaacgccgtgcatctggcccgtgaaggccatacggtcaccgcggtcgattacgccgtggaagggctccgtaagacggaacgtctcgcgacggaagccggggtggaagtcgaagcgattcaagccgatgtgcgtgaatggaagcccgcccgtgcgtgggatgcggtcgtcgtcacgtttctccatcttcccgccgatgaacgtccgggcctgtaccgtctcgttcaacgttgtttgcgtcccggggggcgtctcgtggcggaatggtttcgtccggaacaacgtacggatggctacacgagcggcggcccgcccgatcctgccatgatggtcaccgccgatgaactccgtgggcatttcgccgaagcgggcattgatcatctcgaagcggccgaaccgaccctcgatgaaggcatgcatcgtggccccgcggcgacggttcgtctcgtgtggtgccgtccgtccacctcg

SEQ ID NO: 2 - DNA, 627 bp, Artificial Sequence
  Description of Artificial Sequence Synthetic polynucleotide
atggccgccatgcccaagcccgctgcgttctggaacgaccgctttgccaacgaagaatacgtgtacggcgaagcccccaaccgcttcgtcgcgagcgccgcccggacgtggctgccggaagccggtgaagttctcctgctcggggcgggcgaagggcgcaacgccgtgcacctggcccgggaaggccatacggtcaccgcggtcgactacgccgtggaagggctccgcaagacggaacgcctcgcgacggaagccggggtggaagtcgaagcgatccaggccgatgtgcgcgaatggaagcccgcccgggcgtgggacgcggtcgtcgtcacgtttctccaccttcccgccgacgaacgaccgggcctgtaccgcctcgttcagcgctgtttgcggcccggggggcgcctcgtggcggaatggtttcgcccggaacagcgcacggacggctacacgagcggcggcccgcccgatcctgccatgatggtcaccgccgacgaactccgcgggcacttcgccgaagcgggcatcgaccatctcgaagcggccgaaccgaccctcgacgaaggcatgcaccggggccccgcggcgacggttcgtctcgtgtggtgccggccgtccacctcg

SEQ ID NO: 3 - DNA, 456 bp, Artificial Sequence
  Description of Artificial Sequence Synthetic polynucleotide
atggcgcgttcgatcgattacggcaacctcatgcaccgcgcgatgcgtggcctgattcaaagcgtgctcgaagatgtggccgaacatgggctgcccggcgcgcatcatttcttcattaccttcgatacgacccatcccgatgtggccatggccgattggctccgtgcgcgttatccgcaagaaatgacggtcgtgattcaacattggtacgaaaacctctccgccgatgatcatggcttctcggtcacgctgaacttcggcaaccaacccgaaccgctggtcattcccttcgatgccgtgcgtaccttcgtcgatccgtccgtggaattcggcctccgtttcgaaacccatgaagaagatgaagaagaagaaacgggcggcgatgaagatcccgatggcgatgatgaaccgccgcgtcatgatgcgcaagtcgtgagcctcgataagttccgtaag

SEQ ID NO: 4 - DNA, 456 bp, Artificial Sequence
  Description of Artificial Sequence Synthetic polynucleotide
atggcgcgttcgatcgattacggcaacctcatgcaccgcgcgatgcggggcctgatccagagcgtgctcgaggatgtggccgagcatgggctgcccggcgcgcatcatttcttcatcaccttcgacacgacccatcccgatgtggccatggccgactggctccgcgcgcgctatccgcaggagatgacggtcgtgatccagcattggtacgagaacctctccgccgacgaccatggcttctcggtcacgctgaacttcggcaaccagcccgagccgctggtcatccccttcgatgccgtgcgcaccttcgtcgacccgtccgtggaattcggcctccggttcgagacccatgaggaggacgaggaggaggagacgggcggcgacgaggatcccgacggcgacgacgagccgccgcgccatgacgcgcaggtcgtgagcctcgacaagttccgcaag

SEQ ID NO: 5 - DNA, 774 bp, Artificial Sequence
  Description of Artificial Sequence Synthetic polynucleotide
atgagcacgcggacgaggacgacggaagaacgccggcacgagattgtgcgtgtcgcccgtgccaccggctcggtcgatgtcaccgcgctcgccgccgaactgggcgtcgccaaggaaaccgtacgtcgtgatctgcgtgccctggaagatcatggcctggtccgtcgtacccatggcggcgcctacccggtggaaagcgccggtttcgaaaccacgctcgccttccgtgccaccagccatgtgcccgaaaagcgtcgtattgcgtccgccgccgtcgaactgctcggcgatgcggaaacggtcttcgtcgatgaaggcttcaccccccaactcattgccgaagccctgccccgtgatcgtccgctgaccgtggtcaccgcgtccctgccggtggcgggcgcgctggccgaagcgggcgatacgtccgtcctgctgctcggcggccgtgtccgttcgggcaccctggccaccgtcgatcattggaccacgaagatgctggccggcttcgtcattgatctggcgtacattggcgccaacggcatttcccgtgaacatggtctcaccacacccgatcccgcggtcagcgaagtcaaggcgcaagccgtccgtgccgcccgtcgtacggtgttcgccggcgcgcataccaagttcggggcggtgagcttctgccgtttcgcggaagtcggcgccctggaagccattgtcaccagcacgctgctgccctcggccgaagcccatcgttactccctcctcggcccccaaattattcgtgtc

SEQ ID NO: 6 - DNA, 774 bp, Artificial Sequence
  Description of Artificial Sequence Synthetic polynucleotide
atgagcacgcggacgaggacgacggaagaacgccggcacgagatcgtgcgggtcgcccgcgccaccggctcggtcgacgtcaccgcgctcgccgccgaactgggcgtcgccaaggagaccgtacgacgcgacctgcgcgccctggaggaccatggcctggtccgccgcacccatggcggcgcctacccggtggagagcgccggtttcgagaccacgctcgccttccgcgccaccagccatgtgcccgagaagcgccggatcgcgtccgccgccgtcgaactgctcggcgacgcggagacggtcttcgtcgacgagggcttcaccccccagctcatcgccgaggccctgccccgggaccggccgctgaccgtggtcaccgcgtccctgccggtggcgggcgcgctggccgaggcgggcgacacgtccgtcctgctgctcggcggccgggtccgctcgggcaccctggccaccgtcgaccattggaccacgaagatgctggccggcttcgtcatcgacctggcgtacatcggcgccaacggcatctcccgggagcatggtctcaccacacccgaccccgcggtcagcgaggtcaaggcgcaggccgtccgggccgcccgccgcacggtgttcgccggcgcgcataccaagttcggggcggtgagcttctgccggttcgcggaggtcggcgccctggaggccatcgtcaccagcacgctgctgccctcggccgaggcccatcgctactccctcctcggcccccagatcatccgcgtc

SEQ ID NO: 7 - DNA, 849 bp, Artificial Sequence
  Description of Artificial Sequence Synthetic polynucleotide
atgaagacaattcaggagcagcagatgaagatagttaggaatatgcgtcgtattcgttacaagattgctgttattagcacgaaaggaggtgtggggaaaagctttgttaccgctagcctcgcggcagccctcgctgcggaagggcgtcgtgttggagtttttgatgcagatattagcggtcctagcgttcataaaatgctcggcctccaaacgggcatgggtatgccctcgcaactcgatggcactgtaaagcccgtggaagttcctccgggaattaaagtagctagcattgggctgttgctgcccatggatgaagtgcccctaatttggcgtggggccattaagacgagtgccattcgtgaactgcttgcatacgtcgattggggagaactcgattatctcctcattgatctacctccgggaacaggtgatgaagtcctcacgattacccaaattattcccaacattacgggcttcctggtagtcacgattcccagcgaaattgctaagtctgtcgttaagaaggctgtcagctttgccaagcgtattgaagcccctgtgattggaattgtcgaaaacatgagctactttcgttgtagcgatggatccattcattatattttcggccgtggcgcggctgaagaaattgcgtcacaatatggtattgaactcctcggcaaaattcccattgatcctgcgattcgtgaatcgaacgataaaggcaaaattttcttcctagaaaatccagaaagcgaagcttcgcgtgaattccttaagattgcccgtcgtattattgaaattgttgaaaagctaggcccaaagcctcctgcgtggggtccccaaatggaa

SEQ ID NO: 8 - DNA, 849 bp, Artificial Sequence
  Description of Artificial Sequence Synthetic polynucleotide
atgaagacaattcaggagcagcagatgaagatagttaggaatatgaggaggattaggtacaagattgctgttattagcacgaaaggaggtgtggggaaaagctttgttaccgctagcctcgcggcagccctcgctgcggaggggcgaagggttggagtttttgacgcagatattagcggtcctagcgttcataaaatgctcggcctccagacgggcatgggtatgccctcgcagctcgacggcactgtaaagcccgtggaagttcctccgggaattaaagtagctagcattgggctgttgctgcccatggatgaggtgcccctaatttggagaggggccattaagacgagtgccattagagagctgcttgcatacgtcgactggggagaactcgactatctcctcattgacctacctccgggaacaggtgatgaggtcctcacgattacccagattattcccaacattacgggcttcctggtagtcacgattcccagcgagattgctaagtctgtcgttaagaaggctgtcagctttgccaagaggattgaagcccctgtgattggaattgtcgagaacatgagctactttaggtgtagcgacggatccattcactatattttcggccgcggcgcggctgaggagattgcgtcacagtatggtattgaactcctcggcaaaattcccattgaccctgcgattagagagtcgaacgataaaggcaaaattttcttcctagagaatccagagagcgaagcttcgagagagttccttaagattgcccgcaggattattgagattgttgagaagctaggcccaaagcctcctgcgtggggtccccagatggag

SEQ ID NO: 9 - protein, 217 aa, Artificial Sequence
  Description of Artificial Sequence Synthetic polypeptide
MAAMPKPAAFWNDRFANEEYVYGEAPNRFVASAARTWLPEAGEVLLLGAGEGRNAVHLAREGHTVTAVDYAVEGLRKTERLATEAGVEVEAIQADVREWKPARAWDAVVVTFLHLPADERPGLYRLVQRCLRPGGRLVAEWFRPEQRTDGYTSGGPPDPAMMVTADELRGHFAEAGIDHLEAAEPTLDEGMHRGPAATVRLVWCRPSTSLEHHHHHH

SEQ ID NO: 10 - protein, 163 aa, Artificial Sequence
  Description of Artificial Sequence Synthetic polypeptide
MARSIDYGNLMHRAMRGLIQSVLEDVAEHGLPGAHHFFITFDTTHPDVAMADWLRARYPQEMTVVIQHWYENLSADDHGFSVTLNFGNQPEPLVIPFDAVRTFVDPSVEFGLRFETHEEDEEEETGGDEDPDGDDEPPRHDAQVVSLDKFRKAAALEHHHHHH

SEQ ID NO: 11 - protein, 266 aa, Artificial Sequence
  Description of Artificial Sequence Synthetic polypeptide
MSTRTRTTEERRHEIVRVARATGSVDVTALAAELGVAKETVRRDLRALEDHGLVRRTHGGAYPVESAGFETTLAFRATSHVPEKRRIASAAVELLGDAETVFVDEGFTPQLIAEALPRDRPLTVVTASLPVAGALAEAGDTSVLLLGGRVRSGTLATVDHWTTKMLAGFVIDLAYIGANGISREHGLTTPDPAVSEVKAQAVRAARRTVFAGAHTKFGAVSFCRFAEVGALEAIVTSTLLPSAEAHRYSLLGPQIIRVLEHHHHHH

SEQ ID NO: 12 - protein, 291 aa, Artificial Sequence
  Description of Artificial Sequence Synthetic polypeptide
MKTIQEQQMKIVRNMRRIRYKIAVISTKGGVGKSFVTASLAAALAAEGRRVGVFDADISGPSVHKMLGLQTGMGMPSQLDGTVKPVEVPPGIKVASIGLLLPMDEVPLIWRGAIKTSAIRELLAYVDWGELDYLLIDLPPGTGDEVLTITQIIPNITGFLVVTIPSEIAKSVVKKAVSFAKRIEAPVIGIVENMSYFRCSDGSIHYIFGRGAAEEIASQYGIELLGKIPIDPAIRESNDKGKIFFLENPESEASREFLKIARRIIEIVEKLGPKPPAWGPQMELEHHHHHH

SEQ ID NO: 13 - DNA, 459 bp, Artificial Sequence
  Description of Artificial Sequence Synthetic polynucleotide
atggcgcgttcgatcgattacggcaacctcatgcaccgcgcgatgcggggcctgatccagagcgtgctcgaggatgtggccgagcacgggctgcccggcgcgcaccatttcttcatcaccttcgacacgacccatcccgatgtggccatggccgactggctccgcgcgcgctatccgcaggagatgacggtcgtgatccagcactggtacgagaacctctccgccgacgaccacggcttctcggtcacgctgaacttcggcaaccagcccgagccgctggtcatccccttcgatgccgtgcgcaccttcgtcgacccgtccgtggaattcggcctccggttcgagacccacgaggaggacgaggaggaggagacgggcggcgacgaggatcccgacggcgacgacgagccgccgcgccacgacgcgcaggtcgtgagcctcgacaagttccgcaagtag

SEQ ID NO: 14 - DNA, 459 bp, Artificial Sequence
  Description of Artificial Sequence Synthetic polynucleotide
atggcgcgttcgatcgattacggcaacctcatgcaccgcgcgatgcgtggcctgattcaaagcgtgctcgaagatgtggccgaacatgggctgcccggcgcgcatcatttcttcattaccttcgatacgacccatcccgatgtggccatggccgattggctccgtgcgcgttatccgcaagaaatgacggtcgtgattcaacattggtacgaaaacctctccgccgatgatcatggcttctcggtcacgctgaacttcggcaaccaacccgaaccgctggtcattcccttcgatgccgtgcgtaccttcgtcgatccgtccgtggaattcggcctccgtttcgaaacccatgaagaagatgaagaagaagaaacgggcggcgatgaagatcccgatggcgatgatgaaccgccgcgtcatgatgcgcaagtcgtgagcctcgataagttccgtaagtag

SEQ ID NO: 15 - DNA, 459 bp, Artificial Sequence
  Description of Artificial Sequence Synthetic polynucleotide
atggcgcgttcgatcgattacggcaacctcatgcaccgcgcgatgcggggcctgatccagagcgtgctcgaggatgtggccgagcatgggctgcccggcgcgcatcatttcttcatcaccttcgacacgacccatcccgatgtggccatggccgactggctccgcgcgcgctatccgcaggagatgacggtcgtgatccagcattggtacgagaacctctccgccgacgaccatggcttctcggtcacgctgaacttcggcaaccagcccgagccgctggtcatccccttcgatgccgtgcgcaccttcgtcgacccgtccgtggaattcggcctccggttcgagacccatgaggaggacgaggaggaggagacgggcggcgacgaggatcccgacggcgacgacgagccgccgcgccatgacgcgcaggtcgtgagcctcgacaagttccgcaagtag

SEQ ID NO: 16 - DNA, 777 bp, Artificial Sequence
  Description of Artificial Sequence Synthetic polynucleotide
atgagcacgcggacgaggacgacggaagaacgccggcacgagatcgtgcgggtcgcccgcgccaccggctcggtcgacgtcaccgcgctcgccgccgaactgggcgtcgccaaggagaccgtacgacgcgacctgcgcgccctggaggaccacggcctggtccgccgcacccacggcggcgcctacccggtggagagcgccggtttcgagaccacgctcgccttccgcgccaccagccacgtgcccgagaagcgccggatcgcgtccgccgccgtcgaactgctcggcgacgcggagacggtcttcgtcgacgagggcttcaccccccagctcatcgccgaggccctgccccgggaccggccgctgaccgtggtcaccgcgtccctgccggtggcgggcgcgctggccgaggcgggcgacacgtccgtcctgctgctcggcggccgggtccgctcgggcaccctggccaccgtcgaccactggaccacgaagatgctggccggcttcgtcatcgacctggcgtacatcggcgccaacggcatctcccgggagcacggtctcaccacacccgaccccgcggtcagcgaggtcaaggcgcaggccgtccgggccgcccgccgcacggtgttcgccggcgcgcacaccaagttcggggcggtgagcttctgccggttcgcggaggtcggcgccctggaggccatcgtcaccagcacgctgctgccctcggccgaggcccaccgctactccctcctcggcccccagatcatccgcgtctga

SEQ ID NO: 17 - DNA, 777 bp, Artificial Sequence
  Description of Artificial Sequence Synthetic polynucleotide
atgagcacgcggacgaggacgacggaagaacgccggcacgagattgtgcgtgtcgcccgtgccaccggctcggtcgatgtcaccgcgctcgccgccgaactgggcgtcgccaaggaaaccgtacgtcgtgatctgcgtgccctggaagatcatggcctggtccgtcgtacccatggcggcgcctacccggtggaaagcgccggtttcgaaaccacgctcgccttccgtgccaccagccatgtgcccgaaaagcgtcgtattgcgtccgccgccgtcgaactgctcggcgatgcggaaacggtcttcgtcgatgaaggcttcaccccccaactcattgccgaagccctgccccgtgatcgtccgctgaccgtggtcaccgcgtccctgccggtggcgggcgcgctggccgaagcgggcgatacgtccgtcctgctgctcggcggccgtgtccgttcgggcaccctggccaccgtcgatcattggaccacgaagatgctggccggcttcgtcattgatctggcgtacattggcgccaacggcatttcccgtgaacatggtctcaccacacccgatcccgcggtcagcgaagtcaaggcgcaagccgtccgtgccgcccgtcgtacggtgttcgccggcgcgcataccaagttcggggcggtgagcttctgccgtttcgcggaagtcggcgccctggaagccattgtcaccagcacgctgctgccctcggccgaagcccatcgttactccctcctcggcccccaaattattcgtgtctga

SEQ ID NO: 18 - DNA, 777 bp, Artificial Sequence
  Description of Artificial Sequence Synthetic polynucleotide
atgagcacgcggacgaggacgacggaagaacgccggcacgagatcgtgcgggtcgcccgcgccaccggctcggtcgacgtcaccgcgctcgccgccgaactgggcgtcgccaaggagaccgtacgacgcgacctgcgcgccctggaggaccatggcctggtccgccgcacccatggcggcgcctacccggtggagagcgccggtttcgagaccacgctcgccttccgcgccaccagccatgtgcccgagaagcgccggatcgcgtccgccgccgtcgaactgctcggcgacgcggagacggtcttcgtcgacgagggcttcaccccccagctcatcgccgaggccctgccccgggaccggccgctgaccgtggtcaccgcgtccctgccggtggcgggcgcgctggccgaggcgggcgacacgtccgtcctgctgctcggcggccgggtccgctcgggcaccctggccaccgtcgaccattggaccacgaagatgctggccggcttcgtcatcgacctggcgtacatcggcgccaacggcatctcccgggagcatggtctcaccacacccgaccccgcggtcagcgaggtcaaggcgcaggccgtccgggccgcccgccgcacggtgttcgccggcgcgcataccaagttcggggcggtgagcttctgccggttcgcggaggtcggcgccctggaggccatcgtcaccagcacgctgctgccctcggccgaggcccatcgctactccctcctcggcccccagatcatccgcgtctga

SEQ ID NO: 19 - DNA, 642 bp, Artificial Sequence
  Description of Artificial Sequence Synthetic polynucleotide
atggatgtaacacgacaaatagaattagcgcatcgatatatgaaagattttcataaaagtgattattctggtcatgatgttgcacatgtagaacgtgtaacgtcactagctcaaacaatctctaaatgcgagcaacaaggagaatatttaattatcacattatctgcattacttcatgatgtcattgatgataagttaacaaataaagccaatgctttagatcgtttaaaaacatttttaaagaacattcgcgtatcttctgatcaacaacaaaagattatttacatcattcaacatttaagttatagaaatggacaaaataatcatgtagaccttccaattgaaggacaaattgttagagatgcagatcgactagatgcgattggtgctattggtattgctagagcatttcaattttcaggccattttaatgagccaatgtggacagaatcaccacatagtgacatacctaatattgaaacgattactaatttagaaccttccgctatacgtcacttttatgataaattattaaaattaaaagatttaatgcatactgaaactggtcgaaaattagctagagaaagacatgcgtttatggaacagtttttaaatcaattttataaagaatggcatatataa

SEQ ID NO: 20 - DNA, 642 bp, Artificial Sequence
  Description of Artificial Sequence Synthetic polynucleotide
atggatgtaacacgacaaatagaattagcgcatcgatatatgaaagactttcacaaaagtgactattctggtcacgacgttgcacacgtagagcgcgtaacgtcactagctcagacaatctctaaatgcgagcagcagggagagtatttaatcatcacattatctgcattacttcacgacgtcatcgacgacaagttaacaaataaagccaatgctttagaccgcttaaaaacatttttaaagaacatccgcgtatcttctgaccagcagcagaagatcatctacatcatccagcacttaagttatagaaatggacagaataatcacgtagaccttccaatcgagggacagatcgttagagacgcagaccgactagacgcgatcggtgctatcggtatcgctagagcatttcagttttcaggccactttaatgagccaatgtggacagagtcaccacacagtgacatacctaatatcgagacgatcactaatttagagccttccgctatacgccacttttatgacaaattattaaaattaaaagacttaatgcacactgagactggtcgaaaattagctagagagagacacgcgtttatggagcagtttttaaatcagttttataaagagtggcacatataa

SEQ ID NO: 21 - DNA, 642 bp, Artificial Sequence
  Description of Artificial Sequence Synthetic polynucleotide
atggatgtaacacgacaaatagaattagcgcatcgatatatgaaagattttcacaaaagtgattattctggtcacgatgttgcacacgtagaacgtgtaacgtcactagctcaaacaatctctaaatgcgagcaacaaggagaatatttaattatcacattatctgcattacttcacgatgtcattgatgataagttaacaaataaagccaatgctttagatcgtttaaaaacatttttaaagaacattcgcgtatcttctgatcaacaacaaaagattatttacatcattcaacacttaagttatagaaatggacaaaataatcacgtagaccttccaattgaaggacaaattgttagagatgcagatcgactagatgcgattggtgctattggtattgctagagcatttcaattttcaggccactttaatgagccaatgtggacagaatcaccacacagtgacatacctaatattgaaacgattactaatttagaaccttccgctatacgtcacttttatgataaattattaaaattaaaagatttaatgcacactgaaactggtcgaaaattagctagagaaagacacgcgtttatggaacagtttttaaatcaattttataaagaatggcacatataa

SEQ ID NO: 22 - DNA, 657 bp, Artificial Sequence
  Description of Artificial Sequence Synthetic polynucleotide
atgaaactcattcaaatgtcagaccatatttataaattaaatatacagacaacagttggtatcccgatacaaataaacacttggtttattgtgaatgataacgacgtttatatcatagacacaggtatggatgattatgctgagctacaaatcacgattgctaaatcgctcggtaatcctaaaggcatttttttaacgcatggacatctagatcatatcaatggcgcaaaacgtatttctgaagctttgaaaatacctatctttacatataaaaatgaactcccttatatcaatggtgagctgccttatccaaataaaacgcataccgaaaatacaggtgttcaatacattgttaaacctctagaaactaatacaaatctgcccttcaattattacttaactcctggtcatgcaccaggtcatgtcatctattttcataatcaagataaaattttaatatgcggagatttatttatttcagatgcgcaacatctgcatattcctatcaaaaaattcacttataacatgactgaaaatatcaaaagcggtcaaatcatagataatctttgtcccaaattaattacaacttcacatggcgatgatctatattattcagatgacatttattcaatttataaatttaagtacgaggagtaa

SEQ ID NO: 23 - DNA, 657 bp, Artificial Sequence
  Description of Artificial Sequence Synthetic polynucleotide
atgaaactcattcaaatgtcagaccatatttataaattaaatatacagacaacagttggtatcccgatacagataaacacttggtttatcgtgaatgacaacgacgtttatatcatagacacaggtatggacgactatgctgagctacagatcacgatcgctaaatcgctcggtaatcctaaaggcatctttttaacgcacggacacctagaccacatcaatggcgcaaaacgcatctctgaggctttgaaaatacctatctttacatataaaaatgagctcccttatatcaatggtgagctgccttatccaaataaaacgcacaccgagaatacaggtgttcagtacatcgttaaacctctagagactaatacaaatctgcccttcaattattacttaactcctggtcacgcaccaggtcacgtcatctattttcacaatcaggacaaaatcttaatatgcggagacttatttatctcagacgcgcagcacctgcacatccctatcaaaaaattcacttataacatgactgagaatatcaaaagcggtcagatcatagacaatctttgtcccaaattaatcacaacttcacacggcgacgacctatattattcagacgacatctattcaatctataaatttaagtacgaggagtaa

SEQ ID NO: 24 - DNA, 657 bp, Artificial Sequence
  Description of Artificial Sequence Synthetic polynucleotide
atgaaactcattcaaatgtcagaccatatttataaattaaatatacagacaacagttggtatcccgatacaaataaacacttggtttattgtgaatgataacgacgtttatatcatagacacaggtatggatgattatgctgagctacaaatcacgattgctaaatcgctcggtaatcctaaaggcatttttttaacgcacggacacctagatcacatcaatggcgcaaaacgtatttctgaagctttgaaaatacctatctttacatataaaaatgaactcccttatatcaatggtgagctgccttatccaaataaaacgcacaccgaaaatacaggtgttcaatacattgttaaacctctagaaactaatacaaatctgcccttcaattattacttaactcctggtcacgcaccaggtcacgtcatctattttcacaatcaagataaaattttaatatgcggagatttatttatttcagatgcgcaacacctgcacattcctatcaaaaaattcacttataacatgactgaaaatatcaaaagcggtcaaatcatagataatctttgtcccaaattaattacaacttcacacggcgatgatctatattattcagatgacatttattcaatttataaatttaagtacgaggagtaa

SEQ ID NO: 25 - DNA, 801 bp, Artificial Sequence
  Description of Artificial Sequence Synthetic polynucleotide
gtgaggcggagggctagatggctgaggagggagagggaggaggaagagagggttaaggaccgggacatgtttaagattgtggacgaggttttcgactccataaccctctcccacctctacaggctctactcgcgcaaggtcctcagggagctcaagggctctataagcagcggtaaggagtctaaggtctactggggcgtcgcgtgggataggagcgacgtcgccgttaagatatacctctcgttcacttccgacttcaggaagagcattagaaaatatattgtcggggaccccaggttcgaggacatccccgcaggcaacataaggaggctgatatacgagtgggctaggaaagagtacaggaacctcaggaggatgcgcgagtcgggggtcagggttcccaggcccgtggccgtcgaggcaaacattatagttatggagttcctgggcgagaaggggtacagggcccctaccctggctgaggctgtcgaggagcttgataggggggaggcggaggctatagcggccgaggtcctccgccaggcggaggctatagtatgtagggccaggctcgtgcacgccgacctcagcgagtacaacatactagtctggaggggggagccctggataatagacgtctcccaggcggtgccccacagccacccgaacgctgaggagtttctagagagggacgtggagaacctccacaggttcttgacaggtaagatggggttcgagttcgactttgacgcttatctctctaggctaaaaagctgtatccaccggggtgctaggggttga

SEQ ID NO: 26 - DNA, 801 bp, Artificial Sequence
  Description of Artificial Sequence Synthetic polynucleotide
gtgaggcggagggctagatggctgaggagggagagggaggaggaagaacgtgttaaggatcgtgatatgtttaagattgtggatgaagttttcgattccattaccctctcccatctctaccgtctctactcgcgtaaggtcctccgtgaactcaagggctctattagcagcggtaaggaatctaaggtctactggggcgtcgcgtgggatcgtagcgatgtcgccgttaagatttacctctcgttcacttccgatttccgtaagagcattcgtaaatatattgtcggggatccccgtttcgaagatattcccgcaggcaacattcgtcgtctgatttacgaatgggctcgtaaagaataccgtaacctccgtcgtatgcgtgaatcgggggtccgtgttccccgtcccgtggccgtcgaagcaaacattattgttatggaattcctgggcgaaaaggggtaccgtgcccctaccctggctgaagctgtcgaagaacttgatcgtggggaagcggaagctattgcggccgaagtcctccgtcaagcggaagctattgtatgtcgtgcccgtctcgtgcatgccgatctcagcgaatacaacattctagtctggcgtggggaaccctggattattgatgtctcccaagcggtgccccatagccatccgaacgctgaagaatttctagaacgtgatgtggaaaacctccatcgtttcttgacaggtaagatggggttcgaattcgattttgatgcttatctctctcgtctaaaaagctgtattcatcgtggtgctcgtggttga

SEQ ID NO: 27 - DNA, 801 bp, Artificial Sequence
  Description of Artificial Sequence Synthetic polynucleotide
gtgaggcggagggctagatggctgaggagggagagggaggaggaagaaagggttaaggaccgggacatgtttaagattgtggacgaagttttcgactccataaccctctcccacctctacaggctctactcgcgcaaggtcctcagggaactcaagggctctataagcagcggtaaggaatctaaggtctactggggcgtcgcgtgggataggagcgacgtcgccgttaagatatacctctcgttcacttccgacttcaggaagagcattagaaaatatattgtcggggaccccaggttcgaagacatccccgcaggcaacataaggaggctgatatacgaatgggctaggaaagaatacaggaacctcaggaggatgcgcgaatcgggggtcagggttcccaggcccgtggccgtcgaagcaaacattatagttatggaattcctgggcgaaaaggggtacagggcccctaccctggctgaagctgtcgaagaacttgataggggggaagcggaagctatagcggccgaagtcctccgccaggcggaagctatagtatgtagggccaggctcgtgcacgccgacctcagcgaatacaacatactagtctggaggggggaaccctggataatagacgtctcccaggcggtgccccacagccacccgaacgctgaagaatttctagaaagggacgtggaaaacctccacaggttcttgacaggtaagatggggttcgaattcgactttgacgcttatctctctaggctaaaaagctgtatccaccggggtgctaggggttga

SEQ ID NO: 28 - DNA, 630 bp, Artificial Sequence
  Description of Artificial Sequence Synthetic polynucleotide
atggccgccatgcccaagcccgctgcgttctggaacgaccgctttgccaacgaggagtacgtgtacggcgaggcccccaaccgcttcgtcgcgagcgccgcccggacgtggctgccggaggccggtgaggttctcctgctcggggcgggcgaggggcgcaacgccgtgcacctggcccgggagggccatacggtcaccgcggtcgactacgccgtggaggggctccgcaagacggaacgcctcgcgacggaggccggggtggaggtcgaggcgatccaggccgatgtgcgcgagtggaagcccgcccgggcgtgggacgcggtcgtcgtcacgtttctccaccttcccgccgacgagcgaccgggcctgtaccgcctcgttcagcgctgtttgcggcccggggggcgcctcgtggcggagtggtttcgcccggagcagcgcacggacggctacacgagcggcggcccgcccgatcctgccatgatggtcaccgccgacgagctccgcgggcacttcgccgaggcgggcatcgaccatctcgaagcggccgagccgaccctcgacgagggcatgcaccggggccccgcggcgacggttcgtctcgtgtggtgccggccgtccacctcgtag

SEQ ID NO: 29 - DNA, 630 bp, Artificial Sequence
  Description of Artificial Sequence Synthetic polynucleotide
atggccgccatgcccaagcccgctgcgttctggaacgaccgctttgccaacgaagaatacgtgtacggcgaagcccccaaccgtttcgtcgcgagcgccgcccgtacgtggctgccggaagccggtgaagttctcctgctcggggcgggcgaagggcgtaacgccgtgcatctggcccgtgaaggccatacggtcaccgcggtcgattacgccgtggaagggctccgtaagacggaacgtctcgcgacggaagccggggtggaagtcgaagcgattcaagccgatgtgcgtgaatggaagcccgcccgtgcgtgggatgcggtcgtcgtcacgtttctccatcttcccgccgatgaacgtccgggcctgtaccgtctcgttcaacgttgtttgcgtcccggggggcgtctcgtggcggaatggtttcgtccggaacaacgtacggatggctacacgagcggcggcccgcccgatcctgccatgatggtcaccgccgatgaactccgtgggcatttcgccgaagcgggcattgatcatctcgaagcggccgaaccgaccctcgatgaaggcatgcatcgtggccccgcggcgacggttcgtctcgtgtggtgccgtccgtccacctcgtag

SEQ ID NO: 30 - DNA, 630 bp, Artificial Sequence
  Description of Artificial Sequence Synthetic polynucleotide
atggccgccatgcccaagcccgctgcgttctggaacgaccgctttgccaacgaagaatacgtgtacggcgaagcccccaaccgcttcgtcgcgagcgccgcccggacgtggctgccggaagccggtgaagttctcctgctcggggcgggcgaagggcgcaacgccgtgcacctggcccgggaaggccatacggtcaccgcggtcgactacgccgtggaagggctccgcaagacggaacgcctcgcgacggaagccggggtggaagtcgaagcgatccaggccgatgtgcgcgaatggaagcccgcccgggcgtgggacgcggtcgtcgtcacgtttctccaccttcccgccgacgaacgaccgggcctgtaccgcctcgttcagcgctgtttgcggcccggggggcgcctcgtggcggaatggtttcgcccggaacagcgcacggacggctacacgagcggcggcccgcccgatcctgccatgatggtcaccgccgacgaactccgcgggcacttcgccgaagcgggcatcgaccatctcgaagcggccgaaccgaccctcgacgaaggcatgcaccggggccccgcggcgacggttcgtctcgtgtggtgccggccgtccacctcgtag

SEQ ID NO: 31 - DNA, 951 bp, Artificial Sequence
  Description of Artificial Sequence Synthetic polynucleotide
atgaaataccaagtattactttattacaaatatacaacaattgaagatccagaagcttttgcgaaagagcatctagctttttgcaaatcattaaacttaaaaggccgtattttagtagcgacagaagggattaacggaacgttatctggtactgtcgaagaaacagaaaagtatatggaagcaatgcaagcagatgagcgctttaaggatacattctttaaaattgatccagcagaagaaatggccttccgcaaaatgtttgttcgcccacgttctgaattagtggcgttgaacttagaagaagacgttgatccattagaaacgacggggaaatatttggaacctgcagaatttaaagaagccttattagacgaagacactgttgtaatcgatgctcgtaacgattatgaatatgatttaggtcatttccgtggtgccgtgcgcccagatatccgtagcttccgtgaattaccacaatggattcgcgagaacaaagaaaaatttatggataaaaaaattgttacctattgtactggcgggattcgctgtgaaaaattttctggctggttattaaaagaaggatttgaagatgttgctcaattgcatggtggtatcgccaactatggaaaaaatccagaaacacgtggcgaactttgggacggcaaaatgtatgtctttgatgaccgaatcagtgtcgaaattaatcatgttgataaaaaagttattgggaaagactggtttgatgggacaccttgcgaacgctacattaactgtgcaaacccagaatgtaatcgtcaaatcttaacttcagaagaaaatgaacataaacatttaggtggctgctcattagaatgtagccagcatcctgccaaccgttatgtaaaaaaacataatttaacagaagcagaagttgctgaacgtttagctttgttagaagcggttgaagtataa

SEQ ID NO: 32 - DNA, 951 bp, Artificial Sequence
  Description of Artificial Sequence Synthetic polynucleotide
atgaaataccaagtattactttattacaaatatacaacaattgaggacccagaggcttttgcgaaagagcacctagctttttgcaaatcattaaacttaaaaggccgcatcttagtagcgacagaggggatcaacggaacgttatctggtactgtcgaggagacagagaagtatatggaggcaatgcaggcagacgagcgctttaaggacacattctttaaaatcgacccagcagaggagatggccttccgcaaaatgtttgttcgcccacgctctgagttagtggcgttgaacttagaggaggacgttgacccattagagacgacggggaaatatttggagcctgcagagtttaaagaggccttattagacgaggacactgttgtaatcgacgctcgcaacgactatgagtatgacttaggtcacttccgcggtgccgtgcgcccagacatccgcagcttccgcgagttaccacagtggatccgcgagaacaaagagaaatttatggacaaaaaaatcgttacctattgtactggcgggatccgctgtgagaaattttctggctggttattaaaagagggatttgaggacgttgctcagttgcacggtggtatcgccaactatggaaaaaatccagagacacgcggcgagctttgggacggcaaaatgtatgtctttgacgaccgaatcagtgtcgagatcaatcacgttgacaaaaaagttatcgggaaagactggtttgacgggacaccttgcgagcgctacatcaactgtgcaaacccagagtgtaatcgccagatcttaacttcagaggagaatgagcacaaacacttaggtggctgctcattagagtgtagccagcaccctgccaaccgctatgtaaaaaaacacaatttaacagaggcagaggttgctgagcgcttagctttgttagaggcggttgaggtataa

SEQ ID NO: 33 - DNA, 951 bp, Artificial Sequence
  Description of Artificial Sequence Synthetic polynucleotide
atgaaataccaagtattactttattacaaatatacaacaattgaggatccagaggcttttgcgaaagagcatctagctttttgcaaatcattaaacttaaaaggccgtattttagtagcgacagaggggattaacggaacgttatctggtactgtcgaggagacagagaagtatatggaggcaatgcaagcagatgagcgctttaaggatacattctttaaaattgatccagcagaggagatggccttccgcaaaatgtttgttcgcccacgttctgagttagtggcgttgaacttagaggaggacgttgatccattagagacgacggggaaatatttggagcctgcagagtttaaagaggccttattagacgaggacactgttgtaatcgatgctcgtaacgattatgagtatgatttaggtcatttccgtggtgccgtgcgcccagatatccgtagcttccgtgagttaccacaatggattcgcgagaacaaagagaaatttatggataaaaaaattgttacctattgtactggcgggattcgctgtgagaaattttctggctggttattaaaagagggatttgaggatgttgctcaattgcatggtggtatcgccaactatggaaaaaatccagagacacgtggcgagctttgggacggcaaaatgtatgtctttgatgaccgaatcagtgtcgagattaatcatgttgataaaaaagttattgggaaagactggtttgatgggacaccttgcgagcgctacattaactgtgcaaacccagagtgtaatcgtcaaatcttaacttcagaggagaatgagcataaacatttaggtggctgctcattagagtgtagccagcatcctgccaaccgttatgtaaaaaaacataatttaacagaggcagaggttgctgagcgtttagctttgttagaggcggttgaggtataa

SEQ ID NO: 34 - DNA, 555 bp, Artificial Sequence
  Description of Artificial Sequence Synthetic polynucleotide
atgatatacagatttactatcatatctgatgaagttgacgattttgtcagagaaatacaaattgatccggaagctacatttcttgacttccatgaagcaatactgaaatcagtagggtacacaaacgaccagatgacctccttctttatctgcgatgatgattgggaaaaagaaaaagaagtcactttggaagaaatggacgacaatccggaaatggatagttggataatgaaagagactactatcagcgaactggtagaagatgaaaagcaaaaattgttgtatgtattcgactacatgacagagcgttgcttcttcatcgaattgtctgaaatcatcaccggaaaagatatgaatggtgccaaatgtaccaagaaatcgggtgatgctccgccacaaactgtagattttgaagaaatggctgctgcaagcggttcactcgacctggacgaaaatttctatggtgatcaggactttgatatggaagattttgatcaggaaggcttcgacataggtggtaacgcgggtggctcttatgaagaagagaagttttaa

SEQ ID NO: 35 - DNA, 555 bp, Artificial Sequence
  Description of Artificial Sequence Synthetic polynucleotide
atgatatacagatttactatcatatctgatgaagttgacgattttgtcagagagatacagatcgacccggaggctacatttcttgacttccacgaggcaatactgaaatcagtagggtacacaaacgaccagatgacctccttctttatctgcgacgacgactgggagaaagagaaagaggtcactttggaggagatggacgacaatccggagatggacagttggataatgaaagagactactatcagcgagctggtagaggacgagaagcagaaattgttgtatgtattcgactacatgacagagcgctgcttcttcatcgagttgtctgagatcatcaccggaaaagacatgaatggtgccaaatgtaccaagaaatcgggtgacgctccgccacagactgtagactttgaggagatggctgctgcaagcggttcactcgacctggacgagaatttctatggtgaccaggactttgacatggaggactttgaccaggagggcttcgacataggtggtaacgcgggtggctcttatgaggaggagaagttttaa

SEQ ID NO: 36 - DNA, 555 bp, Artificial Sequence
  Description of Artificial Sequence Synthetic polynucleotide
atgatatacagatttactatcatatctgatgaagttgacgattttgtcagagagatacaaattgatccggaggctacatttcttgacttccatgaggcaatactgaaatcagtagggtacacaaacgaccagatgacctccttctttatctgcgatgatgattgggagaaagagaaagaggtcactttggaggagatggacgacaatccggagatggatagttggataatgaaagagactactatcagcgagctggtagaggatgagaagcaaaaattgttgtatgtattcgactacatgacagagcgttgcttcttcatcgagttgtctgagatcatcaccggaaaagatatgaatggtgccaaatgtaccaagaaatcgggtgatgctccgccacaaactgtagattttgaggagatggctgctgcaagcggttcactcgacctggacgagaatttctatggtgatcaggactttgatatggaggattttgatcaggagggcttcgacataggtggtaacgcgggtggctcttatgaggaggagaagttttaa

SEQ ID NO: 37 - DNA, 852 bp, Artificial Sequence
  Description of Artificial Sequence Synthetic polynucleotide
atgaagacaattcaggagcagcagatgaagatagttaggaatatgaggaggattaggtacaagatagctgttataagcacgaaaggaggtgtggggaaaagctttgttaccgctagcctcgcggcagccctcgctgcggaggggcgaagggttggagtttttgacgcagatataagcggtcctagcgttcataaaatgctcggcctccagacgggcatgggtatgccctcgcagctcgacggcactgtaaagcccgtggaagttcctccgggaattaaagtagctagcatagggctgttgctgcccatggatgaggtgcccctaatctggagaggggccataaagacgagtgccatcagagagctgcttgcatacgtcgactggggagaactcgactatctcctcatagacctacctccgggaacaggtgatgaggtcctcacgataacccagataatacccaacataacgggcttcctggtagtcacgatacccagcgagatagctaagtctgtcgttaagaaggctgtcagctttgccaagaggatagaagcccctgtgataggaatagtcgagaacatgagctactttaggtgtagcgacggatccatacactatatcttcggccgcggcgcggctgaggagatcgcgtcacagtatggtatagaactcctcggcaaaatacccatagaccctgcgataagagagtcgaacgataaaggcaaaatattcttcctagagaatccagagagcgaagcttcgagagagttccttaagatagcccgcaggataatagagatagttgagaagctaggcccaaagcctcctgcgtggggtccccagatggagtag

SEQ ID NO: 38 - DNA, 852 bp, Artificial Sequence
  Description of Artificial Sequence Synthetic polynucleotide
atgaagacaattcaggagcagcagatgaagatagttaggaatatgcgtcgtattcgttacaagattgctgttattagcacgaaaggaggtgtggggaaaagctttgttaccgctagcctcgcggcagccctcgctgcggaagggcgtcgtgttggagtttttgatgcagatattagcggtcctagcgttcataaaatgctcggcctccaaacgggcatgggtatgccctcgcaactcgatggcactgtaaagcccgtggaagttcctccgggaattaaagtagctagcattgggctgttgctgcccatggatgaagtgcccctaatttggcgtggggccattaagacgagtgccattcgtgaactgcttgcatacgtcgattggggagaactcgattatctcctcattgatctacctccgggaacaggtgatgaagtcctcacgattacccaaattattcccaacattacgggcttcctggtagtcacgattcccagcgaaattgctaagtctgtcgttaagaaggctgtcagctttgccaagcgtattgaagcccctgtgattggaattgtcgaaaacatgagctactttcgttgtagcgatggatccattcattatattttcggccgtggcgcggctgaagaaattgcgtcacaatatggtattgaactcctcggcaaaattcccattgatcctgcgattcgtgaatcgaacgataaaggcaaaattttcttcctagaaaatccagaaagcgaagcttcgcgtgaattccttaagattgcccgtcgtattattgaaattgttgaaaagctaggcccaaagcctcctgcgtggggtccccaaatggaatag

SEQ ID NO: 39 - DNA, 852 bp, Artificial Sequence
  Description of Artificial Sequence Synthetic polynucleotide
atgaagacaattcaggagcagcagatgaagatagttaggaatatgaggaggattaggtacaagattgctgttattagcacgaaaggaggtgtggggaaaagctttgttaccgctagcctcgcggcagccctcgctgcggaggggcgaagggttggagtttttgacgcagatattagcggtcctagcgttcataaaatgctcggcctccagacgggcatgggtatgccctcgcagctcgacggcactgtaaagcccgtggaagttcctccgggaattaaagtagctagcattgggctgttgctgcccatggatgaggtgcccctaatttggagaggggccattaagacgagtgccattagagagctgcttgcatacgtcgactggggagaactcgactatctcctcattgacctacctccgggaacaggtgatgaggtcctcacgattacccagattattcccaacattacgggcttcctggtagtcacgattcccagcgagattgctaagtctgtcgttaagaaggctgtcagctttgccaagaggattgaagcccctgtgattggaattgtcgagaacatgagctactttaggtgtagcgacggatccattcactatattttcggccgcggcgcggctgaggagattgcgtcacagtatggtattgaactcctcggcaaaattcccattgaccctgcgattagagagtcgaacgataaaggcaaaattttcttcctagagaatccagagagcgaagcttcgagagagttccttaagattgcccgcaggattattgagattgttgagaagctaggcccaaagcctcctgcgtggggtccccagatggagtag

SEQ ID NO: 40 - DNA, 567 bp, Artificial Sequence
  Description of Artificial Sequence Synthetic polynucleotide
atgggtagtatagaggaggtgcttttggaggagaggctcataggatatctagaccccggagccgagaaagttttagcgaggataaacaggccttcaaaaatagtgtctacaagcagttgtacagggaggataacactgatcgagggcgaggctcactggctcaggaacggggcaagagtagcgtacaagacccatcaccccatatcccggagtgaggttgaaagggttctaaggaggggcttcacaaacctttggctcaaggtgaccggccctatactacatctcagggttgaggggtggcagtgtgcaaagtcccttctcgaggcagctaggagaaacgggttcaagcacagcggagtcataagcatagctgaggattcaagactcgtcatagaaataatgagcagccagagcatgtcagtacctctagttatggagggtgctaggatagtcggcgacgatgccctagatatgctgattgagaaagcaaacactatactagttgagtctagaatcgggctagacacgttttcaagagaggtcgaagagcttgtcgaatgcttttaa

SEQ ID NO: 41 - DNA, 567 bp, Artificial Sequence
  Description of Artificial Sequence Synthetic polynucleotide
atgggtagtatagaggaggtgcttttggaggagaggctcataggatatctagatcccggagccgaaaaagttttagcgcgtattaaccgtccttcaaaaattgtgtctacaagcagttgtacagggcgtattacactgattgaaggcgaagctcattggctccgtaacggggcacgtgtagcgtacaagacccatcatcccatttcccgtagtgaagttgaacgtgttctacgtcgtggcttcacaaacctttggctcaaggtgaccggccctattctacatctccgtgttgaagggtggcaatgtgcaaagtcccttctcgaagcagctcgtcgtaacgggttcaagcatagcggagtcattagcattgctgaagattcacgtctcgtcattgaaattatgagcagccaaagcatgtcagtacctctagttatggaaggtgctcgtattgtcggcgatgatgccctagatatgctgattgaaaaagcaaacactattctagttgaatctcgtattgggctagatacgttttcacgtgaagtcgaagaacttgtcgaatgcttttaa

SEQ ID NO: 42 - DNA, 567 bp, Artificial Sequence
  Description of Artificial Sequence Synthetic polynucleotide
atgggtagtatagaggaggtgcttttggaggagaggctcataggatatctagaccccggagccgagaaagttttagcgaggattaacaggccttcaaaaattgtgtctacaagcagttgtacagggaggattacactgattgagggcgaggctcactggctcaggaacggggcaagagtagcgtacaagacccatcaccccatttcccggagtgaggttgaaagggttctaaggaggggcttcacaaacctttggctcaaggtgaccggccctattctacatctcagggttgaggggtggcagtgtgcaaagtcccttctcgaggcagctaggagaaacgggttcaagcacagcggagtcattagcattgctgaggattcaagactcgtcattgaaattatgagcagccagagcatgtcagtacctctagttatggagggtgctaggattgtcggcgacgatgccctagatatgctgattgagaaagcaaacactattctagttgagtctagaattgggctagacacgttttcaagagaggtcgaagagcttgtcgaatgcttttaa

SEQ ID NO: 43 - DNA, 489 bp, Artificial Sequence
  Description of Artificial Sequence Synthetic polynucleotide
atgactattgaccaaatgactattgaccaaatgactaaaatttttcttgcagataaagagtcaacactcaacttaggtattctcttaggagaaactttaactgctggtagtgtgattttactagaaggtgatttaggtgctggtaaaactactttggtacagggcttgggtaaaggtttaagtattactgaacccattgtcagtcctacttttactctgattaatgagtacacagaaggacgtatacccctttaccatctggatttataccgcttagagccacaagaagtattaagtttaaatttagaaatttattgggaagggattgagataattccgggtattgtagcgattgagtggtcggaacgaatgccctacaagccaagtacctacattaacgtacttttgacttatggcgatgagggcagtcgtcaagccgaaattacaccattcaattgcaccatcagcgatttaattgctaccaagtga

SEQ ID NO: 44 - DNA, 489 bp, Artificial Sequence
  Description of Artificial Sequence Synthetic polynucleotide
atgactattgaccaaatgactattgaccaaatgactaaaatttttcttgcagacaaagagtcaacactcaacttaggtatcctcttaggagagactttaactgctggtagtgtgatcttactagagggtgacttaggtgctggtaaaactactttggtacagggcttgggtaaaggtttaagtatcactgagcccatcgtcagtcctacttttactctgatcaatgagtacacagagggacgcatacccctttaccacctggacttataccgcttagagccacaggaggtattaagtttaaatttagagatctattgggaggggatcgagataatcccgggtatcgtagcgatcgagtggtcggagcgaatgccctacaagccaagtacctacatcaacgtacttttgacttatggcgacgagggcagtcgccaggccgagatcacaccattcaattgcaccatcagcgacttaatcgctaccaagtga

SEQ ID NO: 45 - DNA, 489 bp, Artificial Sequence
  Description of Artificial Sequence Synthetic polynucleotide
atgactattgaccaaatgactattgaccaaatgactaaaatttttcttgcagataaagagtcaacactcaacttaggtatcctcttaggagaaactttaactgctggtagtgtgatcttactagaaggtgatttaggtgctggtaaaactactttggtacagggcttgggtaaaggtttaagtatcactgaacccatcgtcagtcctacttttactctgatcaatgagtacacagaaggacgtatacccctttaccatctggatttataccgcttagagccacaagaagtattaagtttaaatttagaaatctattgggaagggatcgagataatcccgggtatcgtagcgatcgagtggtcggaacgaatgccctacaagccaagtacctacatcaacgtacttttgacttatggcgatgagggcagtcgtcaagccgaaatcacaccattcaattgcaccatcagcgatttaatcgctaccaagtga

SEQ ID NO: 46 - DNA, 549 bp, Artificial Sequence
  Description of Artificial Sequence Synthetic polynucleotide
atgattaaatatagtatccgtggtgaaaacctagaagtaacagaagcaattcgtgattatgtagtttctaaactcgaaaagatcgaaaagtacttccaaccagaacaagagttggatgcccgaattaacttaaaagtttatcgtgaaaaaacggctaaagtggaagtaacgattccgcttggatctattactctccgcgcagaagatgtatctcaagatatgtatggttcaattgaccttgtaactgataaaattgaacgtcagattcgtaaaaataaaacaaaaatcgagcgtaaaaataaaaataaggtagcaactggtcaattatttacagatgctttggtggaagattcaaatattgtccagtctaaagttgttcgttcaaaacaaattgatttaaaaccaatggatttggaagaagcaattctacaaatggatttattggggcatgatttctttatctatgtggatgttgaagatcagacaaccaatgtgatttatcgtcgtgaggatggcgaaattggtttgttagaggttaaagaatcttaa

SEQ ID NO: 47 - DNA, 549 bp, Artificial Sequence
  Description of Artificial Sequence Synthetic polynucleotide
atgattaaatatagtatccgtggtgaaaacctagaagtaacagaggcaatccgcgactatgtagtttctaaactcgagaagatcgagaagtacttccagccagagcaggagttggacgcccgaatcaacttaaaagtttatcgcgagaaaacggctaaagtggaggtaacgatcccgcttggatctatcactctccgcgcagaggacgtatctcaggacatgtatggttcaatcgaccttgtaactgacaaaatcgagcgccagatccgcaaaaataaaacaaaaatcgagcgcaaaaataaaaataaggtagcaactggtcagttatttacagacgctttggtggaggactcaaatatcgtccagtctaaagttgttcgctcaaaacagatcgacttaaaaccaatggacttggaggaggcaatcctacagatggacttattggggcacgacttctttatctatgtggacgttgaggaccagacaaccaatgtgatctatcgccgcgaggacggcgagatcggtttgttagaggttaaagagtcttaa

SEQ ID NO: 48 - DNA, 549 bp, Artificial Sequence
  Description of Artificial Sequence Synthetic polynucleotide
atgattaaatatagtatccgtggtgaaaacctagaagtaacagaagcaatccgtgattatgtagtttctaaactcgaaaagatcgaaaagtacttccaaccagaacaagagttggatgcccgaatcaacttaaaagtttatcgtgaaaaaacggctaaagtggaagtaacgatcccgcttggatctatcactctccgcgcagaagatgtatctcaagatatgtatggttcaatcgaccttgtaactgataaaatcgaacgtcagatccgtaaaaataaaacaaaaatcgagcgtaaaaataaaaataaggtagcaactggtcaattatttacagatgctttggtggaagattcaaatatcgtccagtctaaagttgttcgttcaaaacaaatcgatttaaaaccaatggatttggaagaagcaatcctacaaatggatttattggggcatgatttctttatctatgtggatgttgaagatcagacaaccaatgtgatctatcgtcgtgaggatggcgaaatcggtttgttagaggttaaagaatcttaa

SEQ ID NO: 49 - DNA, 801 bp, Artificial Sequence; identical to SEQ ID NO: 25
  Description of Artificial Sequence Synthetic polynucleotide

SEQ ID NO: 50 - DNA, 801 bp, Artificial Sequence
  Description of Artificial Sequence Synthetic polynucleotide
gtgaggcggagggctagatggctgaggagggagagggaggaggaagagcgtgttaaggaccgtgacatgtttaagattgtggacgaggttttcgactccataaccctctcccacctctaccgtctctactcgcgtaaggtcctccgtgagctcaagggctctataagcagcggtaaggagtctaaggtctactggggcgtcgcgtgggatcgtagcgacgtcgccgttaagatatacctctcgttcacttccgacttccgtaagagcattcgtaaatatattgtcggggacccccgtttcgaggacatccccgcaggcaacatacgtcgtctgatatacgagtgggctcgtaaagagtaccgtaacctccgtcgtatgcgtgagtcgggggtccgtgttccccgtcccgtggccgtcgaggcaaacattatagttatggagttcctgggcgagaaggggtaccgtgcccctaccctggctgaggctgtcgaggagcttgatcgtggggaggcggaggctatagcggccgaggtcctccgtcaggcggaggctatagtatgtcgtgcccgtctcgtgcacgccgacctcagcgagtacaacatactagtctggcgtggggagccctggataatagacgtctcccaggcggtgccccacagccacccgaacgctgaggagtttctagagcgtgacgtggagaacctccaccgtttcttgacaggtaagatggggttcgagttcgactttgacgcttatctctctcgtctaaaaagctgtatccaccgtggtgctcgtggttga

SEQ ID NO: 51 - DNA, 513 bp, Artificial Sequence
  Description of Artificial Sequence Synthetic polynucleotide
gtgaacctggacgccccacgggtcctggtcctcaacgccgcctacgaggtcctgggcctggccagcatcaagcgggccgtgctcctcgtcctcgggggcggggcggagatggtctcggaaagcggcctctacctcaacaccccctccacccggatccccgtccccagcgtcgtccgcctcaagcgcatggtccgccgcaggccggggcgcgttcccttgaaccgcagaaacgtcctccggcgcgaccgctacacctgccagtactgcgggcaaaagggcggggagctcaccgtggaccacgtcctccccaaaagccgcgggggcaagagcacctgggacaacctggtggccgcctgccgcagctgcaacctcaggaagggggaccgcacccccgaggaggcggggatgcgcctcctccgccccccgaagcccccgagggtgcccctcttccttttggacctcaaggaggtccccccggactggcggcccttcgtggagggcctcctcggctag

SEQ ID NO: 52 - DNA, 513 bp, Artificial Sequence
  Description of Artificial Sequence Synthetic polynucleotide
gtgaacctggacgccccacgggtcctggtcctcaacgccgcctacgaagtcctgggcctggccagcattaagcgtgccgtgctcctcgtcctcgggggcggggcggaaatggtctcggaaagcggcctctacctcaacaccccctccacccgtattcccgtccccagcgtcgtccgtctcaagcgtatggtccgtcgtcgtccggggcgtgttcccttgaaccgtcgtaacgtcctccgtcgtgatcgttacacctgccaatactgcgggcaaaagggcggggaactcaccgtggatcatgtcctccccaaaagccgtgggggcaagagcacctgggataacctggtggccgcctgccgtagctgcaacctccgtaagggggatcgtacccccgaagaagcggggatgcgtctcctccgtcccccgaagcccccgcgtgtgcccctcttccttttggatctcaaggaagtccccccggattggcgtcccttcgtggaaggcctcctcggctag

SEQ ID NO: 53 - DNA, 513 bp, Artificial Sequence
  Description of Artificial Sequence Synthetic polynucleotide
gtgaacctggacgccccacgggtcctggtcctcaacgccgcctacgaggtcctgggcctggccagcatcaagcgtgccgtgctcctcgtcctcgggggcggggcggagatggtctcggaaagcggcctctacctcaacaccccctccacccgtatccccgtccccagcgtcgtccgtctcaagcgtatggtccgtcgtcgtccggggcgtgttcccttgaaccgtcgtaacgtcctccgtcgtgaccgttacacctgccagtactgcgggcaaaagggcggggagctcaccgtggaccacgtcctccccaaaagccgtgggggcaagagcacctgggacaacctggtggccgcctgccgtagctgcaacctccgtaagggggaccgtacccccgaggaggcggggatgcgtctcctccgtcccccgaagcccccgcgtgtgcccctcttccttttggacctcaaggaggtccccccggactggcgtcccttcgtggagggcctcctcggctag

SEQ ID NO: 54 - DNA, 549 bp, Artificial Sequence
  Description of Artificial Sequence Synthetic polynucleotide
atgattaaatatagtattcgtggtgaaaacatcgaggtaacagatgcaatccgtaactatgttgagtctaaactcaagaagattgaaaagtatttcaatgctgaacaagagttggatgcacgtatcaatctgaaagtatatcgtgagaaaacagctaaagttgaagtcactattcctcttgctcccgttactcttcgtgcagaggatgtttcacaagatatgtatggttctattgatttagttgttgataagattgaacgtcagattcgtaaaaataaaactaaaattgctaagaagcatcgtgaaaagaaaccagcggcacatgtctttacagctgaatttgaagcagaagagatggaagaggctccagctataaaggttgtcagaaccaaaaacatcactttaaaacctatggatatcgaagaggctcgtttacaaatggatctcttaggtcacgatttcttcatctacacagatgctaatgataatacaacaaatgttctctatcgtcgtgaagatggtaatttgggtcttattgaagcaaaataa

SEQ ID NO: 55 - DNA, 549 bp, Artificial Sequence
  Description of Artificial Sequence Synthetic polynucleotide
atgattaaatatagtattcgtggtgaaaacatcgaggtaacagacgcaatccgcaactatgttgagtctaaactcaagaagatcgagaagtatttcaatgctgagcaggagttggacgcacgcatcaatctgaaagtatatcgcgagaaaacagctaaagttgaggtcactatccctcttgctcccgttactcttcgcgcagaggacgtttcacaggacatgtatggttctatcgacttagttgttgacaagatcgagcgccagatccgcaaaaataaaactaaaatcgctaagaagcaccgcgagaagaaaccagcggcacacgtctttacagctgagtttgaggcagaggagatggaggaggctccagctataaaggttgtcagaaccaaaaacatcactttaaaacctatggacatcgaggaggctcgcttacagatggacctcttaggtcacgacttcttcatctacacagacgctaatgacaatacaacaaatgttctctatcgccgcgaggacggtaatttgggtcttatcgaggcaaaataa

SEQ ID NO: 56 - DNA, 549 bp, Artificial Sequence
  Description of Artificial Sequence Synthetic polynucleotide
atgattaaatatagtattcgtggtgaaaacatcgaggtaacagatgcaatccgcaactatgttgagtctaaactcaagaagattgaaaagtatttcaatgctgaacaagagttggatgcacgcatcaatctgaaagtatatcgcgagaaaacagctaaagttgaagtcactattcctcttgctcccgttactcttcgcgcagaggatgtttcacaagatatgtatggttctattgatttagttgttgataagattgaacgccagattcgcaaaaataaaactaaaattgctaagaagcatcgcgaaaagaaaccagcggcacatgtctttacagctgaatttgaagcagaagagatggaagaggctccagctataaaggttgtcagaaccaaaaacatcactttaaaacctatggatatcgaagaggctcgcttacaaatggatctcttaggtcacgatttcttcatctacacagatgctaatgataatacaacaaatgttctctatcgccgcgaagatggtaatttgggtcttattgaagcaaaataa

SEQ ID NO: 57 - DNA, 663 bp, Artificial Sequence
  Description of Artificial Sequence Synthetic polynucleotide
atgaaacaatctttattcggacgtgtacgcgatgcaattttagctgattttcataacgtgttagatgagaaggaaagaaaaaatccaattgcgatgttaaaccaatatttacgtgatagtgagcgtgaaataacaaaaattgagaagttaattgagcgtcataaaacattaaaatctaattttgctcgtgagcttgagcaagcacgttatttcgttaataaaagatcaaagcaagctatcattgctcaagaagcagacgaattacaattgcacgaacgtgcgttagaagaggtagcttattatgaagggcaagtaactcgattagaagaaatgtatgcaggtgttgtagagcaaattgatgagttagagcgtcgtctttctgaaatgaaaaataaattaaaagaaatgcacgcaaagcgcatggaactaatggcacgtgaaaatatggcacatgcaaatcgtcgtatgaatactgcgatgcataaaatggatgaaaataatccgttcttacgatttgaagagattgaagatcatattcgtgacttagaaactcgtatgaatgaagagcatgagcgtgacacgtttgatatgaaaattgcaaaacttgagcgtgaaatgaaagaaaagaatgatgtatcgttaacgaaagagttaacaaaataa

SEQ ID NO: 58 - DNA, 663 bp, Artificial Sequence
  Description of Artificial Sequence Synthetic polynucleotide
atgaaacaatctttattcggacgtgtacgcgatgcaattttagctgactttcacaacgtgttagacgagaaggagagaaaaaatccaatcgcgatgttaaaccagtatttacgcgacagtgagcgcgagataacaaaaatcgagaagttaatcgagcgccacaaaacattaaaatctaattttgctcgcgagcttgagcaggcacgctatttcgttaataaaagatcaaagcaggctatcatcgctcaggaggcagacgagttacagttgcacgagcgcgcgttagaggaggtagcttattatgaggggcaggtaactcgattagaggagatgtatgcaggtgttgtagagcagatcgacgagttagagcgccgcctttctgagatgaaaaataaattaaaagagatgcacgcaaagcgcatggagctaatggcacgcgagaatatggcacacgcaaatcgccgcatgaatactgcgatgcacaaaatggacgagaataatccgttcttacgatttgaggagatcgaggaccacatccgcgacttagagactcgcatgaatgaggagcacgagcgcgacacgtttgacatgaaaatcgcaaaacttgagcgcgagatgaaagagaagaatgacgtatcgttaacgaaagagttaacaaaataa

SEQ ID NO: 59 - DNA, 663 bp, Artificial Sequence
  Description of Artificial Sequence Synthetic polynucleotide
atgaaacaatctttattcggacgtgtacgcgatgcaattttagctgattttcataacgtgttagatgagaaggaaagaaaaaatccaattgcgatgttaaaccaatatttacgcgatagtgagcgcgaaataacaaaaattgagaagttaattgagcgccataaaacattaaaatctaattttgctcgcgagcttgagcaagcacgctatttcgttaataaaagatcaaagcaagctatcattgctcaagaagcagacgaattacaattgcacgaacgcgcgttagaagaggtagcttattatgaagggcaagtaactcgattagaagaaatgtatgcaggtgttgtagagcaaattgatgagttagagcgccgcctttctgaaatgaaaaataaattaaaagaaatgcacgcaaagcgcatggaactaatggcacgcgaaaatatggcacatgcaaatcgccgcatgaatactgcgatgcataaaatggatgaaaataatccgttcttacgatttgaagagattgaagatcatattcgcgacttagaaactcgcatgaatgaagagcatgagcgcgacacgtttgatatgaaaattgcaaaacttgagcgcgaaatgaaagaaaagaatgatgtatcgttaacgaaagagttaacaaaataa

SEQ ID NO: 60 - DNA, 921 bp, Artificial Sequence
  Description of Artificial Sequence Synthetic polynucleotide
atggctgccccgctcatccccgtcctgactgctcccaccgctgcgggcaaaacggcgctggcgctgcggctggcgcgggagtacggactcgagatcgttgccgccgacgccttcacggtgtaccggggcctcgacctcggcactgccaagccgacgccgcaggagcgggcgagcgtcccccaccatctgcttgacgtggtcgacgtgacgcagagctacgacgtggcgcagtacgcggcgcaggccgaggccgccatcgtggacatcctggcgcgggggcggctgccgctggtcgtgggcggcaccggcttttacctcagtgcgctcagccgggggctgccgctcacgccgccgagtgacccgaagatgcgcgccgccctcgaagccgagttacaggaacgcgggctggacgcgctgctcgccgaaatcgagcaggccaatcctgccgaggccgcccgcatggagcgcaacccacgccgggtggtccgggcgctggaggtctaccgcgctgccgggcgttttcccggtgagttcgggtactcgccacccgctttccagtatcaggtgtttgccttttcgccgcccgccgccgagatggaacagcgggtgcaggagcgcaccgccgccatgctgcgcgccggctggccgcaggaggcgcagtggctcgccgggcaggtgccgccggagcaggagccgcgcccgacggtgtggcaggcgctcgggtacgccgaggcgctggcggtggcgcagggccgcctgagcctcgcaggcgccgagcaagccatcgccctggcgacccggcagtacggcaaacggcagctcacctggatgcgccgtcagctcggggccgaggtgcaatcgccggacgcggcagaggcgcacctgcgggcgtttctggagcgttccggggcgccgagttga

SEQ ID NO: 61 - DNA, 921 bp, Artificial Sequence
  Description of Artificial Sequence Synthetic polynucleotide
atggctgccccgctcatccccgtcctgactgctcccaccgctgcgggcaaaacggcgctggcgctgcgtctggcgcgtgaatacggactcgaaattgttgccgccgatgccttcacggtgtaccgtggcctcgatctcggcactgccaagccgacgccgcaagaacgtgcgagcgtcccccatcatctgcttgatgtggtcgatgtgacgcaaagctacgatgtggcgcaatacgcggcgcaagccgaagccgccattgtggatattctggcgcgtgggcgtctgccgctggtcgtgggcggcaccggcttttacctcagtgcgctcagccgtgggctgccgctcacgccgccgagtgatccgaagatgcgtgccgccctcgaagccgaattacaagaacgtgggctggatgcgctgctcgccgaaattgaacaagccaatcctgccgaagccgcccgtatggaacgtaacccacgtcgtgtggtccgtgcgctggaagtctaccgtgctgccgggcgttttcccggtgaattcgggtactcgccacccgctttccaatatcaagtgtttgccttttcgccgcccgccgccgaaatggaacaacgtgtgcaagaacgtaccgccgccatgctgcgtgccggctggccgcaagaagcgcaatggctcgccgggcaagtgccgccggaacaagaaccgcgtccgacggtgtggcaagcgctcgggtacgccgaagcgctggcggtggcgcaaggccgtctgagcctcgcaggcgccgaacaagccattgccctggcgacccgtcaatacggcaaacgtcaactcacctggatgcgtcgtcaactcggggccgaagtgcaatcgccggatgcggcagaagcgcatctgcgtgcgtttctggaacgttccggggcgccgagttga

SEQ ID NO: 62 - DNA, 921 bp, Artificial Sequence
  Description of Artificial Sequence Synthetic polynucleotide
atggctgccccgctcatccccgtcctgactgctcccaccgctgcgggcaaaacggcgctggcgctgcggctggcgcgggagtacggactcgagatcgttgccgccgacgccttcacggtgtaccggggcctcgacctcggcactgccaagccgacgccgcaagagcgggcgagcgtcccccaccatctgcttgacgtggtcgacgtgacgcaaagctacgacgtggcgcaatacgcggcgcaagccgaggccgccatcgtggacatcctggcgcgggggcggctgccgctggtcgtgggcggcaccggcttttacctcagtgcgctcagccgggggctgccgctcacgccgccgagtgacccgaagatgcgcgccgccctcgaagccgagttacaagaacgcgggctggacgcgctgctcgccgaaatcgagcaagccaatcctgccgaggccgcccgcatggagcgcaacccacgccgggtggtccgggcgctggaggtctaccgcgctgccgggcgttttcccggtgagttcgggtactcgccacccgctttccaatatcaagtgtttgccttttcgccgcccgccgccgagatggaacaacgggtgcaagagcgcaccgccgccatgctgcgcgccggctggccgcaagaggcgcaatggctcgccgggcaagtgccgccggagcaagagccgcgcccgacggtgtggcaagcgctcgggtacgccgaggcgctggcggtggcgcaaggccgcctgagcctcgcaggcgccgagcaagccatcgccctggcgacccggcaatacggcaaacggcaactcacctggatgcgccgtcaactcggggccgaggtgcaatcgccggacgcggcagaggcgcacctgcgggcgtttctggagcgttccggggcgccgagttga

SEQ ID NO: 63 - DNA, 654 bp, Artificial Sequence
  Description of Artificial Sequence Synthetic polynucleotide
atggagtccgtggccctgtacagctttcaggctacagagagcgacgagctggccttcaacaagggagacacactcaagatcctgaacatggaggatgaccagaactggtacaaggccgagctccggggtgtcgagggatttattcccaagaactacatccgcgtcaagccccatccgtggtactcgggcaggatttcccggcagctggccgaagagattctgatgaagcggaaccatctgggagccttcctgatccgggagagtgagagctccccaggggagttctctgtgtctgtgaactatggagaccaggtgcagcacttcaaggtgctgcgtgaggcctcggggaagtacttcctgtgggaggagaagttcaactccctcaacgagctggtcgacttctaccgcaccaccaccatcgccaagaagcggcagatcttcctgcgcgacgaggagcccttgctcaagtcacctggggcctgctttgcccaggcccagtttgacttctcagcccaggacccctcgcagctcagcttccgccgtggcgacatcattgaggtcctggagcgcccagacccccactggtggcggggccggtcctgcgggcgcgttggcttcttcccacggagttacgtgcagcccgtgcacctgtga

SEQ ID NO: 64 - DNA, 654 bp, Artificial Sequence
  Description of Artificial Sequence Synthetic polynucleotide
atggagtccgtggccctgtacagctttcaggctacagagagcgatgaactggccttcaacaagggagatacactcaagattctgaacatggaagatgatcaaaactggtacaaggccgaactccgtggtgtcgaaggatttattcccaagaactacattcgtgtcaagccccatccgtggtactcgggccgtatttcccgtcaactggccgaagaaattctgatgaagcgtaaccatctgggagccttcctgattcgtgaaagtgaaagctccccaggggaattctctgtgtctgtgaactatggagatcaagtgcaacatttcaaggtgctgcgtgaagcctcggggaagtacttcctgtgggaagaaaagttcaactccctcaacgaactggtcgatttctaccgtaccaccaccattgccaagaagcgtcaaattttcctgcgtgatgaagaacccttgctcaagtcacctggggcctgctttgcccaagcccaatttgatttctcagcccaagatccctcgcaactcagcttccgtcgtggcgatattattgaagtcctggaacgtccagatccccattggtggcgtggccgttcctgcgggcgtgttggcttcttcccacgtagttacgtgcaacccgtgcatctgtga

SEQ ID NO: 65 - DNA, 654 bp, Artificial Sequence
  Description of Artificial Sequence Synthetic polynucleotide
atggagtccgtggccctgtacagctttcaggctacagagagcgacgagctggccttcaacaagggagacacactcaagatcctgaacatggaggatgaccaaaactggtacaaggccgagctccggggtgtcgagggatttattcccaagaactacatccgcgtcaagccccatccgtggtactcgggcaggatttcccggcaactggccgaagagattctgatgaagcggaaccatctgggagccttcctgatccgggagagtgagagctccccaggggagttctctgtgtctgtgaactatggagaccaagtgcaacacttcaaggtgctgcgtgaggcctcggggaagtacttcctgtgggaggagaagttcaactccctcaacgagctggtcgacttctaccgcaccaccaccatcgccaagaagcggcaaatcttcctgcgcgacgaggagcccttgctcaagtcacctggggcctgctttgcccaagcccaatttgacttctcagcccaagacccctcgcaactcagcttccgccgtggcgacatcattgaggtcctggagcgcccagacccccactggtggcggggccggtcctgcgggcgcgttggcttcttcccacggagttacgtgcaacccgtgcacctgtga

SEQ ID NO: 66 - DNA, 510 bp, Artificial Sequence
  Description of Artificial Sequence Synthetic polynucleotide
atgcgaacctatgaatcaaaagaagccttgattgaggccattcaaatagcttcacaaaaatatttagctgaatttgcagaaattcctgaaacacttaaagatcaccgaattgaaacagtagctaaaacaccttcagagaacttagcctatcaattaggttggctcaacttgctgctttcttgggaagaacaagaacaacgtggtctgaccgttcaaacgccagctgaaggctataaatggaatcaactgggcgcgctctatcaatcattttatcaaacctatggacaaatgagtttagaaagtcagctgattgcgttgcaagacaccttagaaaaattacttcattggattgactcgctttccgaagacgaattatttttacctcaacaacgggcttgggcgaccaccaaagcacaatggcctctttggaaatggattcacattaatagcgttgccccttttactagtttccgaacgcaaattcgcaaatggaaaaaagcttgtctttaa

SEQ ID NO: 67 - DNA, 510 bp, Artificial Sequence
  Description of Artificial Sequence Synthetic polynucleotide
atgcgaacctatgaatcaaaagaagccttgattgaggccattcagatagcttcacagaaatatttagctgagtttgcagagatccctgagacacttaaagaccaccgaatcgagacagtagctaaaacaccttcagagaacttagcctatcagttaggttggctcaacttgctgctttcttgggaggagcaggagcagcgcggtctgaccgttcagacgccagctgagggctataaatggaatcagctgggcgcgctctatcagtcattttatcagacctatggacagatgagtttagagagtcagctgatcgcgttgcaggacaccttagagaaattacttcactggatcgactcgctttccgaggacgagttatttttacctcagcagcgggcttgggcgaccaccaaagcacagtggcctctttggaaatggatccacatcaatagcgttgccccttttactagtttccgaacgcagatccgcaaatggaaaaaagcttgtctttaa

SEQ ID NO: 68 - DNA, 510 bp, Artificial Sequence
  Description of Artificial Sequence Synthetic polynucleotide
atgcgaacctatgaatcaaaagaagccttgattgaggccattcagatagcttcacagaaatatttagctgaatttgcagaaattcctgaaacacttaaagatcaccgaattgaaacagtagctaaaacaccttcagagaacttagcctatcagttaggttggctcaacttgctgctttcttgggaagaacaggaacagcgtggtctgaccgttcagacgccagctgaaggctataaatggaatcagctgggcgcgctctatcagtcattttatcagacctatggacagatgagtttagaaagtcagctgattgcgttgcaggacaccttagaaaaattacttcattggattgactcgctttccgaagacgaattatttttacctcagcagcgggcttgggcgaccaccaaagcacagtggcctctttggaaatggattcacattaatagcgttgccccttttactagtttccgaacgcagattcgcaaatggaaaaaagcttgtctttaa

SEQ ID NO: 69 - DNA, 495 bp, Artificial Sequence
  Description of Artificial Sequence Synthetic polynucleotide
atggatgtgaaacaaactttggagaaggcgattgcccttcgccaaaataagcgctatcaagagtcgaatgccatccttgtcacactctgtaaggagcatgctcacgatccacaaattctttatcaatgtggctggagctttgatgtactaggattggaagctcaagctgttccttattatgaaaaggcgatcgcatcgggtcttcaaggaaaggacttggcggagtgttatctcgggctaggtagcacatttcgaacgctaggggagtataggaaagcagaagccgttctcgcaaacggcgtgaagcaatttcctaaccatcaggcgctccgtgttttctacgcaatggtcctctacaaccttggtcgctatgagcaaggggtagaattattgctaaaaataatcgctgaaacgagcgatgatgagacgatacaatcttacaagcaagcgattctcttttatgcagataagctagatgaaacgtggaaagcataa

SEQ ID NO: 70 - DNA, 495 bp, Artificial Sequence
  Description of Artificial Sequence Synthetic polynucleotide
atggatgtgaaacaaactttggagaaggcgattgcccttcgccagaataagcgctatcaggagtcgaatgccatccttgtcacactctgtaaggagcacgctcacgacccacagatcctttatcagtgtggctggagctttgacgtactaggattggaggctcaggctgttccttattatgagaaggcgatcgcatcgggtcttcagggaaaggacttggcggagtgttatctcgggctaggtagcacatttcgaacgctaggggagtataggaaagcagaggccgttctcgcaaacggcgtgaagcagtttcctaaccaccaggcgctccgcgttttctacgcaatggtcctctacaaccttggtcgctatgagcagggggtagagttattgctaaaaataatcgctgagacgagcgacgacgagacgatacagtcttacaagcaggcgatcctcttttatgcagacaagctagacgagacgtggaaagcataa

SEQ ID NO: 71 - DNA, 495 bp, Artificial Sequence
  Description of Artificial Sequence Synthetic polynucleotide
atggatgtgaaacaaactttggagaaggcgattgcccttcgccagaataagcgctatcaggagtcgaatgccatccttgtcacactctgtaaggagcatgctcacgatccacagattctttatcagtgtggctggagctttgatgtactaggattggaagctcaggctgttccttattatgaaaaggcgatcgcatcgggtcttcagggaaaggacttggcggagtgttatctcgggctaggtagcacatttcgaacgctaggggagtataggaaagcagaagccgttctcgcaaacggcgtgaagcagtttcctaaccatcaggcgctccgtgttttctacgcaatggtcctctacaaccttggtcgctatgagcagggggtagaattattgctaaaaataatcgctgaaacgagcgatgatgagacgatacagtcttacaagcaggcgattctcttttatgcagataagctagatgaaacgtggaaagcataa

SEQ ID NO: 72 - DNA, 708 bp, Artificial Sequence
  Description of Artificial Sequence Synthetic polynucleotide
atgacggacaaataccgcctccgagagcgcgtctgggacgacctcgaagacagcggcgtggcgcggttcccgttcccgccacacggccgcatcccgaactacgccggtgccgatgaggccgccgcccgcctcaccgaaacggacgtgtggcagcgcgctgagaccgtgaaggcgaaccccgacgccccccagctgccggtgcggcgggcggcgctgcgcgcggggaagacactgtacgcggcggtgccgcggctgcgcgacgaggagtgtttcctgcgcctcgacccaacgaccatcgacgacatcgacgccgccacgacggtgtcggggatcgaggagtacggcgacccggtcggtcccggggacgtcgatcccatcgacctcatcgtgtcggggagcgtcgcggtcaccgaccgcggcgagcgcgtcgggaaaggggaggggtacagcgacctggagttcgcgctgctgcgggcgttcgggcgcgtcgacgacgacaccgcgactgtgacgaccgtccacgagcgccaggtcgtcgacgacgctgtgccgaccgccgcccacgacgtgccgatggagtacgtggtcacgccggaccgaacgatcaccaccacccacgaggatgacacgcccagtggcatcgactgggacgcactggacgagcagcgcctggcggagatcccggtgttggaccgtcgctcgccgtag

SEQ ID NO: 73 - DNA, 708 bp, Artificial Sequence
  Description of Artificial Sequence Synthetic polynucleotide
atgacggacaaataccgcctccgagagcgcgtctgggacgacctcgaagatagcggcgtggcgcgtttcccgttcccgccacatggccgtattccgaactacgccggtgccgatgaagccgccgcccgtctcaccgaaacggatgtgtggcaacgtgctgaaaccgtgaaggcgaaccccgatgccccccaactgccggtgcgtcgtgcggcgctgcgtgcggggaagacactgtacgcggcggtgccgcgtctgcgtgatgaagaatgtttcctgcgtctcgatccaacgaccattgatgatattgatgccgccacgacggtgtcggggattgaagaatacggcgatccggtcggtcccggggatgtcgatcccattgatctcattgtgtcggggagcgtcgcggtcaccgatcgtggcgaacgtgtcgggaaaggggaagggtacagcgatctggaattcgcgctgctgcgtgcgttcgggcgtgtcgatgatgataccgcgactgtgacgaccgtccatgaacgtcaagtcgtcgatgatgctgtgccgaccgccgcccatgatgtgccgatggaatacgtggtcacgccggatcgtacgattaccaccacccatgaagatgatacgcccagtggcattgattgggatgcactggatgaacaacgtctggcggaaattccggtgttggatcgtcgttcgccgtag

SEQ ID NO: 74 - DNA, 708 bp, Artificial Sequence
  Description of Artificial Sequence Synthetic polynucleotide
atgacggacaaataccgcctccgagagcgcgtctgggacgacctcgaagatagcggcgtggcgcggttcccgttcccgccacacggccgcatcccgaactacgccggtgccgatgaggccgccgcccgcctcaccgaaacggatgtgtggcagcgcgctgagaccgtgaaggcgaaccccgatgccccccagctgccggtgcggcgggcggcgctgcgcgcggggaagacactgtacgcggcggtgccgcggctgcgcgatgaggagtgtttcctgcgcctcgatccaacgaccatcgatgatatcgatgccgccacgacggtgtcggggatcgaggagtacggcgatccggtcggtcccggggatgtcgatcccatcgatctcatcgtgtcggggagcgtcgcggtcaccgatcgcggcgagcgcgtcgggaaaggggaggggtacagcgatctggagttcgcgctgctgcgggcgttcgggcgcgtcgatgatgataccgcgactgtgacgaccgtccacgagcgccaggtcgtcgatgatgctgtgccgaccgccgcccacgatgtgccgatggagtacgtggtcacgccggatcgaacgatcaccaccacccacgaggatgatacgcccagtggcatcgattgggatgcactggatgagcagcgcctggcggagatcccggtgttggatcgtcgctcgccgtag

SEQ ID NO: 75 - DNA, 744 bp, Artificial Sequence
  Description of Artificial Sequence Synthetic polynucleotide
atgaacgctcgatccacgctcagtgtgtgtgccgtcgccgccgtcctcgttgtcgccgggatcgcgggcgcgaccgccctcggcatggggccggcgtcggccgacacccacaccaccgactcgaaagccatcacggtgtcggccgccggcaccgtcgacgcaaccgccaaccaggcggtcatcgacgtcgccgtgaccgccagcgggaacgactccaccgcagtccgggagtcgttggcggccgacgtgcagtccgtccgcgacgccctcgccgacgacggcgtccccgccaacaccgtccgcaccacgaacttcgacatccgacagcaacgcgaccgcaccccgaacggcgtcgaatacagcggctaccgcggcgtccacgacctcgaaatcacgaccaacgacacgtccgcggcgggcgaactcatcgacgtcgccgtcaccaacggcgcggacaccatcgacggcacgtcgttcacgctctccgacgccaaacgggaccgcctccacaacgacgcgctgaacaccgcgatggccaacgccagacagcgcgccgacaccctcgcgtccgccggcgggctcggcgtcgccggcgtccacgccatcgactccgcggacacgaccgcccatcctcgcgccgaggccggcgggatggtcccccagagcaccaccgccaccaccatcgactccggcccggtcaccgtcacggcctccgtccaggtgacgtacaacgcgacggcgtag

SEQ ID NO: 76 - DNA, 744 bp, Artificial Sequence
  Description of Artificial Sequence Synthetic polynucleotide
atgaacgctcgatccacgctcagtgtgtgtgccgtcgccgccgtcctcgttgtcgccgggattgcgggcgcgaccgccctcggcatggggccggcgtcggccgatacccataccaccgattcgaaagccattacggtgtcggccgccggcaccgtcgatgcaaccgccaaccaagcggtcattgatgtcgccgtgaccgccagcgggaacgattccaccgcagtccgtgaatcgttggcggccgatgtgcaatccgtccgtgatgccctcgccgatgatggcgtccccgccaacaccgtccgtaccacgaacttcgatattcgtcaacaacgtgatcgtaccccgaacggcgtcgaatacagcggctaccgtggcgtccatgatctcgaaattacgaccaacgatacgtccgcggcgggcgaactcattgatgtcgccgtcaccaacggcgcggataccattgatggcacgtcgttcacgctctccgatgccaaacgtgatcgtctccataacgatgcgctgaacaccgcgatggccaacgcccgtcaacgtgccgataccctcgcgtccgccggcgggctcggcgtcgccggcgtccatgccattgattccgcggatacgaccgcccatcctcgtgccgaagccggcgggatggtcccccaaagcaccaccgccaccaccattgattccggcccggtcaccgtcacggcctccgtccaagtgacgtacaacgcgacggcgtag

SEQ ID NO: 77 - DNA, 744 bp, Artificial Sequence
  Description of Artificial Sequence Synthetic polynucleotide
atgaacgctcgatccacgctcagtgtgtgtgccgtcgccgccgtcctcgttgtcgccgggatcgcgggcgcgaccgccctcggcatggggccggcgtcggccgatacccacaccaccgattcgaaagccatcacggtgtcggccgccggcaccgtcgatgcaaccgccaaccaggcggtcatcgatgtcgccgtgaccgccagcgggaacgattccaccgcagtccgggagtcgttggcggccgatgtgcagtccgtccgcgatgccctcgccgatgatggcgtccccgccaacaccgtccgcaccacgaacttcgatatccgacagcaacgcgatcgcaccccgaacggcgtcgaatacagcggctaccgcggcgtccacgatctcgaaatcacgaccaacgatacgtccgcggcgggcgaactcatcgatgtcgccgtcaccaacggcgcggataccatcgatggcacgtcgttcacgctctccgatgccaaacgggatcgcctccacaacgatgcgctgaacaccgcgatggccaacgccagacagcgcgccgataccctcgcgtccgccggcgggctcggcgtcgccggcgtccacgccatcgattccgcggatacgaccgcccatcctcgcgccgaggccggcgggatggtcccccagagcaccaccgccaccaccatcgattccggcccggtcaccgtcacggcctccgtccaggtgacgtacaacgcgacggcgtag

SEQ ID NO: 78 - DNA, 579 bp, Artificial Sequence
  Description of Artificial Sequence Synthetic polynucleotide
atggaaaacaaaacaaataatacaaaaacagagatcaaaaaaaaggacatgtcaaaaacttttgagactattaaaggagaactattttttgaagataaagtaattcaaaaaataattggtattgcattagatgagattgatggtcttctaacgattgatggaggcttcttctcaaatatagctggaaaactagtaaatacggataacacaacttctggagtggatgttgaagtaggaaaaaaacaagtcgcagtagatctttcaatagtggctgaatatggtaaagatgtaactacaatttatgataaaatgaagcaagttatttcaaatgaagttaagaaaatgactggcctagatgtaattgagattaatgtaaacgtcgtagatgtaaaaacgaaagaacaacatgaaaatgattcagttactctacaagatcatctttccgatgcagcttctgctactggagaatttgcttcaaaacaatttgaaaaatcaaaagaagctttaggcgtagcaagtgaaaaagtaagtgatggtgtacaaaacgtaaaagaagaaactgaacctcgcgtaaaataa

SEQ ID NO: 79 - DNA, 579 bp, Artificial Sequence
  Description of Artificial Sequence Synthetic polynucleotide
atggaaaacaaaacaaataatacaaaaacagagatcaaaaaaaaggacatgtcaaaaacttttgagactatcaaaggagagctattttttgaggacaaagtaatccagaaaataatcggtatcgcattagacgagatcgacggtcttctaacgatcgacggaggcttcttctcaaatatagctggaaaactagtaaatacggacaacacaacttctggagtggacgttgaggtaggaaaaaaacaggtcgcagtagacctttcaatagtggctgagtatggtaaagacgtaactacaatctatgacaaaatgaagcaggttatctcaaatgaggttaagaaaatgactggcctagacgtaatcgagatcaatgtaaacgtcgtagacgtaaaaacgaaagagcagcacgagaatgactcagttactctacaggaccacctttccgacgcagcttctgctactggagagtttgcttcaaaacagtttgagaaatcaaaagaggctttaggcgtagcaagtgagaaagtaagtgacggtgtacagaacgtaaaagaggagactgagcctcgcgtaaaataa

SEQ ID NO: 80 - DNA, 579 bp, Artificial Sequence
  Description of Artificial Sequence Synthetic polynucleotide
atggaaaacaaaacaaataatacaaaaacagagatcaaaaaaaaggacatgtcaaaaacttttgagactattaaaggagaactattttttgaagacaaagtaattcaaaaaataattggtattgcattagacgagattgacggtcttctaacgattgacggaggcttcttctcaaatatagctggaaaactagtaaatacggacaacacaacttctggagtggacgttgaagtaggaaaaaaacaagtcgcagtagacctttcaatagtggctgaatatggtaaagacgtaactacaatttatgacaaaatgaagcaagttatttcaaatgaagttaagaaaatgactggcctagacgtaattgagattaatgtaaacgtcgtagacgtaaaaacgaaagaacaacatgaaaatgactcagttactctacaagaccatctttccgacgcagcttctgctactggagaatttgcttcaaaacaatttgaaaaatcaaaagaagctttaggcgtagcaagtgaaaaagtaagtgacggtgtacaaaacgtaaaagaagaaactgaacctcgcgtaaaataa

SEQ ID NO: 81 - DNA, 585 bp, Artificial Sequence
  Description of Artificial Sequence Synthetic polynucleotide
atgagccaatcgagcgatgcgtcagagaaggaaaaaccgaaagagaaaaaatcgcaagaagagcttgaaaaggaacttgataaggaattgaaaaaaggcggtgagccgaagaccaaaaaagatgatcaaatacataaaataggagaaacatttaaagcaggacatacgaattttacagtgaataaagttgatagagtgcaaaaaggtgaatatatgaatgttggcggagctgtaaatgaggagacaaaaacaataaaagatgatgaggaacggcttattatagaagttacgatggaaaatataggggaagattcaataagctacaattttatcgggtttgatttaagagataagaatgatcaatcagtgcggcctgttttttctatagaagagaagggcagaatccttatgggaggaacactagtatcggggaaaaaggttacaggtgtactcagttatgtcatccctaaaggagaacagaaacattacacactggtatataatccgtttttagctgatacaaatagcagtaatacagaagagagagtaaaggacgatattgattacttggtgaagttagattag

SEQ ID NO: 82 - DNA, 585 bp, Artificial Sequence
  Description of Artificial Sequence Synthetic polynucleotide
atgagccaatcgagcgatgcgtcagagaaggaaaaaccgaaagagaaaaaatcgcaggaggagcttgagaaggagcttgacaaggagttgaaaaaaggcggtgagccgaagaccaaaaaagacgaccagatacacaaaataggagagacatttaaagcaggacacacgaattttacagtgaataaagttgacagagtgcagaaaggtgagtatatgaatgttggcggagctgtaaatgaggagacaaaaacaataaaagacgacgaggagcggcttatcatagaggttacgatggagaatataggggaggactcaataagctacaattttatcgggtttgacttaagagacaagaatgaccagtcagtgcggcctgttttttctatagaggagaagggcagaatccttatgggaggaacactagtatcggggaaaaaggttacaggtgtactcagttatgtcatccctaaaggagagcagaaacactacacactggtatataatccgtttttagctgacacaaatagcagtaatacagaggagagagtaaaggacgacatcgactacttggtgaagttagactag

SEQ ID NO: 83 - DNA, 585 bp, Artificial Sequence
  Description of Artificial Sequence Synthetic polynucleotide
atgagccaatcgagcgatgcgtcagagaaggaaaaaccgaaagagaaaaaatcgcaagaagagcttgaaaaggaacttgacaaggaattgaaaaaaggcggtgagccgaagaccaaaaaagacgaccaaatacataaaataggagaaacatttaaagcaggacatacgaattttacagtgaataaagttgacagagtgcaaaaaggtgaatatatgaatgttggcggagctgtaaatgaggagacaaaaacaataaaagacgacgaggaacggcttattatagaagttacgatggaaaatataggggaagactcaataagctacaattttatcgggtttgacttaagagacaagaatgaccaatcagtgcggcctgttttttctatagaagagaagggcagaatccttatgggaggaacactagtatcggggaaaaaggttacaggtgtactcagttatgtcatccctaaaggagaacagaaacattacacactggtatataatccgtttttagctgacacaaatagcagtaatacagaagagagagtaaaggacgacattgactacttggtgaagttagactag

SEQ ID NO: 84 - protein, 6 aa, Artificial Sequence
  Description of Artificial Sequence Synthetic 6xHis tag
HHHHHH

SEQ ID NO: 85 - DNA, 456 bp, Artificial Sequence; identical to SEQ ID NO: 3
  Description of Artificial Sequence Synthetic polynucleotide

SEQ ID NO: 86 - DNA, 456 bp, Artificial Sequence; identical to SEQ ID NO: 4
  Description of Artificial Sequence Synthetic polynucleotide

SEQ ID NO: 87 - DNA, 774 bp, Artificial Sequence; identical to SEQ ID NO: 5
  Description of Artificial Sequence Synthetic polynucleotide

SEQ ID NO: 88 - DNA, 774 bp, Artificial Sequence; identical to SEQ ID NO: 6
  Description of Artificial Sequence Synthetic polynucleotide

SEQ ID NO: 89 - DNA, 639 bp, Artificial Sequence
  Description of Artificial Sequence Synthetic polynucleotide
atggatgtaacacgacaaatagaattagcgcatcgatatatgaaagactttcacaaaagtgactattctggtcacgacgttgcacacgtagagcgcgtaacgtcactagctcagacaatctctaaatgcgagcagcagggagagtatttaatcatcacattatctgcattacttcacgacgtcatcgacgacaagttaacaaataaagccaatgctttagaccgcttaaaaacatttttaaagaacatccgcgtatcttctgaccagcagcagaagatcatctacatcatccagcacttaagttatagaaatggacagaataatcacgtagaccttccaatcgagggacagatcgttagagacgcagaccgactagacgcgatcggtgctatcggtatcgctagagcatttcagttttcaggccactttaatgagccaatgtggacagagtcaccacacagtgacatacctaatatcgagacgatcactaatttagagccttccgctatacgccacttttatgacaaattattaaaattaaaagacttaatgcacactgagactggtcgaaaattagctagagagagacacgcgtttatggagcagtttttaaatcagttttataaagagtggcacata

SEQ ID NO: 90 - DNA, 639 bp, Artificial Sequence
  Description of Artificial Sequence Synthetic polynucleotide
atggatgtaacacgacaaatagaattagcgcatcgatatatgaaagattttcacaaaagtgattattctggtcacgatgttgcacacgtagaacgtgtaacgtcactagctcaaacaatctctaaatgcgagcaacaaggagaatatttaattatcacattatctgcattacttcacgatgtcattgatgataagttaacaaataaagccaatgctttagatcgtttaaaaacatttttaaagaacattcgcgtatcttctgatcaacaacaaaagattatttacatcattcaacacttaagttatagaaatggacaaaataatcacgtagaccttccaattgaaggacaaattgttagagatgcagatcgactagatgcgattggtgctattggtattgctagagcatttcaattttcaggccactttaatgagccaatgtggacagaatcaccacacagtgacatacctaatattgaaacgattactaatttagaaccttccgctatacgtcacttttatgataaattattaaaattaaaagatttaatgcacactgaaactggtcgaaaattagctagagaaagacacgcgtttatggaacagtttttaaatcaattttataaagaatggcacata

SEQ ID NO: 91 - DNA, 654 bp, Artificial Sequence
  Description of Artificial Sequence Synthetic polynucleotide
atgaaactcattcaaatgtcagaccatatttataaattaaatatacagacaacagttggtatcccgatacagataaacacttggtttatcgtgaatgacaacgacgtttatatcatagacacaggtatggacgactatgctgagctacagatcacgatcgctaaatcgctcggtaatcctaaaggcatctttttaacgcacggacacctagaccacatcaatggcgcaaaacgcatctctgaggctttgaaaatacctatctttacatataaaaatgagctcccttatatcaatggtgagctgccttatccaaataaaacgcacaccgagaatacaggtgttcagtacatcgttaaacctctagagactaatacaaatctgcccttcaattattacttaactcctggtcacgcaccaggtcacgtcatctattttcacaatcaggacaaaatcttaatatgcggagacttatttatctcagacgcgcagcacctgcacatccctatcaaaaaattcacttataacatgactgagaatatcaaaagcggtcagatcatagacaatctttgtcccaaattaatcacaacttcacacggcgacgacctatattattcagacgacatctattcaatctataaatttaagtacgaggag

SEQ ID NO: 92 - DNA, 654 bp, Artificial Sequence
  Description of Artificial Sequence Synthetic polynucleotide
atgaaactcattcaaatgtcagaccatatttataaattaaatatacagacaacagttggtatcccgatacaaataaacacttggtttattgtgaatgataacgacgtttatatcatagacacaggtatggatgattatgctgagctacaaatcacgattgctaaatcgctcggtaatcctaaaggcatttttttaacgcacggacacctagatcacatcaatggcgcaaaacgtatttctgaagctttgaaaatacctatctttacatataaaaatgaactcccttatatcaatggtgagctgccttatccaaataaaacgcacaccgaaaatacaggtgttcaatacattgttaaacctctagaaactaatacaaatctgcccttcaattattacttaactcctggtcacgcaccaggtcacgtcatctattttcacaatcaagataaaattttaatatgcggagatttatttatttcagatgcgcaacacctgcacattcctatcaaaaaattcacttataacatgactgaaaatatcaaaagcggtcaaatcatagataatctttgtcccaaattaattacaacttcacacggcgatgatctatattattcagatgacatttattcaatttataaatttaagtacgaggag

SEQ ID NO: 93 - DNA, 798 bp, Artificial Sequence
  Description of Artificial Sequence Synthetic polynucleotide
atgaggcggagggctagatggctgaggagggagagggaggaggaagaacgtgttaaggatcgtgatatgtttaagattgtggatgaagttttcgattccattaccctctcccatctctaccgtctctactcgcgtaaggtcctccgtgaactcaagggctctattagcagcggtaaggaatctaaggtctactggggcgtcgcgtgggatcgtagcgatgtcgccgttaagatttacctctcgttcacttccgatttccgtaagagcattcgtaaatatattgtcggggatccccgtttcgaagatattcccgcaggcaacattcgtcgtctgatttacgaatgggctcgtaaagaataccgtaacctccgtcgtatgcgtgaatcgggggtccgtgttccccgtcccgtggccgtcgaagcaaacattattgttatggaattcctgggcgaaaaggggtaccgtgcccctaccctggctgaagctgtcgaagaacttgatcgtggggaagcggaagctattgcggccgaagtcctccgtcaagcggaagctattgtatgtcgtgcccgtctcgtgcatgccgatctcagcgaatacaacattctagtctggcgtggggaaccctggattattgatgtctcccaagcggtgccccatagccatccgaacgctgaagaatttctagaacgtgatgtggaaaacctccatcgtttcttgacaggtaagatggggttcgaattcgattttgatgcttatctctctcgtctaaaaagctgtattcatcgtggtgctcgtggt

SEQ ID NO: 94 - DNA, 798 bp, Artificial Sequence
  Description of Artificial Sequence Synthetic polynucleotide
atgaggcggagggctagatggctgaggagggagagggaggaggaagaaagggttaaggaccgggacatgtttaagattgtggacgaagttttcgactccataaccctctcccacctctacaggctctactcgcgcaaggtcctcagggaactcaagggctctataagcagcggtaaggaatctaaggtctactggggcgtcgcgtgggataggagcgacgtcgccgttaagatatacctctcgttcacttccgacttcaggaagagcattagaaaatatattgtcggggaccccaggttcgaagacatccccgcaggcaacataaggaggctgatatacgaatgggctaggaaagaatacaggaacctcaggaggatgcgcgaatcgggggtcagggttcccaggcccgtggccgtcgaagcaaacattatagttatggaattcctgggcgaaaaggggtacagggcccctaccctggctgaagctgtcgaagaacttgataggggggaagcggaagctatagcggccgaagtcctccgccaggcggaagctatagtatgtagggccaggctcgtgcacgccgacctcagcgaatacaacatactagtctggaggggggaaccctggataatagacgtctcccaggcggtgccccacagccacccgaacgctgaagaatttctagaaagggacgtggaaaacctccacaggttcttgacaggtaagatggggttcgaattcgactttgacgcttatctctctaggctaaaaagctgtatccaccggggtgctaggggt

SEQ ID NO: 95 - DNA, 627 bp, Artificial Sequence; identical to SEQ ID NO: 1
  Description of Artificial Sequence Synthetic polynucleotide

SEQ ID NO: 96 - DNA, 627 bp, Artificial Sequence; identical to SEQ ID NO: 2
  Description of Artificial Sequence Synthetic polynucleotide

SEQ ID NO: 97 - DNA, 948 bp, Artificial Sequence
  Description of Artificial Sequence Synthetic polynucleotide
atgaaataccaagtattactttattacaaatatacaacaattgaggacccagaggcttttgcgaaagagcacctagctttttgcaaatcattaaacttaaaaggccgcatcttagtagcgacagaggggatcaacggaacgttatctggtactgtcgaggagacagagaagtatatggaggcaatgcaggcagacgagcgctttaaggacacattctttaaaatcgacccagcagaggagatggccttccgcaaaatgtttgttcgcccacgctctgagttagtggcgttgaacttagaggaggacgttgacccattagagacgacggggaaatatttggagcctgcagagtttaaagaggccttattagacgaggacactgttgtaatcgacgctcgcaacgactatgagtatgacttaggtcacttccgcggtgccgtgcgcccagacatccgcagcttccgcgagttaccacagtggatccgcgagaacaaagagaaatttatggacaaaaaaatcgttacctattgtactggcgggatccgctgtgagaaattttctggctggttattaaaagagggatttgaggacgttgctcagttgcacggtggtatcgccaactatggaaaaaatccagagacacgcggcgagctttgggacggcaaaatgtatgtctttgacgaccgaatcagtgtcgagatcaatcacgttgacaaaaaagttatcgggaaagactggtttgacgggacaccttgcgagcgctacatcaactgtgcaaacccagagtgtaatcgccagatcttaacttcagaggagaatgagcacaaacacttaggtggctgctcattagagtgtagccagcaccctgccaaccgctatgtaaaaaaacacaatttaacagaggcagaggttgctgagcgcttagctttgttagaggcggttgaggta

SEQ ID NO: 98 - DNA, 948 bp, Artificial Sequence
  Description of Artificial Sequence Synthetic polynucleotide
atgaaataccaagtattactttattacaaatatacaacaattgaggatccagaggcttttgcgaaagagcatctagctttttgcaaatcattaaacttaaaaggccgtattttagtagcgacagaggggattaacggaacgttatctggtactgtcgaggagacagagaagtatatggaggcaatgcaagcagatgagcgctttaaggatacattctttaaaattgatccagcagaggagatggccttccgcaaaatgtttgttcgcccacgttctgagttagtggcgttgaacttagaggaggacgttgatccattagagacgacggggaaatatttggagcctgcagagtttaaagaggccttattagacgaggacactgttgtaatcgatgctcgtaacgattatgagtatgatttaggtcatttccgtggtgccgtgcgcccagatatccgtagcttccgtgagttaccacaatggattcgcgagaacaaagagaaatttatggataaaaaaattgttacctattgtactggcgggattcgctgtgagaaattttctggctggttattaaaagagggatttgaggatgttgctcaattgcatggtggtatcgccaactatggaaaaaatccagagacacgtggcgagctttgggacggcaaaatgtatgtctttgatgaccgaatcagtgtcgagattaatcatgttgataaaaaagttattgggaaagactggtttgatgggacaccttgcgagcgctacattaactgtgcaaacccagagtgtaatcgtcaaatcttaacttcagaggagaatgagcataaacatttaggtggctgctcattagagtgtagccagcatcctgccaaccgttatgtaaaaaaacataatttaacagaggcagaggttgctgagcgtttagctttgttagaggcggttgaggta

SEQ ID NO: 99 - DNA, 552 bp, Artificial Sequence
  Description of Artificial Sequence Synthetic polynucleotide
atgatatacagatttactatcatatctgatgaagttgacgattttgtcagagagatacagatcgacccggaggctacatttcttgacttccacgaggcaatactgaaatcagtagggtacacaaacgaccagatgacctccttctttatctgcgacgacgactgggagaaagagaaagaggtcactttggaggagatggacgacaatccggagatggacagttggataatgaaagagactactatcagcgagctggtagaggacgagaagcagaaattgttgtatgtattcgactacatgacagagcgctgcttcttcatcgagttgtctgagatcatcaccggaaaagacatgaatggtgccaaatgtaccaagaaatcgggtgacgctccgccacagactgtagactttgaggagatggctgctgcaagcggttcactcgacctggacgagaatttctatggtgaccaggactttgacatggaggactttgaccaggagggcttcgacataggtggtaacgcgggtggctcttatgaggaggagaagttt

SEQ ID NO: 100 - DNA, 552 bp, Artificial Sequence
  Description of Artificial Sequence Synthetic polynucleotide
atgatatacagatttactatcatatctgatgaagttgacgattttgtcagagagatacaaattgatccggaggctacatttcttgacttccatgaggcaatactgaaatcagtagggtacacaaacgaccagatgacctccttctttatctgcgatgatgattgggagaaagagaaagaggtcactttggaggagatggacgacaatccggagatggatagttggataatgaaagagactactatcagcgagctggtagaggatgagaagcaaaaattgttgtatgtattcgactacatgacagagcgttgcttcttcatcgagttgtctgagatcatcaccggaaaagatatgaatggtgccaaatgtaccaagaaatcgggtgatgctccgccacaaactgtagattttgaggagatggctgctgcaagcggttcactcgacctggacgagaatttctatggtgatcaggactttgatatggaggattttgatcaggagggcttcgacataggtggtaacgcgggtggctcttatgaggaggagaagttt

SEQ ID NO: 101 - DNA, 849 bp, Artificial Sequence; identical to SEQ ID NO: 7
  Description of Artificial Sequence Synthetic polynucleotide

SEQ ID NO: 102 - DNA, 849 bp, Artificial Sequence; identical to SEQ ID NO: 8
  Description of Artificial Sequence Synthetic polynucleotide

SEQ ID NO: 103 - DNA, 564 bp, Artificial Sequence
  Description of Artificial Sequence Synthetic polynucleotide
atgggtagtatagaggaggtgcttttggaggagaggctcataggatatctagatcccggagccgaaaaagttttagcgcgtattaaccgtccttcaaaaattgtgtctacaagcagttgtacagggcgtattacactgattgaaggcgaagctcattggctccgtaacggggcacgtgtagcgtacaagacccatcatcccatttcccgtagtgaagttgaacgtgttctacgtcgtggcttcacaaacctttggctcaaggtgaccggccctattctacatctccgtgttgaagggtggcaatgtgcaaagtcccttctcgaagcagctcgtcgtaacgggttcaagcatagcggagtcattagcattgctgaagattcacgtctcgtcattgaaattatgagcagccaaagcatgtcagtacctctagttatggaaggtgctcgtattgtcggcgatgatgccctagatatgctgattgaaaaagcaaacactattctagttgaatctcgtattgggctagatacgttttcacgtgaagtcgaagaacttgtcgaatgcttt

SEQ ID NO: 104 - DNA, 564 bp, Artificial Sequence
  Description of Artificial Sequence Synthetic polynucleotide
atgggtagtatagaggaggtgcttttggaggagaggctcataggatatctagaccccggagccgagaaagttttagcgaggattaacaggccttcaaaaattgtgtctacaagcagttgtacagggaggattacactgattgagggcgaggctcactggctcaggaacggggcaagagtagcgtacaagacccatcaccccatttcccggagtgaggttgaaagggttctaaggaggggcttcacaaacctttggctcaaggtgaccggccctattctacatctcagggttgaggggtggcagtgtgcaaagtcccttctcgaggcagctaggagaaacgggttcaagcacagcggagtcattagcattgctgaggattcaagactcgtcattgaaattatgagcagccagagcatgtcagtacctctagttatggagggtgctaggattgtcggcgacgatgccctagatatgctgattgagaaagcaaacactattctagttgagtctagaattgggctagacacgttttcaagagaggtcgaagagcttgtcgaatgcttt

SEQ ID NO: 105 - DNA, 765 bp, Artificial Sequence
  Description of Artificial Sequence Synthetic polynucleotide
atgaaacaatcgttacgccatcaaaaaattattaaactggtggagcagtctggctatttaagcacggaggagttggttgctgccttagacgttagccctcagacgatccgccgcgacttgaatatcttggcggagttagacttaatccgccgccaccacggtggtgcggcatcgccatcttctgcagagaattctgactacgtggaccgcaaacagttcttttcattacagaaaaataatatcgcacaggaggttgcgaagttgatccctaacggtgcatcgttgtttatcgacatcggtacgacgccggaggctgtcgccaatgcgttgcttggtcacgagaaactcagaatcgtgacgaacaatctgaatgccgctcaccttttacgccagaatgagagttttgacatcgtcatggcgggcggatcattacgaatggacggtggaatcatcggcgaggctacggtaaattttatctctcagtttcgcctagacttcggtatcttagggatcagtgcgatcgacgcagacggttcattattggactatgactaccacgaggtacaggtaaaacgagcgatcatcgagagttcacgccagaccttattagtggccgaccactctaaatttactcgccaggcgatcgttcgcttgggcgagttaagtgacgtggagtatttgtttacaggtgacgttcctgagggcatcgtcaattatttgaaagagcagaaaacgaaattggttttatgtaatggtaaagtgcgg

SEQ ID NO: 106 - DNA, 765 bp, Artificial Sequence
  Description of Artificial Sequence Synthetic polynucleotide
atgaaacaatcgttacgccatcaaaaaattattaaactggtggaacaatctggctatttaagcacggaagaattggttgctgccttagatgttagccctcaaacgatccgtcgtgatttgaatatcttggcggagttagatttaatccgccgccatcacggtggtgcggcatcgccatcttctgcagaaaattctgattacgtggatcgtaaacaattcttttcattacaaaaaaataatatcgcacaagaagttgcgaagttgatccctaacggtgcatcgttgtttatcgatatcggtacgacgccggaggctgtcgccaatgcgttgcttggtcatgaaaaactcagaatcgtgacgaacaatctgaatgccgctcatcttttacgccaaaatgaaagttttgatatcgtcatggcgggcggatcattacgaatggatggtggaatcatcggcgaagctacggtaaattttatctctcaatttcgcctagatttcggtatcttagggatcagtgcgatcgatgcagatggttcattattggattatgattaccatgaagtacaagtaaaacgagcgatcatcgaaagttcacgtcagaccttattagtggccgatcactctaaatttactcgccaagcgatcgttcgcttgggcgaattaagtgatgtggaatatttgtttacaggtgatgttcctgagggcatcgtcaattatttgaaagagcagaaaacgaaattggttttatgtaatggtaaagtgcgg

SEQ ID NO: 107 - DNA, 546 bp, Artificial Sequence
  Description of Artificial Sequence Synthetic polynucleotide
atgattaaatatagtatccgtggtgaaaacctagaagtaacagaggcaatccgcgactatgtagtttctaaactcgagaagatcgagaagtacttccagccagagcaggagttggacgcccgaatcaacttaaaagtttatcgcgagaaaacggctaaagtggaggtaacgatcccgcttggatctatcactctccgcgcagaggacgtatctcaggacatgtatggttcaatcgaccttgtaactgacaaaatcgagcgccagatccgcaaaaataaaacaaaaatcgagcgcaaaaataaaaataaggtagcaactggtcagttatttacagacgctttggtggaggactcaaatatcgtccagtctaaagttgttcgctcaaaacagatcgacttaaaaccaatggacttggaggaggcaatcctacagatggacttattggggcacgacttctttatctatgtggacgttgaggaccagacaaccaatgtgatctatcgccgcgaggacggcgagatcggtttgttagaggttaaagagtct

SEQ ID NO: 108 - DNA, 546 bp, Artificial Sequence
  Description of Artificial Sequence Synthetic polynucleotide
atgattaaatatagtatccgtggtgaaaacctagaagtaacagaagcaatccgtgattatgtagtttctaaactcgaaaagatcgaaaagtacttccaaccagaacaagagttggatgcccgaatcaacttaaaagtttatcgtgaaaaaacggctaaagtggaagtaacgatcccgcttggatctatcactctccgcgcagaagatgtatctcaagatatgtatggttcaatcgaccttgtaactgataaaatcgaacgtcagatccgtaaaaataaaacaaaaatcgagcgtaaaaataaaaataaggtagcaactggtcaattatttacagatgctttggtggaagattcaaatatcgtccagtctaaagttgttcgttcaaaacaaatcgatttaaaaccaatggatttggaagaagcaatcctacaaatggatttattggggcatgatttctttatctatgtggatgttgaagatcagacaaccaatgtgatctatcgtcgtgaggatggcgaaatcggtttgttagaggttaaagaatct

(19) United States
(12) Patent Application Publication
(10) Pub. No.: US 2016/0186188 A1
(43) Pub. Date: Jun. 30, 2016

(54) METHODS FOR ALTERING POLYPEPTIDE EXPRESSION AND SOLUBILITY

(71) Applicants: John F. Hunt, III, New York, NY (US); William Nicholson Price, II, Barnesville, MD (US); Gaetano T. Montelione, Highland Park, NJ (US); Gregory P. Boel, New York, NY (US); Thomas Acton, Highland Park, NJ (US); Helen Neely, Brooklyn, NY (US)

(72) Inventors: John F. Hunt, III, New York, NY (US); William Nicholson Price, II, Barnesville, MD (US); Gaetano T. Montelione, Highland Park, NJ (US); Gregory P. Boel, New York, NY (US); Thomas Acton, Highland Park, NJ (US); Helen Neely, Brooklyn, NY (US)

(73) Assignees: RUTGERS, THE STATE UNIVERSITY OF NEW JERSEY, New Brunswick, NJ (US); THE TRUSTEES OF COLUMBIA UNIVERSITY IN THE CITY OF NEW YORK, New York, NY (US)

(21) Appl. No.: 13/578,236

(22) PCT Filed: Feb. 9, 2011
(86) PCT No.: PCT/US11/24251

§ 371 Date: Feb. 6, 2014

Related U.S. Application Data
(60) Provisional application No. 61/302,805, filed on Feb. 9, 2010.

(51) Int. Cl. C12N 15/67

(52) U.S. Cl. CPC C12N 15/67

(57) ABSTRACT

The invention is directed to methods and metric suitable for use in determining the solubility, expression and usability of a polypeptide encoded by a nucleic acid sequence. In certain aspects, the invention also relates to methods for introducing modifications in a polypeptide, for example through substitution of one or more codons in the nucleic acid sequence encoding the polypeptide, to increase or decrease the solubility, expression or usability of the polypeptide.

This application claims the benefit of the filing date of U.S. Provisional Patent Application No. 61/302,805, filed Feb. 9, 2010, the contents of which are hereby incorporated by reference in its entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

Overexpression of recombinant polypeptides is a central method in contemporary biochemistry, structural biology, and biotechnology. Many recombinant polypeptides express at low levels or not at all when produced in expression systems. Moreover, polypeptides which express at high levels can form inclusion bodies which cannot be used without applying technically challenging refolding procedures (Makrides (1996) Microbiology and Molecular Biology Reviews 60:512). Industrial applications, such as drug discovery and vaccine preparation, frequently require that large amounts of soluble polypeptide be prepared. Many types of expression systems can be used to synthesize proteins, including mammalian, fungal and bacterial expression systems. However, over-expression of a target recombinant polypeptide can result in the formation of insoluble polypeptide aggregates both before or after steps are undertaken to purify the polypeptide. This inherent limitation to recombinant polypeptide expression presents a problem for the use of such systems where the goal of an expression strategy is to useful yields of a given recombinant polypeptide.

Despite the existence of experimental (Makrides (1996) Microbiology and Molecular Biology Reviews 60:512; Sorensen and Mortensen (2005) Journal of biotechnology 115:113-128; Davis et al. (1999) Biotechnology and bioengineering 65; Trevino et al, (2007) J. Mol. Biol 366:449-460; Yadava and Ockenhouse (2003) Infection and immunity 71:4961-4969; Kudla et al. (2009) Science 324:255) and computational (Wilkinson and Harrison (1991) Nature Biotechnology 9:443-448; Idicula-Thomas and Balaji (2005) Polypeptide Science: A Publication of the Polypeptide Society 14:582; Idicula-Thomas et al. (2006) Bioinformatics 22:278-284; Smialowski et al. (2007) Bioinformatics 23:2536; Magnan et al. (2009) Bioinformatics; Tartaglia et al. (2009) Journal of Molecular Biology.) methods for addressing this variability, the physiochemical parameters and processes that influence polypeptide expression and solubility remain poorly understood and the expression of recombinant polypeptides remains a significant experimental challenge (Makrides (1996) Microbiology and Molecular Biology Reviews 60:512; Sorensen and Mortensen (2005) Journal of Biotechnology 115:113-128; Christen et al. (2009) Polypeptide Expression and Purification). There is a need for methods for identifying polypeptides that have a high probability of being expressed at high soluble levels in cellular expression systems. There is also a need for methods suitable for increasing the expression of a polypeptide encoded by a nucleic acid and for increasing the solubility of such polypeptides. This invention addresses these needs.

### SUMMARY OF THE INVENTION

In one aspect, the invention described herein relates to a method for increasing the solubility of a recombinant polypeptide produced from a nucleic acid in an expression system, the method comprising replacing one or more solubility decreasing codons in the nucleotide sequence encoding the recombinant polypeptide with a synonymous solubility increasing codon. In another aspect, the invention described herein relates to a method for decreasing the solubility of a recombinant polypeptide produced from a nucleic acid in an expression system, the method comprising replacing one or more solubility increasing codons in the nucleotide sequence encoding the recombinant polypeptide with a synonymous solubility decreasing codon. In still another aspect, the invention described herein relates to a method for increasing the expression of a recombinant polypeptide produced from a nucleic acid in an expression system, the method comprising replacing one or more expression decreasing codons in the nucleotide sequence encoding the recombinant polypeptide with a synonymous expression increasing codon. In yet another aspect, the invention described herein relates to a method for decreasing the expression of a recombinant polypeptide produced from a nucleic acid in an expression system, the method comprising replacing one or more expression increasing codons in the nucleotide sequence encoding the recombinant polypeptide with a synonymous expression decreasing codon.

In one embodiment, the solubility decreasing codon is ATA (Ile) and the solubility increasing codon is ATT (Ile). In another embodiment, the solubility decreasing codon is ATC (Ile) and the solubility increasing codon is ATT (Ile). In another embodiment, the solubility decreasing codon is ATC (Ile) and the solubility increasing codon is ATT (Ile). In another embodiment, the solubility decreasing codon is any of AGA (Arg), AGG (Arg), CGA (Arg), or CGC (Arg) and the solubility increasing codon is CTG (Arg). In another embodiment, the solubility decreasing codon is GGG (Gly) and the solubility increasing codon is GGT (Gly). In another embodiment, the solubility decreasing codon is GTG (Val) and the solubility increasing codon is GTT (Val). In another embodiment, the expression decreasing codon is GAG (Glu) and the expression increasing codon is GAA (Glu). In another embodiment, the expression decreasing codon is GAC (Asp) and the expression increasing codon is GAT (Asp). In another embodiment, the expression decreasing codon is CAC (His) and the expression increasing codon is CAT (His). In another embodiment, the expression decreasing codon is CAG (Gln) and the expression increasing codon is CAA (Gln). In another embodiment, the expression decreasing codon is any of AGA (Asn), AGG (Asn), CGT (Asn), CGC (Asn), or CGG (Asn) and the expression increasing codon is CGA (Asn). In another embodiment, the expression decreasing codon is GGG (Gly) and the expression increasing codon is GGT (Gly). In another embodiment, the expression decreasing codon is TTC (Phe) and the expression increasing codon is TTT (Phe). In another embodiment, the expression decreasing codon is CCC (Pro) or CCG (Pro) and the expression increasing codon is CCT (Pro). In another embodiment, the expression decreasing codon is TCC (Ser) or TCG (Ser) and the expression increasing codon is AGT (Ser).

In one aspect, the invention described herein relates to a method for increasing the solubility of a recombinant polypeptide produced from a nucleic acid in an expression system, the method comprising replacing one or more solubility decreasing codons in the nucleotide sequence encoding the recombinant polypeptide with a non-synonymous solubility increasing codon. In another aspect, the invention described herein relates to a method for decreasing the solubility of a recombinant polypeptide produced from a nucleic acid in an expression system, the method comprising replacing one or more solubility increasing codons in the nucleotide sequence encoding the recombinant polypeptide with a non-synonymous solubility decreasing codon. In yet another aspect, the invention described herein relates to a method for increasing the expression of a recombinant polypeptide produced from a nucleic acid in an expression system, the method comprising replacing one or more expression decreasing codons in the nucleotide sequence encoding the recombinant polypeptide with a non-synonymous expression increasing codon. In still another aspect, the invention described herein relates to a method for decreasing the expression of a recombinant polypeptide produced from a nucleic acid in an expression system, the method comprising replacing one or more expression increasing codons in the nucleotide sequence encoding the recombinant polypeptide with a non-synonymous expression decreasing codon. In one embodiment, the solubility decreasing codon is any of TTA (Leu), TTG (Leu), CTT (Leu), CTC (Leu), CTA (Leu), CTG (Leu) and the solubility increasing codon is ATT (Ile). In another embodiment, the expression decreasing codon is any of TTA (Leu), TTG (Leu), CTT (Leu), CTC (Leu), CTA (Leu), CTG (Leu) and the expression increasing codon is ATT (Ile).

In one aspect, the invention described herein relates to a method for increasing the solubility of a recombinant polypeptide produced in an expression system, the method comprising replacing one or more solubility decreasing amino acid residues in the recombinant polypeptide with a solubility increasing amino acid residue. In another aspect, the invention described herein relates to a method for decreasing the solubility of a recombinant polypeptide produced in an expression system, the method comprising replacing one or more solubility increasing amino acid residues in the recombinant polypeptide with a solubility decreasing amino acid residue.

In one embodiment, the solubility decreasing amino acid is arginine and the solubility increasing amino acid is lysine. In another embodiment, the solubility decreasing amino acid is valine and the solubility increasing amino acid is isoleucine. In another embodiment, the solubility decreasing amino acid is leucine and the solubility increasing amino acid is valine. In another embodiment, the solubility decreasing amino acid is leucine and the solubility increasing amino acid is isoleucine. In another embodiment, the solubility decreasing amino acid is phenylalanine and the solubility increasing amino acid is valine. In another embodiment, the solubility decreasing amino acid is phenylalanine and the solubility increasing amino acid is isoleucine. In another embodiment, the solubility decreasing amino acid is cysteine and the solubility increasing amino acid is phenylalanine. In another embodiment, the solubility decreasing amino acid is cysteine and the solubility increasing amino acid is valine. In another embodiment, the solubility decreasing amino acid is cysteine and the solubility increasing amino acid is isoleucine. In another embodiment, the solubility decreasing amino acid is histidine and the solubility increasing amino acid is threonine. In another embodiment, the solubility decreasing amino acid is proline and the solubility increasing amino acid is valine.

In one aspect, the invention described herein relates to a method for increasing the expression of a recombinant polypeptide produced in an expression system, the method comprising replacing one or more expression decreasing amino acid residues in the recombinant polypeptide with an expression increasing amino acid residue. In another aspect, the invention described herein relates to a method for decreasing the expression of a recombinant polypeptide produced in an expression system, the method comprising replacing one or more expression increasing amino acid residues in the recombinant polypeptide with an expression decreasing amino acid residue.

In one embodiment, the expression decreasing amino acid is arginine and the expression increasing amino acid is lysine. In another embodiment, the expression decreasing amino acid is valine and the expression increasing amino acid is isoleucine. In another embodiment, the expression decreasing amino acid is leucine and the expression increasing amino acid is valine. In another embodiment, the expression decreasing amino acid is leucine and the expression increasing amino acid is isoleucine. In another embodiment, the expression decreasing amino acid is cysteine and the expression increasing amino acid is phenylalanine. In another embodiment, the expression decreasing amino acid is alanine and the expression increasing amino acid is methionine. In another embodiment, the expression decreasing amino acid is alanine and the expression increasing amino acid is cysteine. In another embodiment, the expression decreasing amino acid is alanine and the expression increasing amino acid is phenylalanine. In another embodiment, the expression decreasing amino acid is alanine and the expression increasing amino acid is leucine. In another embodiment, the expression decreasing amino acid is alanine and the expression increasing amino acid is valine. In another embodiment, the expression decreasing amino acid is alanine and the expression increasing amino acid is isoleucine. In another embodiment, the expression decreasing amino acid is tryptophan and the expression increasing amino acid is methionine. In another embodiment, the expression decreasing amino acid is arginine and the expression increasing amino acid is isoleucine. In another embodiment, the expression decreasing amino acid is arginine and the expression increasing amino acid is glutamic acid. In another embodiment, the expression decreasing amino acid is arginine and the expression increasing amino acid is aspartic acid. In another embodiment, the expression decreasing amino acid is lysine and the expression increasing amino acid is glutamic acid. In another embodiment, the expression decreasing amino acid is lysine and the expression increasing amino acid is aspartic acid.

In one aspect, the invention described herein relates to a method for increasing the solubility of a recombinant polypeptide produced in an expression system, the method comprising replacing a first type of amino acid at one or more positions in the recombinant polypeptide with a second type of amino acid residue, wherein the second amino acid residue has a greater or equivalent hydrophobicity and a greater solubility predictive value as compared to the first type of amino acid. In another aspect, the invention described herein relates to a method for increasing the expression of a recombinant polypeptide produced in an expression system, the method comprising replacing a first type of amino acid at one or more positions in the recombinant polypeptide with a second type of amino acid residue, wherein the second amino acid residue has a greater expression predictive value as compared to the first amino acid. In one embodiment, the second amino acid residue has a greater or equivalent hydrophobicity compared to the first amino acid. In still another aspect, the invention described herein relates to a method for decreasing the solubility of a recombinant polypeptide produced in an expression system, the method comprising replacing a first type of amino acid at one or more positions in the recombinant polypeptide with a second type of amino acid residue, wherein the second amino acid residue has a greater or equivalent hydrophilicity and a lesser solubility predictive value as compared to the first amino acid. In yet another aspect, the invention described herein relates to a method for decreasing the expression of a recombinant polypeptide produced in an expression system, the method comprising replacing a first type of amino acid at one or more positions in the recombinant polypeptide with a second type of amino acid residue, wherein the second amino acid residue has a lesser expression predictive value as compared to the first amino acid. In one embodiment, the second amino acid residue has a greater or equivalent hydrophobicity compared to the first amino acid.

In one embodiment, the expression system in an in vitro expression system. In another embodiment, the in vitro expression system is a cell-free transcription/translation system. In still another embodiment, the expression system in an in vivo expression system. In yet another embodiment, the in vivo expression system is a bacterial expression system or a eukaryotic expression system. In another embodiment, the in vivo expression system is an E. coli cell. In still another embodiment, the in vivo expression system is a mammalian cell.

In one embodiment, the recombinant polypeptide is a human polypeptide, or a fragment thereof. In another embodiment, the recombinant polypeptide is a viral polypeptide, or a fragment thereof. In another embodiment, the recombinant polypeptide is an antibody, an antibody fragment, an antibody derivative, a diabody, a tribody, a tetrabody, an antibody dimer, an antibody trimer or a minibody. In still another embodiment, the antibody fragment is a Fab fragment, a Fab′ fragment, a F(ab)2 fragment, a Fd fragment, a Fv fragment, or a ScFv fragment. In yet another embodiment, the recombinant polypeptide is a cytokine, an inflammatory molecule, a growth factor, a cytokine receptor, an inflammatory molecule receptor, a growth factor receptor, an oncogene product, or any fragment thereof. In another still embodiment, the recombinant polypeptide is a fusion polypeptide. In one aspect, the invention described herein relates to a recombinant polypeptide produced by the methods described herein. In one aspect, the invention described herein relates to a pharmaceutical composition comprising the recombinant polypeptide produced by the methods described herein. In one aspect, the invention described herein relates to an immunogenic composition comprising the recombinant polypeptide produced by the methods described herein.

In another aspect, the invention described herein relates to a method for predicting whether first polypeptide encoded by a first nucleic acid sequence will have greater solubility than a second polypeptide encoded by a second nucleic acid sequence when expressed in an expression system, the method comprising, a) calculating a value for one or more sequence parameters of the first nucleic acid sequence, b) calculating a value for one or more sequence parameters of the second nucleic acid sequence, c) multiplying the value for each sequence parameter in step (a) by the solubility regression slope of the sequence parameter to determine a combined solubility value for the sequence parameter of the first nucleic acid sequence, d) multiplying the value for each sequence parameter in step (b) by the solubility regression slope of the sequence parameter to determine a combined solubility value for the sequence parameter of the second nucleic acid sequence, e) comparing the combined solubility value for the sequence parameter of the first nucleic acid sequence to the combined solubility value for the sequence parameter of the second nucleic acid sequence, wherein a greater combined solubility value for the sequence parameter of the first nucleic acid sequence as compared to the combined solubility value for the sequence parameter of the second nucleic acid sequence indicates that first polypeptide will have greater solubility than a second polypeptide when expressed in an expression system.

In one aspect, the invention described herein relates to a method for predicting whether first polypeptide encoded by a first nucleic acid sequence will have greater expression than a second polypeptide encoded by a second nucleic acid sequence when expressed in an expression system, the method comprising, a) calculating a value for one or more sequence parameters of the first nucleic acid sequence, b) calculating a value for one or more sequence parameters of the second nucleic acid sequence, c) multiplying the value for each sequence parameter in step (a) by the expression regression slope of the sequence parameter to determine a combined expression value for the sequence parameter of the first nucleic acid sequence, d) multiplying the value for each sequence parameter in step (b) by the expression regression slope of the sequence parameter to determine a combined expression value for the sequence parameter of the second nucleic acid sequence, e) comparing the combined expression value for the sequence parameter of the first nucleic acid sequence to the combined expression value for the sequence parameter of the second nucleic acid sequence, wherein a greater combined expression value for the sequence parameter of the first nucleic acid sequence as compared to the combined expression value for the sequence parameter of the second nucleic acid sequence indicates that first polypeptide will have greater expression than a second polypeptide when expressed in an expression system.

In another aspect, the invention described herein relates to a method for predicting whether first polypeptide encoded by a first nucleic acid sequence will have greater usability than a second polypeptide encoded by a second nucleic acid sequence when expressed in an expression system, the method comprising, a) calculating a value for one or more sequence parameters of the first nucleic acid sequence, b) calculating a value for one or more sequence parameters of the second nucleic acid sequence, c) multiplying the value for each sequence parameter in step (a) by the usability regression slope of the sequence parameter to determine a combined usability value for the sequence parameter of the first nucleic acid sequence, d) multiplying the value for each sequence parameter in step (b) by the usability regression slope of the sequence parameter to determine a combined usability value for the sequence parameter of the second nucleic acid sequence, e) comparing the combined usability value for the sequence parameter of the first nucleic acid sequence to the combined usability value for the sequence parameter of the second nucleic acid sequence, wherein a greater combined usability value for the sequence parameter of the first nucleic acid sequence as compared to the combined usability value for the sequence parameter of the second nucleic acid sequence indicates that first polypeptide will have greater usability than a second polypeptide when expressed in an expression system.

In one embodiment, the sequence parameters in step (b) and step (c) are the same.

In one embodiment, the one or more sequence parameter is selected from the group comprising the fraction of amino acid residues in the polypeptide that are predicted to be disordered; the surface exposure and/or burial status of each residue in the polypeptide; the fractional content of the polypeptide made up by each amino acid; the fractional content of the polypeptide made up by each amino acid predicted to be buried or exposed; the fractional content of the polypeptide made up by each codon; the length of the polypeptide chain; the net charge of the polypeptide; the absolute value of the net charge of the polypeptide; the value for the net charge of the polypeptide divided by the length of the polypeptide; the absolute value of the net charge of the polypeptide divided by the length of the polypeptide; the isoelectric point of the polypeptide; the mean side-chain entropy of the polypeptide; the mean side-chain entropy of all residues predicted to be surface-exposed; and the mean hydrophobicity of the polypeptide. In another embodiment, the one or more sequence parameter is the fractional content of the polypeptide made up by rare codons. In one embodiment, the rare codons are selected from the group comprising AGG(Arg), AGA(Arg), CGG(Arg), CGA(Arg), ATA(Ile), CTA(Leu), and CCC(Pro).

### BRIEF DESCRIPTION OF THE FIGURES

FIG. 1. Distribution of polypeptides by expression and solubility scores. 9,877 polypeptides from the NESG polypeptide production pipeline were independently scored for expression (0-5) and solubility (0-5). FIG. 1A shows the distribution of polypeptides by expression score. FIG. 1B shows the distribution of polypeptides with at least minimal expression by solubility score. FIG. 1C shows a bubble plot of polypeptides by expression and solubility scores. The area of each point is proportional to the number of polypeptides with those expression and solubility scores. 3,880 polypeptides were considered useable for future work, defined as (Expression Score)*(Solubility Score)>11.

FIG. 2. Effects of amino acids and compound parameters on expression and solubility. 9,644 polypeptides from the NESG polypeptide production pipeline were independently scored for expression (E: 0-5) and solubility (S: 0-5), as measured by the size of the overexpressed polypeptide band in SDS-PAGE gels and by proportion of expressed polypeptide appearing in the soluble fraction. Ordinal logistic regressions were calculated between sequence parameters and scores for expression (E: 0-5, N=7733) and solubility (S: 0-5, N=6046, since only polypeptides with E>0 were analyzed). Signed −log(p) is shown for parameters, arranged by their effect on expression and separated into amino acids and compound parameters. A Bonferroni-corrected significance threshold of 0.0015 is indicated by the dotted line. *—The negative effect of net charge is a combination of a positive effect from negatively charged amino acids and a negative effect from positively charged amino acids (see FIG. 4).

FIG. 3. Sample score distributions. Polypeptides with different expression and solubility scores have significantly different distributions of sequence parameters. Distributions of (FIG. 3A) fractional Glu content (p=5.08×10⁻²⁶, N=7,733) and (FIG. 3B) net charge (p=7.32×10⁻³⁴, N=7,733) are shown for polypeptides with each expression score (0-5). FIG. 3C shows the distribution of the fraction of charged residues is shown for polypeptides with each solubility score (0-5) among polypeptides with expression scores above 0 (p=3.76×10⁻³⁹, N=6,046).

FIG. 4. Charge and pI effects. Because net charge is a signed variable, it was disaggregated into two subvariables: net positive charge, defined as net charge if net charge is positive and otherwise zero, and net negative charge, analogously. All variables were divided by chain length to yield fractional variables. Single logistic regressions were calculated for each variable against usability (E*S>11), expression, solubility, and the expression/solubility permissive and enhancement variables; the signed −log(p) values for those regressions, which show effect sign, magnitude, and significance for similarly distributed parameters, are shown (FIG. 4A). Net negative charge has uniformly positive effects on expression and solubility. Net positive charge has negative effects on expression and mixed effects on solubility, probably due to an interrelated rare-codon Arg effect; the effect of net positive charge becomes significantly positive (p=0.00004) when regressed against solubility alongside rare codon and common codon-encoded Arg. Polypeptide isoelectric point, on the other hand, only impacts expression, solubility, or usability at the extremes. FIG. 4B shows the mean expression and solubility scores and the fraction of usable targets for all pI bins, with 95% confidence intervals. For the vast majority of polypeptides between pI's of 4 and 11, pI has essentially no effect on either expression or solubility.

FIG. 5. Effects of rare codons. Four amino acids are commonly considered to be a potential source or rare codon problems: Arg, Ile, Leu, and Pro. For these amino acids, separate analyses were performed for fraction of the amino acid encoded by rare codons and encoded by common codons. Codons considered rare were ATA (Ile), CTA (Leu), CCC (Pro), and AGG, AGA, CGG, and CGA (Arg), each except CCC representing less than 8% of the codons for the corresponding amino acid in the E. coli genome (Nakamura Y, et al. (2000) Nucleic Acids Res 28:292). These two variables were analyzed in double ordinal logistic regressions for their correlation with (FIG. 5A) expression and (FIG. 5B) solubility scores. Signed −log(p) values are shown for the results of these double regressions, as well as the single regression results for total fraction of the amino acid, for comparison. Rare codon-encoded Arg, Ile, and Pro all have significant negative effects on expression, and rare codon-encoded Arg and Pro also have significant negative effects on solubility. The negative expression effect of Leu appears to come entirely from common codons, probably because fewer than 7% of Leu residues are encoded by rare codons; this effect may be a proxy for Leu's influence on solubility.

FIG. 6. Hydrophobicity and predictive value for amino acids. Single logistic regressions were performed to evaluate the correlation between amino acid frequencies and either expression or solubility. The scatterplot above shows the absence of any strong relationship between residue hydrophobicity and its effect on either solubility or expression. Values for amino fractions are shown in solid squares; the ordinate shows the predictive value of the variable in regression, defined as the product of the regression slope and the parameter's standard deviation, which scales for differences in parameter prevalence and variability. Error bars indicate 95% confidence intervals. Amino acid hydrophobicity is not significantly correlated with amino acid predictive value for expression (p=0.098) or solubility (p=0.23). In addition to the amino acid fraction values, the four amino acids commonly considered to have rare codons were separated into fractions encoded by rare codons and common codons. These are shown as hollow triangles, pointed up for common codons and down for rare codons.

FIG. 7. Segregation of amino acid variables by predicted surface exposure. Amino acid content was divided into predicted buried and exposed fractions. Ordinal logistic regressions were calculated between all sequence parameters listed in Table 8 and scores for expression and solubility as described herein. Redundant variables (e.g., a [ala]=ae [exposed ala]+ab [buried ala]) were culled separately for expression and solubility as described in Methods. Signed −log(p) values are shown for the remaining parameters which correlated with either expression or solubility significantly, according to a Bonferroni-corrected p value of 0.0007. Separation by predicted solvent exposure increased predictive power for eight expression effects but only two solubility effects.

FIG. 8: Correlations between sequence parameters and usability. Logistic regressions were calculated between many sequence parameters and practical polypeptide usability, defined as (E*S>11). Signed −log(p) values for parameters significant in individual regressions at the Bonferroni-corrected p<0.0007 level are shown in light gray. A stepwise Akaike Information Criterion multiple logistic regression was calculated to determine statistically redundant signal; parameters remaining significant after this regression are shown in dark gray.

FIG. 9. Performance of a combined predictor of polypeptide usability. The significant factors remaining after stepwise AIC multiple regression were used to create a predictive metric, where Pr(E*S>11)=1/(1+exp(−θ)), and θ is a linear combination of the significant parameters. This metric models the development set closely up to a 65% probability of polypeptide usability (p=3.7×10-111, N=7733). The metric was tested on a set of 1911 polypeptides randomly held separate from the development set and predicts those polypeptides nearly as well (θ′=0.85*θ-0.06, p=6.8×10-16, N=1911). The graph shows model performance based on ten bins at equal intervals of 0.1. Squares represent the fraction of usable polypeptides in each bin and error bars represent 95% confidence limits calculated from counting statistics using the numbers in each bin.

FIG. 10. Performance of a combined predictor of polypeptide usability with rare codon effects included. For each of the four amino acids with rare codons (Arg, Ile, Leu, and Pro), the total fractional amino acid was replaced with rare and common codon-coded fractions in the initial predictive model; stepwise regression was performed as above (FIG. 3) to create a final predictive model. FIG. 10A shows model performance based on ten bins of equal size (773 polypeptides each for the development set, 191 for the test set), showing the expected and observed fractions of usable polypeptides in each bin. Error bars represent 95% confidence limits calculated from counting statistics using the numbers in each bin. FIG. 10B shows model performance for ten bins at equal intervals. The model describes the data somewhat better than the amino acid sequence based model without codon frequency information (p=9.2×10⁻¹³⁷); it also significantly performs well on the 1,911 test polypeptides withheld from the model development process (p=3.3×10⁻¹⁹).

FIG. 11A-D. Performance of combined predictors of polypeptide expression and solubility. Combined predictive metrics were developed for expression and solubility. Because the outcome of an ordinal logistic regression is a set of probabilities for each outcome, and not simply a single probability, the graphs do not show a single evaluative measure. Rather, for each metric, the relevant polypeptides were divided into 10 rank-ordered bins with equal numbers of polypeptides. Each bin therefore has an expected number of polypeptides at each score; the highest ranked bin has a high proportion of polypeptides expected to score 5, a lower expected number of 4's, and so on. The graph shows expected vs. observed percentages of polypeptides in each bin at each score (e.g., in expression bin 1, 60% of polypeptides were expected to score 5 for expression, and 58% did.) Each of the 10 bins has 6 data points, indicating the expected and observed percentage of polypeptides at each score. Bins are indicated by color, ranging from red (low) through green (medium) to violet and pink (high), and the score considered is indicated by the shape of the data point. All metrics very significantly describe the data, with the development correlations unsurprisingly higher than the test correlations (p_{EXP-DEV}=4.9×10⁻¹¹⁰, p_{EXP-TEST}=6.1×10⁻¹⁷, p_{SOL-DEV}=4.0×10⁻¹⁰⁹, p_{SOL-TEST}=7.4×10⁻¹⁵).

FIG. 12. Different parameter effects at the permissive vs. enhancement levels. Some parameters appear to function differently as gatekeepers or enhancers of expression or solubility. For each parameter, binary logistic regressions were calculated for correlation with the binary outcome of some vs. no expression or solubility (i.e., a score of 0 vs. a score above 0), and separately with the binary outcome of some vs. the most expression or solubility (i.e., a score below 5 vs. a score of 5). A Brant test (Brant R (1990) Biometrics 46:1171-1178) was used to determine whether the slopes were significantly different (i.e., whether the ordinal regression model violated the parallel proportional odds assumption); signed −log(p) values are shown for each significantly predictive parameter, sorted, by the significance of their Brant test. Dotted lines indicate statistical significance thresholds, of p<0.05 for individual Brant statistics, and p<0.0007 for Bonferroni-corrected single logistic regressions. FIG. 12A shows expression regressions. FIG. 12B shows solubility regressions.

FIG. 13. Opposing parameter effects on polypeptide expression/solubility and crystallization propensity. All factors which were analyzed in an earlier study of crystallization propensity (pXS) (Price W N et al. (2009) Nat. Biotechnol 27:51-57) were logistically regressed against usability (E*S>11; pES). The graph displays the predictive value for each parameter, defined as the product of the parameter standard deviation and the logistic regression slope. Predictive value is shown because the sample sizes differ by an order of magnitude (679 vs. 9,866), and therefore statistical-significance-based metrics are not directly comparable. Parameters significant at the indicated Bonferroni-corrected p-values in either analysis are shown; nearly every significant parameter has opposing influences on crystallization and expression/solubility.

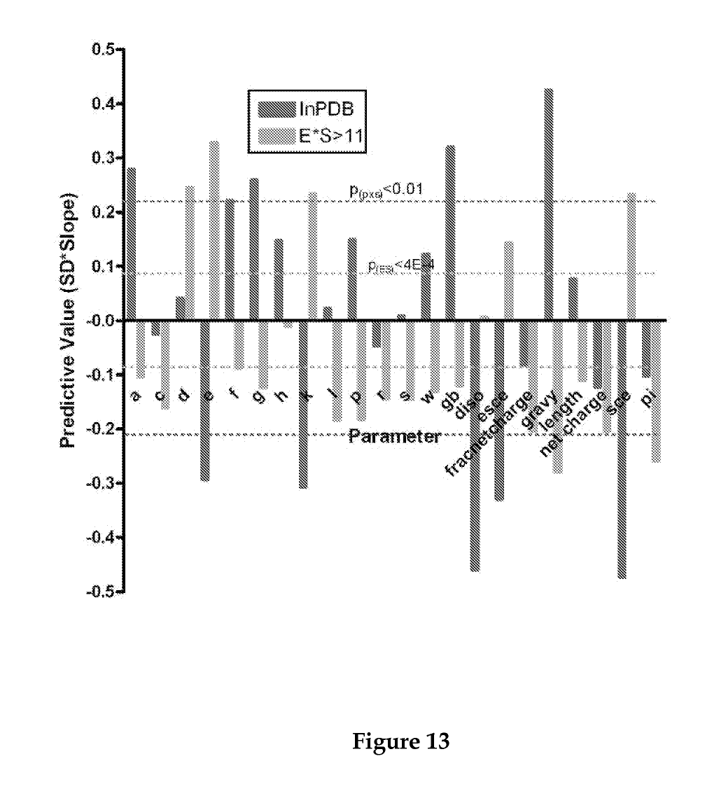

FIG. 14. Usability predictions and polypeptide structure solution. Polypeptides which proceeded completely through the pipeline to structure determination either by x-ray crystallography or nuclear magnetic resonance have significantly different predictive metric distributions than polypeptides which did not yield solved structures. FIG. 14A shows a scatterplot of polypeptides by probability of usability (p_{ES}) and probability of crystal structure solution (p_{XS}). Polypeptides which were not solved (NS) are shown in black (N=9,178), polypeptides with solved crystal structures (XS) are shown in red (N=354), and polypeptides with solved NMR structures (NMR) are shown in blue (N=251). FIG. 14B shows a scaled histogram of polypeptides by p_{ES}. The distributions are significantly different for NS vs. XS (p=6.9×10⁻¹³), NS vs. NMR (p=6.9×10⁻⁴³), and XS vs. NMR (p=6.1×10⁻¹⁵) (unpaired heteroskedastic T-test).

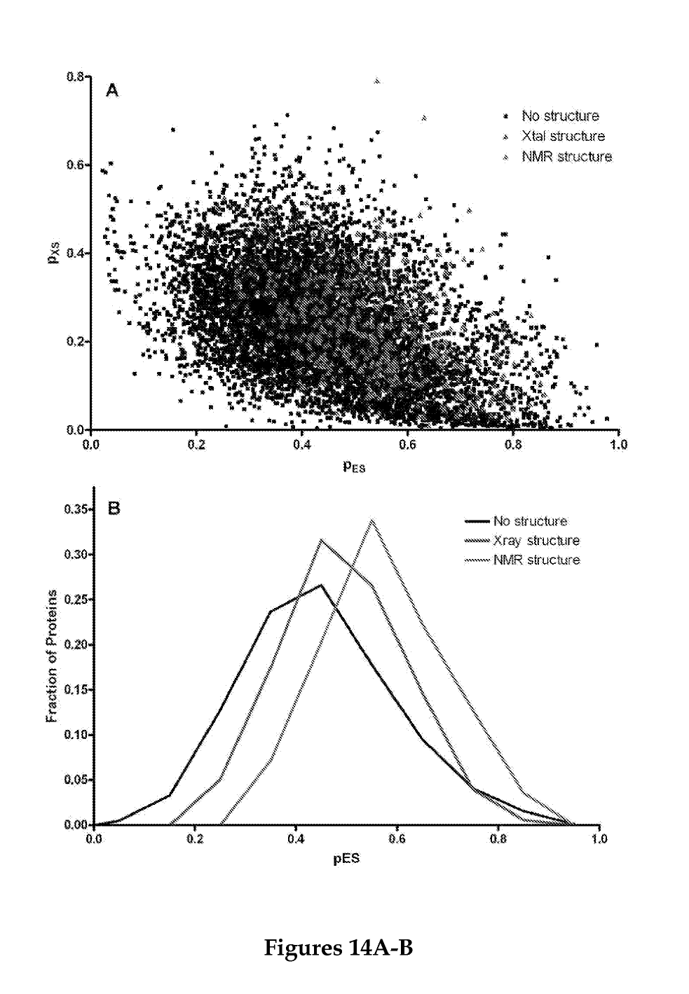

FIG. 15. Correlations between sequence parameters and NMR HSQC screening score. HSQC screening was performed on 982 expressed and soluble polypeptides. Spectra were scored as unfolded, poor, promising, good, or excellent. Scores of poor through excellent were converted to numerical scores and correlated with sequence parameters as in the analyses of expression, solubility, and usability presented herein. FIG. 15A shows the negative log p values for factors remaining after the initial parameter culling described in the methods, and the three parameters remaining after stepwise logistic regression. FIG. 15B shows metric predictive performance among 10 bins of polypeptides for each of the four score possibilities, and significantly classifies polypeptide groups (N=781, p=1.5×10⁻¹¹). FIG. 15C shows the metric's statistically marginal performance in a set of test polypeptides (N=201, p=0.07).

FIG. 16: Codons for the same amino acid have substantially different effects on both expression and solubility. In a set of 9,644 polypeptides expressed through the same NESG pipeline and systematically evaluated for expression and solubility, the frequencies of many codons showed significant correlations with expression (FIG. 16A) and solubility (FIG. 16B) when analyzed using ordinal logistic regression. Graphs show the predictive value, defined as the product of the regression slope and the variable standard deviation, for the amino acid frequency on the abscissa and the codon frequency on the ordinate. Bars indicate 95% confidence intervals, and one-letter amino acid codes are provided. Codon effects varied significantly within some amino acids, most notably in isoleucine and arginine, each of which had very broad differences between codons with positive and negative correlations; and the set of glutamine, histidine, aspartic acid and glutamic acid, each of which has two codons, with one significantly positively impacting expression, and one showing no statistically significant effect.

FIG. 17. Relationship between codon and tRNA frequency and expression/solubility effects. No significant relationship was observed between a codon's correlation with expression or solubility and either its genomic frequency (FIG. 17A) or the abundance of matching tRNA molecules (FIG. 17B) in E. coli. Data points show the predictive value of the codon, with bars indicating 95% confidence intervals.

FIG. 18. Codon GC content and effects on expression and solubility. The predictive value (Slope*SD) is shown for each codon grouped by the number of guanine or cysteine bases in the codon on expression (FIG. 18A) and solubility (FIG. 18B). Predictive values are also shown for codons grouped by whether the base in the wobble position is an A/T or a G/C (C,D). Finally, the average expression and solubility scores are shown for polypeptides binned by fraction GC, with error bars indicating 95% confidence intervals based on the numbers of polypeptides in the bin (FIG. 18E).

FIG. 19. Matching analyses to control for GC content and amino acid biochemical properties. To determine the effects of individual codons, it is necessary to control for the GC content of the codon (see FIG. 3) and the biochemical effect of the amino acid itself. Polypeptides were grouped into sets with matched distributions of the controlled parameter (either the relevant amino acid or GC content) but significant variation in the codon content. The expression and solubility score distributions for those matched sets was evaluated for statistical significance using a matched heteroskedastic T-test; results are shown for codon impact on expression (FIG. 19, Top Panel) and solubility (FIG. 19, Bottom Panel).

FIG. 20. Codon expression effects localized within the transcript. To determine whether codon effects were position specific, the each target transcript was divided into 50 codon sections (i.e., codons 1-50, codons 51-100, up to 300 codons, and then one category for codons after 300), and the fractional content of each codon was calculated for each section. These position-specific codon fractions were then regressed against expression score using ordinal logistic regression. The signed −log(p) for each regression is shown. Many negative codon effects are localized to the first 50 codons, indicating an effect on the initiation of translation, while many positive codon effects are localized to codons 51-200, indicating an effect on ongoing translational speed.

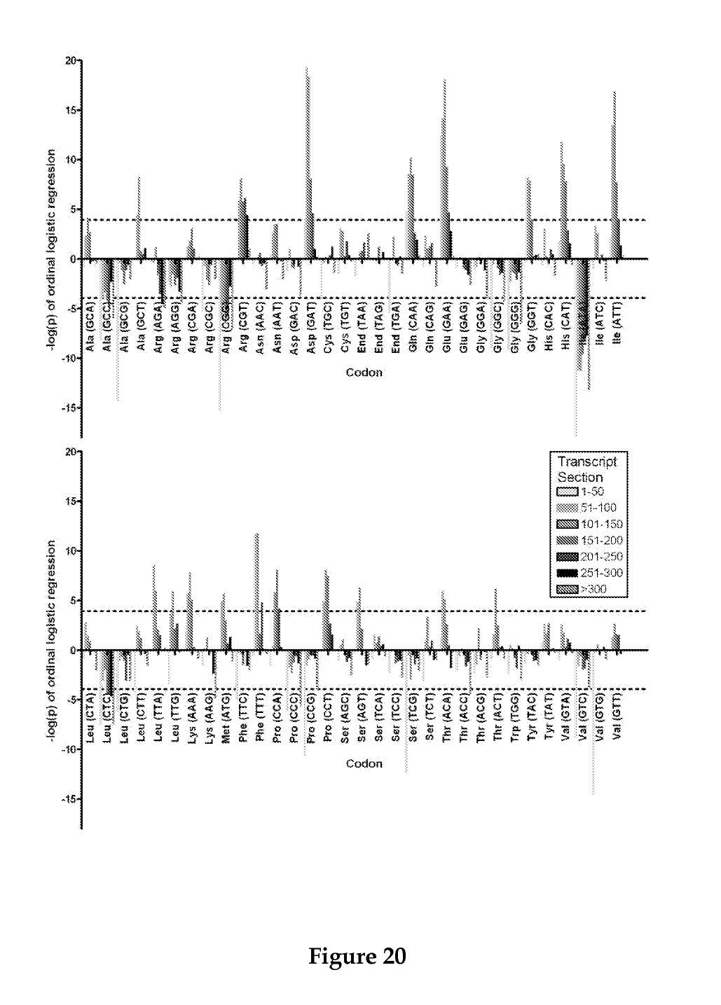

FIG. 21. Codon solubility effects localized within the transcript. To determine if codon effects were position specific, the each target transcript was divided into 50 codon sections (i.e., codons 1-50, codons 51-100, up to 300 codons, and then one category for codons after 300), and the fractional content of each codon was calculated for each section. These position-specific codon fractions were then regressed against solubility score using ordinal logistic regression. The signed −log(p) for each regression is shown.

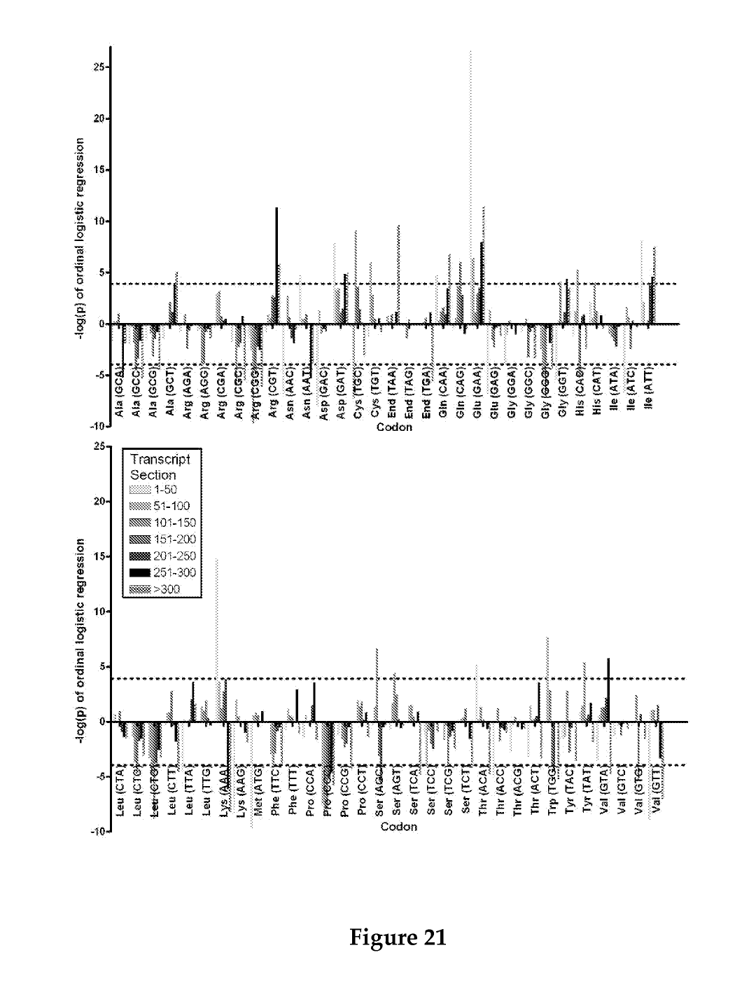

FIG. 22. Correlations between sequence parameters, expression, and solubility. Ordinal logistic regressions were calculated between sequence parameters and scores for expression (0-5, N=7733) and solubility (0-5, N=6046: only exp>0). Z scores are shown for parameters which correlated with either expression or solubility significantly, determined by a Bonferroni-corrected p value of 0.0007.

FIG. 23. Correlations between sequence parameters and usability. Logistic regressions were calculated between sequence parameters and practical polypeptide usability, defined as (E*S>11). Parameters significant in individual regressions at the p<0.0007 level are shown in light gray. A stepwise Akaike Information Criterion (Akaike, 1974) multiple logistic regression was calculated to determine statistically redundant signal; parameters remaining significant after this regression are shown in dark gray.

FIG. 24. Combined metric predicting usability: performance and validation. The significant factors remaining after stepwise AIC multiple regression were used to create a predictive metric, where prob(E*S>11)=1/(1+exp(−θ)), and θ is a linear combination of the significant parameters. This metric models the development set closely up to a 65% probability of polypeptide usability (p=3.7×10-111, N=7733). The metric was tested on a set of 1911 polypeptides randomly held separate from the development set; it predicts those polypeptides nearly as well (θ′=0.85*θ-0.06, p=6.8×10-16, N=1911).

FIG. 25. Opposing parameter influence on expression/solubility and crystallization. All factors which were analyzed in an earlier study of crystallization propensity (Price et al., 2009) were logistically regressed against usability (E*S>11). Parameters significant in either analysis are shown; nearly every significant parameter has opposing influences on crystallization and expression/solubility.

FIG. 26. Protein toxicity measure by cell growth. Cell growth during protein expression was monitored by measuring the cell density (OD600) over time. FIG. 26A shows that prior to codon optimization, cells expressing the wild-type protein (blue squares) do not grow as well as cells that were not-induced (red circles), indicating that protein expression was toxic to the host cell. FIG. 26B shows that expression of the codon optimized gene RR161-1.10 (blue squares) relieved toxicity and cells grew as well as cells that were not-induced (red circles). Error bars represent standard deviation of independent duplicate measurements.

FIG. 27. RR162 protein expression levels. Equivalent volumes of cell lysate were loaded in all lanes on an SDS-PAGE gel after cell lysis. Molecular weight markers were ran in the second lane and are labeled in kDa. The arrow represents the band corresponding to the expressed RR162 protein. Lane NI-WT.1 shows the proteins in the not-induced cell lysate. Lanes WT.1 and WT.2 are from two different cultures expressing RR162 prior to codon optimization. Lanes 1.3 and 1.10 represent protein expression of cells transformed with two fully codon optimized constructs. No improvement in protein expression is observed despite codon optimization.

FIG. 28. SrR141 protein toxicity measured by cell growth. Cell growth during protein expression was monitored by measuring the cell density (OD600) over time. FIG. 28A shows that prior to codon optimization, cells expressing the wild-type gene construct (blue squares) exhibit impaired growth over time compared to cells that were not-induced (red circles). FIG. 28B shows that expression of the codon optimized gene SrR141-1.16 (blue squares) relieved toxicity and cells grew as well as cells that were not-uninduced (red circles). Error bars represent standard deviation of duplicate independent measurements.

FIG. 29. SrR141 protein expression levels. Equivalent volumes of cell lysate were loaded in all lanes on an SDS-PAGE gel after cell lysis. Lane NI-WT.1 shows the cellular proteins in the not-induced cell lysate. Lanes WT.1 and WT.2 are from two different cultures expressing SrR141 prior to codon optimization. Lanes 1.16 and 1.17 represent protein expression of cells transformed with two fully codon optimized constructs. Molecular weight markers were ran in the first lane and are labeled in kDa. The arrows represent the band corresponding to the expressed SrR141 protein. SrR141 expression is low in all induced cell cultures.

FIG. 30. XR92 protein toxicity measured by cell growth. Cell growth during protein expression was monitored by measuring the cell density (OD600) over time. FIG. 30A shows that prior to codon optimization, cells expressing the wild-type protein (blue squares) exhibit impaired growth over time compared to cells that were not-induced (red circles). FIG. 30B shows that expression of the codon optimized gene XR92-1.9 (blue squares) partially relieved toxicity and cells grew as well as cells that were non-induced (red circles). Error bars represent standard deviation of independent duplicate measurements.

FIG. 31. XR92 protein expression levels. Equivalent volumes of cell lysate were loaded in all lanes on an SDS-PAGE gel after cell lysis. Molecular weight markers were ran in the first lane and are labeled in kDa. The arrow at 31 kDa represents the band corresponding to the expressed XR92 protein. Lanes WT1 and WT2 are from two different cultures expressing XR92 prior to codon optimization. No expression of XR92 is observed. Lanes 1.9 and 1.15 represent protein expression of cells transformed with two fully codon optimized constructs. Expression of XR92 is greatly improved.

FIG. 32. RhR13 protein toxicity measured by cell growth. Cell growth during protein expression was monitored by measuring the cell density (OD600) over time. FIG. 32A shows that prior to codon optimization, there is no difference in cell growth in the induced (blue squares) and not-induced (red circles) cultures, indicating that expression of RhR13 is not toxic to the host cell. FIG. 32B shows that expression of the codon optimized gene RhR13-1.4 (blue squares) had significant impact on cell growth compared to cells that were not-induced (red circles). Error bars represent standard deviation of duplicate independent measurements.

FIG. 33. RhR13 protein expression levels. Equivalent volumes of cell lysate were loaded in all lanes on an SDS-PAGE gel after cell lysis. Molecular weight markers were ran in the first lane and are labeled. The arrow at 18.5 kDa represents the band corresponding to the expressed RhR13 protein. Lane NI-WT.7 shows the cellular proteins in the not-induced cell lysate. Lanes WT.7 and WT.8 are from two different cultures expressing RhR13 prior to codon optimization. No significant expression of RhR13 is observed. Lanes 1.3 and 1.4 represent protein expression of cells transformed with two fully codon optimized constructs. Expression of RhR is greatly improved.

### DETAILED DESCRIPTION OF THE INVENTION

The issued patents, applications, and other publications that are cited herein are hereby incorporated by reference to the same extent as if each was specifically and individually indicated to be incorporated by reference.

Overexpression of recombinant polypeptides is an important step in a variety of biotechnology applications, however poor solubility and expression of recombinant polypeptides can be problematic for polypeptide related applications. For example, industrial and commercial applications such as food production, drug discovery and drug production often require preparation of soluble polypeptides and/or that the polypeptides be expressed at high levels. Methods to alter polypeptide solubility and expression without affecting the function are highly needed. The methods described herein are based in part on large scale data mining based algorithms suitable for targeted mutagenesis and codon selection to alter expression and/or solubility of a recombinant polypeptide. In certain aspects, the methods described herein can be used to substitute amino acids and codons according to the correlation of their effects on polypeptide expression and solubility. In one embodiment, the methods described herein are useful for altering the expression or solubility of a recombinant polypeptide without altering amino acid sequence of the polypeptide. In other embodiments, the methods described herein are useful for altering the expression or solubility of a recombinant polypeptide by making one or more conservative substitutions in the amino acid sequence of the polypeptide. In other embodiments, the methods described herein are useful for altering the expression or solubility of a recombinant polypeptide by making one or more amino acid substitutions in the amino acid sequence of the polypeptide.

The methods described herein are based on advances in understanding of the physiochemical properties influencing polypeptide expression and solubility obtained by statistical data mining from thousands of unique polypeptides expressed in an expression system. In one aspect, the methods described herein relate to a metric suitable for predicting the solubility, expression or usability of a polypeptide encoded by a nucleic acid sequence wherein logistic regression is used to determine the relationship between continuous independent variables in the nucleic acid sequence or the polypeptide sequence to ranked categorical dependent variables. The relationship between continuous independent variables and ranked categorical dependent variables can be determined by converting output variables into an odds ratio for each outcome and performing a linear regression against the logarithm of that parameter. The continuous independent variables (e.g. sequence parameters) subject to analysis can include the fractional content of each amino acid as well as a additional aggregate parameters, including, but not limited to the isoelectric point, polypeptide length, mean side chain entropy, GRAVY as well as electrostatic charge variables (see, for example Table 8). Accordingly, the methods described herein demonstrate that the solubility or expression of a polypeptide can depend on the presence or frequency or specific codons in the nucleic acid encoding the polypeptide. For example, the results described herein show that the presence and/or frequency of certain codons and amino acid residues have statistically positive effects on polypeptide solubility and/or expression when the polypeptide is produced in an expression system. Further, provided by the invention are methods for altering the expression or solubility properties of a polypeptide by substituting particular codons with other codon types within the in open reading frame of the nucleic acid sequence encoding the polypeptide. Surprisingly, the codon specific effects described herein can be independent on the abundance of cognate tRNAs in the expression system.

In certain aspects, the methods described herein relate to the finding that polypeptide hydrophobicity is not a dominant determinant of polypeptide solubility. In certain aspects, a correlation with hydrophobicity in the results described herein can be a surrogate for the beneficial effect of some charged amino acids. In another aspect, the methods described herein are related to the finding that amino acids with similar hydrophobicities can have divergent effects on polypeptide solubility. The basic physiochemical properties of proteins are invariant irrespective of the expression system in which they are produced. E. coli has served as a model system for characterizing basic cellular biochemistry for more than 50 years, and significant insight into the biochemistry of other organisms including humans derives from studies conducted in E. coli. Therefore, results obtained from the E. coli data mining studies described herein can also be applied to protein expression in any living cell or in ribosome-based in vitro translation systems.

In one aspect, the methods described herein relate methods altering the solubility of a recombinant polypeptide by altering one or more codons in a nucleic acid sequence with a solubility enhancing codon. In anther aspect, the methods described herein relate to methods for altering the expression of a recombinant polypeptide by altering one or more codons in a nucleic acid sequence with an expression enhancing codon. Described herein are methods for altering the yields of soluble recombinantly expressed polypeptides. Also described herein are methods for indentifying efficacious codons for improving expression and solubility of a polypeptide.

In other aspects, the methods described herein are based on the finding that arginine content of a polypeptide is correlated with decreased expression and solubility even in cases where one or more arginines in the polypeptide are encoded by common codons even though arginine is charged and among the least hydrophobic amino acids.

The singular forms “a,” “an,” and “the” include plural references unless the content clearly dictates otherwise. Thus, for example, reference to a “virus” includes a plurality of such viruses.

In some embodiments, recombinant polypeptides exist in solution in the cytoplasm of a host cell or in solution in an extracellular preparation of the recombinant polypeptide. In some embodiments, recombinant polypeptide exists in an insoluble form in a host cell (e.g. in inclusion bodies) or in an extracellular preparation of the recombinant polypeptide. An insoluble recombinant polypeptide found inside an inclusion body may be solubilized (i.e., rendered into a soluble form) by treating purified inclusion bodies with denaturants such as guanidine hydrochloride, urea or sodium dodecyl sulfate (SDS). A method of testing whether a polypeptide is soluble or insoluble is described in U.S. Pat. No. 5,919,665, which is incorporated by reference.

The solubility of polypeptides depends in part on the distribution of hydrophilic and hydrophobic amino acid residues on the surface of the polypeptide. Low solubility is correlated with polypeptides having a relatively high content of hydrophobic amino acids on their surfaces. Conversely, charged and polar surface residues interact with ionic groups in the solvent and are correlated with greater solubility. With respect to polypeptide expression, specific amino acid residues in a polypeptide chain are encoded by codons in a nucleic acid sequence encoding the polypeptide. There are 64 possible triplets encoding 20 amino acids, and three translation termination (nonsense) codons. Different organisms often show particular preferences for one of the several codons that encode the same amino acid. Further, proteins containing rare codons may be inefficiently expressed and that rare codons can cause premature termination of the synthesized polypeptide or misincorporation of amino acids. Like mammals, the genetic code of E. coli comprises redundant codons wherein a single amino acid within a polypeptide sequence can be encoded by more than one type of codon. For example, in the case of serine, the TCT, TCC, TCA and TCG codons are said to be synonymous because they can independently direct the addition of a serine residue in a polypeptide during polypeptide translation. Accordingly, altering a nucleic acid sequence such that one codon is replaced with a synonymous codon is termed a synonymous mutation or a silent mutation.

Polypeptides can aggregate and form inclusion bodies if improper folding occurs during polypeptide translation. This effect can be a significant problem a polypeptide from one organism is expressed in a second, divergent organism (e.g. expression of a human polypeptide in a bacterial cell). Polypeptide aggregation during recombinant expression can occur as a result of misfolding or of formation of specious interactions between proteins.

The invention described herein relates in part to methods for modifying a nucleotide sequence for enhanced expression and/or solubility of its polypeptide or polypeptide product when produced in an expression system. In addition, the methods also relate to methods for the design of synthetic genes, de novo, and for enhanced accumulation and solubility of its encoded polypeptide or the polypeptide product in a host cell.

The methods described herein are based in part on the finding that synonymous codons can have a differential effect on polypeptide expression and/or solubility of an encoded polypeptide. In one embodiment, the methods described herein can be useful for producing a polypeptide for commercial applications which include, but are not limited to the production of vaccines, pharmaceutically valuable recombinant polypeptides (e.g. growth factors, or other medically useful polypeptides), reagents that may enable advances in drug discovery research and basic proteomic research. Thus, the present invention is drawn to a method for modifying a nucleic acid sequence encoding a polypeptide to enhance accumulation and/or solubility of the polypeptide, the method comprising determining the amino acid sequence of the polypeptide encoded by a nucleic acid sequence and introducing one or more solubility and/or expression altering modifications in the nucleic acid sequence by substituting codons in the coding sequence with one or more solubility or expression altering codons which will code for the same amino acid.

In certain aspects, the methods described herein are based on the results of a large scale data mining study of polypeptides expressed under constant expression conditions, where it was found that several amino acids and codons, including some synonymous codons, have surprising and significant correlations with higher expression and solubility in E. coli and likely all other organisms. The finding that synonymous codons can have differential effects on the solubility and expression of a recombinant polypeptide produced in an expression system provides new opportunities for the production of scientifically, commercially, therapeutically and industrially relevant recombinant polypeptides. Such applications are described greater detail herein.

In one aspect, the present invention is directed to a nucleic acid encoding a recombinant polypeptide, such as for example an antigen or industrially useful polypeptide, that has been mutated to change one or more codons to a synonymous codon wherein the mutation is a solubility or expression altering modification. In another embodiment, the methods described herein are directed to methods of making such mutations. Such mutations may be made anywhere in the coding region of a nucleic acid including any portions of the encoded polypeptide that are subsequently modified or removed from the mature polypeptide. For example, in one embodiment, the solubility or expression altering modification is located in a region of the nucleic acid that corresponds to a portion of the polypeptide that is retained in the polypeptide upon post-translational modification. In another embodiment, the solubility or expression altering modification is located in a region of the nucleic acid that corresponds to a portion of the polypeptide that is not retained in the polypeptide upon post-translational modification (e.g. in a signal sequence peptide).

In one embodiment, the methods described herein can be used to design a modified gene comprising one or more expression and/or solubility altering modifications wherein the modification causes the greater expression of a polypeptide encoded by the gene or causes the polypeptide encoded by the gene to have altered solubility.

In embodiments where the solubility or expression altering modification in a coding region of a nucleic acid sequence, the solubility or expression altering modification can replace a codon sequence such that the modification does not alter the amino acid(s) encoded by the nucleic acid. For example, in the event that the solubility or expression increasing modification is a CTG codon, and the coding sequence being replaced by the mutation can be any of AGA, AGG, CGA, CGC or CGG codon, each of which also encode arginine. In the event that the solubility or expression increasing modification is a GCG codon, and the coding sequence being replaced by the mutation can be any of GCT, GCA, or GCC codon, each of which also encode alanine. In the event that the solubility or expression increasing modification is a GGG codon, and the coding sequence being replaced by the mutation can be any of GGT, GGA, or GGC codon, each of which also encode glycine. One of skill in the art can readily determine how to change one or more of the nucleotide positions within a codon without altering the amino acid(s) encoded, by referring to the genetic code, or to RNA or DNA codon tables. Canonical amino acids and their three letter and one-letter abbreviations are Alanine (Ala) A, Glutamine (Gln) Q, Leucine (Leu) L, Serine (Ser) S, Arginine (Arg) R, Glutamic Acid (Glu) E, Lysine (Lys) K, Threonine (Thr) T, Asparagine (Asn) N, Glycine (Gly) G, Methionine (Met) M, Tryptophan (Trp) W, Aspartic Acid (Asp) D, Histidine (His) H, Phenylalanine (Phe) F, Tyrosine (Tyr) Y, Cysteine (Cys) C, Isoleucine (Ile) I, Proline (Pro) P, Valine (Val) V

In some embodiments the solubility or expression altering modification may be a modification that does affect the amino acid sequence encoded by the nucleic acid sequence. Such mutations may result in one or more different amino acids being encoded, or may result in one or more amino acids being deleted or added to the amino acid sequence. If the solubility or expression altering modification does affect the amino acid(s) encoded, it is possible to make one of more amino acid changes that do not adversely affect the structure, function or immunogenicity of the polypeptide encoded. For example, the mutant polypeptide encoded by the mutant nucleic acid can have substantially the same structure and/or function and/or immunogenicity as the wild-type polypeptide. It is possible that some amino acid changes may lead to altered immunogenicity and artisans skilled in the art will recognize when such modifications are or are not appropriate.

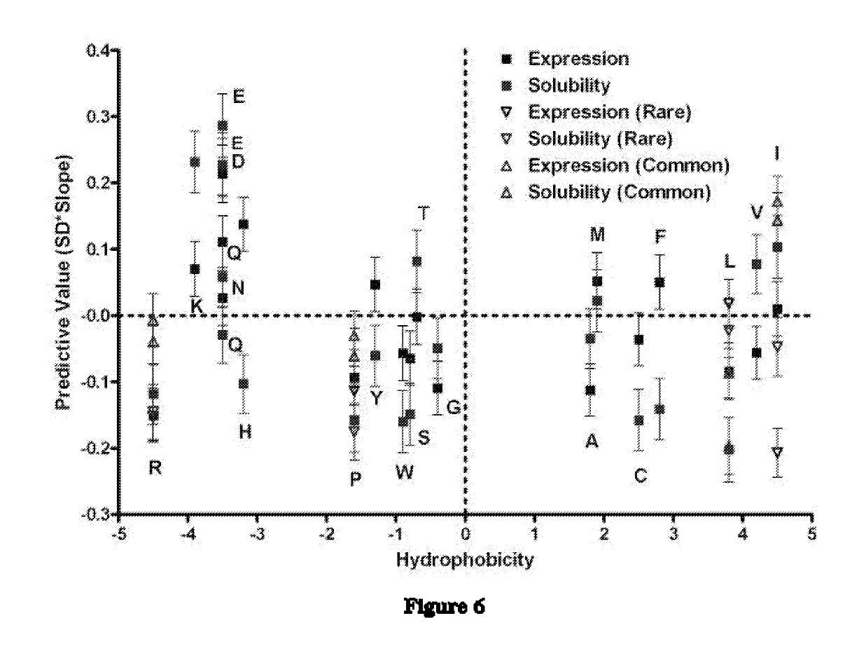

Increasing polypeptide solubility by replacing one or more amino acids in the polypeptide with a more hydrophilic amino acids is a traditional approach for increasing protein solubility. Surprisingly, as shown, inter alia, in FIG. 6, the results described herein show that protein solubility can be increased by substituting one or more amino acids in a polypeptide sequence (at one or more locations in the polypeptide sequence) with a second amino acid. In one embodiment, the second amino acid can have an equivalent or greater hydrophobicity as compared to the substituted amino acid. Thus, in one embodiment, the methods described herein relate to the finding that substitution of a first type of amino acid in a polypeptide with a second type of amino acid having equivalent or greater hydrophobicity and a greater solubility predictive value (defined as the product of the solubility regression slope and the variable standard deviation) than the first amino acid can increase the solubility of the polypeptide. In another embodiment, the methods described herein can be used to increase the solubility of a polypeptide by making one or more modifications in the amino acid sequence of the polypeptide by substituting a first amino acid at one or more positions in the polypeptide sequence with a second amino acid, wherein the second amino acid has the same hydrophilicity and a greater a solubility predictive value as compared to the first amino acid. In another embodiment, the methods described herein can be used to increase the solubility of a polypeptide by making one or more modifications in the amino acid sequence of the polypeptide by substituting a first amino acid at one or more positions in the polypeptide sequence with a second amino acid, wherein the second amino acid has a greater a solubility predictive value as compared to the first amino acid.

In one embodiment the solubility of a recombinant polypeptide expressed in an expression system (e.g. an in vitro expression system, a bacterial expression system, an insect expression system or mammalian expression system expression system) can be increased by substituting one or more arginine residues in the polypeptide sequence with lysine residues.

In another embodiment the solubility of a recombinant polypeptide expressed in an expression system can be increased by substituting one or more valine residues in the polypeptide sequence with isoleucine residues.

In another embodiment the solubility of a recombinant polypeptide expressed in an expression system can be increased by substituting one or more leucine residues in the polypeptide sequence with valine residues.

In another embodiment the solubility of a recombinant polypeptide expressed in an expression system can be increased by substituting one or more leucine residues in the polypeptide sequence with isoleucine amino acid residues.

In another embodiment the solubility of a recombinant polypeptide expressed in an expression system can be increased by substituting one or more phenylalanine residues in the polypeptide sequence with valine residues.

In another embodiment the solubility of a recombinant polypeptide expressed in an expression system can be increased by substituting one or more phenylalanine residues in the polypeptide sequence with isoleucine residues.

In another embodiment the solubility of a recombinant polypeptide expressed in an expression system can be increased by substituting one or more cysteine residues in the polypeptide sequence with phenylalanine residues.

In another embodiment the solubility of a recombinant polypeptide expressed in an expression system can be increased by substituting one or more cysteine residues in the polypeptide sequence with valine residues.

In another embodiment the solubility of a recombinant polypeptide expressed in an expression system can be increased by substituting one or more cysteine residues in the polypeptide sequence with isoleucine residues.

In another embodiment the solubility of a recombinant polypeptide expressed in an expression system can be increased by substituting one or more histidine residues in the polypeptide sequence with threonine residues.

In another embodiment the solubility of a recombinant polypeptide expressed in an expression system can be increased by substituting one or more proline residues in the polypeptide sequence with valine residues.

In another embodiment the solubility of a recombinant polypeptide expressed in an expression system can be increased by substituting one or more glutamine residues in the polypeptide sequence with asparagine residues.

In another embodiment the solubility of a recombinant polypeptide expressed in an expression system can be increased by substituting one or more glutamine residues in the polypeptide sequence with aspartic acid residues.

In another embodiment the solubility of a recombinant polypeptide expressed in an expression system can be increased by substituting one or more glutamine residues in the polypeptide sequence with glutamic acid residues.

In another embodiment the solubility of a recombinant polypeptide expressed in an expression system can be increased by substituting one or more asparagine residues in the polypeptide sequence with aspartic acid residues.

In another embodiment the solubility of a recombinant polypeptide expressed in an expression system can be increased by substituting one or more asparagine residues in the polypeptide sequence with glutamic acid residues.

In another embodiment the solubility of a recombinant polypeptide expressed in an expression system can be increased by substituting one or more aspartic acid residues in the polypeptide sequence with glutamic acid residues.

In one embodiment, the solubility of a recombinant polypeptide expressed in an expression system can be increased by substituting one or more arginine residues in the polypeptide sequence with lysine residues.

Exemplary amino acid substitutions that can be used to increase the solubility of a polypeptide through the substitution of a first type of amino acid with a second type of amino acid in one or more positions in a polypeptide sequence, wherein the second amino acid has a greater relative solubility predictive value are provided in Table 1.

TABLE 1
Exemplary combinations of solubility increasing modifications
between amino acids.

| Amino Acid | Solubility Increasing Replacement Amino Acid |
|---|---|
| Arginine | Lysine, Aspartic Acid, Glutamic Acid, Glutamine, |
| | Asparagine, Histidine, Tyrosine, Threonine, Glycine, |
| | Alanine, Methionine, Valine, Isoleucine |
| Lysine | Glutamic Acid |
| Glutamine | Threonine, Methionine, Valine, Isoleucine, Asparagine, |
| | Aspartic Acid, Glutamic Acid |
| Asparagine | Methionine, Valine, Isoleucine, Aspartic Acid, Glutamic |
| | Acid |
| Aspartic Acid | Glutamic Acid |
| Glutamic Acid | |
| Histidine | Tyrosine, Threonine, Glycine, Alanine, Methionine, |
| | Valine, Isoleucine |
| Proline | Tyrosine, Threonine, Glycine, Alanine, Methionine, |
| | Valine, Isoleucine |
| Tyrosine | Threonine, Alanine, Methionine, Valine, Isoleucine |
| Tryptophan | Serine, Threonine, Glycine, Alanine, Methionine, Valine, |
| | Isoleucine |
| Serine | Threonine, Glycine, Alanine, Methionine, Valine, |
| | Isoleucine |
| Threonine | Isoleucine |
| Glycine | Methionine, Valine, Isoleucine |
| Alanine | Methionine, Valine, Isoleucine |
| Methionine | Valine, Isoleucine |
| Cysteine | Phenylalanine, Valine, Isoleucine |
| Phenylalanine | Valine, Isoleucine |
| Leucine | Valine, Isoleucine |
| Valine | Isoleucine |
| Isoleucine | |

Exemplary amino acid substitutions that can be used to decrease the solubility of a polypeptide through the substitution of a first type of amino acid with a second type of amino acid in one or more positions in a polypeptide sequence, wherein the second amino acid has a lower relative solubility predictive value are provided in Table 2.

TABLE 2
Exemplary combinations of solubility decreasing modifications
between amino acids.

| Amino Acid | Solubility Decreasing Replacement Amino Acid |
|---|---|
| Arginine | |
| Lysine | Arginine |
| Glutamine | Arginine |
| Asparagine | Glutamine, Arginine |
| Aspartic Acid | Asparagine, Glutamine, Arginine |
| Glutamic Acid | Aspartic Acid, Asparagine, Arginine, Lysine |
| Histidine | Arginine |
| Proline | |
| Tyrosine | Proline, Histidine, Arginine |
| Tryptophan | |
| Serine | Tryptophan |
| Threonine | Serine, Tryptophan, Tyrosine, Proline, Histidine, |
| | Asparagine, Glutamine, Arginine |
| Glycine | Serine, Tryptophan, Proline, Tyrosine, Histidine, |
| | Arginine |
| Alanine | Glycine, Serine, Tryptophan, Proline, Tyrosine, |
| | Histidine, Arginine |
| Methionine | Alanine, Glycine, Serine, Tryptophan, Proline, Tyrosine, |
| | Histidine, Glutamine, Arginine |
| Cysteine | |
| Phenylalanine | Cysteine, Serine, Tryptophan, Proline |
| Leucine | |
| Valine | Leucine, Phenylalanine, Cysteine, Methionine, Alanine, |
| | Glycine, Serine, Tryptophan, Tyrosine, Proline, |
| | Histidine, Asparagine, Glutamine, Arginine |
| Isoleucine | Valine, Leucine, Phenylalanine, Cysteine, Methionine, |
| | Alanine, Glycine, Threonine, Serine, Tryptophan, |
| | Tyrosine, Proline, Histidine, Asparagine, Glutamine, |
| | Arginine |

In another aspect, the present invention relates to the finding that the presence of leucine amino acids in a polypeptide is negatively correlated with solubility of a polypeptide when the polypeptide is produced in an expression system (e.g. E. coli or eukaryotic cells). It is known to one skilled in the art that a polypeptide having one or more conservative amino acid substitutions will not necessarily result in the polypeptide having a significantly different activity, function or immunogenicity relative to a wild type polypeptide. A conservative amino acid substitution occurs when one amino acid residue is replaced with another that has a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), aliphatic side chains (e.g., glycine, alanine, valine, leucine, isoleucine), and sulfur-containing side chains (methionine, cysteine). Substitutions can also be made between acidic amino acids and their respective amides (e.g., asparagine and aspartic acid, or glutamine and glutamic acid). For example, replacement of a leucine with an isoleucine may not have a major effect on the properties of the modified recombinant polypeptide relative to the non-modified recombinant polypeptide.

As described herein, the presence of isoleucine residues in polypeptide, when encoded by ATT codons, has a positive effect on solubility. Accordingly, in one embodiment according to the methods and findings described herein, the one or more solubility altering modifications in the nucleic acid sequence encoding the polypeptide can comprise a conservative substitution of one or more leucine codons in the nucleic acid sequence encoding the polypeptide with an isoleucine codon. While such a substitution has been can be used to conserve function, the results described herein show that it can systematically influence other practically important properties like expression or solubility. In still a further embodiment, the one or more solubility altering modifications in the nucleic acid sequence encoding the polypeptide comprises a selective replacement of leucine codons in the nucleic acid sequence encoding the polypeptide with an isoleucine codon wherein the isoleucine codon is an ATT codon such that solubility of the polypeptide is increased. In still another embodiment, the one or more solubility altering modifications in the nucleic acid sequence encoding the polypeptide comprises a selective replacement of an ATT isoleucine codon with a leucine codon in the nucleic acid sequence encoding the polypeptide such that solubility of the polypeptide is decreased.

In another embodiment according to the methods and findings described herein, the one or more expression altering modifications in the nucleic acid sequence encoding the polypeptide can comprise a conservative substitution of one or more leucine codons in the nucleic acid sequence encoding the polypeptide with an isoleucine codon. In still a further embodiment, the one or more expression altering modifications in the nucleic acid sequence encoding the polypeptide comprises a selective replacement of leucine codons in the nucleic acid sequence encoding the polypeptide with an isoleucine codon wherein the isoleucine codon is an ATT codon such that expression of the polypeptide is increased. In still another embodiment, the one or more expression altering modifications in the nucleic acid sequence encoding the polypeptide comprises a selective replacement of an ATT isoleucine codon with a leucine codon in the nucleic acid sequence encoding the polypeptide such that expression of the polypeptide is decreased.

In another aspect, the methods described herein relate to the finding that substitution of a first type of amino acid in a polypeptide with a second type of amino acid with a greater expression predictive value (defined as the product of the expression regression slope and the variable standard deviation) than the first amino acid can increase the expression of the polypeptide. For example, in one embodiment the methods described herein can be used to increase the expression of a polypeptide by making one or more modifications in the amino acid sequence of the polypeptide by substituting a first amino acid at one or more positions in the polypeptide sequence with a second amino acid, wherein the second amino acid has a greater a expression predictive value as compared to the first amino acid. In another embodiment the methods described herein can be used to increase the expression of a polypeptide by making one or more modifications in the amino acid sequence of the polypeptide by substituting a first amino acid at one or more positions in the polypeptide sequence with a second amino acid, wherein the second amino acid has is less hydrophobic and has a greater a expression predictive value as compared to the first amino acid.

In another embodiment the methods described herein can be used to increase the expression of a polypeptide by making one or more modifications in the amino acid sequence of the polypeptide by substituting a first amino acid at one or more positions in the polypeptide sequence with a second amino acid, wherein the second amino acid has the same hydrophilicity and a greater a expression predictive value as compared to the first amino acid.

In one embodiment, the expression of a recombinant polypeptide expressed in an expression system can be increased by substituting one or more arginine residues in the polypeptide sequence with lysine residues.

In another embodiment, the expression of a recombinant polypeptide expressed in an expression system can be increased by substituting one or more valine residues in the polypeptide sequence with isoleucine residues.

In another embodiment, the expression of a recombinant polypeptide expressed in an expression system can be increased by substituting one or more leucine residues in the polypeptide sequence with valine residues.

In another embodiment, the expression of a recombinant polypeptide expressed in an expression system can be increased by substituting one or more leucine residues in the polypeptide sequence with isoleucine residues.

In another embodiment, the expression of a recombinant polypeptide expressed in an expression system can be increased by substituting one or more cysteine residues in the polypeptide sequence with phenylalanine residues.

In another embodiment, the expression of a recombinant polypeptide expressed in an expression system can be increased by substituting one or more alanine residues in the polypeptide sequence with methionine residues.

In another embodiment, the expression of a recombinant polypeptide expressed in an expression system can be increased by substituting one or more alanine residues in the polypeptide sequence with cysteine residues.

In another embodiment, the expression of a recombinant polypeptide expressed in an expression system can be increased by substituting one or more alanine residues in the polypeptide sequence with phenylalanine residues.

In another embodiment, the expression of a recombinant polypeptide expressed in an expression system can be increased by substituting one or more alanine residues in the polypeptide sequence with leucine residues.

In another embodiment, the expression of a recombinant polypeptide expressed in an expression system can be increased by substituting one or more alanine residues in the polypeptide sequence with valine residues.

In another embodiment, the expression of a recombinant polypeptide expressed in an expression system can be increased by substituting one or more alanine residues in the polypeptide sequence with isoleucine residues.

In another embodiment, the expression of a recombinant polypeptide expressed in an expression system can be increased by substituting one or more tryptophan residues in the polypeptide sequence with methionine residues.

In another embodiment, the expression of a recombinant polypeptide expressed in an expression system can be increased by substituting one or more arginine residues in the polypeptide sequence with isoleucine residues.

In another embodiment, the expression of a recombinant polypeptide expressed in an expression system can be increased by substituting one or more arginine or lysine residues in the polypeptide sequence with aspartic acid or glutamic acid residues.

In another embodiment, the expression of a recombinant polypeptide expressed in an expression system can be increased by substituting one or more glutamine residues in the polypeptide sequence with asparagine residues.

In another embodiment, the expression of a recombinant polypeptide expressed in an expression system can be increased by substituting one or more glutamine residues in the polypeptide sequence with glutamic acid residues.

In another embodiment, the expression of a recombinant polypeptide expressed in an expression system can be increased by substituting one or more asparagine residues in the polypeptide sequence with glutamine residues.

In another embodiment, the expression of a recombinant polypeptide expressed in an expression system can be increased by substituting one or more asparagine residues in the polypeptide sequence with aspartic acid residues.

In another embodiment, the expression of a recombinant polypeptide expressed in an expression system can be increased by substituting one or more asparagine residues in the polypeptide sequence with glutamic acid residues.

In another embodiment, the expression of a recombinant polypeptide expressed in an expression system can be increased by substituting one or more aspartic Acid residues in the polypeptide sequence with glutamic acid residues.

Exemplary amino acid substitutions that can be used to increase the expression of a polypeptide through the substitution of a first type of amino acid with a second type of amino acid in one or more positions in a polypeptide sequence, wherein the second amino acid has a greater relative expression predictive value are provided in Table 3.

TABLE 3
Exemplary combinations of expression increasing modifications
between amino acids.

| Amino Acid | Expression Increasing Replacement Amino Acid |
|---|---|
| Arginine | Lysine, Glutamic Acid, Glutamine, Asparagine, Aspartic |
| | Acid, Histidine, Proline, Tyrosine, Tryptophan, Serine, |
| | Threonine, Glycine, Alanine, Methionine, Cysteine, |
| | Phenylalanine, Leucine, Valine, Isoleucine |
| Lysine | Aspartic Acid, Glutamine, Glutamic Acid, Histidine |
| Glutamine | Asparagine, Glutamic Acid |
| Asparagine | Tyrosine, Methionine, Phenylalanine, Glutamine, |
| | Aspartic Acid, Glutamic Acid |
| Aspartic Acid | Glutamic Acid |
| Glutamic Acid | |
| Histidine | |
| Proline | Tyrosine, Tryptophan, Serine, Threonine, Cysteine, |
| | Phenylalanine, Valine, Isoleucine |
| Tyrosine | Methionine, Phenylalanine |
| Tryptophan | Threonine, Methionine, Cysteine, Phenylalanine, |
| | Isoleucine |
| Serine | Threonine, Methionine, Cysteine, Phenylalanine, |
| | Isoleucine |
| Threonine | Methionine, Phenylalanine, Isoleucine |
| Glycine | Methionine, Cysteine, Phenylalanine, Leucine, Valine, |
| | Isoleucine |
| Alanine | Methionine, Cysteine, Phenylalanine, Leucine, Valine, |
| | Isoleucine |
| Methionine | |
| Cysteine | Phenylalanine, Isoleucine |
| Phenylalanine | |
| Leucine | Valine, Isoleucine |
| Valine | Isoleucine |
| Isoleucine | |

Exemplary amino acid substitutions that can be used to decrease the expression of a polypeptide through the substitution of a first type of amino acid with a second type of amino acid in one or more positions in a polypeptide sequence, wherein the second amino acid has a lower relative expression predictive value are provided in Table 4.

TABLE 4
Exemplary combinations of expression decreasing modifications
between amino acids.

| Amino Acid | Solubility Decreasing Replacement Amino Acid |
|---|---|
| Arginine | |
| Lysine | Arginine |
| Glutamine | Asparagine, Lysine, Arginine |
| Asparagine | Arginine |
| Aspartic Acid | Asparagine, Glutamine, Lysine, Arginine |
| Glutamic Acid | Aspartic Acid, Asparagine, Glutamine, Lysine, Arginine |
| Histidine | Glutamine, Asparagine, Lysine, Arginine |
| Proline | Arginine |
| Tyrosine | Asparagine, Arginine |
| Tryptophan | Proline, Arginine |
| Serine | Proline, Arginine |
| Threonine | Serine, Tryptophan, Proline, Arginine |
| Glycine | Arginine |
| Alanine | Arginine |
| Methionine | Alanine, Glycine, Threonine, Serine, Tryptophan, |
| | Tyrosine, Proline, Asparagine, Arginine |
| Cysteine | Alanine, Serine, Tryptophan, Proline, Arginine |
| Phenylalanine | Cysteine, Alanine, Glycine, Threonine, Serine, |
| | Tryptophan, Tyrosine, Proline, Arginine |
| Leucine | Alanine, Proline, Glycine, Arginine |
| Valine | Leucine, Alanine, Glycine, Serine, Tryptophan, Proline, |
| | Arginine |
| Isoleucine | Valine, Leucine, Cysteine, Alanine, Glycine, Threonine, |
| | Serine, Tryptophan, Proline, Arginine |

In certain aspects, the present invention relates to the finding that synonymous codons can differentially impact the solubility of a polypeptide encoded by a nucleic acid sequence in an expression system. For example, in certain respects, the methods described herein are based on the finding that the solubility of a polypeptide depends on the relative frequency of different synonymous codons in the nucleotide sequence encoding the polypeptide. Thus, in certain embodiments the solubility of a recombinant polypeptide expressed in an expression system can be altered by introducing one or more solubility altering modifications in the nucleic acid sequence encoding the recombinant polypeptide.

The methods described herein are based, in part, on the finding that synonymous codons can differentially impact the solubility of a recombinant polypeptide when said recombinant polypeptide is produced in an expression system. For example, the ATA and ATT codons both encode isoleucine residues, however, the presence of an ATT codon in a nucleic acid sequence encoding a recombinant polypeptide has a statistically positive effect on polypeptide solubility when the polypeptide is produced in an expression system, whereas the presence of a ATA codons in the nucleic acid sequence encoding a recombinant polypeptide has a statistically negative effect on polypeptide solubility when the polypeptide is produced in an expression system. In some embodiments, a solubility increasing codon can be a codon which, when present in a nucleic acid encoding a recombinant polypeptide, has a positive correlation with the solubility of the recombinant polypeptide when the recombinant polypeptide is produced in an expression system. In some embodiments, a solubility decreasing codon can be a codon which, when present in a nucleic acid encoding a recombinant polypeptide, has a negative correlation with the solubility of the recombinant polypeptide when the recombinant polypeptide is produced in an expression system. Examples of solubility increasing codons include, but are not limited to, ATT (Ile), CTG (Arg), GGT (Gly), GTA (Val), and GTT (Val). Examples of solubility decreasing codons include, but are not limited to, ATA (Ile), ATC (Ile), AGA (Arg), AGG (Arg), CGA (Arg), CGC (Arg), CGG (Arg), GGG (Gly), and GTG (Val).

In one embodiment according to the methods and findings described herein, the one or more solubility altering modifications in the nucleic acid sequence encoding a polypeptide comprises a selective modification one or more isoleucine codons in the nucleic acid sequence encoding the polypeptide from an ATA codon to an ATT codon such that solubility of the polypeptide is increased. In another embodiment according to the methods and findings described herein, the one or more solubility altering modifications in the nucleic acid sequence encoding a polypeptide comprises a selective modification one or more isoleucine codons in the nucleic acid sequence encoding the polypeptide from an ATT codon to an ATA codon such that solubility of the polypeptide is decreased.

In one embodiment according to the methods and findings described herein, the one or more solubility altering modifications in the nucleic acid sequence encoding a polypeptide comprises a selective modification one or more isoleucine codons in the nucleic acid sequence encoding the polypeptide from an ATC codon to an ATT codon such that the solubility of the polypeptide is increased. In another embodiment according to the methods and findings described herein, the one or more solubility altering modifications in the nucleic acid sequence encoding a polypeptide comprises a selective modification one or more isoleucine codons in the nucleic acid sequence encoding the polypeptide from an ATT codon to an ATC codon such that solubility of the polypeptide is decreased.

In still a further embodiment according to the methods and findings described herein, the one or more solubility altering modifications in the nucleic acid sequence encoding a polypeptide comprises a selective modification one or more arginine codons in the nucleic acid sequence encoding the polypeptide from any of an AGA, AGG, CGA, CGC or CGG codon to a CTG codon such that solubility of the polypeptide is increased. In another embodiment according to the methods and findings described herein, the one or more solubility altering modifications in the nucleic acid sequence encoding a polypeptide comprises a selective modification one or more arginine codons in the nucleic acid sequence encoding the polypeptide from a CTG codon to any of an AGA, AGG, CGA, CGC or CGG codon such that solubility of the polypeptide is increased.

In still yet another embodiment according to the methods and findings described herein, the one or more solubility altering modifications in the nucleic acid sequence encoding a polypeptide comprises a selective modification one or more glycine codons in the nucleic acid sequence encoding the polypeptide from a GGG codon to a GGT codon such that solubility of the polypeptide is increased. In another embodiment according to the methods and findings described herein the one or more solubility altering modifications in the nucleic acid sequence encoding a polypeptide comprises a selective modification one or more glycine codons in the nucleic acid sequence encoding the polypeptide from a GGT codon to a GGG codon such that solubility of the polypeptide is decreased.

In another embodiment according to the methods and findings described herein, the one or more solubility altering modifications in the nucleic acid sequence encoding a polypeptide comprises a selective modification one or more valine codons in the nucleic acid sequence encoding the polypeptide from a GTG codon to a GTA or a GTT codon such that solubility of the polypeptide is increased. In another embodiment according to the methods and findings described herein the one or more solubility altering modifications in the nucleic acid sequence encoding a polypeptide comprises a selective modification one or more valine codons in the nucleic acid sequence encoding the polypeptide from a GTA or a GTT codon to a GTG codon such that solubility of the polypeptide is decreased.

Synonymous codon substitutions that can be used to increase the solubility of a polypeptide through the substitution of a first type of codon with a second synonymous codon, in one or more positions in a polypeptide sequence, wherein the first codon has a greater relative solubility predictive value are provided in Table 5.

TABLE 5
Exemplary combinations of solubility increasing or decreasing
synonymous codon substitutions.

| | Solubility Increasing | Solubility Decreasing |
|---|---|---|
| Amino Acid | Replacement Synonymous | Replacement Synonymous |
| Codon | Codon | Codon |
| Ala (GCT) | | Ala (GCA) Ala (GCC) Ala (GCG) |
| Ala (GCA) | Ala (GCT) | Ala (GCC) Ala (GCG) |
| Ala (GCC) | Ala (GCT) Ala (GCA) | Ala (GCG) |
| Ala (GCG) | Ala (GCT) Ala (GCA) Ala (GCC) | |
| Arg (CGT) | | Arg (AGA) Arg (CGC) Arg |
| | | (AGG) Arg (CGA) Arg (CGG) |
| Arg (AGA) | Arg (CGT) | Arg (CGC) Arg (AGG) Arg (CGA) |
| | | Arg (CGG) |
| Arg (CGC) | Arg (CGT) Arg (AGA) | Arg (AGG) Arg (CGA) Arg |
| | | (CGG) |
| Arg (AGG) | Arg (CGT) Arg (AGA) Arg | Arg (CGA) Arg (CGG) |
| | (CGC) | |
| Arg (CGA) | Arg (CGT) Arg (AGA) Arg | Arg (CGG) |
| | (CGC) Arg (AGG) | |
| Arg (CGG) | Arg (CGT) Arg (AGA) Arg | |
| | (CGC) Arg (AGG) Arg (CGA) | |
| Asn (AAC) | | Asn (AAT) |
| Asn (AAT) | Asn (AAC) | |
| Asp (GAT) | | Asp (GAC) |
| Asp (GAC) | Asp (GAT) | |
| Cys (TGT) | | Cys (TGC) |
| Cys (TGC) | Cys (TGT) | |
| Gln (CAA) | | Gln (CAG) |
| Gln (CAG) | Gln (CAA) | |
| Glu (GAA) | | Glu (GAG) |
| Glu (GAG) | Glu (GAA) | |
| Gly (GGT) | | Gly (GGA) Gly (GGC) Gly (GGG) |
| Gly (GGA) | Gly (GGT) | Gly (GGC) Gly (GGG) |
| Gly (GGC) | Gly (GGT) Gly (GGA) | Gly (GGG) |
| Gly (GGG) | Gly (GGT) Gly (GGA) Gly (GGC) | |
| His (CAT) | | His (CAC) |
| His (CAC) | His (CAT) | |
| Ile (ATT) | | Ile (ATA) Ile (ATC) |
| Ile (ATC) | Ile (ATT) | Ile (ATA) |
| Ile (ATA) | Ile (ATT) Ile (ATC) | |
| Leu (TTA) | | Leu (CTT) Leu (CTA) Leu (CTG) |
| | | Leu (TTG) Leu (CTC) |
| Leu (CTT) | Leu (TTA) | Leu (CTT) Leu (CTA) Leu (CTG) |
| | | Leu (TTG) |
| Leu (CTA) | Leu (TTA) Leu (CTT) | Leu (CTT) Leu (CTA) Leu (CTG) |
| Leu (CTG) | Leu (TTA) Leu (CTT) Leu (CTA) | Leu (CTT) Leu (CTA) |
| Leu (TTG) | Leu (TTA) Leu (CTT) Leu (CTA) | Leu (CTT) |
| | Leu (CTG) | |
| Leu (CTC) | Leu (TTA) Leu (CTT) Leu (CTA) | |
| | Leu (CTG) Leu (TTG) | |
| Lys (AAA) | | Lys (AAG) |
| Lys (AAG) | Lys (AAA) | |
| Met (ATG) | | |
| Phe (TTT) | | Phe (TTC) |
| Phe (TTC) | Phe (TTT) | |
| Pro (CCA) | | Pro (CCG) Pro (CCT) Pro (CCG) |
| Pro (CCG) | Pro (CCA) | Pro (CCG) Pro (CCT) |
| Pro (CCT) | Pro (CCA) Pro (CCG) | Pro (CCG) |
| Pro (CCC) | Pro (CCA) Pro (CCG) Pro (CCT) | |
| Ser (TCT) | | Ser (TCA) Ser (AGT) Ser (AGC) |
| | | Ser (TCC) Ser (TCG) |
| Ser (TCA) | Ser (TCT) | Ser (AGT) Ser (AGC) Ser (TCC) |
| | | Ser (TCG) |
| Ser (AGT) | Ser (TCT) Ser (TCA) | Ser (AGC) Ser (TCC) Ser (TCG) |
| Ser (AGC) | Ser (TCT) Ser (TCA) Ser (AGT) | Ser (TCC) Ser (TCG) |
| Ser (TCC) | Ser (TCT) Ser (TCA) Ser (AGT) | Ser (TCG) |
| | Ser (AGC) | |
| Ser (TCG) | Ser (TCT) Ser (TCA) Ser (AGT) | |
| | Ser (AGC) Ser (TCC) | |
| Thr (ACA) | | Thr (ACT) Thr (ACG) Thr (ACC) |
| Thr (ACT) | Thr (ACA) | Thr (ACG) Thr (ACC) |
| Thr (ACG) | Thr (ACA) Thr (ACT) | Thr (ACC) |
| Thr (ACC) | Thr (ACA) Thr (ACT) Thr (ACG) | |
| Trp (TGG) | | |
| Tyr (TAT) | | Tyr (TAC) |
| Tyr (TAC) | Tyr (TAT) | |
| Val (GTA) | | Val (GTT) Val (GTC) Val (GTG) |
| Val (GTT) | Val (GTA) | Val (GTC) Val (GTG) |
| Val (GTC) | Val (GTA) Val (GTT) | Val (GTG) |
| Val (GTG) | Val (GTA) Val (GTT) Val (GTC) | |

In certain aspects, the present invention relates to the finding that synonymous codons can differentially impact the expression of a polypeptide encoded by a nucleic acid sequence in an expression system (e.g., a bacterial expression system such as E. coli, a mammalian cell expression system, an in vivo expression system or an in-vitro translation system and the like). For example, in certain respects, the methods described herein are based on the finding that the expression of a polypeptide depends on the frequency of different synonymous codons in the nucleotide sequence encoding a polypeptide, and expression can be increased by substitution of some synonymous codons with equal or lower frequency in open reading frames in the genome or equal or lower abundance of cognate tRNAs in the cytosol. Thus, in certain embodiments the expression of a recombinant polypeptide expressed in expression system can be altered by introducing one or more expression altering modifications in the nucleic acid sequence encoding the recombinant polypeptide. In one embodiment, such changes do not involve removal of rare codons.

The methods described herein are based, in part, on the finding that synonymous codons can differentially impact the expression of a recombinant polypeptide when said recombinant polypeptide is produced in an expression system. For example, the GAG and GAA codons both encode glutamic acid residues, however, the presence of an GAA codon in a nucleic acid sequence encoding a recombinant polypeptide has a positive effect on polypeptide expression when the polypeptide is produced in an expression system, whereas the presence of an ATA codon in the nucleic acid sequence encoding a recombinant polypeptide has a negative effect on polypeptide expression when the polypeptide is produced in an expression system.

In some embodiments, an expression increasing codon can be a codon which, when present in a nucleic acid encoding a recombinant polypeptide, has a positive correlation with the expression of the recombinant polypeptide when the recombinant polypeptide is produced in an expression system. In some embodiments, a solubility decreasing codon can be a codon which, when present in a nucleic acid encoding a recombinant polypeptide, has a negative correlation with the expression of the recombinant polypeptide when the recombinant polypeptide is produced in an expression system. Examples of expression increasing codons include, but are not limited to, GAA (Glu), GAT (Asp), CAT (His), CAA (Gln), CGA (Asn), GGT (Gly), TTT (Phe), CCT (Pro), and AGT (Ser). Examples of expression decreasing codons include, but are not limited to, GAG (Glu), GAC (Asp), CAC (His), CAG (Gln), AGA (Asn), AGG (Asn), CGT (Asn), CGC(Asn), CGG (Asn), GGG (Gly), TTC (Phe), CCC (Pro), CCG (Pro), TCC (Ser), and TCG (Ser).

In one embodiment according to the methods and findings described herein, the one or more expression altering modifications in the nucleic acid sequence encoding a polypeptide comprises a selective modification one or more glutamic acid codons in the nucleic acid sequence encoding the polypeptide from an GAG codon to a GAA codon such that expression of the polypeptide is increased. In another embodiment according to the methods and findings described herein the one or more expression altering modifications in the nucleic acid sequence encoding a polypeptide comprises a selective modification one or more glutamic acid codons in the nucleic acid sequence encoding the polypeptide from an GAA codon to a GAG codon such that expression of the polypeptide is decreased.

In another embodiment according to the methods and findings described herein, the one or more expression altering modifications in the nucleic acid sequence encoding a polypeptide comprises a selective modification one or more aspartic acid codons in the nucleic acid sequence encoding the polypeptide from an GAC codon to a GAT codon such that expression of the polypeptide is increased. In another embodiment according to the methods and findings described herein the one or more expression altering modifications in the nucleic acid sequence encoding a polypeptide comprises a selective modification one or more aspartic acid codons in the nucleic acid sequence encoding the polypeptide from an GAT codon to a GAC codon such that expression of the polypeptide is decreased.

In another embodiment according to the methods and findings described herein, the one or more expression altering modifications in the nucleic acid sequence encoding a polypeptide comprises a selective modification one or more histidine codons in the nucleic acid sequence encoding the polypeptide from an CAC codon to an CAT codon such that expression of the polypeptide is increased. In another embodiment according to the methods and findings described herein the one or more expression altering modifications in the nucleic acid sequence encoding a polypeptide comprises a selective modification one or more histidine codons in the nucleic acid sequence encoding the polypeptide from an CAT codon to an CAC codon such that expression of the polypeptide is decreased.

In another embodiment according to the methods and findings described herein, the one or more expression altering modifications in the nucleic acid sequence encoding a polypeptide comprises a selective modification one or more glutamine codons in the nucleic acid sequence encoding the polypeptide from an CAG codon to an CAA codon such that expression of the polypeptide is increased. In another embodiment according to the methods and findings described herein the one or more expression altering modifications in the nucleic acid sequence encoding a polypeptide comprises a selective modification one or more glutamine codons in the nucleic acid sequence encoding the polypeptide from an CAA codon to an CAG codon such that expression of the polypeptide is decreased.

In still a further embodiment according to the methods and findings described herein, the one or more expression altering modifications in the nucleic acid sequence encoding a polypeptide comprises a selective modification one or more arginine codons in the nucleic acid sequence encoding the polypeptide from any of an AGA, AGG, CGT, CGC or CGG codon to a CGA codon such that expression of the polypeptide is increased. In another embodiment according to the methods and findings described herein the one or more expression altering modifications in the nucleic acid sequence encoding a polypeptide comprises a selective modification one or more arginine codons in the nucleic acid sequence encoding the polypeptide from a CGA codon to any of an AGA, AGG, CGT, CGC or CGG codon such that expression of the polypeptide is decreased.

In another embodiment according to the methods and findings described herein, the one or more expression altering modifications in the nucleic acid sequence encoding a polypeptide comprises a selective modification one or more glycine codons in the nucleic acid sequence encoding the polypeptide from a GGG codon to a GGT codon such that expression of the polypeptide is increased. In another embodiment according to the methods and findings described herein the one or more expression altering modifications in the nucleic acid sequence encoding a polypeptide comprises a selective modification one or more glycine codons in the nucleic acid sequence encoding the polypeptide from a GGT codon to a GGG codon such that expression of the polypeptide is decreased.

In another embodiment according to the methods and findings described herein, the one or more expression altering modifications in the nucleic acid sequence encoding a polypeptide comprises a selective modification one or more phenylalanine codons in the nucleic acid sequence encoding the polypeptide from a TTC codon to a TTT codon such that expression of the polypeptide is increased. In another embodiment according to the methods and findings described herein the one or more expression altering modifications in the nucleic acid sequence encoding a polypeptide comprises a selective modification one or more phenylalanine codons in the nucleic acid sequence encoding the polypeptide from a TTT codon to a TTC codon such that expression of the polypeptide is decreased.

In another embodiment according to the methods and findings described herein, the one or more expression altering modifications in the nucleic acid sequence encoding a polypeptide comprises a selective modification one or more proline codons in the nucleic acid sequence encoding the polypeptide from a CCC or CCG codon to a CCT codon such that expression of the polypeptide is increased. In another embodiment according to the methods and findings described herein the one or more expression altering modifications in the nucleic acid sequence encoding a polypeptide comprises a selective modification one or more proline codons in the nucleic acid sequence encoding the polypeptide from a CCT codon to a CCC or CCG codon such that expression of the polypeptide is decreased.

In another embodiment according to the methods and findings described herein, the one or more expression altering modifications in the nucleic acid sequence encoding a polypeptide comprises a selective modification one or more serine codons in the nucleic acid sequence encoding the polypeptide from a TCC or TCG codon to an AGT codon such that expression of the polypeptide is increased. In another embodiment according to the methods and findings described herein the one or more expression altering modifications in the nucleic acid sequence encoding a polypeptide comprises a selective modification one or more serine codons in the nucleic acid sequence encoding the polypeptide from an AGT codon to a TCC or TCG codon such that expression of the polypeptide is decreased.

Synonymous codon substitutions that can be used to increase the expression of a polypeptide through the substitution of a first type of codon with a second synonymous codon, in one or more positions in a polypeptide sequence, wherein the first codon has a greater relative expression predictive value are provided in Table 6.

TABLE 6
Exemplary combinations of expression increasing or decreasing
synonymous codon substitutions.

| Amino | Expression Increasing | Expression Decreasing |
|---|---|---|
| Acid | Replacement Synonymous | Replacement |
| Codon | Codon | Synonymous Codon |
| Ala (GCT) | | Ala (GCA) Ala (GCC) Ala |
| | | (GCG) |
| Ala (GCA) | Ala (GCT) | Ala (GCC) Ala (GCG) |
| Ala (GCC) | Ala (GCT) Ala (GCA) | Ala (GCG) |
| Ala (GCG) | Ala (GCT) Ala (GCA) Ala (GCC) | |
| Arg (CGA) | | Arg (CGT) Arg (AGA) |
| | | Arg (CGC) Arg (AGG) |
| | | Arg (CGG) |
| Arg (CGT) | Arg (CGA) | Arg (AGA) Arg (CGC) |
| | | Arg (AGG) Arg (CGG) |
| Arg (AGA) | Arg (CGA) Arg (CGT) | Arg (CGC) Arg (AGG) |
| | | Arg (CGG) |
| Arg (CGC) | Arg (CGA) Arg (CGT) Arg (AGA) | Arg (AGG) Arg (CGG) |
| Arg (AGG) | Arg (CGA) Arg (CGT) Arg (AGA) | Arg (CGG) |
| | Arg (CGC) | |
| Arg (CGG) | Arg (CGA) Arg (CGT) Arg (AGA) | |
| | Arg (CGC) Arg (AGG) | |
| Asn (AAT) | | Asn (AAC) |
| Asn (AAC) | Asn (AAT) | |
| Asp (GAT) | | Asp (GAC) |
| Asp (GAC) | Asp (GAT) | |
| Cys (TGT) | | Cys (TGC) |
| Cys (TGC) | Cys (TGT) | |
| Gln (CAA) | | Gln (CAG) |
| Gln (CAG) | Gln (CAA) | |
| Glu (GAA) | | Glu (GAG) |
| Glu (GAG) | Glu (GAA) | |
| Gly (GGT) | | Gly (GGA) Gly (GGC) |
| | | Gly (GGG) |
| Gly (GGA) | Gly (GGT) | Gly (GGC) Gly (GGG) |
| Gly (GGC) | Gly (GGT) Gly (GGA) | Gly (GGG) |
| Gly (GGG) | Gly (GGT) Gly (GGA) Gly (GGC) | |
| His (CAT) | | His (CAC) |
| His (CAC) | His (CAT) | |
| Ile (ATT) | | Ile (ATA) Ile (ATC) |
| Ile (ATC) | Ile (ATT) | Ile (ATA) |
| Ile (ATA) | Ile (ATT) Ile (ATC) | |
| Leu (TTA) | | Leu (TTG) Leu (CTA) Leu |
| | | (CTT) Leu (CTG) Leu |
| | | (CTC) |
| Leu (TTG) | Leu (TTA) | Leu (CTA) Leu (CTT) Leu |
| | | (CTG) Leu (CTC) |
| Leu (CTA) | Leu (TTA) Leu (TTG) | Leu (CTT) Leu (CTG) Leu |
| | | (CTC) |
| Leu (CTT) | Leu (TTA) Leu (TTG) Leu (CTA) | Leu (CTG) Leu (CTC) |
| Leu (CTG) | Leu (TTA) Leu (TTG) Leu (CTA) | Leu (CTC) |
| | Leu (CTT) | |
| Leu (CTC) | Leu (TTA) Leu (TTG) Leu (CTA) | |
| | Leu (CTT) Leu (CTG) | |
| Lys (AAA) | | Lys (AAG) |
| Lys (AAG) | Lys (AAA) | |
| Met (ATG) | | |
| Phe (TTT) | | Phe (TTC) |
| Phe (TTC) | Phe (TTT) | |
| Pro (CCT) | | Pro (CCA) Pro (CCG) Pro |
| | | (CCC) |
| Pro (CCA) | Pro (CCT) | Pro (CCG) Pro (CCC) |
| Pro (CCG) | Pro (CCT) Pro (CCA) | Pro (CCC) |
| Pro (CCC) | Pro (CCT) Pro (CCA) Pro (CCG) | |
| Ser (AGT) | | Ser (TCA) Ser (TCT) Ser |
| | | (AGC) Ser (TCC) Ser |
| | | (TCG) |
| Ser (TCA) | Ser (AGT) | Ser (TCT) Ser (AGC) Ser |
| | | (TCC) Ser (TCG) |
| Ser (TCT) | Ser (AGT) Ser (TCA) | Ser (AGC) Ser (TCC) Ser |
| | | (TCG) |
| Ser (AGC) | Ser (AGT) Ser (TCA) Ser (TCT) | Ser (TCC) Ser (TCG) |
| Ser (TCC) | Ser (AGT) Ser (TCA) Ser (TCT) | Ser (TCG) |
| | Ser (AGC) | |
| Ser (TCG) | Ser (AGT) Ser (TCA) Ser (TCT) | |
| | Ser (AGC) Ser (TCC) | |
| Thr (ACA) | | Thr (ACT) Thr (ACC) Thr |
| | | (ACG) |
| Thr (ACT) | Thr (ACA) | Thr (ACC) Thr (ACG) |
| Thr (ACC) | Thr (ACA) Thr (ACT) | Thr (ACG) |
| Thr (ACG) | Thr (ACA) Thr (ACT) Thr (ACC) | |
| Trp (TGG) | | |
| Tyr (TAT) | | Tyr (TAC) |
| Tyr (TAC) | Tyr (TAT) | |
| Val (GTT) | | Val (GTA) Val (GTG) Val |
| | | (GTC) |
| Val (GTA) | Val (GTT) | Val (GTG) Val (GTC) |
| Val (GTG) | Val (GTT) Val (GTA) | Val (GTC) |
| Val (GTC) | Val (GTT) Val (GTA) Val (GTG) | |

In certain aspects, the present invention relates to the finding that different codons can differentially impact the solubility of a polypeptide encoded by a nucleic acid sequence in an expression system. In one embodiment, the methods described herein can involve the introduction of one or more nucleic acid substitutions in a nucleic acid sequence encoding a polypeptide that preserve or change the identity of one or more amino acids in the encoded polypeptide. For example, in certain respects, the methods described herein are based on the finding that the solubility or expression of a polypeptide depends on the presence or frequency or specific codons in the nucleic acid encoding the polypeptide. Thus, in certain embodiments the solubility or expression of a recombinant polypeptide expressed in an expression system can be altered by introducing one or more solubility altering modifications in the nucleic acid sequence encoding the recombinant polypeptide. One skilled in the art will readily be able to design modifications that introduce conservative substitutions in the sequence of a polypeptide, or modifications in the amino acid sequence of the polypeptide that do not adversely affect the sequence, structure, function or immunogenicity of the polypeptide.

In certain aspects, the present invention relates to the finding that different codons can differentially impact the solubility of a polypeptide encoded by a nucleic acid sequence in an expression system. For example, in certain respects, the methods described herein are based on the finding that the solubility of a polypeptide depends on the relative frequency of different codons in the nucleotide sequence encoding the polypeptide. Thus, in certain embodiments the solubility of a recombinant polypeptide expressed with an expression system can be altered by introducing one or more solubility altering modifications in the nucleic acid sequence encoding the recombinant polypeptide. In one embodiment, the solubility altering codon can involve substitution of a first codon in the nucleic acid sequence encoding a polypeptide with a second solubility increasing codon wherein the amino acid encoded by said solubility increasing codon has an equivalent or greater hydrophobicity and a greater solubility predictive value (defined as the product of the solubility regression slope and the variable standard deviation) than the first codon. For example, in certain embodiments according to the methods described herein, an alanine (GCA) codon in a nucleic acid sequence encoding a polypeptide is replaced at one or more location with a different codon (or more than one different types of codons) selected from the group consisting of Met(ATG) Ile(ATC) Ala(GCT) Leu(TTA) Ile(ATT) Val(GTT) and Val(GTA).

In certain aspects, the present invention relates to the finding that codons can differentially impact the expression of a polypeptide encoded by a nucleic acid sequence in an expression system. For example, in certain respects, the methods described herein are based on the finding that the expression of a polypeptide depends on the relative frequency of different codons in the nucleotide sequence encoding the polypeptide. Thus, in certain embodiments the expression level of a recombinant polypeptide expressed in an expression system can be altered by introducing one or more expression altering modifications in the nucleic acid sequence encoding the recombinant polypeptide. In one embodiment, the expression altering codon can involve substitution of a first codon in the nucleic acid sequence encoding a polypeptide with a second expression increasing codon wherein said expression increasing codon has an equivalent or greater hydrophobicity and a greater expression predictive value (defined as the product of the expression regression slope and the variable standard deviation) than the first codon, irrespective of the relative frequency these codons in the genome or the relative abundance of cognate tRNAs in the tRNA pool.

In one embodiment, the expression altering codon can involve substitution of a first codon in the nucleic acid sequence encoding a polypeptide with a second expression increasing codon wherein said expression increasing codon has a greater expression predictive value than the first codon, irrespective of the relative frequency these codons in the genome or the relative abundance of cognate tRNAs in the tRNA pool.

For example, in certain embodiments according to the methods described herein, an alanine (GCA) codon in a nucleic acid sequence encoding a polypeptide is replaced at one or more location with a different codon (or more than one different types of codons) selected from the group consisting of Leu(TTG) Leu(TTA) Ala(GCT) Phe(TTT) Met(ATG) Ile(ATT).

Codon substitutions that can be used to increase the solubility or expression of a polypeptide through the substitution of a first type of codon with a second codon, in one or more positions in a polypeptide sequence, wherein the first codon has a greater relative solubility or expression predictive value are provided in Table 7.

TABLE 7
Exemplary combinations of solubility or expression
increasing or codon substitutions.

| Amino | Solubility Increasing | Expression Increasing |
|---|---|---|
| Acid | Codon | Codon |
| Ala(GCA) | Met(ATG) Ile(ATC) Ala(GCT) | Leu(TTG) Leu(TTA) Ala(GCT) |
| | Leu(TTA) Ile(ATT) Val(GTT) | Phe(TTT) Met(ATG) Ile(ATT) |
| | Val(GTA) | |
| Ala(GCC) | Leu(CTT) Val(GTC) Ala(GCA) | Val(GTG) Leu(CTG) Leu(CTT) |
| | Met(ATG) Ile(ATC) Ala(GCT) | Ile(ATC) Leu(CTA) Val(GTA) |
| | Leu(TTA) Ile(ATT) Val(GTT) | Cys(TGT) Val(GTT) Ala(GCA) |
| | Val(GTA) | Leu(TTG) Leu(TTA) Ala(GCT) |
| | | Phe(TTT) Met(ATG) Ile(ATT) |
| Ala(GCG) | Phe(TTT) Ala(GCC) Leu(CTT) | Ala(GCC) Val(GTG) Leu(CTG) |
| | Val(GTC) Ala(GCA) Met(ATG) | Leu(CTT) Ile(ATC) Leu(CTA) |
| | Ile(ATC) Ala(GCT) Leu(TTA) | Val(GTA) Cys(TGT) Val(GTT) |
| | Ile(ATT) Val(GTT) Val(GTA) | Ala(GCA) Leu(TTG) Leu(TTA) |
| | | Ala(GCT) Phe(TTT) Met(ATG) |
| | | Ile(ATT) |
| Ala(GCT) | Leu(TTA) Ile(ATT) Val(GTT) | Phe(TTT) Met(ATG) Ile(ATT) |
| | Val(GTA) | |
| Arg(AGA) | Ser(TCT) Thr(ACC) Gly(GGA) | Gly(GGC) Gly(GGA) Leu(CTG) |
| | Ala(GCA) Glu(GAG) Asn(AAT) | Asn(AAC) Asp(GAC) Ser(AGC) |
| | Gln(CAA) Met(ATG) Ile(ATC) | Glu(GAG) Lys(AAG) Leu(CTT) |
| | Ala(GCT) Leu(TTA) Asp(GAC) | Ser(TCT) His(CAC) Ile(ATC) |
| | Thr(ACG) Thr(ACT) Asn(AAC) | Gln(CAG) Leu(CTA) Ser(TCA) |
| | Pro(CCA) Thr(ACA) Arg(CGT) | Val(GTA) Cys(TGT) Asn(AAT) |
| | Lys(AAG) Ile(ATT) Gly(GGT) | Val(GTT) Lys(AAA) Ala(GCA) |
| | Lys(AAA) Val(GTT) Val(GTA) | Tyr(TAT) Leu(TTG) Thr(ACT) |
| | Asp(GAT) Glu(GAA) | Pro(CCA) Leu(TTA) Arg(CGT) |
| | | Ala(GCT) Phe(TTT) Arg(CGA) |
| | | Met(ATG) Gly(GGT) Ser(AGT) |
| | | Thr(ACA) Ile(ATT) Gln(CAA) |
| | | Pro(CCT) Glu(GAA) Asp(GAT) |
| | | His(CAT) |
| Arg(AGG) | Gln(CAG) Val(GTG) Leu(CTG) | Cys(TGC) Phe(TTC) Thr(ACG) |
| | Tyr(TAC) His(CAT) Pro(CCG) | Ala(GCG) Ala(GCC) Arg(CGC) |
| | Ile(ATA) Leu(CTA) Arg(CGC) | Tyr(TAC) Thr(ACC) Trp(TGG) |
| | Ser(TCA) Gly(GGC) Tyr(TAT) | Val(GTG) Arg(AGA) Gly(GGC) |
| | Ala(GCG) Phe(TTT) Ala(GCC) | Gly(GGA) Leu(CTG) Asn(AAC) |
| | Leu(CTT) Val(GTC) Arg(AGA) | Asp(GAC) Ser(AGC) Glu(GAG) |
| | Ser(TCT) Thr(ACC) Gly(GGA) | Lys(AAG) Leu(CTT) Ser(TCT) |
| | Ala(GCA) Glu(GAG) Asn(AAT) | His(CAC) Ile(ATC) Gln(CAG) |
| | Gln(CAA) Met(ATG) Ile(ATC) | Leu(CTA) Ser(TCA) Val(GTA) |
| | Ala(GCT) Leu(TTA) Asp(GAC) | Cys(TGT) Asn(AAT) Val(GTT) |
| | Thr(ACG) Thr(ACT) Asn(AAC) | Lys(AAA) Ala(GCA) Tyr(TAT) |
| | Pro(CCA) Thr(ACA) Arg(CGT) | Leu(TTG) Thr(ACT) Pro(CCA) |
| | Lys(AAG) Ile(ATT) Gly(GGT) | Leu(TTA) Arg(CGT) Ala(GCT) |
| | Lys(AAA) Val(GTT) Val(GTA) | Phe(TTT) Arg(CGA) Met(ATG) |
| | Asp(GAT) Glu(GAA) | Gly(GGT) Ser(AGT) Thr(ACA) |
| | | Ile(ATT) Gln(CAA) Pro(CCT) |
| | | Glu(GAA) Asp(GAT) His(CAT) |
| Arg(CGA) | His(CAC) Ser(TCG) Ser(TCC) | Met(ATG) Gly(GGT) Ser(AGT) |
| | Phe(TTC) Ser(AGC) Leu(CTC) | Thr(ACA) Ile(ATT) Gln(CAA) |
| | Leu(TTG) Pro(CCT) Ser(AGT) | Pro(CCT) Glu(GAA) Asp(GAT) |
| | Arg(AGG) Gln(CAG) Val(GTG) | His(CAT) |
| | Leu(CTG) Tyr(TAC) His(CAT) | |
| | Pro(CCG) Ile(ATA) Leu(CTA) | |
| | Arg(CGC) Ser(TCA) Gly(GGC) | |
| | Tyr(TAT) Ala(GCG) Phe(TTT) | |
| | Ala(GCC) Leu(CTT) Val(GTC) | |
| | Arg(AGA) Ser(TCT) Thr(ACC) | |
| | Gly(GGA) Ala(GCA) Glu(GAG) | |
| | Asn(AAT) Gln(CAA) Met(ATG) | |
| | Ile(ATC) Ala(GCT) Leu(TTA) | |
| | Asp(GAC) Thr(ACG) Thr(ACT) | |
| | Asn(AAC) Pro(CCA) Thr(ACA) | |
| | Arg(CGT) Lys(AAG) Ile(ATT) | |
| | Gly(GGT) Lys(AAA) Val(GTT) | |
| | Val(GTA) Asp(GAT) Glu(GAA) | |
| Arg(CGC) | Ser(TCA) Gly(GGC) Tyr(TAT) | Tyr(TAC) Thr(ACC) Trp(TGG) |
| | Ala(GCG) Phe(TTT) Ala(GCC) | Val(GTG) Arg(AGA) Gly(GGC) |
| | Leu(CTT) Val(GTC) Arg(AGA) | Gly(GGA) Leu(CTG) Asn(AAC) |
| | Ser(TCT) Thr(ACC) Gly(GGA) | Asp(GAC) Ser(AGC) Glu(GAG) |
| | Ala(GCA) Glu(GAG) Asn(AAT) | Lys(AAG) Leu(CTT) Ser(TCT) |
| | Gln(CAA) Met(ATG) Ile(ATC) | His(CAC) Ile(ATC) Gln(CAG) |
| | Ala(GCT) Leu(TTA) Asp(GAC) | Leu(CTA) Ser(TCA) Val(GTA) |
| | Thr(ACG) Thr(ACT) Asn(AAC) | Cys(TGT) Asn(AAT) Val(GTT) |
| | Pro(CCA) Thr(ACA) Arg(CGT) | Lys(AAA) Ala(GCA) Tyr(TAT) |
| | Lys(AAG) Ile(ATT) Gly(GGT) | Leu(TTG) Thr(ACT) Pro(CCA) |
| | Lys(AAA) Val(GTT) Val(GTA) | Leu(TTA) Arg(CGT) Ala(GCT) |
| | Asp(GAT) Glu(GAA) | Phe(TTT) Arg(CGA) Met(ATG) |
| | | Gly(GGT) Ser(AGT) Thr(ACA) |
| | | Ile(ATT) Gln(CAA) Pro(CCT) |
| | | Glu(GAA) Asp(GAT) His(CAT) |
| Arg(CGG) | Arg(CGA) His(CAC) Ser(TCG) | Gly(GGG) Ile(ATA) Pro(CCC) |
| | Ser(TCC) Phe(TTC) Ser(AGC) | Leu(CTC) Pro(CCG) Val(GTC) |
| | Leu(CTC) Leu(TTG) Pro(CCT) | Ser(TCC) Arg(AGG) Cys(TGC) |
| | Ser(AGT) Arg(AGG) Gln(CAG) | Phe(TTC) Thr(ACG) Ala(GCG) |
| | Val(GTG) Leu(CTG) Tyr(TAC) | Ala(GCC) Arg(CGC) Tyr(TAC) |
| | His(CAT) Pro(CCG) Ile(ATA) | Thr(ACC) Trp(TGG) Val(GTG) |
| | Leu(CTA) Arg(CGC) Ser(TCA) | Arg(AGA) Gly(GGC) Gly(GGA) |
| | Gly(GGC) Tyr(TAT) Ala(GCG) | Leu(CTG) Asn(AAC) Asp(GAC) |
| | Phe(TTT) Ala(GCC) Leu(CTT) | Ser(AGC) Glu(GAG) Lys(AAG) |
| | Val(GTC) Arg(AGA) Ser(TCT) | Leu(CTT) Ser(TCT) His(CAC) |
| | Thr(ACC) Gly(GGA) Ala(GCA) | Ile(ATC) Gln(CAG) Leu(CTA) |
| | Glu(GAG) Asn(AAT) Gln(CAA) | Ser(TCA) Val(GTA) Cys(TGT) |
| | Met(ATG) Ile(ATC) Ala(GCT) | Asn(AAT) Val(GTT) Lys(AAA) |
| | Leu(TTA) Asp(GAC) Thr(ACG) | Ala(GCA) Tyr(TAT) Leu(TTG) |
| | Thr(ACT) Asn(AAC) Pro(CCA) | Thr(ACT) Pro(CCA) Leu(TTA) |
| | Thr(ACA) Arg(CGT) Lys(AAG) | Arg(CGT) Ala(GCT) Phe(TTT) |
| | Ile(ATT) Gly(GGT) Lys(AAA) | Arg(CGA) Met(ATG) Gly(GGT) |
| | Val(GTT) Val(GTA) Asp(GAT) | Ser(AGT) Thr(ACA) Ile(ATT) |
| | Glu(GAA) | Gln(CAA) Pro(CCT) Glu(GAA) |
| | | Asp(GAT) His(CAT) |
| Arg(CGT) | Lys(AAG) Ile(ATT) Gly(GGT) | Ala(GCT) Phe(TTT) Arg(CGA) |
| | Lys(AAA) Val(GTT) Val(GTA) | Met(ATG) Gly(GGT) Ser(AGT) |
| | Asp(GAT) Glu(GAA) | Thr(ACA) Ile(ATT) Gln(CAA) |
| | | Pro(CCT) Glu(GAA) Asp(GAT) |
| | | His(CAT) |
| Asn(AAC) | Pro(CCA) Thr(ACA) Ile(ATT) | Asp(GAC) Ser(AGC) Glu(GAG) |
| | Gly(GGT) Val(GTT) Val(GTA) | Leu(CTT) Ser(TCT) His(CAC) |
| | Asp(GAT) Glu(GAA) | Ile(ATC) Gln(CAG) Leu(CTA) |
| | | Ser(TCA) Val(GTA) Cys(TGT) |
| | | Asn(AAT) Val(GTT) Ala(GCA) |
| | | Tyr(TAT) Leu(TTG) Thr(ACT) |
| | | Pro(CCA) Leu(TTA) Ala(GCT) |
| | | Phe(TTT) Met(ATG) Gly(GGT) |
| | | Ser(AGT) Thr(ACA) Ile(ATT) |
| | | Gln(CAA) Pro(CCT) Glu(GAA) |
| | | Asp(GAT) His(CAT) |
| Asn(AAT) | Gln(CAA) Met(ATG) Ile(ATC) | Val(GTT) Ala(GCA) Tyr(TAT) |
| | Ala(GCT) Leu(TTA) Asp(GAC) | Leu(TTG) Thr(ACT) Pro(CCA) |
| | Thr(ACG) Thr(ACT) Asn(AAC) | Leu(TTA) Ala(GCT) Phe(TTT) |
| | Pro(CCA) Thr(ACA) Ile(ATT) | Met(ATG) Gly(GGT) Ser(AGT) |
| | Gly(GGT) Val(GTT) Val(GTA) | Thr(ACA) Ile(ATT) Gln(CAA) |
| | Asp(GAT) Glu(GAA) | Pro(CCT) Glu(GAA) Asp(GAT) |
| | | His(CAT) |
| Asp(GAC) | Thr(ACG) Thr(ACT) Asn(AAC) | Ser(AGC) Glu(GAG) Leu(CTT) |
| | Pro(CCA) Thr(ACA) Ile(ATT) | Ser(TCT) His(CAC) Ile(ATC) |
| | Gly(GGT) Val(GTT) Val(GTA) | Gln(CAG) Leu(CTA) Ser(TCA) |
| | Asp(GAT) Glu(GAA) | Val(GTA) Cys(TGT) Asn(AAT) |
| | | Val(GTT) Ala(GCA) Tyr(TAT) |
| | | Leu(TTG) Thr(ACT) Pro(CCA) |
| | | Leu(TTA) Ala(GCT) Phe(TTT) |
| | | Met(ATG) Gly(GGT) Ser(AGT) |
| | | Thr(ACA) Ile(ATT) Gln(CAA) |
| | | Pro(CCT) Glu(GAA) Asp(GAT) |
| | | His(CAT) |
| Asp(GAT) | Glu(GAA) | His(CAT) |
| Cys(TGC) | Cys (TGT) Phe(TTC) Leu(CTC) | Phe(TTC) Val(GTG) Leu(CTG) |
| | Leu(TTG) Val(GTG) Leu(CTG) | Leu(CTT) Ile(ATC) Leu(CTA) |
| | Ile(ATA) Leu(CTA) Phe(TTT) | Val(GTA) Cys (TGT) Val(GTT) |
| | Leu(CTT) Val(GTC) Ile(ATC) | Leu(TTG) Leu(TTA) Phe(TTT) |
| | Leu(TTA) Ile(ATT) Val(GTT) | Ile(ATT) |
| | Val(GTA) | |
| Cys(TGT) | Phe(TTC) Leu(CTC) Leu(TTG) | Val(GTT) Leu(TTG) Leu(TTA) |
| | Val(GTG) Leu(CTG) Ile(ATA) | Phe(TTT) Ile(ATT) |
| | Leu(CTA) Phe(TTT) Leu(CTT) | |
| | Val(GTC) Leu(TTA) Ile(ATT) | |
| | Val(GTT) Val(GTA) | |
| Gln(CAA) | Met(ATG) Ile(ATC) Ala(GCT) | Pro(CCT) Glu(GAA) Asp(GAT) |
| | Leu(TTA) Asp(GAC) Thr(ACG) | His(CAT) |
| | Thr(ACT) Asn(AAC) Pro(CCA) | |
| | Thr(ACA) Ile(ATT) Gly(GGT) | |
| | Val(GTT) Val(GTA) Asp(GAT) | |
| | Glu(GAA) | |
| Gln(CAG) | Val(GTG) Leu(CTG) Tyr(TAC) | Leu(CTA) Ser(TCA) Val(GTA) |
| | His(CAT) Pro(CCG) Ile(ATA) | Cys(TGT) Asn(AAT) Val(GTT) |
| | Leu(CTA) Ser(TCA) Gly(GGC) | Ala(GCA) Tyr(TAT) Leu(TTG) |
| | Tyr(TAT) Ala(GCG) Phe(TTT) | Thr(ACT) Pro(CCA) Leu(TTA) |
| | Ala(GCC) Leu(CTT) Val(GTC) | Ala(GCT) Phe(TTT) Met(ATG) |
| | Ser(TCT) Thr(ACC) Gly(GGA) | Gly(GGT) Ser(AGT) Thr(ACA) |
| | Ala(GCA) Glu(GAG) Asn(AAT) | Ile(ATT) Gln(CAA) Pro(CCT) |
| | Gln(CAA) Met(ATG) Ile(ATC) | Glu(GAA) Asp(GAT) His(CAT) |
| | Ala(GCT) Leu(TTA) Asp(GAC) | |
| | Thr(ACG) Thr(ACT) Asn(AAC) | |
| | Pro(CCA) Thr(ACA) Ile(ATT) | |
| | Gly(GGT) Val(GTT) Val(GTA) | |
| | Asp(GAT) Glu(GAA) | |
| Glu(GAA) | | Asp(GAT) His(CAT) |
| Glu(GAG) | Asn(AAT) Gln(CAA) Met(ATG) | Leu(CTT) Ser(TCT) His(CAC) |
| | Ile(ATC) Ala(GCT) Leu(TTA) | Ile(ATC) Gln(CAG) Leu(CTA) |
| | Asp(GAC) Thr(ACG) Thr(ACT) | Ser(TCA) Val(GTA) Cys(TGT) |
| | Asn(AAC) Pro(CCA) Thr(ACA) | Asn(AAT) Val(GTT) Ala(GCA) |
| | Ile(ATT) Gly(GGT) Val(GTT) | Tyr(TAT) Leu(TTG) Thr(ACT) |
| | Val(GTA) Asp(GAT) Glu(GAA) | Pro(CCA) Leu(TTA) Ala(GCT) |
| | | Phe(TTT) Met(ATG) Gly(GGT) |
| | | Ser(AGT) Thr(ACA) Ile(ATT) |
| | | Gln(CAA) Pro(CCT) Glu(GAA) |
| | | Asp(GAT) His(CAT) |
| Gly(GGA) | Ala(GCA) Asn(AAT) Met(ATG) | Leu(CTG) Asn(AAC) Leu(CTT) |
| | Ile(ATC) Ala(GCT) Leu(TTA) | Ile(ATC) Leu(CTA) Val(GTA) |
| | Asn(AAC) Ile(ATT) Gly(GGT) | Cys(TGT) Asn(AAT) Val(GTT) |
| | Val(GTT) Val(GTA) | Ala(GCA) Leu(TTG) Ala(GCT) |
| | | Phe(TTT) Met(ATG) Gly(GGT) |
| Gly(GGC) | Ala(GCG) Phe(TTT) Ala(GCC) | Gly(GGA) Leu(CTG) Asn(AAC) |
| | Leu(CTT) Val(GTC) Gly(GGA) | Leu(CTT) Ile(ATC) Leu(CTA) |
| | Ala(GCA) Asn(AAT) Met(ATG) | Val(GTA) Cys(TGT) Asn(AAT) |
| | Ile(ATC) Ala(GCT) Leu(TTA) | Val(GTT) Ala(GCA) Leu(TTG) |
| | Asn(AAC) Ile(ATT) Gly(GGT) | Leu(TTA) Ala(GCT) Phe(TTT) |
| | Val(GTT) Val(GTA) | Met(ATG) Gly(GGT) Ile(ATT) |
| Gly(GGG) | Cys(TGT) Phe(TTC) Leu(CTC) | Ile(ATA) Leu(CTC) Val(GTC) |
| | Leu(TTG) Val(GTG) Leu(CTG) | Cys(TGC) Phe(TTC) Ala(GCG) |
| | Ile(ATA) Leu(CTA) Gly(GGC) | Ala(GCC) Val(GTG) Gly(GGC) |
| | Ala(GCG) Phe(TTT) Ala(GCC) | Gly(GGA) Leu(CTG) Asn(AAC) |
| | Leu(CTT) Val(GTC) Gly(GGA) | Leu(CTT) Ile(ATC) Leu(CTA) |
| | Ala(GCA) Asn(AAT) Met(ATG) | Val(GTA) Cys(TGT) Asn(AAT) |
| | Ile(ATC) Ala(GCT) Leu(TTA) | Val(GTT) Ala(GCA) Leu(TTG) |
| | Asn(AAC) Ile(ATT) Gly(GGT) | Leu(TTA) Ala(GCT) Phe(TTT) |
| | Val(GTT) Val(GTA) | Met(ATG) Gly(GGT) Ile(ATT) |
| Gly(GGT) | Val(GTT) Val(GTA) | Ile(ATT) |
| His(CAC) | Ser(TCG) Ser(TCC) Phe(TTC) | Ile(ATC) Leu(CTA) Ser(TCA) |
| | Ser(AGC) Leu(CTC) Leu(TTG) | Val(GTA) Cys(TGT) Val(GTT) |
| | Pro(CCT) Ser(AGT) Val(GTG) | Ala(GCA) Tyr(TAT) Leu(TTG) |
| | Leu(CTG) Tyr(TAC) His(CAT) | Thr(ACT) Pro(CCA) Leu(TTA) |
| | Pro(CCG) Ile(ATA) Leu(CTA) | Ala(GCT) Phe(TTT) Met(ATG) |
| | Ser(TCA) Gly(GGC) Tyr(TAT) | Gly(GGT) Ser(AGT) Thr(ACA) |
| | Ala(GCG) Phe(TTT) Ala(GCC) | Ile(ATT) Pro(CCT) His(CAT) |
| | Leu(CTT) Val(GTC) Ser(TCT) | |
| | Thr(ACC) Gly(GGA) Ala(GCA) | |
| | Met(ATG) Ile(ATC) Ala(GCT) | |
| | Leu(TTA) Thr(ACG) Thr(ACT) | |
| | Pro(CCA) Thr(ACA) Ile(ATT) | |
| | Gly(GGT) Val(GTT) Val(GTA) | |
| His(CAT) | Pro(CCG) Ile(ATA) Leu(CTA) | |
| | Ser(TCA) Gly(GGC) Tyr(TAT) | |
| | Ala(GCG) Phe(TTT) Ala(GCC) | |
| | Leu(CTT) Val(GTC) Ser(TCT) | |
| | Thr(ACC) Gly(GGA) Ala(GCA) | |
| | Met(ATG) Ile(ATC) Ala(GCT) | |
| | Leu(TTA) Thr(ACG) Thr(ACT) | |
| | Pro(CCA) Thr(ACA) Ile(ATT) | |
| | Gly(GGT) Val(GTT) Val(GTA) | |
| Ile(ATA) | Ile(ATC)) Ile(ATT) | Ile(ATC) Ile(ATT) |
| Ile(ATC) | Ile(ATT) | Ile(ATT) |
| Ile(ATT) | | |
| Leu(CTA) | Leu(CTT) Val(GTC) Ile(ATC) | Val(GTA) Val(GTT) Leu(TTG) |
| | Leu(TTA) Ile(ATT) Val(GTT) | Leu(TTA) Ile(ATT) |
| | Val(GTA) | |
| Leu(CTC) | Leu(TTG) Val(GTG) Leu(CTG) | Val(GTC) Val(GTG) Leu(CTG) |
| | Ile(ATA) Leu(CTA) Leu(CTT) | Leu(CTT) Ile(ATC) Leu(CTA) |
| | Val(GTC) Ile(ATC) Leu(TTA) | Val(GTA) Val(GTT) Leu(TTG) |
| | Ile(ATT) Val(GTT) Val(GTA) | Leu(TTA) Ile(ATT) |
| Leu(CTG) | Ile(ATA) Leu(CTA) Leu(CTT) | Leu(CTT)) Ile(ATC) Leu(CTA) |
| | Val(GTC) Ile(ATC) Leu(TTA) | Val(GTA) Val(GTT) Leu(TTG)) |
| | Ile(ATT) Val(GTT) Val(GTA) | Leu(TTA) Ile(ATT) |
| Leu(CTT) | Val(GTC) Ile(ATC) Leu(TTA) | Ile(ATC) Leu(CTA) Val(GTA) |
| | Ile(ATT) Val(GTT) Val(GTA) | Val(GTT) Leu(TTG) Leu(TTA) |
| | | Ile(ATT) |
| Leu(TTA) | Ile(ATT) Val(GTT) Val(GTA) | Ile(ATT) |
| Leu(TTG) | Val(GTG) Leu(CTG) Ile(ATA) | Leu(TTA) Ile(ATT) |
| | Leu(CTA) Leu(CTT) Val(GTC) | |
| | Ile(ATC) Leu(TTA) Ile(ATT) | |
| | Val(GTT) Val(GTA) | |
| Lys(AAA) | Val(GTT) Val(GTA) Asp(GAT) | Ala(GCA) Tyr(TAT) Leu(TTG) |
| | Glu(GAA) | Thr(ACT) Pro(CCA) Leu(TTA) |
| | | Ala(GCT) Phe(TTT) Met(ATG) |
| | | Gly(GGT) Ser(AGT) Thr(ACA) |
| | | Ile(ATT) Gln(CAA) Pro(CCT) |
| | | Glu(GAA) Asp(GAT) His(CAT) |
| Lys(AAG) | Ile(ATT) Gly(GGT) Lys(AAA) | Leu(CTT) Ser(TCT) His(CAC) |
| | Val(GTT) Val(GTA) Asp(GAT) | Ile(ATC) Gln(CAG) Leu(CTA) |
| | Glu(GAA) | Ser(TCA) Val(GTA) Cys(TGT) |
| | | Asn(AAT) Val(GTT) Lys(AAA) |
| | | Ala(GCA) Tyr(TAT) Leu(TTG) |
| | | Thr(ACT) Pro(CCA) Leu(TTA)) |
| | | Ala(GCT) Phe(TTT) Met(ATG) |
| | | Gly(GGT) Ser(AGT) Thr(ACA) |
| | | Ile(ATT) Gln(CAA) Pro(CCT) |
| | | Glu(GAA) Asp(GAT) His(CAT) |
| Met(ATG) | Ile(ATC) Leu(TTA) Ile(ATT) | Ile(ATT) |
| | Val(GTT) Val(GTA) | |
| Phe(TTC) | Leu(CTC) Leu(TTG) Val(GTG) | Val(GTG) Leu(CTG) Leu(CTT) |
| | Leu(CTG)) Ile(ATA) Leu(CTA) | Ile(ATC) Leu(CTA) Val(GTA) |
| | Phe(TTT) Leu(CTT) Val(GTC) | Val(GTT) Leu(TTG) Leu(TTA) |
| | Ile(ATC) Leu(TTA) Ile(ATT) | Phe(TTT) Ile(ATT) |
| | Val(GTT) Val(GTA) | |
| Phe(TTT) | Leu(CTT) Val(GTC) Ile(ATC) | Ile(ATT) |
| | Leu(TTA) Ile(ATT) Val(GTT) | |
| | Val(GTA) | |
| Pro(CCA) | Thr(ACA) Ile(ATT) Gly(GGT) | Leu(TTA) Ala(GCT) Phe(TTT) |
| | Val(GTT) Val(GTA) | Met(ATG) Gly(GGT) Ser(AGT) |
| | | Thr(ACA) Ile(ATT) Pro(CCT) |
| Pro(CCC) | Gly(GGG) Cys(TGT) Ser(TCG) | Leu(CTC) Pro(CCG) Val(GTC) |
| | Ser(TCC) Phe(TTC) Ser(AGC) | Ser(TCC)) Cys(TGC) Phe(TTC) |
| | Leu(CTC) Leu(TTG) Pro(CCT) | Thr(ACG) Ala(GCG) Ala(GCC) |
| | Ser(AGT) Val(GTG) Leu(CTG) | Tyr(TAC) Thr(ACC) Trp(TGG) |
| | Tyr(TAC) Pro(CCG) Ile(ATA) | Val(GTG) Gly(GGC) Gly(GGA) |
| | Leu(CTA) Ser(TCA) Gly(GGC) | Leu(CTG) Ser(AGC) Leu(CTT) |
| | Tyr(TAT) Ala(GCG) Phe(TTT) | Ser(TCT) Ile(ATC) Leu(CTA) |
| | Ala(GCC) Leu(CTT) Val(GTC) | Ser(TCA) Val(GTA) Cys(TGT) |
| | Ser(TCT) Thr(ACC) Gly(GGA) | Val(GTT) Ala(GCA) Tyr(TAT) |
| | Ala(GCA) Met(ATG) Ile(ATC) | Leu(TTG) Thr(ACT) Pro(CCA) |
| | Ala(GCT) Leu(TTA) Thr(ACG) | Leu(TTA) Ala(GCT) Phe(TTT) |
| | Thr(ACT) Pro(CCA) Thr(ACA) | Met(ATG) Gly(GGT) Ser(AGT) |
| | Ile(ATT) Gly(GGT) Val(GTT) | Thr(ACA) Ile(ATT) Pro(CCT) |
| | Val(GTA) | |
| Pro(CCG) | Ile(ATA) Leu(CTA) Ser(TCA) | Val(GTC) Ser(TCC) Cys(TGC) |
| | Gly(GGC) Tyr(TAT) Ala(GCG) | Phe(TTC) Thr(ACG) Ala(GCG) |
| | Phe(TTT) Ala(GCC) Leu(CTT) | Ala(GCC) Tyr(TAC) Thr(ACC) |
| | Val(GTC) Ser(TCT) Thr(ACC) | Trp(TGG) Val(GTG) Gly(GGC) |
| | Gly(GGA) Ala(GCA) Met(ATG) | Gly(GGA) Leu(CTG) Ser(AGC) |
| | Ile(ATC) Ala(GCT) Leu(TTA) | Leu(CTT) Ser(TCT) Ile(ATC) |
| | Thr(ACG) Thr(ACT) Pro(CCA) | Leu(CTA) Ser(TCA) Val(GTA) |
| | Thr(ACA) Ile(ATT) Gly(GGT) | Cys(TGT) Val(GTT) Ala(GCA) |
| | Val(GTT) Val(GTA) | Tyr(TAT) Leu(TTG) Thr(ACT) |
| | | Pro(CCA) Leu(TTA) Ala(GCT) |
| | | Phe(TTT) Met(ATG) Gly(GGT) |
| | | Ser(AGT) Thr(ACA) Ile(ATT) |
| | | Pro(CCT) |
| Pro(CCT) | Ser(AGT) Val(GTG) Leu(CTG) | |
| | Tyr(TAC) Pro(CCG) Ile(ATA) | |
| | Leu(CTA) Ser(TCA) Gly(GGC) | |
| | Tyr(TAT) Ala(GCG) Phe(TTT) | |
| | Ala(GCC) Leu(CTT) Val(GTC) | |
| | Ser(TCT) Thr(ACC) Gly(GGA) | |
| | Ala(GCA) Met(ATG) Ile(ATC) | |
| | Ala(GCT) Leu(TTA) Thr(ACG) | |
| | Thr(ACT) Pro(CCA) Thr(ACA) | |
| | Ile(ATT) Gly(GGT) Val(GTT) | |
| | Val(GTA) | |
| Ser(AGC) | Leu(CTC) Leu(TTG) Ser(AGT) | Leu(CTT) Ser(TCT) Ile(ATC) |
| | Val(GTG) Leu(CTG) Ile(ATA) | Leu(CTA) Ser(TCA) Val(GTA) |
| | Leu(CTA) Ser(TCA) Gly(GGC) | Cys(TGT) Val(GTT) Ala(GCA) |
| | Ala(GCG) Phe(TTT) Ala(GCC) | Leu(TTG) Thr(ACT) Leu(TTA) |
| | Leu(CTT) Val(GTC) Ser(TCT) | Ala(GCT) Phe(TTT) Met(ATG) |
| | Thr(ACC) Gly(GGA) Ala(GCA) | Gly(GGT) Ser(AGT) Thr(ACA) |
| | Met(ATG) Ile(ATC) Ala(GCT) | Ile(ATT) |
| | Leu(TTA) Thr(ACG) Thr(ACT) | |
| | Thr(ACA) Ile(ATT) Gly(GGT) | |
| | Val(GTT) Val(GTA) | |
| Ser(AGT) | Val(GTG) Leu(CTG) Ile(ATA) | Thr(ACA) Ile(ATT) |
| | Leu(CTA) Ser(TCA) Gly(GGC) | |
| | Ala(GCG) Phe(TTT) Ala(GCC) | |
| | Leu(CTT) Val(GTC) Ser(TCT) | |
| | Thr(ACC) Gly(GGA) Ala(GCA) | |
| | Met(ATG) Ile(ATC) Ala(GCT) | |
| | Leu(TTA) Thr(ACG) Thr(ACT) | |
| | Thr(ACA) Ile(ATT) Gly(GGT) | |
| | Val(GTT) Val(GTA) | |
| Ser(TCA) | Gly(GGC) Ala(GCG) Phe(TTT) | Val(GTA) Cys(TGT) Val(GTT) |
| | Ala(GCC) Leu(CTT) Val(GTC) | Ala(GCA) Leu(TTG) Thr(ACT) |
| | Ser(TCT) Thr(ACC) Gly(GGA) | Leu(TTA) Ala(GCT) Phe(TTT) |
| | Ala(GCA) Met(ATG) Ile(ATC) | Met(ATG) Gly(GGT) Ser(AGT) |
| | Ala(GCT) Leu(TTA) Thr(ACG) | Thr(ACA) Ile(ATT) |
| | Thr(ACT) Thr(ACA) Ile(ATT) | |
| | Gly(GGT) Val(GTT) Val(GTA) | |
| Ser(TCC) | Phe(TTC) Ser(AGC) Leu(CTC) | Cys(TGC) Phe(TTC) Thr(ACG) |
| | Leu(TTG) Ser(AGT) Val(GTG) | Ala(GCG) Ala(GCC) Thr(ACC) |
| | Leu(CTG) Ile(ATA) Leu(CTA) | Val(GTG) Gly(GGC) Gly(GGA) |
| | Ser(TCA) Gly(GGC) Ala(GCG) | Leu(CTG) Ser(AGC) Leu(CTT) |
| | Phe(TTT) Ala(GCC) Leu(CTT) | Ser(TCT) Ile(ATC) Leu(CTA) |
| | Val(GTC) Ser(TCT) Thr(ACC) | Ser(TCA) Val(GTA) Cys(TGT) |
| | Gly(GGA) Ala(GCA) Met(ATG) | Val(GTT) Ala(GCA) Leu(TTG) |
| | Ile(ATC) Ala(GCT) Leu(TTA) | Thr(ACT) Leu(TTA) Ala(GCT) |
| | Thr(ACG) Thr(ACT) Thr(ACA) | Phe(TTT) Met(ATG) Gly(GGT) |
| | Ile(ATT) Gly(GGT) Val(GTT) | Ser(AGT) Thr(ACA) Ile(ATT) |
| | Val(GTA) | |
| Ser(TCG) | Ser(TCC) Phe(TTC) Ser(AGC) | Gly(GGG) Ile(ATA) Leu(CTC) |
| | Leu(CTC) Leu(TTG) Ser(AGT) | Val(GTC) Ser(TCC) Cys(TGC) |
| | Val(GTG) Leu(CTG) Ile(ATA) | Phe(TTC) Thr(ACG) Ala(GCG) |
| | Leu(CTA) Ser(TCA) Gly(GGC) | Ala(GCC) Thr(ACC) Val(GTG) |
| | Ala(GCG) Phe(TTT) Ala(GCC) | Gly(GGC) Gly(GGA) Leu(CTG) |
| | Leu(CTT) Val(GTC) Ser(TCT) | Ser(AGC) Leu(CTT) Ser(TCT) |
| | Thr(ACC) Gly(GGA) Ala(GCA) | Ile(ATC) Leu(CTA) Ser(TCA) |
| | Met(ATG) Ile(ATC) Ala(GCT) | Val(GTA) Cys(TGT) Val(GTT) |
| | Leu(TTA) Thr(ACG) Thr(ACT) | Ala(GCA) Leu(TTG) Thr(ACT) |
| | Thr(ACA) Ile(ATT) Gly(GGT) | Leu(TTA) Ala(GCT) Phe(TTT) |
| | Val(GTT) Val(GTA) | Met(ATG) Gly(GGT) Ser(AGT) |
| | | Thr(ACA) Ile(ATT) |
| Ser(TCT) | Thr(ACC) Gly(GGA) Ala(GCA) | Ile(ATC) Leu(CTA) Ser(TCA) |
| | Met(ATG) Ile(ATC) Ala(GCT) | Val(GTA) Cys(TGT) Val(GTT) |
| | Leu(TTA) Thr(ACG) Thr(ACT) | Ala(GCA) Leu(TTG) Thr(ACT) |
| | Thr(ACA) Ile(ATT) Gly(GGT) | Leu(TTA) Ala(GCT) Phe(TTT) |
| | Val(GTT) Val(GTA) | Met(ATG) Gly(GGT) Ser(AGT) |
| | | Thr(ACA) Ile(ATT) |
| Thr(ACA) | Ile(ATT) Gly(GGT) Val(GTT) | Ile(ATT) |
| | Val(GTA) | |
| Thr(ACC) | Gly(GGA) Ala(GCA) Met(ATG) | Val(GTG) Gly(GGC) Gly(GGA) |
| | Ile(ATC) Ala(GCT) Leu(TTA) | Leu(CTG) Leu(CTT) Ile(ATC) |
| | Thr(ACG) Thr(ACT) Thr(ACA) | Leu(CTA) Val(GTA) Cys(TGT) |
| | Ile(ATT) Gly(GGT) Val(GTT) | Val(GTT) Ala(GCA) Leu(TTG) |
| | Val(GTA) | Thr(ACT) Leu(TTA) Ala(GCT) |
| | | Phe(TTT) Met(ATG) Gly(GGT) |
| | | Thr(ACA) Ile(ATT) |
| Thr(ACG) | Thr(ACT) Thr(ACA) Ile(ATT) | Ala(GCG) Ala(GCC)) Thr(ACC) |
| | Gly(GGT) Val(GTT) Val(GTA) | Val(GTG) Gly(GGC) Gly(GGA) |
| | | Leu(CTG) Leu(CTT) Ile(ATC) |
| | | Leu(CTA) Val(GTA) Cys(TGT) |
| | | Val(GTT) Ala(GCA) Leu(TTG) |
| | | Thr(ACT) Leu(TTA) Ala(GCT) |
| | | Phe(TTT) Met(ATG) Gly(GGT) |
| | | Thr(ACA) Ile(ATT) |
| Thr(ACT) | Thr(ACA) Ile(ATT) Gly(GGT) | Leu(TTA) Ala(GCT) Phe(TTT) |
| | Val(GTT) Val(GTA) | Met(ATG) Gly(GGT) Thr(ACA) |
| | | Ile(ATT) |
| Trp(TGG) | Cys(TGC) Gly(GGG) Cys(TGT) | Val(GTG) Gly(GGC) Gly(GGA) |
| | Ser(TCG) Ser(TCC) Phe(TTC) | Leu(CTG) Ser(AGC) Leu(CTT) |
| | Ser(AGC) Leu(CTC) Leu(TTG) | Ser(TCT) Ile(ATC) Leu(CTA) |
| | Ser(AGT) Val(GTG) Leu(CTG) | Ser(TCA) Val(GTA) Cys(TGT) |
| | Ile(ATA) Leu(CTA) Ser(TCA) | Val(GTT)) Ala(GCA) Leu(TTG) |
| | Gly(GGC) Ala(GCG) Phe(TTT) | Thr(ACT) Leu(TTA) Ala(GCT) |
| | Ala(GCC) Leu(CTT) Val(GTC) | Phe(TTT) Met(ATG) Gly(GGT) |
| | Ser(TCT) Thr(ACC) Gly(GGA) | Ser(AGT) Thr(ACA) Ile(ATT) |
| | Ala(GCA) Met(ATG) Ile(ATC) | |
| | Ala(GCT) Leu(TTA) Thr(ACG) | |
| | Thr(ACT) Thr(ACA) Ile(ATT) | |
| | Gly(GGT) Val(GTT) Val(GTA) | |
| Tyr(TAC) | Ile(ATA) Leu(CTA) Ser(TCA) | Thr(ACC) Trp(TGG) Val(GTG) |
| | Gly(GGC) Tyr(TAT) Ala(GCG) | Gly(GGC) Gly(GGA) Leu(CTG) |
| | Phe(TTT) Ala(GCC) Leu(CTT) | Ser(AGC) Leu(CTT) Ser(TCT) |
| | Val(GTC) Ser(TCT) Thr(ACC) | Ile(ATC) Leu(CTA) Ser(TCA) |
| | Gly(GGA) Ala(GCA) Met(ATG) | Val(GTA) Cys(TGT) Val(GTT) |
| | Ile(ATC) Ala(GCT) Leu(TTA) | Ala(GCA) Tyr(TAT) Leu(TTG) |
| | Thr(ACG) Thr(ACT) Thr(ACA) | Thr(ACT) Leu(TTA) Ala(GCT) |
| | Ile(ATT) Gly(GGT) Val(GTT) | Phe(TTT) Met(ATG) Gly(GGT) |
| | Val(GTA) | Ser(AGT) Thr(ACA) Ile(ATT) |
| Tyr(TAT) | Ala(GCG) Phe(TTT) Ala(GCC) | Leu(TTG) Thr(ACT) Leu(TTA) |
| | Leu(CTT) Val(GTC) Ser(TCT) | Ala(GCT) Phe(TTT)) Met(ATG) |
| | Thr(ACC) Gly(GGA) Ala(GCA) | Gly(GGT) Ser(AGT) Thr(ACA) |
| | Met(ATG) Ile(ATC) Ala(GCT) | Ile(ATT) |
| | Leu(TTA) Thr(ACG) Thr(ACT) | |
| | Thr(ACA) Ile(ATT) Gly(GGT) | |
| | Val(GTT) Val(GTA) | |
| Val(GTA) | | Val(GTT) Ile(ATT) |
| Val(GTC) | Ile(ATC) Ile(ATT) Val(GTT) | Val(GTG) Ile(ATC) Val(GTA) |
| | Val(GTA) | Val(GTT) Ile(ATT) |
| Val(GTG) | Ile(ATA) Val(GTC) Ile(ATC) | Ile(ATC) Val(GTA) Val(GTT) |
| | Ile(ATT) Val(GTT) Val(GTA) | Ile(ATT) |
| Val(GTT) | Val(GTA) | Ile(ATT) |

The methods described herein can be use to increase or decrease the expression, solubility or usability of a polypeptide expressed in any type of expression system known in the art. Expression systems suitable for use with the methods described herein include, but are not limited to in vitro expression systems and in vivo expression systems. Exemplary in vitro expression systems include, but are not limited to, cell-free transcription/translation systems (e.g., ribosome based protein expression systems). Several such systems are known in the art (see, for example, Tymms (1995) In vitro Transcription and Translation Protocols: Methods in Molecular Biology Volume 37, Garland Publishing, NY).

Exemplary in vivo expression systems include, but are not limited to prokaryotic expression systems such as bacteria (e.g., E. coli and B. subtilis), and eukaryotic expression systems including yeast expression systems (e.g., Saccharomyces cerevisiae), worm expression systems (e.g. Caenorhabditis elegans), insect expression systems (e.g. Sf9 cells), plant expression systems, amphibian expression systems (e.g. melanophore cells), vertebrate including human tissue culture cells, and genetically engineered or virally infected whole animals.

In another embodiment, the present invention is directed to a mutant cell having a genome that has been mutated to comprise one or more one or more expression and/or solubility altering modifications as described herein. In yet another embodiment, the present invention is directed to a recombinant cell (e.g. a prokaryotic cell or a eukaryotic cell) that contains a nucleic acid sequence comprising one or more expression and/or solubility altering modifications as described herein.

In another embodiment, the present invention is directed to a modified nucleic acid sequence capable of higher polypeptide expression or exhibits higher solubility than the corresponding wild-type nucleic acid sequence, wherein the modified nucleic acid sequence comprises one or more expression and/or solubility altering modifications as described herein.

The methods described herein may also be used in conjunction with, or as an improvement to any type of nucleic acid sequence modification known or described in the art. In one embodiment, the methods described herein can be used in conjunction with one or more additional nucleic acid modifications that alter the solubility or expression of a polypeptide encoded by the nucleic acid. For example, polypeptides produced according to the methods described herein may contain one or more modified amino acids. In certain non-limiting embodiments, modified amino acids may be included in a polypeptide produced according to the methods described herein to (a) increase serum half-life of the polypeptide, (b) reduce antigenicity or the polypeptide, (c) increase storage stability of the polypeptide, or (d) alter the activity or function of the polypeptide. Amino acids can be modified, for example, co-translationally or post-translationally during recombinant production (e.g., N-linked glycosylation at N-X-S/T motifs during expression in mammalian cells) or modified by synthetic means. Examples of modified amino acids suitable for use with the methods described herein include, but are not limited to, glycosylated amino acids, sulfated amino acids, prenlyated (e.g., farnesylated, geranylgeranylated) amino acids, acetylated amino acids, PEG-ylated amino acids, biotinylated amino acids, carboxylated amino acids, phosphorylated amino acids, and the like. Exemplary protocol and additional amino acids can be found in Walker (1998) Protein Protocols on CD-ROM Human Press, Towata, N.J.

Also suitable for use with the methods described herein is any technique known in the art for altering the expression or solubility of a recombinant polypeptide in an expression system (e.g. expression of a human polypeptide in a bacterial cell). Techniques that have been developed to facilitate expression and solubility generally focus on optimization of factors extrinsic to the target polypeptide itself (Makrides (1996) Microbiology and Molecular Biology Reviews 60:512; Sorensen and Mortensen (2005) Journal of biotechnology 115:113-128). Techniques for altering expression are known in the art, include, but are not limited to, co-expression of fusion partners (including MBP (Kapust and Waugh (1999) PRS 8:1668-1674), smt (Lee et al. (2008) Polypeptide Sci. 17:1241-1248), and Mistic (Kefala et al. (2007) Journal of Structural and Functional Genomics 8:167-172)), codon enhancement (Carstens (2003) Methods in Molecular Biology 205:225-234; Christen et al. (2009) Polypeptide Expression and Purification), or optimization (Gustafsson et al. (2004) Trends in biotechnology 22:346-353; Kim et al. (1997) Gene 199:293-301; Hatfield G W, Roth D A (2007) Biotechnol Annu Rev 13:27-42) (including removal of 5′ RNA secondary structure (Etchegaray and Inouye (1999) Journal of Biological Chemistry 274:10079-10085)), and the use of protease deficient strains (Gottesman (1990) Methods in enzymology 185:119). Techniques that have been developed specifically to improve solubility of recombinant polypeptides include chaperone co-expression (Tresaugues et al. (2004) Journal of Structural and Functional Genomics 5:195-204; Mogk et al. 2002 Chembiochem 3, 807; Buchner, Faseb J. 1996 10, 10; Beissinger and Buchner, 1998. J. Biol. Chem. 379, 245)), fusion to solubility-enhancing tags or polypeptide domains (Kapust and Waugh (1999) PRS 8:1668-1674; Davis et al. (1999) Biotechnology and bioengineering 65), expression at lower temperature (Makrides (1996) Microbiology and Molecular Biology Reviews 60:512), heat shock (Chen et al. (2002) Journal of molecular microbiology and biotechnology 4:519-524), expression in a different growth medium (Makrides (1996) Microbiology and Molecular Biology Reviews 60:512; Georgiou and Valax (1996) Current Opinion in Biotechnology 7:190-197), reduction of polypeptide expression level (e.g., by using less inducer or a weaker promoter (Wagner et al. (2008) Proc. Natl. Acad. Sci. U.S.A 105:14371-14376)), directed evolution (Pédelacq et al. (2002) Nature biotechnology 20:927-932; Waldo (2003) Current opinion in chemical biology 7:33-38), and rational mutagenesis (Dale et al. (1994) Polypeptide Engineering Design and Selection 7:933-939). Of these methods, only rational mutagenesis relies on understanding the properties of the polypeptide itself, rather than on modifying an external factor. Intrinsic biophysical features influencing polypeptide solubility have received relatively little systematic study, perhaps because of the experimental difficulties involved in accurate solubility quantifications. Other techniques include directing localization or accumulation a polypeptide into the non-reducing environment of the periplasmic space of bacterial cell. This can be performed by adding a signal- or leader-peptides to direct secretion of the polypeptide.

In addition to these techniques for improving expression and solubility, difficult polypeptides can be avoided in favor of homologous proteins with similarly useful properties (Campbell et al. (1972) Cold Spring Harb. Symp. Quant. Biol 36:165-170). Therefore, the ability to identify challenging or promising polypeptides from primary sequence analysis alone would be of substantial value. The methods described herein provide a metric to guide this selection process and streamline identification of practically useful homologous proteins. Codon usage can have an effect on polypeptide expression and RNA secondary structure (Kudla et al. (2009) Science 324:255; Kim et al. (1997) Gene 199:293-301; Wu et al. (2004) Biochemical and Biophysical Research Communications 313:89-96; Wilkinson and Harrison (1991) Nature Biotechnology 9:443-448; Idicula-Thomas and Balaji (2005) Polypeptide Science: A Publication of the Polypeptide Society 14:582; Idicula-Thomas et al. (2006) Bioinformatics 22:278-284). Computational methods can make extraction of mechanistic inferences difficult in large data sets even though they may perform well as predictors (Smialowski et al. (2007) Bioinformatics 23:2536; Magnan et al. (2009) Bioinformatics). Substantial uncertainty remains concerning the physical and biochemical factors that influence heterologous polypeptide expression.

As described herein, methods for altering polypeptide solubility include linkage of a heterologous fusion polypeptides to the polypeptide of interest. In certain embodiments, the methods described herein for modifying a nucleic acid sequence to comprise one or more expression and/or solubility altering modifications as described herein can be used to alter the solubility of a heterologous fusion polypeptide. Examples of heterologous fusion polypeptides suitable for use in conjunction with the methods described herein include, but are not limited to, Glutathione-S-Transferase (GST), Polypeptide Disulfide Isomerase (PDI), Thioredoxin (TRX), Maltose Binding Polypeptide (MBP), His6 tag, Chitin Binding Domain (CBD) and Cellulose Binding Domain (CBD) (Sahadev et al. 2007, Mol. Cell. Biochem.; Dysom et al. 2004, BMC Biotechnol, 14, 32).

Other methods for altering the solubility of a recombinant polypeptide include recovering insoluble polypeptides from inclusion bodies with chaotropic agents. Dilution or dialysis can then be used to promote refolding of the polypeptide in a selected refolding buffer.

Methods for determining the solubility of a polypeptide are known in the art. For example, a recombinant polypeptide can be isolated from a host cell by expressing the recombinant polypeptide in the cell and releasing the polypeptide from within the cell by any method known in the art, including, but not limited to lysis by homogenization, sonication, French press, microfluidizer, or the like, or by using chemical methods such as treatment of the cells with EDTA and a detergent (see Falconer et al., Biotechnol. Bioengin. 53:453-458 [1997]). Bacterial cell lysis can also be obtained with the use of bacteriophage polypeptides having lytic activity (Crabtree and Cronan, J. E., J. Bact., 1984, 158:354-356).

Soluble materials can be separated form insoluble materials by centrifugation of cell lysates (e.g. 18,000×G for about 20 minutes). After separation of lysed materials into soluble and insoluble fractions, soluble polypeptide can be visualized by using denaturing gel electrophoresis. For example, equivalent amount of material from the soluble and insoluble fractions can be migrated through the gel. Polypeptides in both fractions can then be detected by any method known in the art, including, but not limited to staining or by Western blotting using an antibody or any reagent that recognizes the recombinant polypeptide.

Polypeptides can also be isolated from cellular lysates (e.g. prokaryotic cell lysates or eukaryotic cell lysates) by using any standard technique known in the art. For example, recombinant polypeptides can be engineered to comprise an epitope tag such as a Hexahistidine (“hexaHis”) tag or other small peptide tag such as myc or FLAG. Purification can be achieved by immunoprecipitation using antibodies specific to the recombinant peptide (or any epitope tag comprised in the amino sequence of the recombinant polypeptide) or by running the lysate solution through a an affinity column that comprises a matrix for the polypeptide or for any epitope tag comprised in the recombinant polypeptide (see for example, Ausubel et al., eds., Current Protocols in Molecular Biology, Section 10.11.8, John Wiley & Sons, New York [1993]).

Other methods for purifying a recombinant polypeptide include, but are not limited to ion exchange chromatography, hydroxylapatite chromatography, hydrophobic interaction chromatography, preparative isoelectric focusing chromatography, molecular sieve chromatography, HPLC, native gel electrophoresis in combination with gel elution, affinity chromatography, and preparative isoelectric. See, for example, Marston et al. (Meth. Enz., 182:264-275 [1990]).

The methods described herein can also be used to predict the usability (e.g., expression in a useful form at practically useful levels), expression, or solubility characteristics of a polypeptide when expressed in an expression system (e.g., E. coli or human cells).

In one embodiment, the solubility of a polypeptide expressed in an expression system can be predicted by: 1) calculating one or more sequence parameters of a polypeptide sequence, wherein the one or more sequence parameters include, but are not limited to:
(a) the fraction of amino acid residues in the polypeptide that are predicted to be disordered;
(b) the surface exposure and/or burial status of each residue in the polypeptide;
(c) the fractional content of the polypeptide made up by
   i) each amino acid,
   ii) each amino acid predicted to be buried (i.e., what fraction of the polypeptide is ‘predicted buried alanine’) or exposed, and
   iii) each codon, including but not limited to the fraction of the polypeptide made up of “rare” codons for the 4 amino acids Arg (AGG, AGA, CGG, and CGA), Ile (ATA), Leu (CTA), and Pro (CCC);
d) the length of the polypeptide chain;
e) the net charge of the polypeptide;
f) the absolute value of the net charge of the polypeptide;
g) the value for the net charge of the polypeptide divided by the length of the polypeptide;
h) the absolute value of the net charge of the polypeptide divided by the length of the polypeptide;
i) the isoelectric point of the polypeptide;
j) the mean side-chain entropy of the polypeptide (as given by the Creamer scale);
k) the mean side-chain entropy of all residues predicted to be surface-exposed; and
l) the mean hydrophobicity of the polypeptide.
   2) Determining the combined solubility value of each sequence parameter by multiplying the value for each sequence parameter by its solubility regression slope as provided in Tables 8-12 (such that different weights are provided for different outcome models and parameters with no weight provided have a weight of 0), wherein a polypeptide with one or more higher combined solubility values is predicted to be better expressed compared to a polypeptide with a lower combined solubility value.

In another embodiment, the expression of a polypeptide expressed in an expression system (e.g., E. coli or human cells) can be predicted by: 1) calculating one or more sequence parameters of a polypeptide sequence, wherein the one or more sequence parameters include, but are not limited to:
(a) the fraction of amino acid residues in the polypeptide that are predicted to be disordered;
(b) the surface exposure and/or burial status of each residue in the polypeptide;
(c) the fractional content of the polypeptide made up by
   i) each amino acid,
   ii) each amino acid predicted to be buried (i.e., what fraction of the polypeptide is ‘predicted buried alanine’) or exposed, and
   iii) each codon, including but not limited to the fraction of the polypeptide made up of “rare” codons for the 4 amino acids Arg (AGG, AGA, CGG, and CGA), Ile (ATA), Leu (CTA), and Pro (CCC);
d) the length of the polypeptide chain;
e) the net charge of the polypeptide;
f) the absolute value of the net charge of the polypeptide;
g) the value for the net charge of the polypeptide divided by the length of the polypeptide;
h) the absolute value of the net charge of the polypeptide divided by the length of the polypeptide;
i) the isoelectric point of the polypeptide;
j) the mean side-chain entropy of the polypeptide (as given by the Creamer scale);
k) the mean side-chain entropy of all residues predicted to be surface-exposed; and
l) the mean hydrophobicity of the polypeptide.
   2) Determining the combined solubility value of each sequence parameter by multiplying the value for each sequence parameter by its expression regression slope as provided in Tables 8-12 (such that different weights are provided for different outcome models and parameters with no weight provided have a weight of 0), wherein a polypeptide with one or more higher combined expression values is predicted to be better expressed compared to a polypeptide with a lower combined expression value.

In another embodiment, the usability of a polypeptide expressed in an expression system (e.g., E. coli or human cells) can be predicted by: 1) calculating one or more sequence parameters of a polypeptide sequence, wherein the one or more sequence parameters include, but are not limited to:
(a) the fraction of amino acid residues in the polypeptide that are predicted to be disordered;
(b) the surface exposure and/or burial status of each residue in the polypeptide;
(c) the fractional content of the polypeptide made up by
   i) each amino acid,
   ii) each amino acid predicted to be buried (i.e., what fraction of the polypeptide is ‘predicted buried alanine’) or exposed, and
   iii) each codon, including but not limited to the fraction of the polypeptide made up of “rare” codons for the 4 amino acids Arg (AGG, AGA, CGG, and CGA), Ile (ATA), Leu (CTA), and Pro (CCC);
d) the length of the polypeptide chain;
e) the net charge of the polypeptide;
f) the absolute value of the net charge of the polypeptide;
g) the value for the net charge of the polypeptide divided by the length of the polypeptide;
h) the absolute value of the net charge of the polypeptide divided by the length of the polypeptide;
i) the isoelectric point of the polypeptide;
j) the mean side-chain entropy of the polypeptide (as given by the Creamer scale);
k) the mean side-chain entropy of all residues predicted to be surface-exposed; and
l) the mean hydrophobicity of the polypeptide.
   2) Determining the combined usability value of each sequence parameter by multiplying the value for each sequence parameter by its usability regression slope as provided in Tables 8-12 (such that different weights are provided for different outcome models and parameters with no weight provided have a weight of 0), wherein a polypeptide with a higher combined usability value is more likely to produce a more useable polypeptide relative to a polypeptide with a lower combined usability value.

Methods for determining the fraction of amino acid residues in a polypeptide that are predicted to be disordered include any methods or algorithms known in the art. Examples of such methods or algorithms include, but are not limited to Disopred2, Globplot, Disembl., PONDR, IUPred, RONN, Prelink, Foldindex, and NORSp.

Methods for predicting the surface exposure and/or burial status of each residue in the polypeptide include any methods or algorithms known in the art. Examples of such methods or algorithms include, but are not limited to, PHD/PROF, Porter, SSPro2, PSIPRED, Pred2ary, Jpred2, PHDpsi, Predator, HMMSTR, NNSSP, MULPRED, ZPRED, JNET, COILS, and MULTICOIL.

The present invention encompasses any and all nucleic acids encoding a recombinant polypeptide which have been mutated to comprise a solubility or expression altering modification as described herein and any and all methods of making such mutations, regardless of whether that nucleic acid is present in a virus, a plasmid, an expression vector, as a free nucleic acid molecule, or elsewhere.

The methods described herein can be used to generate recombinant polypeptides having altered solubility. The present invention encompasses any and all types of recombinant polypeptides that encoded by a nucleic acid comprising one or more expression and/or solubility altering modifications as described herein. Several different types of recombinant polypeptides are described herein. However, one of skill in the art will recognize that there are other types of recombinant polypeptides can be produced using the methods described herein. The present invention is not limited to any specific types of recombinant polypeptide described here. Instead, it encompasses any and all recombinant polypeptides encoded by a nucleic acid comprising one or more expression and/or solubility altering modifications as described herein.

The expression or solubility of any polypeptide or polypeptide can be modified according to the methods described herein. Polypeptides that can be produced using the methods described herein can be from any source or origin and can include a polypeptide found in prokaryotes, viruses, and eukaryotes, including fungi, plants, yeasts, insects, and animals, including mammals (e.g., humans). Polypeptides that can be produced using the methods described herein include, but are not limited to any polypeptide sequences, known or hypothetical or unknown, which can be identified using common sequence repositories. Examples of such sequence repositories, include, but are not limited to GenBank EMBL, DDBJ and the NCBI. Other repositories can easily be identified by searching on the internet. Polypeptides that can be produced using the methods described herein also include polypeptides have at least about 30% or more identity to any known or available polypeptide (e.g., a therapeutic polypeptide, a diagnostic polypeptide, an industrial enzyme, or portion thereof, and the like).

Polypeptides that can be produced using the methods described herein also include polypeptides comprising one or more non-natural amino acids. As used herein, a non-natural amino acid can be, but is not limited to, an amino acid comprising a moiety where a chemical moiety is attached, such as an aldehyde- or keto-derivatized amino acid, or a non-natural amino acid that includes a chemical moiety. A non-natural amino acid can also be an amino acid comprising a moiety where a saccharide moiety can be attached, or an amino acid that includes a saccharide moiety.

Exemplary polypeptides that can be produced using the methods described herein include but are not limited to, cytokines, inflammatory molecules, growth factors, their receptors, and oncogene products or portions thereof. Examples of cytokines, inflammatory molecules, growth factors, their receptors, and oncogene products include, but are not limited to e.g., alpha-1 antitrypsin, Angiostatin, Antihemolytic factor, antibodies (including an antibody or a functional fragment or derivative thereof selected from: Fab, Fab′, F(ab)2, Fd, Fv, ScFv, diabody, tribody, tetrabody, dimer, trimer or minibody), angiogenic molecules, angiostatic molecules, Apolipopolypeptide, Apopolypeptide, Asparaginase, Adenosine deaminase, Atrial natriuretic factor, Atrial natriuretic polypeptide, Atrial peptides, Angiotensin family members, Bone Morphogenic Polypeptide (BMP-1, BMP-2, BMP-3, BMP-4, BMP-5, BMP-6, BMP-7, BMP-8a, BMP-8b, BMP-10, BMP-15, etc.); C-X-C chemokines (e.g., T39765, NAP-2, ENA-78, Gro-a, Gro-b, Gro-c, IP-10, GCP-2, NAP-4, SDF-1, PF4, MIG), Calcitonin, CC chemokines (e.g., Monocyte chemoattractant polypeptide-1, Monocyte chemoattractant polypeptide-2, Monocyte chemoattractant polypeptide-3, Monocyte inflammatory polypeptide-1 alpha, Monocyte inflammatory polypeptide-1 beta, RANTES, 1309, R83915, R91733, HCC1, T58847, D31065, T64262), CD40 ligand, C-kit Ligand, Ciliary Neurotrophic Factor, Collagen, Colony stimulating factor (CSF), Complement factor 5a, Complement inhibitor, Complement receptor 1, cytokines, (e.g., epithelial Neutrophil Activating Peptide-78, GRO alpha/MGSA, GRO beta, GRO gamma, MIP-1 alpha, MIP-1 delta, MCP-1), deoxyribonucleic acids, Epidermal Growth Factor (EGF), Erythropoietin (“EPO”, representing a preferred target for modification by the incorporation of one or more non-natural amino acid), Exfoliating toxins A and B, Factor IX, Factor VII, Factor VIII, Factor X, Fibroblast Growth Factor (FGF), Fibrinogen, Fibronectin, G-CSF, GM-CSF, Glucocerebrosidase, Gonadotropin, growth factors, Hedgehog polypeptides (e.g., Sonic, Indian, Desert), Hemoglobin, Hepatocyte Growth Factor (HGF), Hepatitis viruses, Hirudin, Human serum albumin, Hyalurin-CD44, Insulin, Insulin-like Growth Factor (IGF-I, IGF-II), interferons (e.g., interferon-alpha, interferon-beta, interferon-gamma, interferon-epsilon, interferon-zeta, interferon-eta, interferon-kappa, interferon-lambda, interferon-T, interferon-zeta, interferon-omega), glucagon-like peptide (GLP-1), GLP-2, GLP receptors, glucagon, other agonists of the GLP-1R, natriuretic peptides (ANP, BNP, and CNP), Fuzeon and other inhibitors of HIV fusion, Hurudin and related anticoagulant peptides, Prokineticins and related agonists including analogs of black mamba snake venom, TRAIL, RANK ligand and its antagonists, calcitonin, amylin and other glucoregulatory peptide hormones, and Fc fragments, exendins (including exendin-4), exendin receptors, interleukins (e.g., IL-1, IL-2, IL-3, IL-4, IL-5, IL-6, IL-7, IL-8, IL-9, IL-10, IL-11, IL-12, etc.), I-CAM-1/LFA-1, Keratinocyte Growth Factor (KGF), Lactoferrin, leukemia inhibitory factor, Luciferase, Neurturin, Neutrophil inhibitory factor (NIF), oncostatin M, Osteogenic polypeptide, Parathyroid hormone, PD-ECSF, PDGF, peptide hormones (e.g., Human Growth Hormone), Oncogene products (Mos, Rel, Ras, Raf, Met, etc.), Pleiotropin, Polypeptide A, Polypeptide G, Pyrogenic exotoxins A, B, and C, Relaxin, Renin, ribonucleic acids, SCF/c-kit, Signal transcriptional activators and suppressors (p53, Tat, Fos, Myc, Jun, Myb, etc.), Soluble complement receptor 1, Soluble I-CAM 1, Soluble interleukin receptors (IL-1, 2, 3, 4, 5, 6, 7, 9, 10, 11, 12, 13, 14, 15), soluble adhesion molecules, Soluble TNF receptor, Somatomedin, Somatostatin, Somatotropin, Streptokinase, Superantigens, i.e., Staphylococcal enterotoxins (SEA, SEB, SEC1, SEC2, SEC3, SED, SEE), Steroid hormone receptors (such as those for estrogen, progesterone, testosterone, aldosterone, LDL receptor ligand and corticosterone), Superoxide dismutase (SOD), Toll-like receptors (such as Flagellin), Toxic shock syndrome toxin (TSST-1), Thymosin a 1, Tissue plasminogen activator, transforming growth factor (TGF-alpha, TGF-beta), Tumor necrosis factor beta (TNF beta), Tumor necrosis factor receptor (TNFR), Tumor necrosis factor-alpha (TNF alpha), transcriptional modulators (for example, genes and transcriptional modular polypeptides that regulate cell growth, differentiation and/or cell regulation), Vascular Endothelial Growth Factor (VEGF), virus-like particle, VLA-4NCAM-1, Urokinase, signal transduction molecules, estrogen, progesterone, testosterone, aldosterone, LDL, corticosterone.

Additional polypeptides that can be produced using the methods described herein include but are not limited to enzymes (e.g., industrial enzymes) or portions thereof. Examples of enzymes include, but are not limited to amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, and nucleases.

Other polypeptides that that can be produced using the methods described herein include, but are not limited to, agriculturally related polypeptides such as insect resistance polypeptides (e.g., Cry polypeptides), starch and lipid production enzymes, plant and insect toxins, toxin-resistance polypeptides, Mycotoxin detoxification polypeptides, plant growth enzymes (e.g., Ribulose 1,5-Bisphosphate Carboxylase/Oxygenase), lipoxygenase, and Phosphoenolpyruvate carboxylase.

Polypeptides that that can be produced using the methods described herein include, but are not limited to, antibodies, immunoglobulin domains of antibodies and their fragments. Examples of antibodies include, but are not limited to antibodies, antibody fragments, antibody derivatives, Fab fragments, Fab′ fragments, F(ab)2 fragments, Fd fragments, Fv fragments, single-chain Fv fragments (scFv), diabodies, tribodies, tetrabodies, dimers, trimers, and minibodies.

Polypeptides that that can be produced using the methods described herein can be a prophylactic vaccine or therapeutic vaccine polypeptides. A prophylactic vaccine is one administered to subjects who are not infected with a condition against which the vaccine is designed to protect. In certain embodiments, a preventive vaccine will prevent a virus from establishing an infection in a vaccinated subject, i.e. it will provide complete protective immunity. However, even if it does not provide complete protective immunity, a prophylactic vaccine may still confer some protection to a subject. For example, a prophylactic vaccine may decrease the symptoms, severity, and/or duration of the disease. A therapeutic vaccine, is administered to reduce the impact of a viral infection in subjects already infected with that virus. A therapeutic vaccine may decrease the symptoms, severity, and/or duration of the disease.

As described herein, vaccine polypeptides include polypeptides, or polypeptide fragments from infectious fungi (e.g., Aspergillus, Candida species) bacteria (e.g. E. coli, Staphylococci aureus)), or Streptococci (e.g., pneumoniae); protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picornaviruses, e.g., polio; Togaviruses, e.g., rubella; Flaviviruses, e.g., HCV; and Coronaviruses), (−) RNA viruses (e.g., Rhabdoviruses, e.g., VSV; Paramyxovimses, e.g., RSV; Orthomyxovimses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B

In yet another aspect, the methods described herein relate to a method for immunizing a subject against a virus comprising administering to the subject an effective amount of a recombinant polypeptide encoded by a nucleic acid sequence comprising one or more expression and/or solubility altering modifications as described herein. In one embodiment, the invention is directed to a method for immunizing a subject against a virus, comprising administering to the subject an effective amount of recombinant polypeptide encoded by a nucleic acid sequence comprising one or more expression and/or solubility altering modifications as described herein.

In another embodiment, the invention is directed to a composition comprising a recombinant polypeptide encoded by a nucleic acid sequence comprising one or more expression and/or solubility altering modifications as described herein, and an additional component selected from the group consisting of pharmaceutically acceptable diluents, carriers, excipients and adjuvants.

Any recombinant polypeptide encoded by a nucleic acid sequence comprising one or more expression and/or solubility altering modifications as described herein can have one or more altered therapeutic, diagnostic, or enzymatic properties. Examples of therapeutically relevant properties include serum half-life, shelf half-life, stability, immunogenicity, therapeutic activity, detectability (e.g., by the inclusion of reporter groups (e.g., labels or label binding sites)) in the non-natural amino acids, specificity, reduction of LD50 or other side effects, ability to enter the body through the gastric tract (e.g., oral availability), or the like. Examples of relevant diagnostic properties include shelf half-life, stability (including thermostability), diagnostic activity, detectability, specificity, or the like. Examples of relevant enzymatic properties include shelf half-life, stability, specificity, enzymatic activity, production capability, resistance to at least one protease, tolerance to at least one non-aqueous solvent, or the like.

Polypeptides that that can be produced using the methods described herein can also further comprise a chemical moiety selected from the group consisting of: cytotoxins, pharmaceutical drugs, dyes or fluorescent labels, a nucleophilic or electrophilic group, a ketone or aldehyde, azide or alkyne compounds, photocaged groups, tags, a peptide, a polypeptide, a polypeptide, an oligosaccharide, polyethylene glycol with any molecular weight and in any geometry, polyvinyl alcohol, metals, metal complexes, polyamines, imidizoles, carbohydrates, lipids, biopolymers, particles, solid supports, a polymer, a targeting agent, an affinity group, any agent to which a complementary reactive chemical group can be attached, biophysical or biochemical probes, isotypically-labeled probes, spin-label amino acids, fluorophores, aryl iodides and bromides.

The nucleic acid sequences comprising one or more expression and/or solubility altering modifications as described herein may also be incorporated into a vector suitable for expressing a recombinant polypeptide in an expression system. The nucleic acid sequences comprising one or more expression and/or solubility altering modifications as described herein may encode any type of recombinant polypeptide, including, but not limited to immunogenic polypeptides, antibodies, hormones, receptors, ligands and the like as well as fragments, variants, homologues and derivatives thereof.

The expression or solubility altering modifications may be made by any suitable mutagenesis method known in the art, including, but are not limited to, site-directed mutagenesis, oligonucleotide-directed mutagenesis, positive antibiotic selection methods, unique restriction site elimination (USE), deoxyuridine incorporation, phosphorothioate incorporation, and PCR-based mutagenesis methods. Details of such methods can be found in, for example, Lewis et al. (1990) Nucl. Acids Res. 18, p 3439; Bohnsack et al. (1996) Meth. Mol. Biol. 57, p 1; Vavra et al. (1996) Promega Notes 58, 30; Altered SitesII in vitro Mutagenesis Systems Technical Manual #TM001, Promega Corporation; Deng et al. (1992) Anal. Biochem. 200, p 81; Kunkel et al. (1985) Proc. Natl. Acad. Sci. USA 82, p 488; Kunke et al. (1987) Meth. Enzymol. 154, p 367; Taylor et al. (1985) Nucl. Acids Res. 13, p 8764; Nakamaye et al. (1986) Nucl. Acids Res. 14, p 9679; Higuchi et al. (1988) Nucl. Acids Res. 16, p 7351; Shimada et al. (1996) Meth. Mol. Biol. 57, p 157; Ho et al. (1989) Gene 77, p 51; Horton et al. (1989) Gene 77, p 61; and Sarkar et al. (1990) BioTechniques 8, p 404. Numerous kits for performing site-directed mutagenesis are commercially available, such as the QuikChange II Site-Directed Mutagenesis Kit from Stratgene Inc. and the Altered Sites II in vitro mutagenesis system from Promega Inc. Such commercially available kits may also be used to mutate AGG motifs to non-AGG sequences. Other techniques that can be used to generate nucleic acid sequences comprising one or more expression and/or solubility altering modifications as described herein are well known to those of skill in the art. See for example Sambrook et al. (2001) Molecular Cloning: A Laboratory Manual, 3rd Ed., Cold Spring Harbor Laboratory, Cold Spring Harbor, N.Y (“Sambrook”).

Any plasmid or expression vector may be used to express a recombinant polypeptide as described herein. One skilled in the art will readily be able to generate or identify a suitable expression vector that contains a promoter to direct expression of the recombinant polypeptide in the desired expression system. For example, if the polypeptide is to be produced in bacterial or human cells, a promoter capable of directing expression in, respectively, bacterial or human cells should be used. Commercially available expression vectors which already contain a suitable promoter and a cloning site for addition of exogenous nucleic acids may also be used. One of skill in the art can readily select a suitable vector and insert the mutant nucleic acids of the invention into such a vector. The mutant nucleic acid should be under the control of a suitable promoter for directing expression of the recombinant polypeptide in an expression system. A promoter that is already present in the vector may be used. Alternatively, an exogenous promoter may be used. Examples of suitable promoters include any promoter known in the art capable of directing expression of a recombinant polypeptide in an expression system. For example, in bacterial systems, any suitable promoter, including the T7 promoter, pL of bacteriophage lambda, plac, ptrp, ptac (ptrp-lac hybrid promoter) and the like may be used. Other elements important for expression of a recombinant polypeptide from an expression vector include, but are not limited to the presence of least origin of replication on the expression vector, a transcription termination element (e.g. G-C rich fragment followed by a poly T sequence in prokaryotic cells), a selectable marker (e.g., ampicillin, tetracycline, chloramphenicol, or kanamycin for prokaryotic host cells), a ribosome binding element (e.g. a Shine-Dalgarno sequence in prokaryotes). One skilled in the art will readily be able to construct an expression vector comprising elements sufficient to direct expression of a recombinant polypeptide in an expression system.

Methods for transforming cells with an expression vector are well characterized, and include, but are not limited to calcium phosphate precipitation methods and or electroporation methods. Exemplary host cells suitable for expressing the recombinant polypeptides described herein include, but are not limited to any number of E. coli strains (e.g., BL21, HB101, JM109, DH5alpha, DH10, and MC1061) and vertebrate tissue culture cells.

The following examples illustrate the present invention, and are set forth to aid in the understanding of the invention, and should not be construed to limit in any way the scope of the invention as defined in the claims which follow thereafter.

### EXAMPLES

### Example 1

### Large Scale Studies Show Unexpected Amino Acid Effects on Polypeptide Expression and Solubility

Statistical analyses on 9,644 consistently expressed and purified polypeptides from the Northeast Structural Genomics Consortium's polypeptide-production pipeline was performed and each were scored independently for expression and solubility levels in order to analyze the amino acid sequence features correlated with high expression and solubility.

Logistic regressions were used to determine the expression and solubility effects of fractional amino acid composition and several bulk sequence parameters including hydrophobicity, side-chain entropy, electrostatic charge, and predicted backbone disorder. Decreasing hydrophobicity correlated with higher expression and solubility. This correlation was derived from the beneficial effect of charged amino acids. Outcome was not otherwise correlated with hydrophobicity. In fact, the three most hydrophobic residues showed different correlations with solubility. Leu showed the strongest negative correlation among amino acids, while Ile showed a significant positive correlation. Several other amino acids also had unexpected effects. Notably, Arg correlated with decreased expression and, most surprisingly, solubility. This effect was only partially attributable to rare codons, although rare codons did significantly reduce expression despite use of a codon-enhanced strain. Additional analyses show that positively but not negatively charged amino acids reduce translation efficiency irrespective of codon usage. These results were used to construct and validate predictors of expression, solubility, and overall polypeptide usability.

In one aspect, the methods described herein are useful for understanding of the physical and chemical mechanisms that influence polypeptide overexpression and solubility.

Results from the polypeptide production pipeline of the Northeast Structural Genomics Consortium (NESG—www nesg.org) were examined. Over 16,000 polypeptide targets have been taken through the same cloning and expression pipeline (Goh et al. (2003) Nucleic acids research 31:283) by NESG and independently scored for the expression level in E. coli and the solubility of the expressed polypeptide. The uniform processing of thousands of targets (Goh et al. (2003) Nucleic acids research 31:283; Goh et al. (2004) Journal of molecular biology 336:115-130) removes methodological variances that can impact polypeptide expression and solubility and effects inherent to the polypeptide sequence itself can be clearly observed. Some determinants of experimental performance (Goh et al. (2004) Journal of Molecular Biology 336:115-130; Price et al. (2009) Nat. Biotechnol 27:51-57) have been elucidated in the NESG pipeline. Provided herein is a statistical analyses of a larger number of observations from the high-throughput experimental pipeline to examine amino acid sequence properties that influence polypeptide expression and solubility. The results described herein show a number of surprising physical and biochemical effects that have evaded characterization via traditional mechanistic experimentation.

Correlation Between Expression and Solubility Levels.

Analyses were performed on 9,644 unique polypeptide targets taken through the uniform polypeptide production and purification pipeline of the NESG between 2001 and mid-2008. These targets did not include polypeptides with large low-complexity regions, predicted transmembrane α-helices, or predicted signal peptides. Some targets were individual domains of multi-domain polypeptides. Polypeptides were expressed from a T7-polymerase-based pET vector carrying short hexa-histidine tags (Acton T B et al. Methods in Enzymology 394:210-243). A subset of 7,733 polypeptides was used for model development and initial regressions, while the remaining 1,911 polypeptides were set aside for use solely in model validation. Polypeptides were assigned integer scores from 0 to 5 independently for expression (E), based on the total amount of polypeptide as shown on SDS-PAGE gels, and for solubility (S), based on the fraction of polypeptide appearing in the soluble fraction after centrifugation to remove insoluble material. These results described herein can be used to develop predictors of polypeptide solubility. Further, these results provide more detail than previous datasets where polypeptides were segregated based on binary criteria (such as the absence or presence of inclusion bodies) (Wilkinson D L, Harrison R G (1991) Nature Biotechnology 9:443-448; Smialowski et al. (2007) Bioinformatics 23:2536; Magnan et al. (2009) Bioinformatics). A third characteristic, practical utility or “usability,” was defined as having E*S>11, which is the operational requirement for polypeptide scale-up and purification by the NESG.

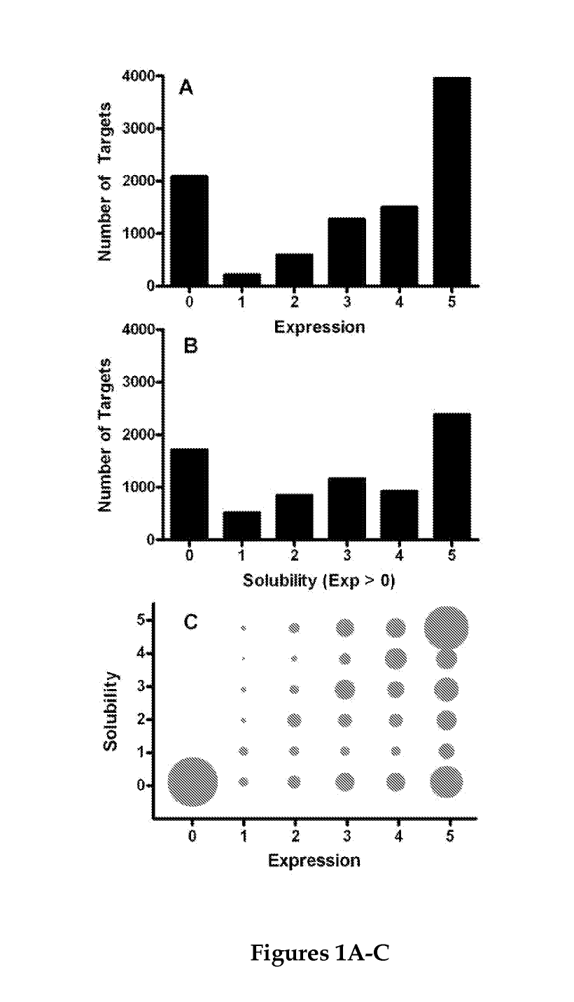

Although all combinations of expression/solubility scores were observed, the majority of polypeptides scored at the extremes of both score ranges (FIG. 1). Higher expression level correlates strongly with higher solubility in this dataset. Expression level predicted solubility level more significantly (p=4.5×10⁻⁶⁷) than any of the sequence parameters evaluated herein when polypeptides showing no expression are excluded. While individual polypeptides can have decreased solubility and improper folding when translational pause sites are removed to accelerate translation (Crombie et al. (1992) J. Mol. Biol 228:7-12; Komar (2009) Trends Biochem. Sci 34:16-24), a negative correlation between polypeptide aggregation tendencies and mRNA expression levels has also been reported (Tartaglia et al. (2009) Journal of Molecular Biology). The results described herein are consistent with the latter observation and show a strong positive correlation between higher translation levels and increased solubility. This relationship can be the result of different molecular mechanisms including, but not limited to degradation of aggregated polypeptides, inhibition of translation upon polypeptide aggregation, decreased cell growth rate upon polypeptide aggregation, or even increased folding efficiency with more rapid translation). The strong correlation makes it difficult to deconvolute effects on expression vs. solubility for parameters that have a consistent effect on both. However, parameters showing a stronger effect on one of the two scores are more likely to act mechanistically on the related biochemical process (i.e., translation efficiency vs. polypeptide solubility), while parameters showing opposite effects on the two scores can be the result of opposing effects on these processes.

Framework for Evaluating Sequence Effects on Expression and Solubility.

Because expression and solubility scores are non-continuous, ordinary least squares regressions are not appropriate to evaluate the relationship between sequence parameters and expression/solubility scores. Therefore, logistic regressions were used to determine which sequence parameters significantly predict expression, solubility, or usability. Logistic regression determines the relationship between continuous independent variables and ranked categorical dependent variables by converting the output variables into an odds ratio for each outcome and performing a linear regression against the logarithm of that parameter (Hosmer and Lemeshow S (2004) Applied logistic regression (Wiley-Interscience)). As opposed to a standard logistic regression, which applies this analysis to a single binary outcome, an ordinal logistic regression applies a similar analysis to the probability of being at or below the value in successive parameter bins (Hosmer and Lemeshow (2004) Applied logistic regression (Wiley-Interscience)). The sequence parameters (continuous independent variables) initially analyzed included the fractional content of each amino acid and twelve aggregate parameters, including isoelectric point, polypeptide length, mean side chain entropy (SCE) (for all residues and those predicted to be surface-exposed by PHD/PROF), GRAVY (the GRand AVerage of hydropathY (Kyte J, Doolittle R F (1982) Journal of Molecular Biology 157:105)), and six electrostatic charge variables (Table 8).

TABLE 8
Parameter names and formulae.

| Variable Name | Parameter | Parameter Formula |
|---|---|---|
| x (e.g., a, c) | Fractional content of residue x | (count of residue x)/(chain length) |
| xb (e.g., cb, db) | predicted buried amino acid | (number of residue x predicted |
| | fraction | buried by PHD/PROF (Rost B |
| | | (2005) The proteomics protocols |
| | | handbook. Totowa (New Jersey): |
| | | Humana: 875-901))/(chain length) |
| xe (e.g., de, ee) | predicted exposed amino acid | (number of residue x predicted |
| | fraction | exposed by PHD/PROF)/(chain |
| | | length) |
| gravy | GRAVY/hydrophobicity | mean residue hydrophobicity (Kyte J, |
| | | Doolittle RF (1982) Journal of |
| | | Molecular Biology 157: 105) |
| sce | side-chain entropy | mean side-chain entropy of all |
| | | residues (Creamer TP (2000) |
| | | Polypeptides: Structure, Function, |
| | | and Genetics 40) |
| esce | predicted exposed side-chain | mean side-chain entropy of residues |
| | entropy | predicted exposed by PHD/PROF |
| numcharge | number of charged residues | R + K + D + E |
| netcharge | net charge | R + K − D − E |
| absnetcharge | absolute net charge | \|R + K − D − E\| |
| fracnumcharge | fraction of charged residues | (R + D + D + E)/(chain length) |
| fracnetcharge | fractional net charge | (R + K − D − E)/(chain length) |
| fracabsnetcharge | fractional absolute net charge | \|R + K − D − E\|/(chain length) |
| diso | fraction predicted disordered | (number of residues predicted |
| | residues | disordered by DISOPRED2 (Ward JJ, |
| | | et al. (2004) The DISOPRED |
| | | server for the prediction of |
| | | polypeptide disorder (Oxford Univ |
| | | Press)))/(chain length) |
| length | chain length | number of residues |
| pi | isoelectric point | EMBOSS algorithm (Rice P, et al. |
| | | (2000) Trends in genetics 16: 276-277) |
| | | at ExPASY (Appel RD, et al. |
| | | (1994) Trends in Biochemical |
| | | Sciences 19: 258) |

Sequence parameters analyzed for correlation with expression, solubility, and usability. Sixty amino acid variables were considered, including the fraction of each amino acid, the predicted buried fraction of each amino acid, and the predicted exposed fraction of each amino acid. Twelve compound variables were also considered, including GRAVY/hydrophobicity, mean side-chain entropy among all or only predicted exposed residues, several charge variables, fraction of residues predicted disordered by DISOPRED2, chain length, and isoelectric point.

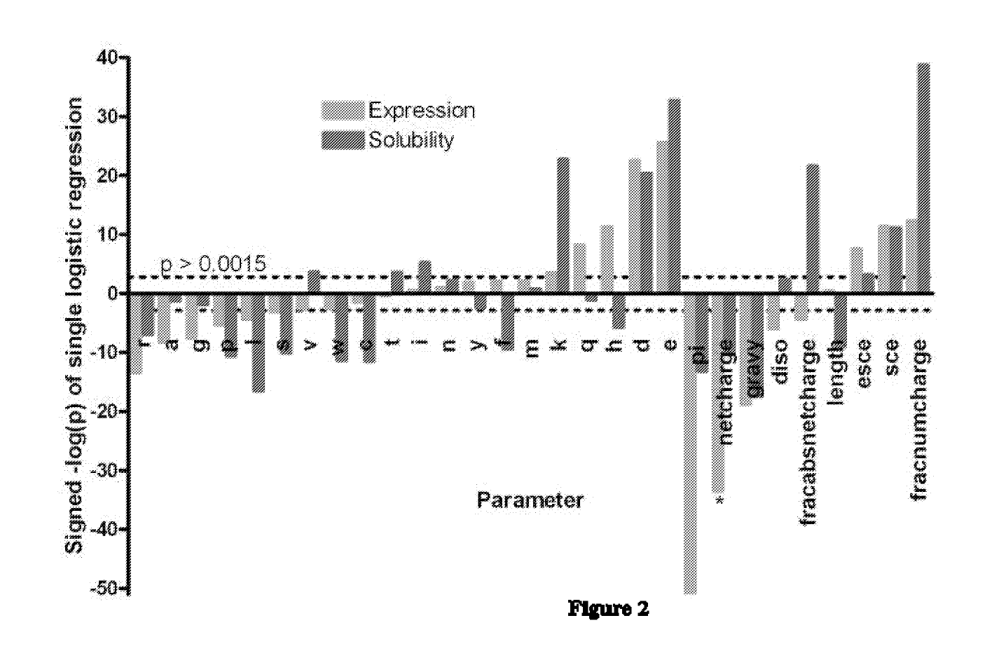

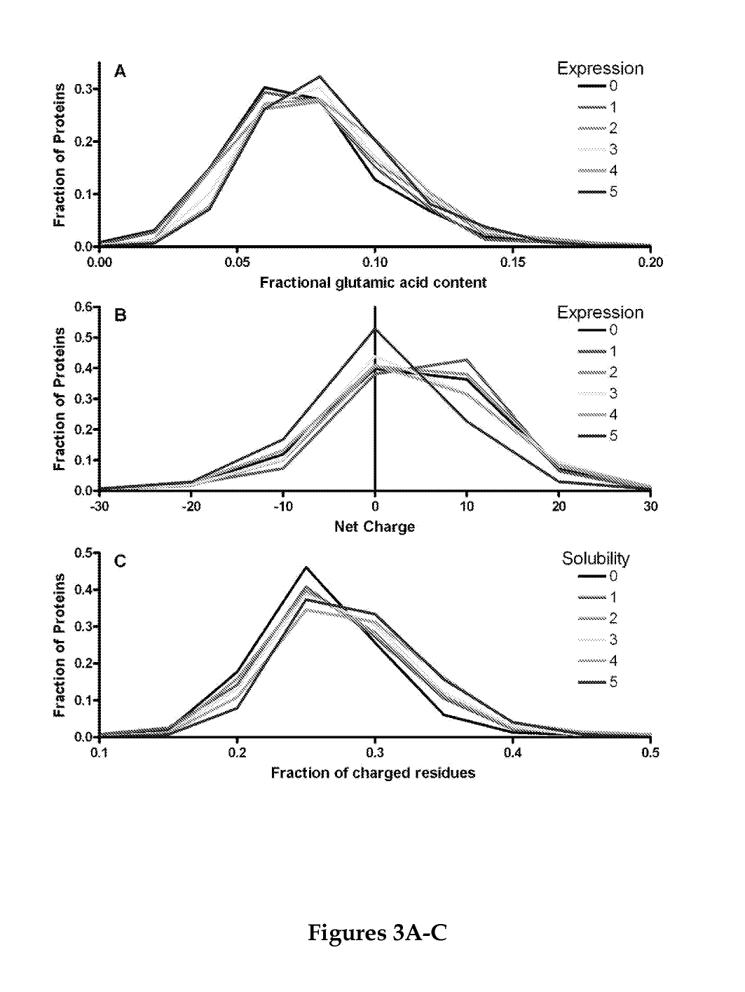

Many parameters had significant effects on each of the output (dependent) variables. FIG. 2 shows the statistical significance and the direction of the correlation with each of the indicated sequence parameters. The plotted value is the negative of the logarithm of the p-value for the ordinal logistic regression against each parameter multiplied by the sign of slope of this regression, so positive correlations yield positive values on this graph. This plotted value scales monotonically with the “predictive value” of the parameter, which is defined as the product of the regression slope (which measures the size of the effect) and the parameter's standard deviation (which normalizes for its range in the dataset). Sample distributions are shown for three significant effects in FIG. 3.

Electrostatic Charge has a Dominant Effect on Expression and Solubility.

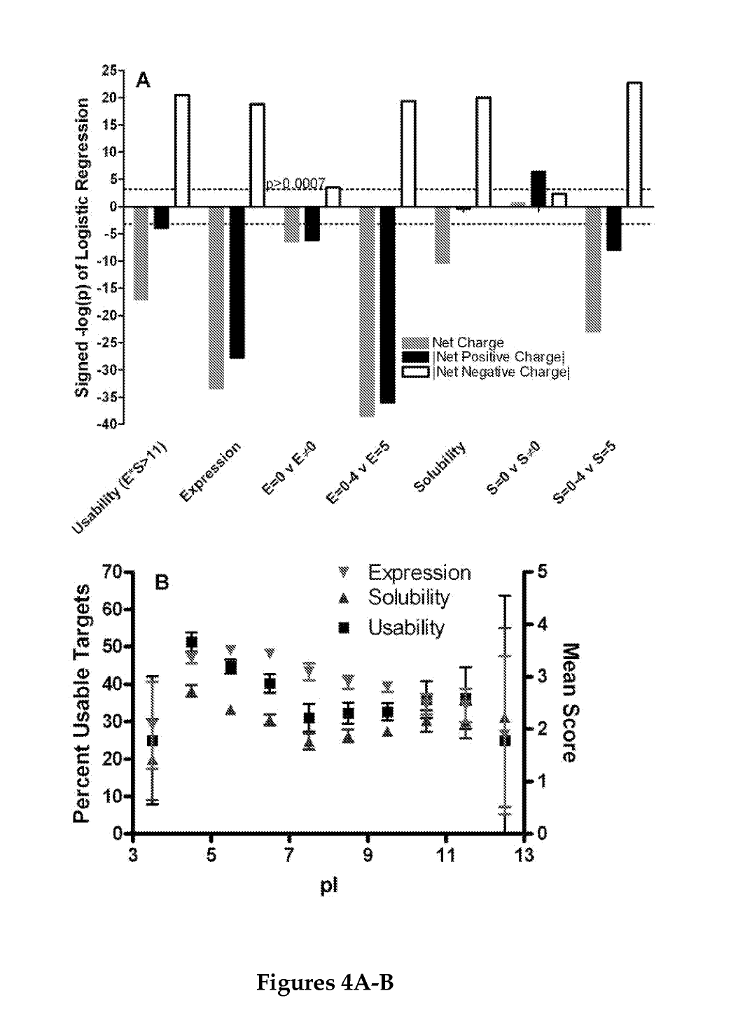

Among the analyzed sequence parameters, the most salient effects are from parameters related to electrostatic charge (FIG. 2). Considering individual amino acids, the fractional content of three of the charged amino acids, Glu, Asp, and Lys, strongly correlates with higher solubility, and Glu and Asp content show similarly strong correlations with higher expression. The fractional content of Arg shows the opposite effect, i.e., significant negative correlations with solubility and especially expression. In spite of the contrary effects of arginine, the length-normalized total charge (fraction of Asp+Glu+Arg+Lys, fracnumcharge) is the strongest positive predictor of solubility among the sequence parameters evaluated, while the length-normalized absolute value of net charge (fracabsnetcharge) is the second strongest positive predictor of solubility among aggregate sequence parameters (right side of FIG. 2). In contrast, net charge has the opposite effect and is a negative predictor of both expression and solubility. This trend derives from two mutually reinforcing sources. Negatively charged residues have a beneficial influence on expression (FIG. 4), which produces a negative regression slope due to the negative mathematical values of the charge parameter. In the case of expression, this effect is reinforced by positively charged residues, which have a deleterious effect (FIG. 4) that also produces a negative regression slope for this mathematically positive parameter. The deleterious influence of isoelectric point (pI) on expression and solubility is attributable to similar causes (FIGS. 2 & 4).

Closer examination of the data shows that positively charged residues can impede translation but negatively charged residues do not. Both Glu and Asp have very strong and similar positive effects on expression and solubility (FIG. 2). Lys and Arg, the other charged amino acids, would naïvely be expected to have similar effects. Instead, Lys has a very strong positive effect on solubility but a much smaller effect on expression, while Arg has significant negative effects on both outcomes. Given the strong correlation between expression and solubility, and the statistical and probably mechanistic dominance of charge on solubility, the simplest explanation for this observation is that positively charged residues reduce translation efficiency. Such an effect, which can derive from their electrostatic attraction to rRNA (Sanbonmatsu, et al. (2005) Proceedings of the National Academy of Sciences of the United States of America 102:15854-15859), been observed for one Arg codon (Pedersen (1984) The EMBO Journal 3:2895). Alternative explanations, including an influence on polypeptide degradation rates, also exist. The opposing effects of positively and negatively charged residues on expression also explain the weaker influence of fracnumcharge on expression than on solubility.

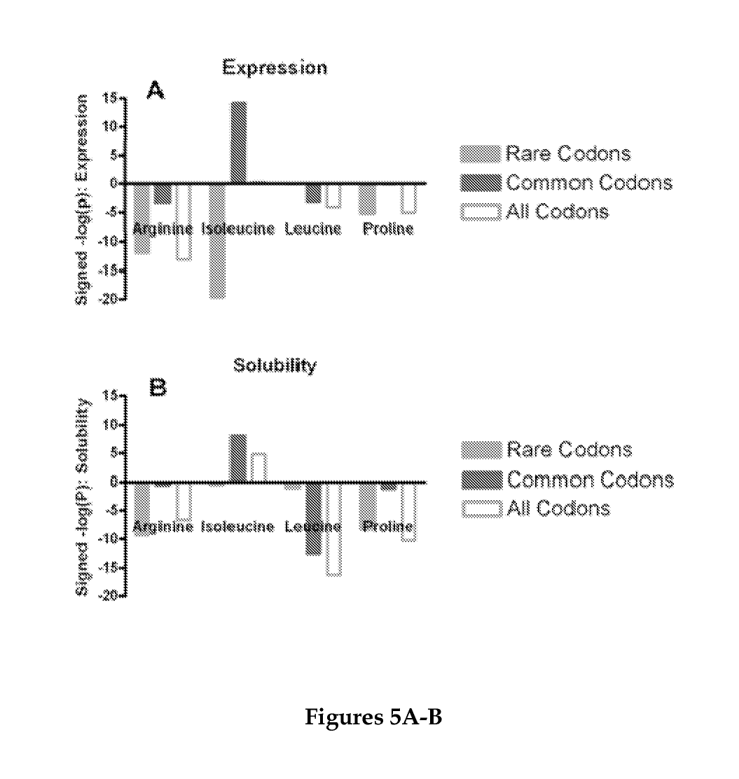

The negative effect of Arg on solubility (FIG. 2) was surprising. Arg is encoded in part by rare codons, which are known to impede expression in some cases (Gustafsson, et al. (2004) Trends in biotechnology 22:346-353). To determine if rare codon effects might be the cause of the negative correlation between Arg and solubility, the fractional content of Arg was split into residues encoded by rare codons and those encoded by common codons. Common Arg had no effect on solubility. This result is in contrast to Lys, which has a positive solubility effect (FIG. 5). Therefore, Arg has one or more biochemical properties which can reduce solubility, despite its positive charge. Arg residues encoded by both rare and common codons have negative effects on expression (FIG. 5), though the effect of rare codon Arg is much more significant, suggesting a combined negative effect on expression from codon rarity and biochemical properties.

Hydrophobicity is not a Dominant Determinant of Expression or Solubility.

Several of the results described herein were unexpected. First, Arg, the most hydrophilic amino acid, was negatively correlated with solubility. Second, Ile, the most hydrophobic amino acid, had a positive correlation with solubility. These observations show that that the influence of side-chain hydrophobicity on solubility is not straightforward. Although mean hydrophobicity is a negative predictor of both expression and solubility (FIG. 2), this effect comes primarily from the positive effects of the charged residues Asp, Glu, and Lys (FIG. 6). Of the seven residues with positive hydrophobicities, four have negative effects on solubility, and three have positive effects. The two most hydrophobic residues, Val and Ile, have positive effects on solubility. It is also possible that the positive effect of some hydrophobic residues is actually a substitution effect (i.e., Ile being less deleterious than Leu at positions constrained to be hydrophobic).

Some other residues have unexpected effects. Ala and Gly both have negative effects on expression but not solubility, which can result from enhanced proteolysis of Ala/Gly-rich sequences. Ser and His both have negative impacts on solubility, but little impact on expression.

Solvent Exposure Predictions Usefully Segregate Amino Acid Parameters.

To determine whether the individual amino acid effects on solubility are influenced by predicted surface exposure even where the expression effects of the same amino acids are be independent of solvent exposure, the fractional amino acid content was divided by whether the amino acid was predicted to be buried or exposed and the same set of ordinal and binary logistic regressions on the separated categories were run for each amino acid. Burial or exposure predictions were obtained with the PhD/PROF program (Rost (2005) The proteomics protocols handbook. Totowa (New Jersey): Humana:875-901). The results of these 72 logistic regressions are shown in Tables 9 & 10.

TABLE 9
Amino Acid Single Logistic Regressions^{a}.

| | Expression | | Solubility | | Usability | |
|---|---|---|---|---|---|---|
| Parameter | Slope | P-Value | Slope | P-Value | Slope | P-value |
| a | −3.07 | 1.27E−08 | −0.96 | 0.119 | −2.71 | 9E−06 |
| ab | −4.83 | 6.3E−08 | −5.88 | 7.04E−09 | −8.09 | 2.19E−15 |
| ae | −2.44 | 0.0009 | 2.20 | 0.0083 | 0.45 | 0.582 |
| c | −2.54 | 0.069 | −11.1 | 6.89E−12 | −11.2 | 3.17E−10 |
| cb | −2.58 | 0.093 | −9.94 | 1.7E−08 | −10.4 | 1.61E−07 |
| ce | −3.73 | 0.384 | −26.1 | 8.8E−08 | −22.9 | 5.12E−06 |
| d | 10.4 | 6.2E−23 | 11.06 | 8.76E−21 | 12.3 | 4.18E−25 |
| db | 15.3 | 7.82E−05 | −8.78 | 0.039 | −3.33 | 0.441 |
| de | 9.65 | 2.97E−19 | 12.1 | 9.19E−24 | 13.0 | 5.93E−27 |
| e | 8.14 | 5.08E−26 | 10.4 | 3.55E−33 | 12.0 | 1.34E−42 |
| eb | 12.3 | 0.029 | −33.9 | 4.25E−08 | −21.6 | 0.0007 |
| ee | 7.80 | 2.44E−24 | 10.9 | 1.12E−36 | 12.2 | 1.18E−44 |
| f | 2.90 | 0.014 | −8.14 | 9.36E−10 | −4.99 | 0.0002 |
| fb | 3.05 | 0.017 | −9.76 | 1.2E−11 | −6.71 | 3.84E−06 |
| fe | 1.84 | 0.529 | 1.41 | 0.674 | 4.12 | 0.204 |
| g | −4.32 | 5.96E−08 | −1.96 | 0.030 | −4.78 | 1.22E−07 |
| gb | −0.82 | 0.465 | −6.40 | 4.9E−07 | −6.56 | 3.06E−07 |
| ge | −5.97 | 1.28E−09 | 1.93 | 0.084 | −2.33 | 0.037 |
| h | 10.1 | 9.76E−12 | −7.56 | 3.48E−06 | −0.75 | 0.645 |
| hb | 12.5 | 3.16E−06 | −12.3 | 2.92E−05 | −5.50 | 0.067 |
| he | 9.51 | 1.61E−07 | −5.66 | 0.0044 | 1.35 | 0.502 |
| i | 0.39 | 0.624 | 4.06 | 1.24E−05 | 3.14 | 0.0005 |
| ib | 1.49 | 0.101 | 3.44 | 0.001 | 2.90 | 0.0042 |
| ie | −4.95 | 0.015 | 8.54 | 0.0003 | 5.66 | 0.013 |
| k | 1.99 | 0.0006 | 6.56 | 3.77E−23 | 6.67 | 1.69E−23 |
| kb | −2.84 | 0.741 | −9.32 | 0.342 | −12.8 | 0.186 |
| ke | 2.03 | 0.0005 | 6.67 | 1.25E−23 | 6.83 | 3.31E−24 |
| l | −2.93 | 8.49E−05 | −7.07 | 6.83E−17 | −6.56 | 9.19E−15 |
| lb | −2.40 | 0.0025 | −7.22 | 1.35E−15 | −6.53 | 4.83E−13 |
| le | −3.61 | 0.020 | −3.20 | 0.069 | −3.87 | 0.029 |
| m | 4.06 | 0.014 | 1.73 | 0.342 | 0.60 | 0.741 |
| mb | 9.08 | 1.03E−05 | −5.78 | 0.010 | −3.66 | 0.111 |
| me | −4.05 | 0.103 | 12.9 | 4.43E−06 | 6.59 | 0.016 |
| n | 1.25 | 0.201 | 2.79 | 0.012 | 2.77 | 0.011 |
| nb | 2.04 | 0.569 | −17.2 | 2.24E−05 | −17.2 | 2.14E−05 |
| ne | 1.19 | 0.242 | 4.38 | 0.0001 | 4.38 | 0.0001 |
| p | −4.25 | 9.42E−06 | −7.19 | 5.03E−11 | −8.52 | 2.17E−14 |
| pb | −1.96 | 0.395 | −21.7 | 3.46E−17 | −20.1 | 1.72E−14 |
| pe | −4.67 | 8.2E−06 | −3.91 | 0.0011 | −5.84 | 1.44E−06 |
| q | 5.47 | 1.2E−08 | −1.44 | 0.171 | 3.06 | 0.0043 |
| qb | 8.22 | 0.057 | −21.0 | 1.24E−05 | −15.9 | 0.0011 |
| qe | 5.24 | 7.87E−08 | −0.45 | 0.674 | 3.95 | 0.0003 |
| r | −5.13 | 8.65E−14 | −4.04 | 2.1E−07 | −4.93 | 1.2E−09 |
| rb | 2.53 | 0.484 | −11.6 | 0.0039 | −9.57 | 0.018 |
| re | −5.40 | 1.16E−14 | −3.72 | 2.48E−06 | −4.74 | 1E−08 |
| s | −2.90 | 0.0017 | −6.72 | 1.66E−10 | −6.55 | 1.06E−09 |
| sb | −1.22 | 0.522 | −15.6 | 3.87E−13 | −15.4 | 1.44E−12 |
| se | −2.77 | 0.0036 | −3.17 | 0.0033 | −2.99 | 0.0063 |
| t | −0.09 | 0.928 | 3.99 | 0.0005 | 2.90 | 0.0128 |
| tb | 1.85 | 0.294 | −11.7 | 3.03E−09 | −10.3 | 2.34E−07 |
| te | −0.79 | 0.465 | 8.81 | 6.02E−13 | 7.11 | 6.25E−09 |
| v | −2.29 | 0.0047 | 3.16 | 0.0005 | 1.20 | 0.190 |
| vb | −1.30 | 0.168 | 1.32 | 0.204 | −0.36 | 0.741 |
| ve | −4.51 | 0.0024 | 7.64 | 6.8E−06 | 5.01 | 0.0031 |
| w | −5.45 | 0.0058 | −15.4 | 8.49E−12 | −12.5 | 4.25E−08 |
| wb | −4.97 | 0.030 | −16.5 | 1.46E−10 | −14.6 | 3.02E−08 |
| we | −9.42 | 0.041 | −15.4 | 0.0040 | −8.62 | 0.105 |
| y | 2.67 | 0.023 | −3.47 | 0.0083 | −0.93 | 0.478 |
| yb | 4.89 | 0.0012 | −4.77 | 0.0042 | −1.66 | 0.327 |
| ye | −0.97 | 0.624 | −1.52 | 0.497 | 0.25 | 0.912 |

Results of single logistic regressions against expression, solubility, and usability for amino acids fractions. Slope and p value are shown. P-values below the Bonferroni threshold of 0.0007 are bold.

TABLE 10
Compound Sequence Parameter Single Logistic Regressions

| | Expression | | Solubility | | Usability | |
|---|---|---|---|---|---|---|
| Parameter | Slope | P-value | Slope | P-value | Slope | P-value |
| netcharge | −0.026 | 7.32E−34 | −0.015 | 8.58E−11 | −0.021 | 1.74E−17 |
| numcharge | 0.0018 | 0.0037 | −0.0007 | 0.327 | 0.0006 | 0.412 |
| absnetcharge | −0.00004 | 0.992 | 0.029 | 1.74E−17 | 0.022 | 1.05E−10 |
| fracnetcharge | −4.78 | 1.05E−30 | −2.86 | 5.65E−10 | −4.13 | 8.80E−17 |
| fracnumcharge | 2.75 | 1.08E−12 | 5.77 | 3.76E−39 | 6.36 | 5.81E−45 |
| fracabsnetcharge | −2.21 | 8.15E−05 | 6.56 | 4.92E−22 | 3.8 | 5.88E−09 |
| sce | 1.46 | 9.10E−12 | 1.62 | 1.70E−11 | 2.39 | 6.85E−23 |
| esce | 0.91 | 5.33E−08 | 0.61 | 0.0013 | 1.17 | 8.25E−10 |
| gravy | −0.62 | 3.55E−19 | −0.68 | 7.31E−18 | −0.93 | 2.04E−31 |
| length | 0.00007 | 0.66 | −0.0011 | 2.23E−09 | −0.0009 | 2.25E−06 |
| diso | −0.67 | 2.14E−06 | 0.41 | 0.0096 | 0.043 | 0.795 |
| pi | −0.16 | 1.20E−51 | −0.09 | 7.43E−14 | −0.13 | 2.77E−27 |

Results of single logistic regressions against expression and solubility for compound sequence parameters. Slope, standard error, Z score, and p-value are shown. P-values below the Bonferroni threshold of 0.0007 are bold.

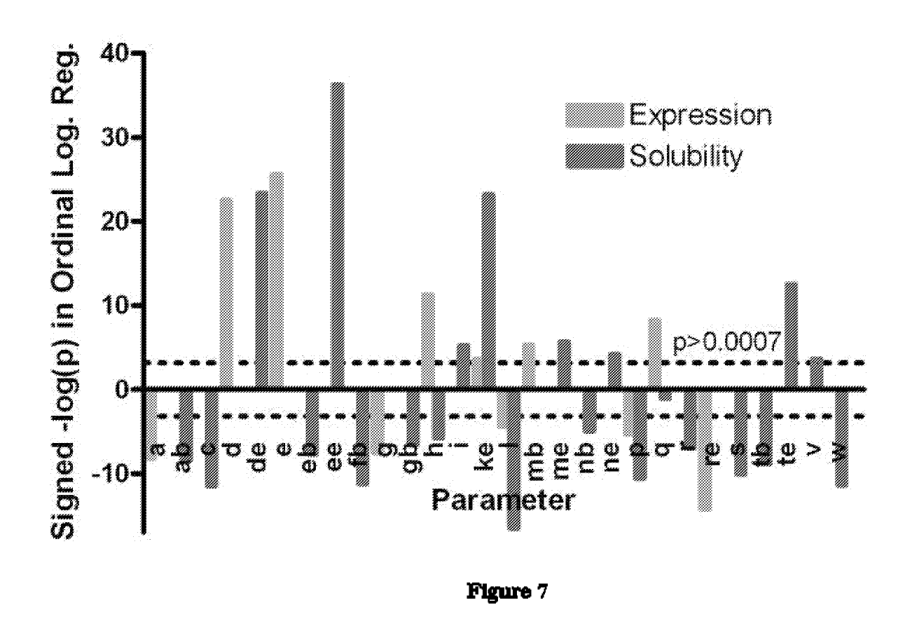

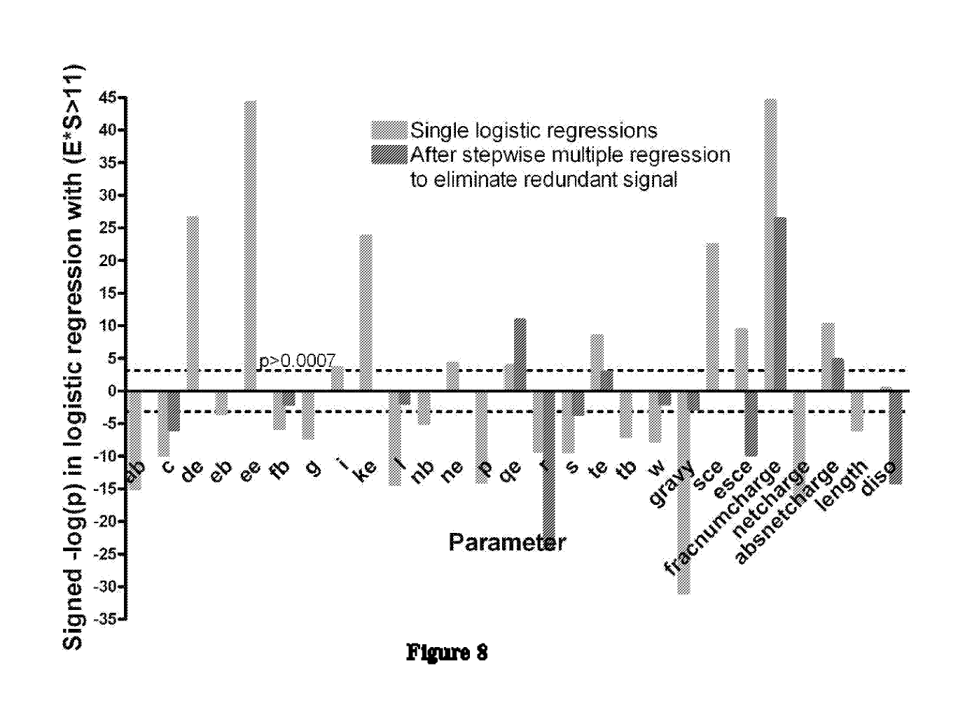

Because some parameters are related and therefore provide redundant signal (e.g., a=ab+ae), parameter divisions are kept only if buried vs. exposed have statistically significant effects with opposite signs (FIGS. 7 and 8). This division of amino acid content shows significant differences for eight amino acids in predicting solubility, but for only two amino acids in predicting expression. In particular, the positive solubility effects of Asp, Glu, and Lys, and to a lesser extent Asn, Met, and Thr, are derived from surface-exposed residues. Beyond supporting the hypothesis that surface localization can mediate amino acid influences on solubility, this analysis shows that the analytical approach described herein can provide insight into differential effects on polypeptide expression vs. solubility, even though the two outcomes are significantly correlated in the dataset.

Combining Parameters for Outcome Prediction.

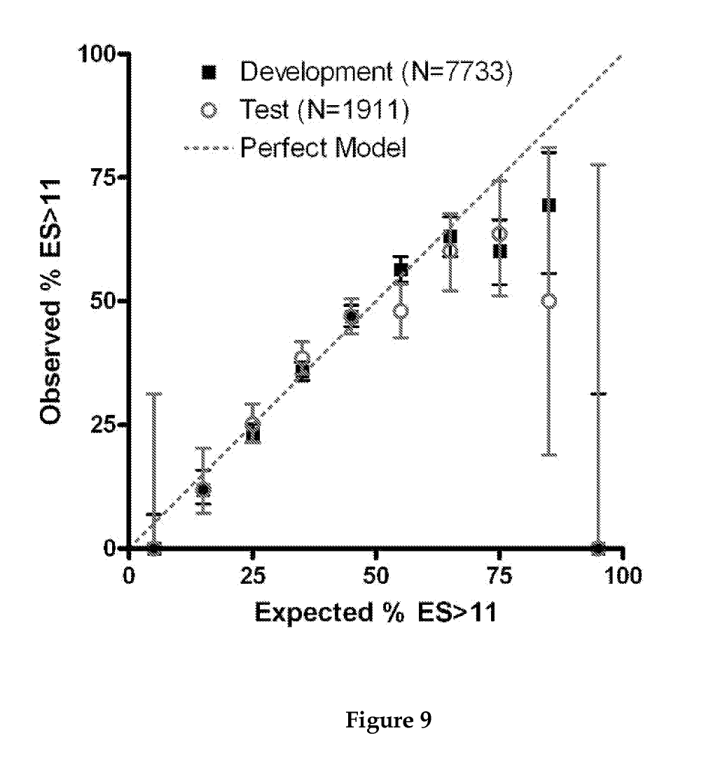

In addition to understanding the mechanistic impact on expression and solubility of different sequence parameters, the methods described herein can be used to create overall predictors based on polypeptide sequence. Unlike other predictors of expression and solubility which report two possible outcomes (i.e., low or high expression, the presence of inclusion bodies), three predictors can be used to report the probability of producing usable (E*S>11) polypeptide and the probability of observing each possible expression or solubility score. Stepwise multiple regressions were used to create multifactorial models, starting with all significant parameters and removing or re-introducing parameters individually as they became statistically insignificant or regained significance. The slopes and significance of parameters remaining after this process are summarized in Table 11; for comparison to the original significant parameters, the parameters remaining in the usability model are also shown in FIG. 9.

TABLE 11
Parameter coefficients in final predictive models.

| | | | Usability w/rare | | | | | |
|---|---|---|---|---|---|---|---|---|
| | Usability | | codons | | Expression | | Solubility | |
| Parameter | Slope | P-value | Slope | P-value | Slope | P-value | Slope | P-value |
| ab | | | | | | | −4.82 | 0.0012 |
| c | −8.5 | 2.14E− | −6.54 | 0.0005 | | | −13.73 | 5.03E− |
| e | | | | | 2.75 | 0.028 | | |
| fb | −3.88 | 0.0198 | −4.17 | 0.015 | | | −10.67 | 3.39E− |
| h | | | | | 12.71 | 2.74E− | 10.81 | 6.70E− |
| i | | | | | | | −5.7 | 0.0056 |
| ke | | | | | 6.05 | 1.36E− | | |
| l | −2.23 | 0.0308 | | | | | −10.38 | 3.64E− |
| mb | | | | | 7.89 | 0.00027 | | |
| nb | | | | | | | 15.6 | 0.0028 |
| ne | | | | | | | 12.64 | 1.45E− |
| p | | | | | | | 4.16 | 0.01 |
| q | | | | | 9.73 | 7.25E− | | |
| qe | 9.86 | 2.74E− | 8.44 | 1.44E− | | | 15.43 | 9.75E− |
| r | −9.82 | 1.18E− | | | | | −7.24 | 2.56E− |
| s | −4.33 | 0.0006 | −3.2 | 0.015 | | | | |
| te | 4.36 | 0.0026 | 5.13 | 0.00037 | | | 8.16 | 3.39E− |
| v | | | | | | | −8.21 | 1.19E− |
| w | −6 | 0.0226 | | | | | | |
| fracnumcharge | 9.65 | 6.60E−27 | 12.11 | 3.67E−24 | 3.7 | 4.31E−05 | 20.27 | 2.12E−37 |
| absnetcharge | 0.015 | 3.18E−05 | 0.011 | 0.0018 | | | | |
| fracabsnetcharge | | | | | −4.88 | 3.73E−14 | 4.01 | 1.44E−07 |
| netcharge | | | | | −0.025 | 5.19E− | | |
| gravy | −0.45 | 0.0037 | −0.78 | 1.44E− | −0.55 | 2.14E− | 1.72 | 3.01E− |
| sce | | | −4.13 | 1.10E− | | | −4.88 | 9.17E− |
| esce | −1.9 | 3.17E− | | | −1.4 | 7.42E− | | |
| diso | −1.73 | 1.72E− | −1.59 | 4.52E− | −1.73 | 3.39E− | −1.09 | 2.47E− |

Rare Codons

| | | |
|---|---|---|
| rare r | −11.33 | 2.38E− |
| common r | −9 | 3.59E− |
| rare i | −13.75 | 9.80E− |
| common i | 8.74 | 8.92E− |
| rare p | −6.84 | 0.0093 |

Score Cutpoints

| | | |
|---|---|---|
| 0 to 1 | −6.682 | −2.095 |
| 1 to 2 | −0.548 | −1.728 |
| 2 to 3 | −0.233 | −1.201 |
| 3 to 4 | 0.375 | −0.532 |
| 4 to 5 | 1.0468 | 0.041 |

Variable coefficients and p-values for final predictors for usability, usability including rare codon effects, expression, and solubility. The cut-points between the 6 category outcomes (scores 0-5) are indicated are indicated for the ordinal logistic models for expression and solubility. A description of outcome probability calculations in logistic models is provided herein.

For usability, positive effects remain for exposed Gln, exposed Thr, absolute net charge, and, by far the most significant, fraction of charged residues. Negative effects remain for Cys, buried Phe, Trp, GRAVY, disorder, and, most significant, Arg. Exposed SCE shifts from a positive effect in single regression to a negative effect in multiple regressions. SCE may initially function as a proxy for Lys and Glu content: both carry electrostatic charge, which improves both solubility and usability, and both also have high SCE. When their charge effect is included in the multiple regression via the fracnumcharge parameter, the influence of SCE on usability becomes negative. This effect can result from parameter interdependence.

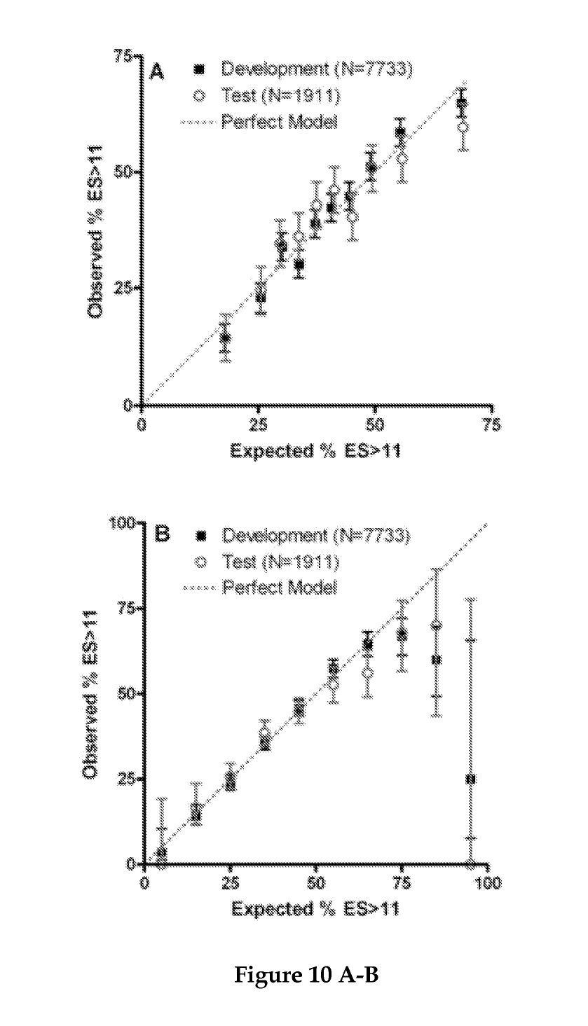

The combined usability metric (called pES, the probability of Expressed and Soluble polypeptide) models the development set closely up to a 65% probability of polypeptide usability (p=3.7×10⁻¹¹¹, N=7733) (FIG. 9). The metric was also tested on a set of 1911 polypeptides randomly held separate from the development set; it predicts those polypeptides nearly as well (p=6.8×10⁻¹⁶). Using a cutoff of pES>0.3, the rate of usable polypeptides could be increased by 13% while keeping 80% of targets; using a cutoff of 0.4 would increase rates by 29% retaining 46% of targets, and a cutoff of 0.5 would increase rates by 45% while retaining 20% of targets. A usability metric which includes the rare codon effects shown in FIG. 5 was also developed (FIG. 10). The model describes the data better than the amino acid sequence based model without codon frequency information (p=9.2×10⁻¹³⁷). It also performs well on the 1911 test polypeptides withheld from the model development process (p=3.3×10⁻¹⁹).

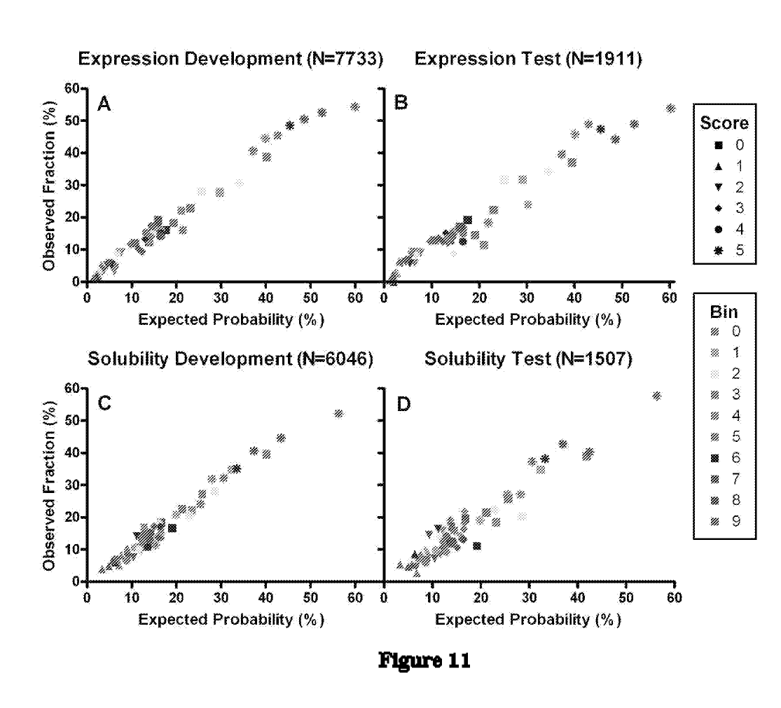

Separate predictive metrics for expression and solubility using the same process of stepwise logistic regression (with ordinal instead of binary logistic regression) were also developed. The slopes and parameters retained in these regressions are reported in Table 11. Ordinal logistic regressions provide probabilities of scoring each of the possible outcomes (0-5). They perform well in predicting the distribution of scores observed in the ensemble of polypeptides in both the development and test sets (FIG. 11). Note that their performance in predicting the result observed with a single polypeptide is difficult to interpret. The scores observed in the dataset are primarily either 0 or 5, however, the probability-weighted average of the predicted scores for a single polypeptide tends to fall near 3, in spite of the fact that this value is seldom observed. Therefore, ensemble-based evaluations are more appropriate. The amino-acid based predictors are available at http://nmrcabm.rutgers.edu:8080/PES/.

Permissive and Enhancing Parameters.

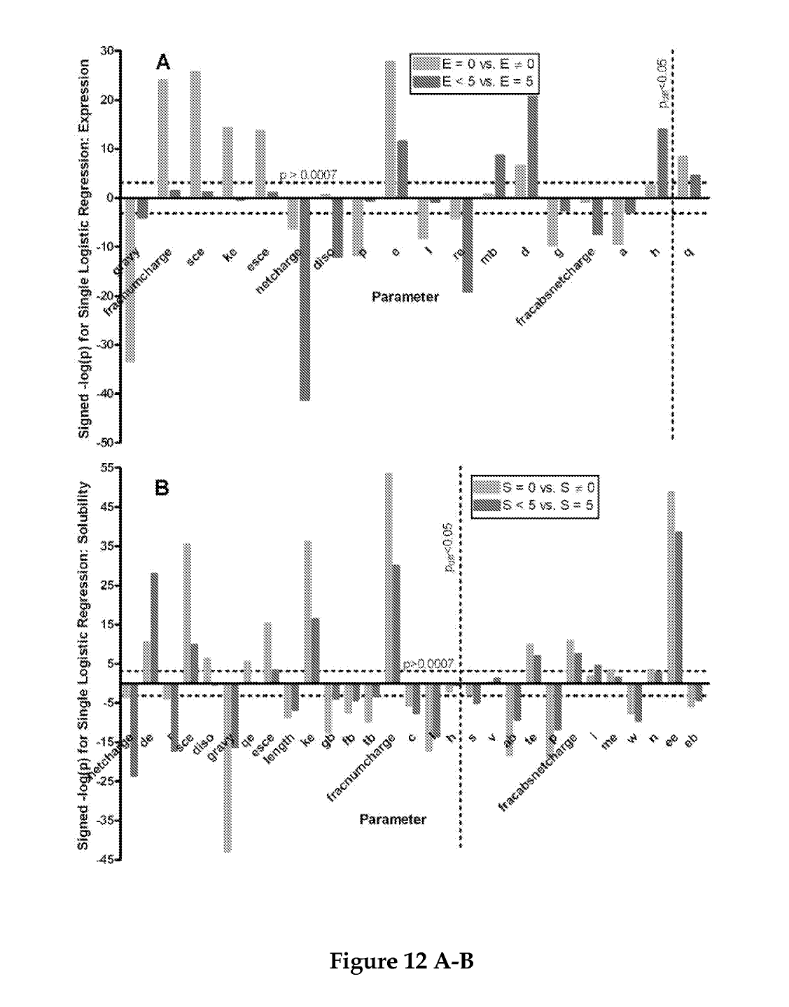

To examine the related mechanistic effects, the impact of individual parameters was examined to determine whether some parameters influenced outcomes at the low end of the score range (i.e., no expression (E=0) vs. any expression at all (E>0)—“permissive” factors) or at the high end of the range (i.e., very high expression (E=5) vs. lesser expression (E<5)—“enhancing” factors). Many parameters have such disparate impacts (FIG. 12). Notably for expression, parameters related to the content of charged or hydrophobic residues are primarily permissive, while net charge is primarily enhancing. Similar patterns exist for solubility, but in this case most significantly permissive factors were also significantly enhancing.

Mechanistic and Engineering Implications.

The methods described herein relate to the biophysics of polypeptide translation and solubility through a data mining approach grounded in the large-scale systematically controlled datasets created through structural genomics efforts. Positively charged residues have a negative impact on polypeptide translation, due, in part, to electrostatic attraction to the negatively charged RNA of the ribosome (Sanbonmatsu, et al. (2005) Proceedings of the National Academy of Sciences of the United States of America 102:15854-15859; Pedersen (1984) The EMBO Journal 3:2895). Negatively charged residues, in contrast, have a strong positive impact on both expression and solubility. Arg content has a negative effect on both expression and solubility that is only partially attributable to rare codons. Other amino acids with rare codons also show differential effects between rare and common codons even in a so-called codon-optimized strain. Hydrophobicity appears not to be a dominant factor in polypeptide solubility; while mean chain hydrophobicity negatively correlates with solubility, a residue-by-residue analysis (FIG. 6) shows that this effect is primarily due to charged amino acids. Phe (Lewis et al. (2005) Journal of Biological Chemistry 280:1346-1353) and Leu show negative effects on solubility, while Ile and Val both have moderate but significant positive effects on solubility. These effects potentially reflect side-chain contour—Leu and Phe both protrude more from the backbone and likely have increased potential to lodge in hydrophobic grooves. Overall, the effect of hydrophobic residues on polypeptide solubility is more complex than previously thought.

The predictors for expression and solubility described herein can be used to increase the likelihood of expressing high quantities of soluble polypeptides. Target selection necessitates a tradeoff between a higher rate of success with retained targets and discarding a higher proportion of the initial set. Use of the metric described herein with a reasonable cutoff of pES>0.4, a 29% increase in usable targets can be expected while discarding 54% of the pool. This approach can prove useful for high-throughput studies.

The results described herein show new approaches to engineering polypeptides to increase both expression and solubility. While the substitution of common Arg for rare Arg is commonly used to improve expression, results the results described herein show that the substitution of Lys for any Arg can be used to improve solubility and also expression. More broadly, the addition of Lys, Gln, and Glu can be used to improve both solubility and expression, as can the removal of predicted disordered segments.

Some of these strategies have been pioneered by case studies in the past (Trevino S R, Scholtz J M, Pace C N (2007) J. Mol. Biol 366:449-460; Tanha J et al. (2006) Polypeptide Eng. Des. Sel 19:503-509), but the analysis described herein provides statistical support in a large set of diverse targets and also establishes novel substitutions that enhance protein expression and solubility in the large-scale experimental dataset described herein.

The following methods can be used to produce and/or analyze the results described herein and may be used in connection with certain embodiments of the invention.

Target Selection and Classification.

9644 polypeptide target sequences expressed between 2001 and June 2008 were selected from the SPINE database (Bertone P et al. (2001) Nucleic acids research 29:2884; Goh C S et al. (2003) Nucleic acids research 31:2833). Polypeptide sequences were randomly assigned at a 4:1 ratio (7733:1911) to training or validation sets. Polypeptides with transmembrane α-helices predicted by TMMHMM (Krogh A, et al. (2001) Journal of Molecular Biology 305:567-580) or >20% low complexity sequence are routinely excluded from the pipeline, and therefore were not included in the analysis.

Polypeptide Expression & Purification.

Polypeptides were expressed, purified, and analyzed as previously described (Acton T B et al. Robotic Cloning and Polypeptide Production Platform of the Northeast Structural Genomics Consortium).

Data Mining Variables.

Data mining analyses were conducted on native sequences with tags removed. Three outcome variables were considered: independent 0-5 integer scores for expression and solubility, as evaluated by Coomassie-stained gel electrophoresis, and the binary variable of usability, defined as having a product of expression and solubility scores of 12 or higher. Input variables included the frequency of each amino acid, either total or predicted to be buried or exposed by PHD/PROF (60 variables in total), and the compound sequence metrics of charge, pI, GRAVY, SCE, length, and DISOPRED. Charge parameters were calculated as signed or unsigned sums of the frequencies of appropriate combinations of Arg, Lys, Glu, and Asp residues, and were considered as both whole and fractional values; the number and fraction of charged residues were also calculated. Isoelectric point was calculated using the EMBOSS algorithm (Rice P, et al. (2000) Trends in genetics 16:276-277) at ExPASy (Appel R D, et al. (1994) Trends in Biochemical Sciences 19:258). GRAVY was calculated using the Kyte-Doolittle hydropathy parameters (Kyte J, Doolittle R F (1982) Journal of Molecular Biology 157:105). The Creamer scale (Creamer T P (2000) Polypeptides: Structure, Function, and Genetics 40) was used for the SCE values of the individual amino acids. DISOPRED scores were calculated using DISOPRED2 (Ward J J, et al. (2004) The DISOPRED server for the prediction of polypeptide disorder (Oxford Univ Press)) with a 5% false positive rate. Calculations of predicted burial/exposure and secondary structure were performed with the PHD/PROF algorithms (Rost B (2005) The proteomics protocols handbook. Totowa (New Jersey): Humana:875-901) from the PredictPolypeptide server (Rost B, et al. (2004) Nucleic Acids Research 32:W321). Mean exposed SCE was calculated as the mean for all residues predicted to be exposed, while all calculations based on secondary structure class used total chain length as the denominator.

Regressions and Model Building.

For each of the three outcome variables (expression, solubility, and usability), single logistic regressions were run to evaluate potential correlations between the outcome variable and the 72 input variables calculated from the polypeptide sequence. Proportional odds ordinal logistic regressions were used for expression and solubility, and binary logistic regression for usability (Hosmer D W, Lemeshow S (2004) Applied logistic regression (Wiley-Interscience)). In binary logistic regression, the probability of a positive outcome is given by the function Pr(Y=1)=eθ/(1+eθ), where θ is the linear combination of predictive variable values and their slopes. For ordinal logistic regression, the probability that the outcome is less than or equal to a value j is given by the function Pr(Y≦j)=e^{(tj-θ)}/(1+e^{(tj-θ)}, with the added parameter tj, a threshold value for each value of the outcome variable. Among the three variables for each amino acid (total fraction, predicted buried fraction, and predicted exposed fraction), the buried/exposed variables were retained if they had opposite-signed slopes in single logistic regressions, otherwise the total fraction was retained. For charge variables, the more significant of the whole or fractional versions of each variable was kept. All variables which were not significant at the Bonferroni-adjusted p-value of 0.00069 (0.05/72) were dropped. Combined models were built by stepwise forward/reverse logistic regression with p-value cutoffs of 0.05 for removal and 0.049 for addition. Each variable in the resulting model was individually removed to check for improvement in Akaike's Information Criterion (AIC) (Akaike H (1974) IEEE transactions on automatic control 19:716-723). Any variable whose removal improved the AIC was discarded from the model.

Statistical Analyses.

Logistic regressions were performed in STATA (Statacorp, College Station, Tex.) with significance determined from Z-scores for individual variables and chi-squared distributions for models. Counting-statistics-based 95% confidence intervals were calculated using Bayesian maximum likelihood estimates of the binomial distribution.

Details on Permissive v. Enhancing Parameters.

Factors can operate in different ways across the range of expression and solubility values. A factor could operate equally across the range: in that case, an increase in the parameter (for a positively correlated parameter) would have the same effect on the odds of a polypeptide scoring 0 vs. 1 for expression as for that polypeptide scoring 3 vs. 4. Alternately, factors could operate differently at different ends of the score spectrum, so that, for instance, the fraction of an amino acid has a large impact on whether a polypeptide scores 0 vs. 1 or higher but has less impact among the scores above 0 (a “permissive” factor) or a large impact on whether a polypeptide scores 5 vs. something below 5, but makes less difference among the sub-5 scores (an “enhancement” factor). This issue can be addressed by examining whether the slopes of the paired binary logistic regressions between adjacent scores differ significantly as the scores change. This difference was examined both by calculating the Brant statistic (Brant R (1990) Biometrics 46:1171-1178), which evaluates the likelihood that the true slopes between different outcome steps in an ordinal logistic regression are equal given the regression outcome, and by running the individual binary logistic regressions for permissive (0 vs. not-0) and enhancement (0-4 vs. 5). Signed negative log(p) values are shown for these regressions for all factors which were significant predictors of expression or solubility, sorted by the significance of their Brant statistic (FIG. 4).

The majority of expression-predicting parameters differed significantly across the range of expression scores. GRAVY, Pro, Leu, Gly, and Ala primarily have negative effects at the permissive level; fractional number of charges, SCE, exposed Lys, exposed SCE, and Glu primarily have positive effects at the permissive level. Net charge, fractional disorder, exposed Arg, and fractional absolute net charge primarily have negative effects at the enhancement level, while Asp, buried Met and His primarily have positive effects at the enhancement level. Gln showed no significant difference, and a few parameters (GRAVY, net charge, Glu, exposed Arg, Asp, and Ala) showed lesser but still significant effects at the second level (i.e., enhancement if their most significant effect was permissive). No parameter had opposite signed effects at the two levels.

For solubility, only disorder and exposed Gln had significant effects at only one level—both are positive at the permissive level. All other effects were significant at both levels, but SCE and exposed SCE, exposed Lys, and fraction of charged residues were primarily positive permitters; GRAVY, length, buried Gly, buried Phe, buried Thr, Cys, and Ile were primarily negative permitters. Exposed Asp was the only primarily positive enhancer, and net charge, and Arg were the only primarily negative enhancers. All other significant predictors did not differ significantly between the permissive and enhancement levels.

The results described herein show that amino acid sequence features correlate with high expression and solubility. Surprising findings include the observations that (1) hydrophobicity is unexpectedly not a dominant factor in determining solubility, but functions instead as a surrogate for charge; (2) isoleucine can be expression and solubility enhancing; and (3) arginine, even when encoded by common codons, can be detrimental to both expression and solubility. These findings show that positively but not negatively charged amino acids can slow translation due to electrostatic interactions with ribosomal RNA.

These results also show that novel engineering approaches using amino acid substitutions, such as isoleucine for leucine and lysine for arginine can be used to improve the usability, solubility and expression of proteins. Engineering evaluation will be performed by mutating proteins with expression or solubility problems to introduce more favorable residues (e.g., Ile for Leu or Lys for Arg) in homology-allowed locations.

### Example 2

### Codon Effects on Polypeptide Expression & Solubility

Knowledge of codon usage effects on protein expression and solubility is relevant both for understanding biological regulation and for overexpressing recombinant proteins. To better understand these effects, the impact of codon frequency on experimentally observed protein expression and solubility was examined in 9,644 proteins produced in the uniform protein production pipeline of the Northeast Structural Genomics Consortium. Significant correlations were observed between several codons and protein expression and solubility. Asp, Glu, Gln, and His each showed one codon significantly correlated with higher expression and one codon without a significant correlation. Ile's three codons showed one positive, one negative, and one insignificant correlation. Codon correlations were not primarily attributable to genomic codon frequency, the prevalence of isoacceptor tRNA molecules, GC content within the codon, or the biochemical properties of the encoded amino acid.

The effects of codon usage on protein expression are important both for understanding of in vivo biological regulation (Gouy and Gautier, Nucleic Acids Research 10, 7055 (1982); Sharp et al, Nucleic Acids Research 14, 7737 (1986); Sharp and Li, Nucleic Acids Research 15, 1281 (1987); Bulmer, Genetics 129, 897 (1991)) and for the ability to overexpress proteins for biochemical and structural studies (Gustafsson et al, Trends in biotechnology 22, 346-353 (2004); Wu et al, Biochemical and Biophysical Research Communications 313, 89-96 (2004); Angov et al, PLoS ONE. 3, e2189 (2008); Hatfield and Roth, Biotechnol Annu Rev 13, 27-42 (2007)). Theoretical calculations (Bulmer, Genetics 129, 897 (1991); Grosjean and Fiers, Gene 18, 199 (1982)), correlations with small- and large-scale expression datasets (Gustafsson et al, Trends in biotechnology 22, 346-353 (2004); de Sousa Abreu, et al, Global signatures of protein and mRNA expression levels. Mol. BioSyst. (2009); Hoekema, et al, Mol. Cell. Biol. 7, 2914-2924 (1987)), and direct experimentation (Kudla et al, Science 324, 255-8 (2009); Kim et al, Gene 199, 293-301 (1997); Hoekema et al, Mol. Cell. Biol. 7, 2914-2924 (1987); Hale et al, Protein expression and purification 12, 185-188 (1998)) have been used to examine the effects of codon usage. Conflicting results (Kudla et al, Science 324, 255-8 (2009); Sharp and Li, Nucleic acids research 15, 1281 (1987); Bulmer, 129, 897 (1991)), have left unclear the in vivo and in vitro impacts of codon frequency on the production of proteins.

Large-scale experimental data from the uniform protein-production pipeline of the Northeast Structural Genomics Consortium (NESG) (Acton et al, Methods in Enzymology 394, 210-243 (2005)) was used to determine statistically significant correlations between codon usage in a protein target and that protein's experimentally observed expression and solubility characteristics. This approach allows evaluation of the magnitude and significance of these effects in an environment isolated from the variations in experimental procedure endemic to publicly available large datasets, while retaining the ability to observe smaller significant effects provided by thousands of experimental observations.

The experimental results of 9,644 polypeptides which were expressed in the NESG polypeptide production pipeline were analyzed. These targets did not include polypeptides with large low-complexity regions, predicted transmembrane α-helices, or predicted signal peptides; some targets are individual domains of multi-domain polypeptides. Polypeptides were expressed from a T7-polymerase-based pET vector carrying short hexa-histidine tags (Acton T B et al. (2005) Methods in Enzymology 394:210-243). All polypeptides were independently scored for expression (0-5), based on the total amount of polypeptide in SDS-PAGE gels, and solubility (0-5) based the fraction of polypeptide appearing in the soluble fraction after centrifugation to remove inclusion bodies. Logistic regression analysis was used to examine the relationship between the fractional content of each codon in the transcript and the experimental outcomes of expression or solubility. Ordinal logistic regressions determine the strength and statistical significance of the relationship between a continuous independent variable (e.g., the fractional content of a particular codon) and a stepwise dependent variable (e.g., expression or solubility level).

Different Effects of Synonymous Codons on Expression and Solubility.

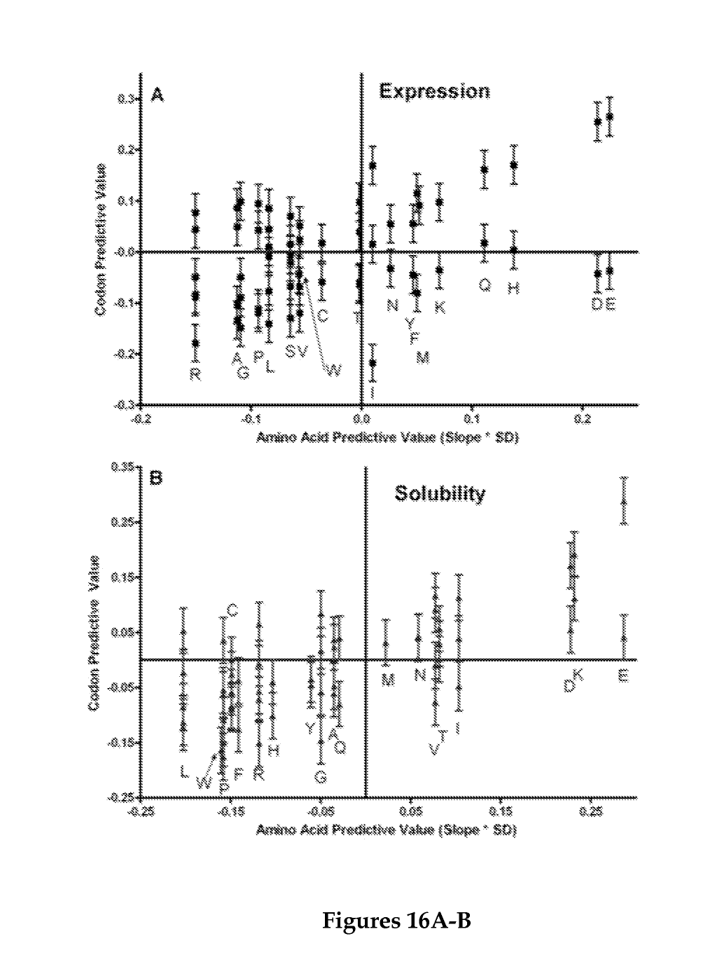

For several different amino acids, synonymous codons showed different correlations with experimentally observed expression and solubility (FIG. 16, Table 12).

TABLE 12

| Amino | | #/1000 | # tRNA/ | Exp. | Exp. | Exp. P | Sol. | Sol. | Sol. P. |
|---|---|---|---|---|---|---|---|---|---|
| Acid | codon | codons | 1000 | Slope | S.E. | Value | Slope | S.E. | Value |
| Ala | GCA | 20.69 | 50.4 | 3.70 | 1.37 | 0.0071 | 1.70 | 1.53 | 0.088 |
| Ala | GCC | 25.25 | 9.5 | −4.96 | 0.69 | 6.02E−13 | −2.26 | 0.79 | 0.024 |
| Ala | GCG | 32.22 | 50.4 | −5.02 | 0.89 | 1.6E−08 | −2.30 | 1.01 | 0.021 |
| Ala | GCT | 15.4 | 50.4 | 6.43 | 1.37 | 2.6E−06 | 2.74 | 1.51 | 0.0062 |
| Arg | AGA | 3.01 | 13.4 | −3.89 | 1.44 | 0.0067 | −0.50 | 1.65 | 0.62 |
| Arg | AGG | 1.94 | 6.5 | −6.67 | 1.45 | 4.43E−06 | −5.77 | 1.66 | 7.83E−09 |
| Arg | CGA | 3.92 | 73.7 | 7.02 | 2.89 | 0.015 | −11.23 | 3.15 | 2.87E−29 |
| Arg | CGC | 20.9 | 73.7 | −4.24 | 0.87 | 1.12E−06 | −2.72 | 0.99 | 0.0064 |
| Arg | CGG | 6.35 | 9.9 | −14.17 | 1.42 | 2.28E−23 | −12.00 | 1.68 | 3.6E−33 |
| Arg | CGT | 20.26 | 73.7 | 5.71 | 1.34 | 2.04E−05 | 4.80 | 1.45 | 1.6E−06 |
| Asn | AAC | 21.61 | 18.5 | −2.55 | 1.46 | 0.080 | 3.40 | 1.63 | 0.00067 |
| Asn | AAT | 19.08 | 18.5 | 3.00 | 1.01 | 0.0029 | 2.17 | 1.15 | 0.030 |
| Asp | GAC | 19.17 | 37.2 | −2.15 | 0.94 | 0.023 | 2.80 | 1.08 | 0.0051 |
| Asp | GAT | 32.78 | 37.2 | 13.51 | 1.00 | 9.08E−42 | 9.05 | 1.10 | 1.41E−19 |
| Cys | TGC | 6.42 | 24.6 | −6.07 | 1.84 | 0.0010 | −15.48 | 2.22 | 4.46E−54 |
| Cys | TGT | 5.3 | 24.6 | 2.04 | 2.09 | 0.33 | −12.53 | 2.36 | 4.93E−36 |
| Gln | CAA | 14.6 | 11.8 | 9.80 | 1.13 | 3.62E−18 | 2.40 | 1.21 | 0.016 |
| Gln | CAG | 29.52 | 13.6 | 1.06 | 1.10 | 0.33 | −4.78 | 1.22 | 1.72E−06 |
| Glu | GAA | 39.2 | 73.2 | 10.79 | 0.77 | 1.18E−44 | 11.76 | 0.85 | 6.41E−32 |
| Glu | GAG | 18.89 | 73.2 | −1.84 | 0.92 | 0.046 | 2.04 | 1.03 | 0.041 |
| Gly | GGA | 8.97 | 33.1 | −3.63 | 1.35 | 0.0074 | 1.20 | 1.55 | 0.23 |
| Gly | GGC | 27.87 | 67.6 | −3.85 | 0.80 | 1.44E−06 | −2.50 | 0.91 | 0.013 |
| Gly | GGG | 11.91 | 33.1 | −14.14 | 1.74 | 4.66E−16 | −13.94 | 2.03 | 3.82E−44 |
| Gly | GGT | 24.12 | 67.6 | 7.54 | 1.42 | 1.04E−07 | 6.39 | 1.57 | 1.63E−10 |
| His | CAC | 9.34 | 9.9 | 0.37 | 1.80 | 0.84 | −9.90 | 2.04 | 4.18E−23 |
| His | CAT | 12.78 | 9.9 | 16.03 | 1.77 | 1.09E−19 | −3.77 | 1.89 | 0.00017 |
| Ile | ATA | 5.61 | 53.9 | −13.36 | 1.11 | 3.15E−33 | −2.93 | 1.37 | 0.0034 |
| Ile | ATC | 23.76 | 53.9 | 1.00 | 1.21 | 0.41 | 2.57 | 1.33 | 0.010 |
| Ile | ATT | 29.41 | 53.9 | 8.73 | 0.96 | 1.09E−19 | 5.83 | 1.06 | 5.43E−09 |
| Leu | CTA | 3.88 | 10.3 | 1.26 | 2.32 | 0.59 | −2.90 | 2.61 | 0.0037 |
| Leu | CTC | 10.46 | 14.6 | −9.35 | 1.22 | 1.59E−14 | −7.51 | 1.39 | 5.86E−14 |
| Leu | CTG | 50.85 | 79.7 | −2.71 | 0.65 | 3.18E−05 | −4.31 | 0.74 | 1.62E−05 |
| Leu | CTT | 11.44 | 14.6 | −0.76 | 1.56 | 0.62 | −1.90 | 1.77 | 0.057 |
| Leu | TTA | 13.78 | 16 | 4.46 | 0.96 | 3.32E−06 | 2.75 | 1.06 | 0.0059 |
| Leu | TTG | 12.89 | 45.7 | 3.71 | 1.57 | 0.018 | −7.12 | 1.78 | 1.07E−12 |
| Lys | AAA | 33.96 | 29.7 | 3.31 | 0.62 | 9.82E−08 | 6.50 | 0.70 | 8.15E−11 |
| Lys | AAG | 11.14 | 29.7 | −1.81 | 0.92 | 0.049 | 5.72 | 1.03 | 1.07E−08 |
| Met | ATG | 27.1 | 40.8 | 7.26 | 1.48 | 9.58E−07 | 2.49 | 1.64 | 0.013 |
| Phe | TTC | 15.78 | 16 | −6.03 | 1.38 | 1.19E−05 | −9.44 | 1.54 | 3.73E−21 |
| Phe | TTT | 22.15 | 16 | 6.93 | 1.13 | 7.75E−10 | −2.27 | 1.25 | 0.023 |
| Pro | CCA | 8.4 | 9 | 4.28 | 1.85 | 0.020 | 3.55 | 2.08 | 0.00039 |
| Pro | CCC | 5.62 | 11.1 | −9.58 | 1.59 | 1.86E−09 | −15.10 | 1.84 | 1.61E−51 |
| Pro | CCG | 22.47 | 22.8 | −8.07 | 1.25 | 1.12E−10 | −3.74 | 1.41 | 0.00018 |
| Pro | CCT | 7.3 | 20.1 | 10.49 | 2.07 | 4.19E−07 | −6.96 | 2.30 | 3.29E−12 |
| Ser | AGC | 16.03 | 21.8 | −1.91 | 1.72 | 0.27 | −8.51 | 1.91 | 1.67E−17 |
| Ser | AGT | 9.44 | 21.8 | 7.70 | 2.04 | 0.00016 | −6.42 | 2.27 | 1.33E−10 |
| Ser | TCA | 8.25 | 20.1 | 1.54 | 1.83 | 0.40 | −2.59 | 2.05 | 0.0097 |
| Ser | TCC | 9.01 | 11.8 | −7.64 | 2.08 | 0.00024 | −9.50 | 2.35 | 2.04E−21 |
| Ser | TCG | 8.77 | 25.4 | −14.58 | 2.06 | 1.55E−12 | −9.65 | 2.35 | 5.13E−22 |
| Ser | TCT | 8.73 | 31.9 | −0.58 | 1.86 | 0.76 | 0.03 | 2.10 | 0.98 |
| Thr | ACA | 8.23 | 14.2 | 8.24 | 1.56 | 1.36E−07 | 4.76 | 1.73 | 1.96E−06 |
| Thr | ACC | 22.66 | 18.6 | −4.15 | 1.20 | 0.00056 | 0.10 | 1.37 | 0.92 |
| Thr | ACG | 15.08 | 22.6 | −5.68 | 1.74 | 0.0011 | 2.85 | 1.96 | 0.0044 |
| Thr | ACT | 9.06 | 32.8 | 3.94 | 1.82 | 0.031 | 2.88 | 2.05 | 0.0040 |
| Trp | TGG | 15.32 | 14.6 | −4.14 | 1.78 | 0.020 | −15.85 | 2.02 | 1.44E−56 |
| Tyr | TAC | 12.29 | 31.4 | −4.16 | 1.72 | 0.015 | −4.21 | 1.92 | 2.51E−05 |
| Tyr | TAT | 16.52 | 31.4 | 3.70 | 1.22 | 0.0024 | −2.34 | 1.38 | 0.019 |
| Val | GTA | 10.89 | 59.6 | 2.02 | 1.48 | 0.17 | 7.37 | 1.65 | 1.65E−13 |
| Val | GTC | 14.71 | 19.5 | −7.83 | 1.21 | 9.17E−11 | −0.66 | 1.38 | 0.51 |
| Val | GTG | 26.15 | 59.6 | −4.05 | 1.10 | 0.00023 | −4.60 | 1.26 | 4.14E−06 |
| Val | GTT | 18.04 | 79.1 | 3.22 | 1.14 | 0.0048 | 7.26 | 1.27 | 3.81E−13 |

^{a}Ordinal logistic regressions were performed to evaluate the correlations between the fractional content of each codon in the transcript and the experimental outcomes of expression (scored 0-5) and solubility (0-5). The table reports the number of times each codon appears in the E. coli genome per 1000 codons (Nakamura et al, Nucleic Acids Res 28, 292 (2000)) and the number of isoacceptor tRNA molecules per 1000 present in cells (Dong et al, Journal of Molecular Biology 260, 649-663 (1996)). The results of the logistic regressions are also shown, with slope, standard error, and P value shown for both expression (N = 9,644) and solubility (N = 7,548) regressions. P-values below the Bonferroni-adjusted threshold of 0.0008 are shown in boldface type.

Four amino acids showed a distinct and surprising pattern in their correlations with expression. Asp, Gln, Glu, and His each have two codons, and for each amino acid, one codon showed no significant correlation with expression (GAC, CAG, GAG, and CAC, respectively), while one codon showed a significant positive correlation with increased expression (GAT, CAA, GAA, and CAT, respectively). This effect has been previously noted for Glu in a study on a single model polypeptide, where GAA has been experimentally observed to be translated significantly more rapidly than GAG (Krüger M K, et al. (1998) Journal of Molecular Biology 284:621-631). Two other amino acids showed notable though less unexpected patterns. Four Arg codons had negative expression correlations, and two had positive correlations. Finally, among the three Ile codons, one (ATA) showed a significant negative correlation with expression, one (ATC) showed no significant relationship, and one (ATT) showed a significant positive correlation.

Codon Effects do not Correlate with Codon Frequency or Cognate tRNA Abundance.

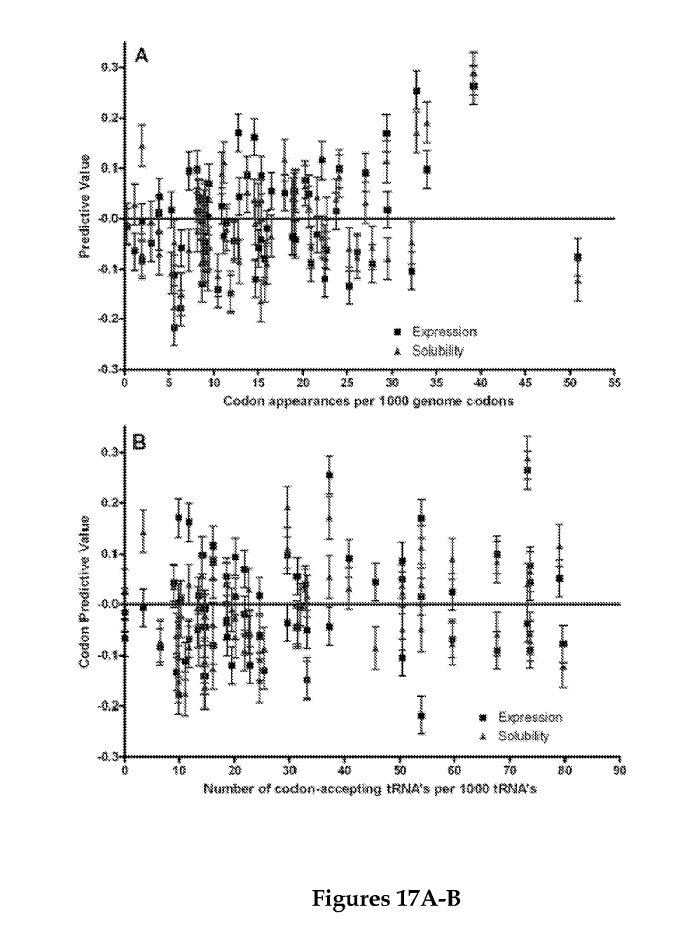

Although codon frequency can be a source of the observed differences in synonymous codons, no significant relationship between the frequency with which a codon appeared in the E. coli genome and the codon's correlation to expression or solubility was observed (FIG. 17A). The codon effects shown herein reinforce this finding. For the four two-codon amino acids discussed, Asp, Glu, and His show positive effects for the more common codon, but Gln shows a positive expression correlation with the less prevalent codon. Similarly, Arg has two common codons, one positive and one negative, and four rare codons, three negative and one positive. While it is impossible to rule out genomic codon frequency as a determinant of codon effect on expression, the results described herein indicate that it is unlikely to be a dominant factor.

A related but more specific view in the field holds that the deleterious effects of rare codons on polypeptide expression are essentially a kinetic effect of the low prevalence of cognate tRNAs, which correlates strongly but not precisely with genomic codon frequency. Again, the results described herein show a significantly different pattern—no strong relationship is observed between isoacceptor tRNA abundance and codon frequency correlations with either expression or solubility (FIG. 17B).

Codon Effects are not Solely Based on GC Content or Amino Acid Physical Properties.

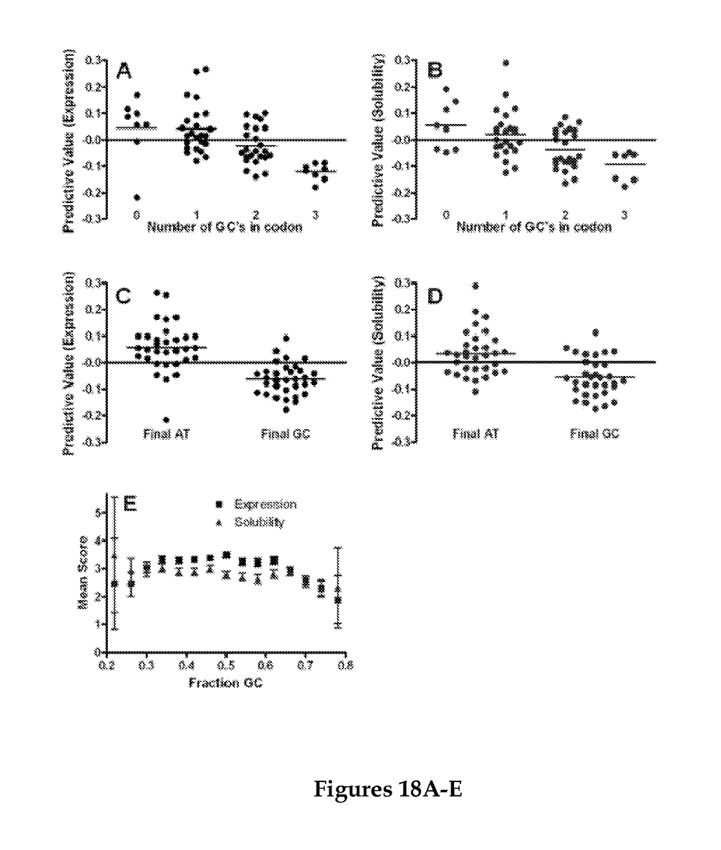

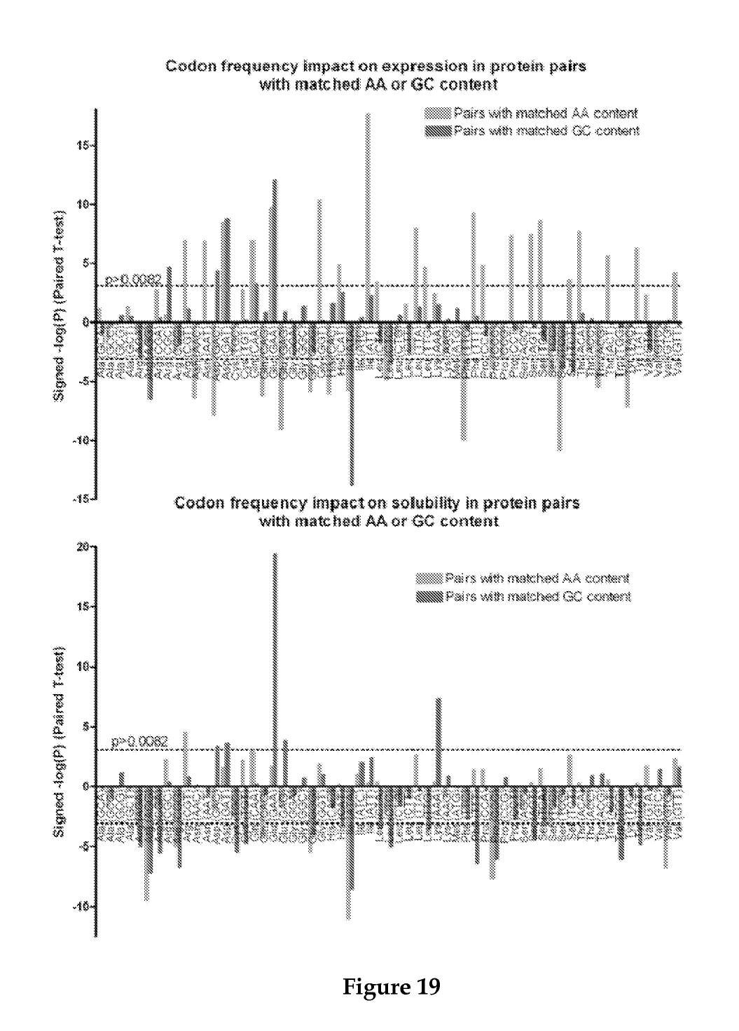

Alternately, some effects of codons on expression can be based on the physical properties of either the codon or the amino acid encoded. Higher GC content within a codon can make transcriptional DNA unwinding slower or less efficient, and can also result in an increased prevalence of stable RNA secondary structure, which has been shown to reduce translation. Significant trends in this direction, where GC content within a codon predicted the codon's correlation with expression (and, to a lesser extent, solubility), both generally (FIG. 18A, B) and in the wobble position (FIG. 18C, D) were observed in the results described herein. Overall GC content also showed a relationship to expression but not solubility (FIG. 18E). To determine whether GC content was a primary determinant of codon effect, matching sets of polypeptides were created so that they had the same fractional GC content but differing contents of the codon in question. The means of these matched polypeptide distributions were then compared via a heteroskedastic paired T-test to determine which codons still significantly effected expression when GC content was controlled. The majority of codon effects remained significant in this analysis (FIG. 19). In particular, the positive expression codon effects for Asp, Gln, and Glu all remained significantly positive, although the effect for His dropped below the Bonferroni-corrected statistical significance threshold.

In addition to the GC content of the codon, the physical properties of the amino acid encoded can have effects on translation efficiency or polypeptide degradation, which would impact expression results. It is possible that positively but not negatively charged amino acids can impede translational efficiency. This effect cannot be responsible for the differences in synonymous codons, but can show trends among all the codons for an amino acid. To address this concern, a similar matching analysis was performed, holding amino acid fraction constant while varying the fraction of the relevant codon. Met and Trp were excluded from this analysis, as each amino acid is encoded by only one codon. All of the effects noted above remain consistent, with one exception and one caveat (FIG. 19). For Arg, only CGT remained significant. More salient is the change in the four significantly different amino acids with exactly two codons. For these amino acids, the positively correlated codon remained positive but the uncorrelated codon acquired a strong negative correlation with expression. This effect is almost certainly an arithmetical artifact: with two codons and a constant amino acid fraction, an increase in a neutral codon is necessarily a decrease in a positive codon—and therefore has an overall negative correlation with higher expression.

Different results were observed for codon effects on solubility. Since much though not all of a polypeptide's solubility can be mediated after the process of translation has been completed, many but not all codon effects on solubility can become insignificant when the relevant amino acid fraction is constant (FIG. 19B).

Data mining studies of a large uniform expression and solubility dataset revealed significant correlations between those experimental outcomes and the prevalence of different synonymous codons in the gene transcript. These effects were not attributable solely to the GC content of the codon, the genomic frequency of the codon or the scarcity of isoaccepting tRNA molecules, or the physiochemical properties of the encoded amino acid. Instead, at least some of the codon effects observed can be the result of functionally based regulons. Such regulons can operate at two levels. One mechanism of codon frequency-based regulation can involve isoacceptor tRNA modification. tRNA modifications have been shown to change tRNA specificity (Soma et al, Molecular cell 12, 689-698 (2003); Ikeuchi et al, Molecular cell 19, 235-246 (2005)) and, in specific cases, to differentially change the in vivo rate of translation of short sequences rich in alternate synonymous codons (Pedersen, The EMBO Journal 3, 2895-8 (1984); Krüger et al, Journal of molecular biology 284, 621-631 (1998)). Functionally, this form of translational regulation can involve, for example, encoding genes most relevant for a specific set of environmental circumstances with a higher proportion of codons which are normally translated more slowly, and then increasing the prevalence of a modified tRNA isoacceptor to upregulate those genes when those conditions are encountered. The validity of this hypothesis can be tested by examining the expression of genes rich in alternate synonymous codons in cell lines with various non-essential tRNA modification enzymes knocked-out, and testing whether expression is differentially altered based on codon frequency. A more robust methodology can involve using gene synthesis to change the frequency of the relevant codon in both wildtype and knocked-out lines to test whether the tRNA modification enzyme differentially altered gene expression level when codon frequency is changed.

Alternately, regulation can be accomplished by different codon usage patterns affecting mRNA transcript lifetime. This alternative mechanism can be examined by directly evaluating the lifetime of mRNA molecules with differing codon frequencies.

Codon-specific effects can be used in engineering efforts to increase protein expression and potentially even solubility in ribosome-based expression systems. Codons correlated with high expression (e.g., GAA or ATT), can replace synonymous codons with no expression correlations (GAG or ATC) or correlations with low expression (ATA). Since this does not alter the protein sequence, the protein will be biochemically identical once expressed, though in some unusual cases there is the potential for altered protein folding (Komar et al, Trends Biochem. Sci 34, 16-24 (2009); de Ciencias et al, Biotechnology Journal 3, 1047-1057; Rosano and Ceccarelli, Microbial Cell Factories 8, 41 (2009)). A high correlation between increased expression and increased solubility (FIG. 5), as well as the beneficial effect of some codons on both parameters observed in this analysis (FIG. 16), indicate that such an approach can also improve protein solubility. The introduce of any such modifications that introduce strong secondary structure in the first 34 base pairs can be avoided as this has been shown to inhibit expression (Kudla et al, Science 324, 255-8 (2009)). This approach is in contrast to other codon optimization approaches that often rely on matching codon usage to observed genomic frequencies (i.e., attempting to shift the Codon Adaptation Index (Sharp and Li, Nucleic acids research 15, 1281 (1987)) towards 1) or on simply using the most common codons (http://www encorbio.com/protocols/Codon.htm). Since it is based on large-scale experimental results across a wide range of targets in a uniform experimental pipeline, it can provide more broadly applicable results than have been observed for other codon-optimization protocols.

Significant correlations between codon usage and both expression and solubility in the data set. In general, codon effects were not primarily attributable to genomic codon frequency, isoacceptor tRNA prevalence, GC content within the codon, or biochemical properties of the encoded amino acid. These observations show that translational regulons based on codon usage can occur and that they can be mediated by tRNA modification.

To evaluate whether codon changes can alter expression and solubility in a predictable fashion, proteins with low expression and a high fraction of “bad” codons will be silently mutated to include a high fraction of “good” codons and then be examined for changes in expression. A matched set of high-expressing genes with many “good” codons will be mutated in parallel to have more “bad” codons, with an expectation of decreased expression. Testing whether the codon effects are mediated by tRNA modification requires the further step of expressing these proteins, both wild-type and mutant, in strains missing potentially relevant tRNA modification enzymes. If the tRNA modification enzyme in question influences the codon effect, differential expression of the two versions of the target gene will be observed in cells differing in the expression or activity of this tRNA modification enzyme.

The results described herein demonstrate the potential of large uniform datasets from structural genomics effort. These data have been used to probe both methodological and biological questions of significant import to structural biologists and to the larger biology community. The results described herein counter long-held dogmas in the field of protein production,

The following methods can be used to produce and/or analyze the results described herein and may be used in connection with certain embodiments of the invention.

Target Selection and Classification.

9,644 polypeptide sequences were selected from the SPINE database (Bertone P et al. (2001) Nucleic acids research 29:2884; Goh C S et al. (2003) Nucleic acids research 31:2833-8). Polypeptide sequences were randomly assigned at a 4:1 ratio to training or validation sets. Polypeptides with transmembrane α-helices predicted by TMMHMM (Krogh A, et al. (2001) J Mol Biol 305:567-580) or >20% low complexity sequence are routinely excluded from the pipeline, and therefore were not included in the analysis.

Polypeptide Expression and Purification.

Polypeptides were expressed and purified as previously described (Acton T B et al. (2005) Methods in Enzymology 394:210-243).

Fractional Codon Counting.

The content of each codon was calculated as the number of that codon appearing in the chain divided by the overall number of codons in the chain. For location-specific counting, the transcript was divided into up to seven 50-codon sections (codons 1-50, 51-100, 101-150, 151-200, 201-250, 251-300, and 301 and higher). Transcripts under 300 codons had fewer sections, depending on their length (i.e., no entirely empty sections were counted). Fractional codon content was calculated as the number of times that codon appeared within the segment divided by the number of codons in the entire chain, to avoid excessively high values (e.g., a fractional content of 1 for the 101^{st} codon in a transcript 101 codons in length).

Generation of Sets with Matched Amino Acid or GC Content.

Polypeptides were ordered by the parameter to be controlled in the analysis. Polypeptides were grouped into bins in increments of 0.01% of that parameter—i.e., polypeptides with GC content between 53.00% and 53.01%. In every bin with more than one member, the bin was sorted according to the fractional content of the codon of interest. In bins with odd numbers of polypeptides, the median polypeptide was discarded, as were any pairs of polypeptides with the same fractional content of the codon of interest. The bin was then divided in half based on fractional codon content, and the polypeptides were added to the overall “high” or “low” distributions. The final resulting sets of polypeptides had nearly identical distributions of the controlled parameter but significant variation in the fractional content of the codon of interest. Heteroskedastic matched T-tests were used to determine the significance of the difference in the expression and solubility score distributions for those polypeptide sets.

Statistical Analyses.

Logistic regressions were performed in STATA with significance determined from Z-scores for individual variables and chi-squared distributions for models. Counting-statistics-based 95% confidence intervals were calculated using Bayesian maximum likelihood estimates of the binomial distribution.

Evaluation of Prediction of NMR Success.

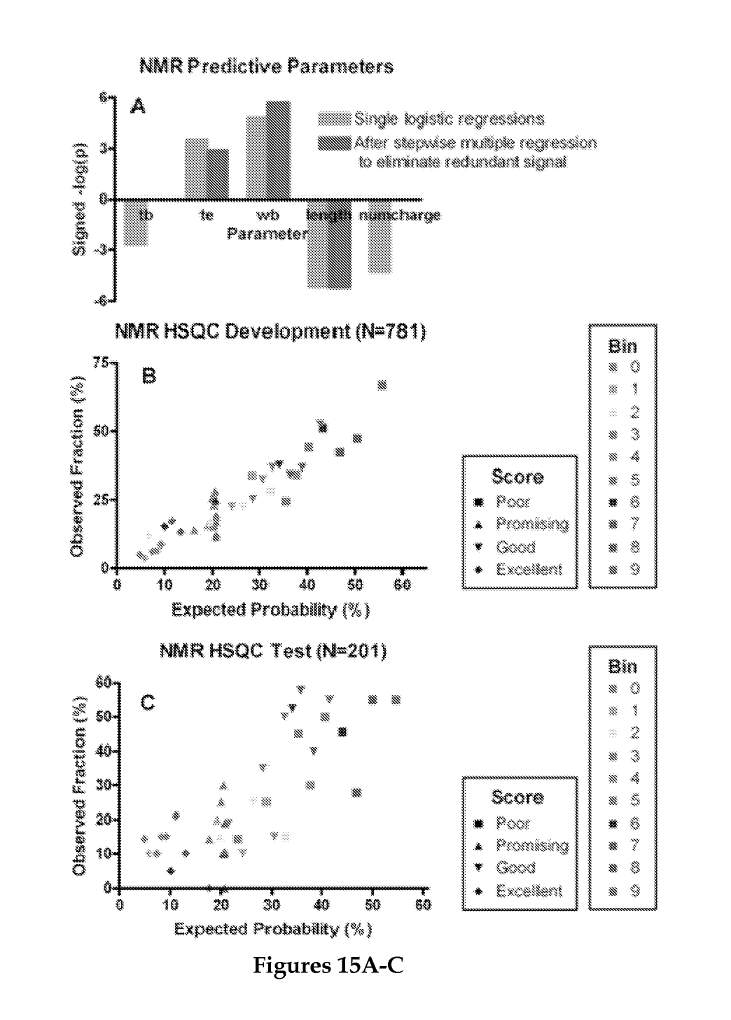

Nearly 1,000 polypeptides under 200 amino acids long which were suitably expressed and soluble were also screened for NMR suitability (Liu G et al. (2005) Proceedings of the National Academy of Sciences of the United States of America 102:10487). NMR spectra were subjectively scored as unfolded, poor, promising, good, or excellent. By converting evaluations from “poor” to “excellent” into numerical scores, the same analyses as described above was performed. Individual regressions revealed some moderate effects (FIG. 15A) (e.g. the negative effect of chain length), but the combined predictor was only moderately significant in describing the test set (FIGS. 15B & C). The major sequence determinants of NMR success are those related to the prerequisite task of obtaining well expressed and soluble polypeptide.

Details on NMR Prediction.

After single regressions and parameter culling (FIG. 15A), significant positive effects were observed for exposed Thr and buried tryptophan. Significant negative effects were observed for polypeptide length, number of charged residues, and buried Thr. However, when the predictors were combined using stepwise ordinal logistic regression, only length, exposed Thr, and buried tryptophan remained significant (FIG. 15A). The number of charged residues most likely served as a surrogate for the dominant length effect; the elimination of buried Thr remains puzzling. The overall predictor was significant in the development set of 781 polypeptides (p=1.5×10¹¹), but of only marginal significance for the test set of 201 polypeptides (p=0.07) (FIGS. 15B & C). The most significant sequence parameters for NMR success have to do with providing expressed and soluble polypeptide, so that when only those polypeptides are considered, the remaining simple sequence property differences are relatively insignificant.

Statistical analyses were performed on 9,644 polypeptides which were cloned and expressed in E. coli in the NESG polypeptide-production pipeline and systematically scored for expression and solubility levels. Secondary structure and disorder predictions were run for all polypeptides, and logistic regressions calculated to relate sequence properties (including amino acid frequencies, charge variables, hydrophobicity, and side chain entropy) to expression and solubility scores. Results from these regressions are useful both for an increased understanding of expression/solubility mechanism and for the practical purpose of predicting from sequence alone which polypeptide targets are likely to be practically usable.

Methods

7733 NESG targets were cloned, expressed, & scored for: expression (E: 0-5), solubility (S: 0-5) and usability (E*S>11).

Logistic regressions (continuous input, binary or stepwise output) were performed between E, S, or (E*S>11) and (1) Amino acid frequency (total, predicted buried, or exposed), (2) hydrophobicity (gravy), (3) total or predicted exposed side chain entropy, (4) fractional number of charged residues, (5) whole and fractional signed and absolute net charge, (5) length, and (6) fraction residues predicted disordered by DISOPRED2

Data Mining/Regression Analysis.

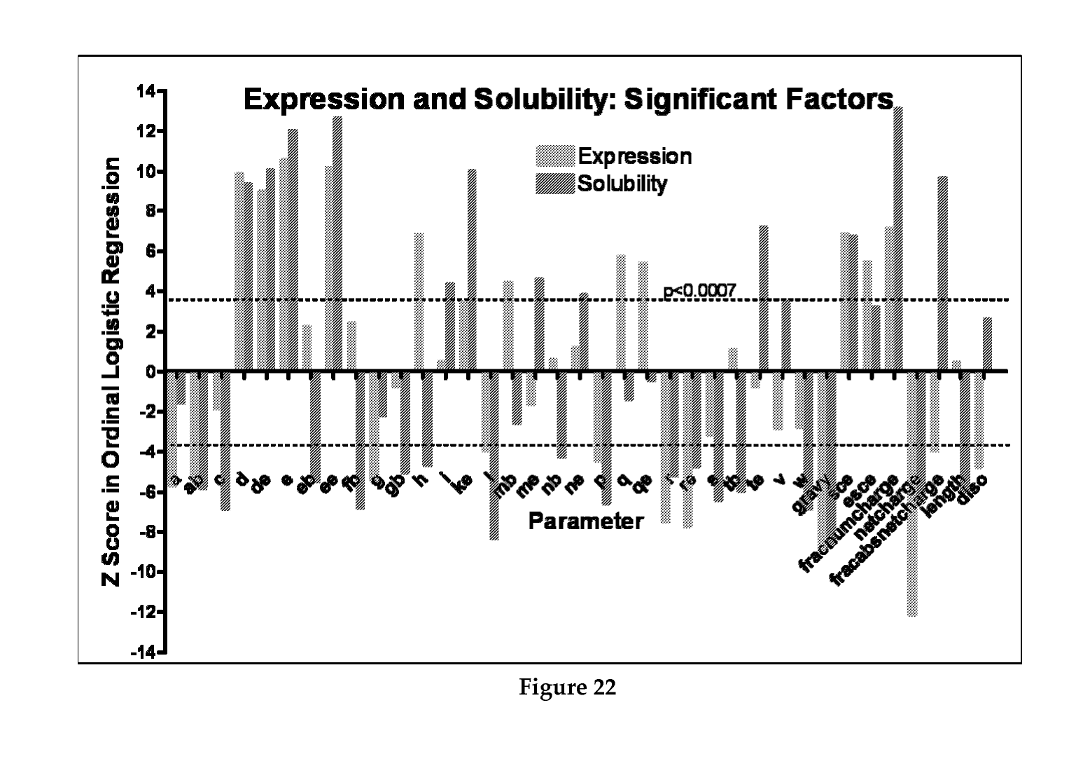

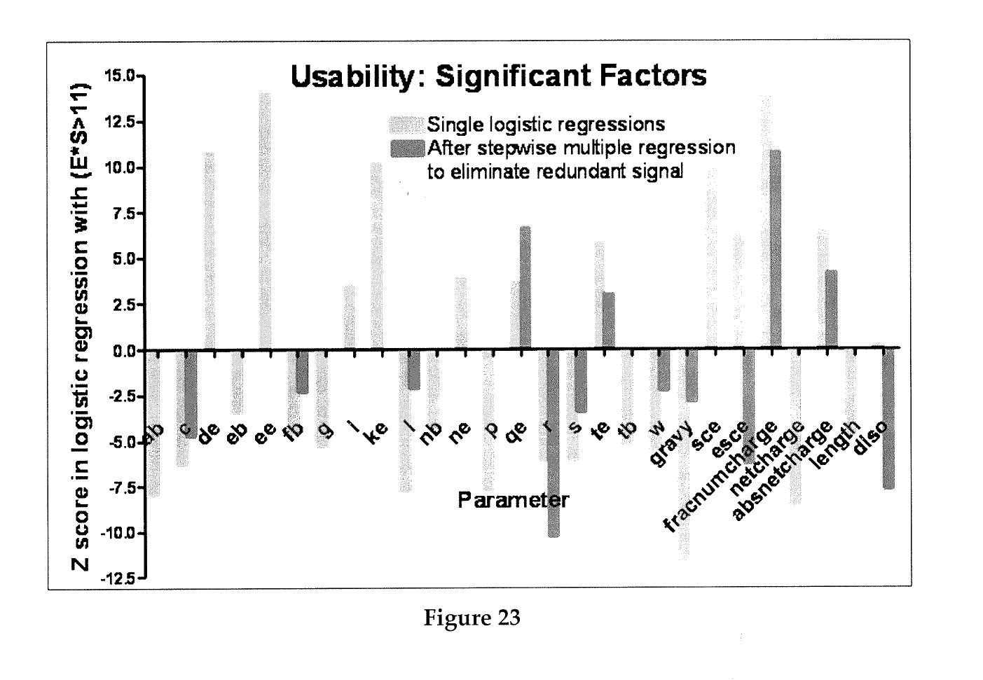

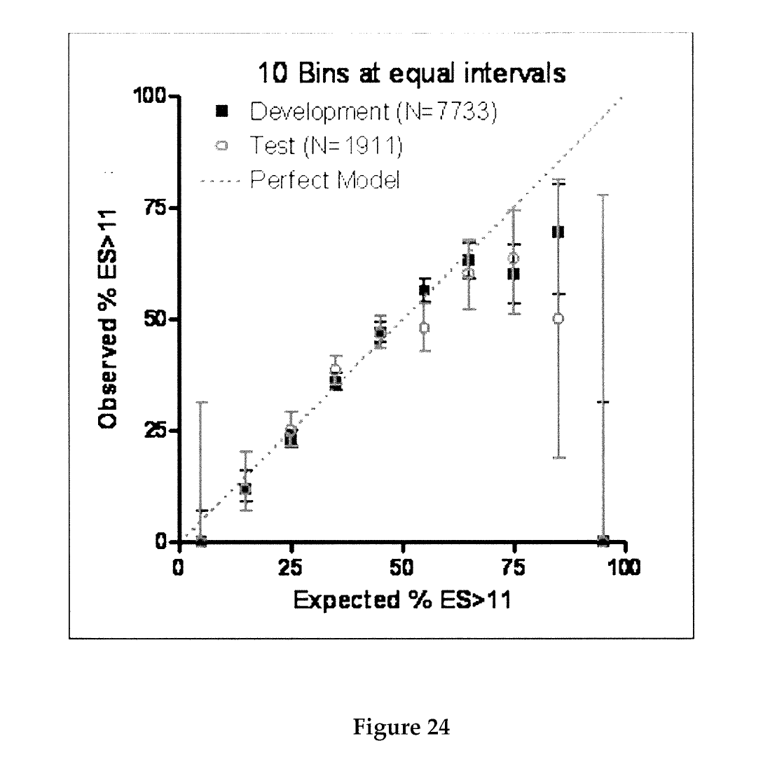

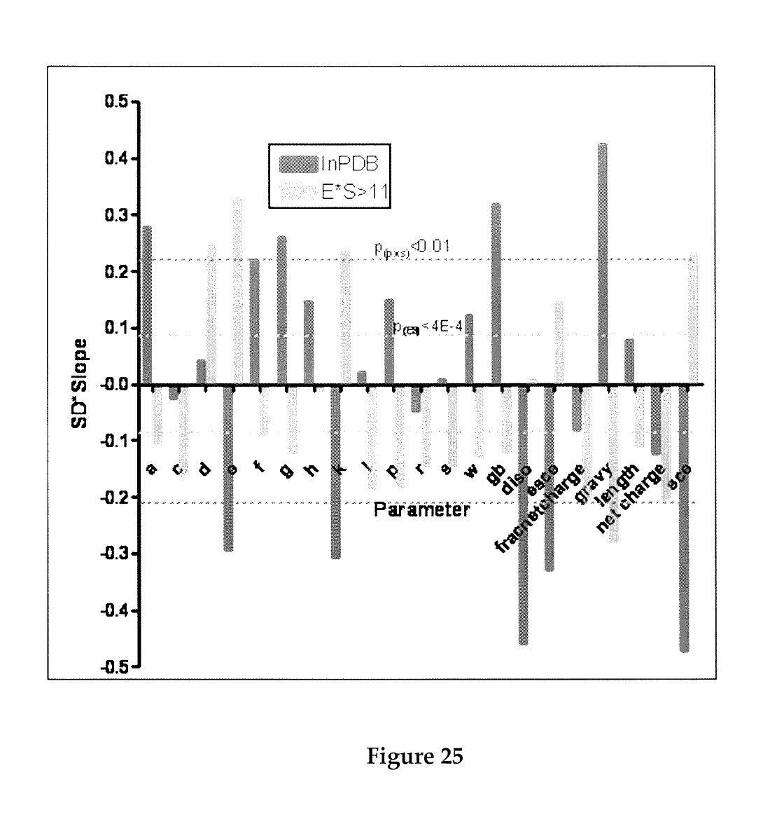

As shown in FIGS. 22-29, 9,644 polypeptides were taken from NESG pipeline data; only one construct of each polypeptide was considered. Polypeptides were manually scored for expression and (expression-independent) solubility based on Coomassie gels. GRAVY was calculated using the Kyte-Doolittle values of hydropathy (1982). SCE values for the individual amino acids were taken from Creamer (2000). DISOPRED scores were calculated locally using the DISOPRED2 program with a 2% false positive rate (Ward et al. 2004). Calculations of predicted burial/exposure and secondary structure were performed with PhD/PROF (Rost, Yachdav & Liu, 2004). Binary and ordinal logistic regressions were performed using STATA (StataCorp, College Station, Tex.).

NMR Structure Solution.

NMR structure solution was performed as previously described (Liu G et al. (2005) Proceedings of the National Academy of Sciences of the United States of America 102:10487).

### REFERENCES

Acton T B et al. (2005) Robotic cloning and polypeptide production platform of the Northeast Structural Genomics Consortium. Methods in Enzymology 394:210-243.
Akaike H (1974) A new look at the statistical model identification. IEEE transactions on automatic control 19:716-723.
Appel R D, Bairoch A, Hochstrasser D F (1994) A new generation of information retrieval tools for biologists: the example of the ExPASy WWW server. Trends in Biochemical Sciences 19:258.
Bertone P et al. (2001) SPINE: an integrated tracking database and data mining approach for identifying feasible targets in high-throughput structural proteomics. Nucleic acids research 29:2884.
Brant R (1990) Assessing proportionality in the proportional odds model for ordinal logistic regression. Biometrics 46:1171-1178.
Campbell J W et al. (1972) X-ray diffraction studies on enzymes in the glycolytic pathway. Cold Spring Harb. Symp. Quant. Biol 36:165-170.
Carstens C P (2003) Use of tRNA-supplemented host strains for expression of heterologous genes in E. coli. Methods in Molecular Biology 205:225-234.
Chen J, Acton T B, Basu S K, Montelione G T, Inouye M (2002) Enhancement of the solubility of polypeptides overexpressed in Escherichia coli by heat shock. Journal of molecular microbiology and biotechnology 4:519-524.
Chen L, Oughtred R, Berman H M, Westbrook J (2004) TargetDB: a target registration database for structural genomics projects (Oxford Univ Press).
Christen E H et al. (2009) A general strategy for the production of difficult-to-express inducer-dependent bacterial repressor polypeptides in Escherichia coli. Polypeptide Expression and Purification.
Creamer T P (2000) Side-chain conformational entropy in polypeptide unfolded states. Polypeptides: Structure, Function, and Genetics 40.
Crombie T, Swaffield J C, Brown A J (1992) Polypeptide folding within the cell is influenced by controlled rates of polypeptide elongation. J. Mol. Biol 228:7-12.
Dale G E, Broger C, Langen H, Arcy A D, Stüber D (1994) Improving polypeptide solubility through rationally designed amino acid replacements: solubilization of the trimethoprim-resistant type 51 dihydrofolate reductase. Polypeptide Engineering Design and Selection 7:933-939.
Davis G D, Elisee C, Newham D M, Harrison R G (1999) New fusion polypeptide systems designed to give soluble expression in Escherichia coli. Biotechnology and bioengineering 65.
De Bernardez Clark E (1998) Refolding of recombinant polypeptides. Current Opinion in Biotechnology 9:157-163.
Derewenda Z S (2004) Rational polypeptide crystallization by mutational surface engineering. Structure 12:529-535.
Etchegaray J P, Inouye M (1999) Translational enhancement by an element downstream of the initiation codon in Escherichia coli. Journal of Biological Chemistry 274:10079-10085.
Georgiou G, Valax P (1996) Expression of correctly folded polypeptides in Escherichia coli. Current Opinion in Biotechnology 7:190-197.
Goh C S et al. (2003) SPINE 2: a system for collaborative structural proteomics within a federated database framework. Nucleic acids research 31:2833.
Goh C S et al. (2004) Mining the structural genomics pipeline: identification of polypeptide properties that affect high-throughput experimental analysis. Journal of molecular biology 336:115-130.
Gottesman S (1990) Minimizing proteolysis in Escherichia coli: genetic solutions. Methods in enzymology 185:119.
Gustafsson C, Govindarajan S, Minshull J (2004) Codon bias and heterologous polypeptide expression. Trends in biotechnology 22:346-353.
Hatfield G W, Roth D A (2007) Optimizing scaleup yield for polypeptide production: Computationally Optimized DNA Assembly (CODA) and Translation Engineering. Biotechnol Annu Rev 13:27-42.
Hosmer D W, Lemeshow S (2004) Applied logistic regression (Wiley-Interscience).
Idicula-Thomas S, Balaji P V (2005) Understanding the relationship between the primary structure of polypeptides and its propensity to be soluble on overexpression in Escherichia coli. Polypeptide Science: A Publication of the Polypeptide Society 14:582.
Idicula-Thomas S, Kulkarni A J, Kulkarni B D, Jayaraman V K, Balaji P V (2006) A support vector machine-based method for predicting the propensity of a polypeptide to be soluble or to form inclusion body on overexpression in Escherichia coli. Bioinformatics 22:278-284.
Kapust R B, Waugh D S (1999) Escherichia coli maltose-binding polypeptide is uncommonly effective at promoting the solubility of polypeptides to which it is fused. PRS 8:1668-1674.
Kefala G, Kwiatkowski W, Esquivies L, Maslennikov I, Choe S (2007) Application of Mistic to improving the expression and membrane integration of histidine kinase receptors from Escherichia coli. Journal of Structural and Functional Genomics 8:167-172.
Kim C H, Oh Y, Lee T H (1997) Codon optimization for high-level expression of human erythropoietin (EPO) in mammalian cells. Gene 199:293-301.
Komar A A (2009) A pause for thought along the co-translational folding pathway. Trends Biochem. Sci 34:16-24.
Krogh A, Larsson B, Von Heijne G, Sonnhammer E L L (2001) Predicting transmembrane polypeptide topology with a hidden Markov model: application to complete genomes. J Mol Biol 305:567-580.
Krüger M K, Pedersen S, Hagervall T G, Sorensen M A (1998) The modification of the wobble base of tRNAGlu modulates the translation rate of glutamic acid codons in vivo. Journal of molecular biology 284:621-631.
Kudla G, Murray A W, Tollervey D, Plotkin J B (2009) Coding-sequence determinants of gene expression in Escherichia coli. science 324:255.
Kyte J, Doolittle R F (1982) A simple method for displaying the hydropathic character of a polypeptide. Journal of Molecular Biology 157:105.
Lee C et al. (2008) An improved SUMO fusion polypeptide system for effective production of native polypeptides. Polypeptide Sci. 17:1241-1248.
Lewis H A et al. (2005) Impact of the {Delta} F 508 mutation in first nucleotide-binding domain of human cystic fibrosis transmembrane conductance regulator on domain folding and structure. Journal of Biological Chemistry 280:1346-1353.
Liu G et al. (2005) NMR data collection and analysis protocol for high-throughput polypeptide structure determination. Proceedings of the National Academy of Sciences of the United States of America 102:10487.
Luft J R et al. (2003) A deliberate approach to screening for initial crystallization conditions of biological macromolecules. Journal of Structural Biology 142:170-179.
Magnan C N, Randall A, Baldi P (2009) SOLpro: accurate sequence-based prediction of polypeptide solubility. Bioinformatics.
Makrides S C (1996) Strategies for achieving high-level expression of genes in Escherichia coli. Microbiology and Molecular Biology Reviews 60:512.
Nakamura Y, Gojobori T, Ikemura T (2000) Codon usage tabulated from international DNA sequence databases: status for the year 2000. Nucleic Acids Res 28:292.
Pédelacq J D et al. (2002) Engineering soluble polypeptides for structural genomics. Nature biotechnology 20:927-932.
Pedersen S (1984) Escherichia coli ribosomes translate in vivo with variable rate. The EMBO Journal 3:2895.
Price W N et al. (2009) Understanding the physical properties that control polypeptide crystallization by analysis of large-scale experimental data. Nat. Biotechnol 27:51-57.
Rice P, Longden I, Bleasby A (2000) EMBOSS: the European molecular biology open software suite. Trends in genetics 16:276-277.
Rost B (2005) How to use polypeptide 1D structure predicted by PROFphd. The proteomics protocols handbook. Totowa (New Jersey): Humana:875-901.
Rost B, Yachdav G, Liu J (2004) The predictpolypeptide server. Nucleic Acids Research 32:W321.
Sanbonmatsu K Y, Joseph S, Tung C (2005) Simulating movement of tRNA into the ribosome during decoding. Proceedings of the National Academy of Sciences of the United States of America 102:15854-15859.
Slabinski, L., L. Jaroszewski, et al. (2007). “The challenge of polypeptide structure determination—lessons from structural genomics.” Polypeptide Sci 16(11): 2472-82.
Smialowski P et al. (2007) Polypeptide solubility: sequence based prediction and experimental verification. Bioinformatics 23:2536.
Sorensen H P, Mortensen K K (2005) Advanced genetic strategies for recombinant polypeptide expression in Escherichia coli. Journal of biotechnology 115:113-128.
Tanha J et al. (2006) Improving solubility and refolding efficiency of human V(H)s by a novel mutational approach. Polypeptide Eng. Des. Sel 19:503-509.
Tartaglia G G, Pechmann S, Dobson C M, Vendruscolo M (2009) A Relationship between mRNA Expression Levels and Polypeptide Solubility in E. coli. Journal of Molecular Biology.
Tresaugues L et al. (2004) Refolding strategies from inclusion bodies in a structural genomics project. Journal of Structural and Functional Genomics 5:195-204.
Trevino S R, Scholtz J M, Pace C N (2007) Amino acid contribution to polypeptide solubility: Asp, Glu, and Ser contribute more favorably than the other hydrophilic amino acids in RNase Sa. J. Mol. Biol 366:449-460.
Wagner S et al. (2008) Tuning Escherichia coli for membrane polypeptide overexpression. Proc. Natl. Acad. Sci. U.S.A 105:14371-14376.
Waldo G S (2003) Genetic screens and directed evolution for polypeptide solubility. Current opinion in chemical biology 7:33-38.
Wang and Dunbrack, Jr. (2003). “PISCES: a polypeptide sequence culling server.” Bioinformatics 19:1589-1591.
Ward J J, McGuffin U, Bryson K, Buxton B F, Jones D T (2004) The DISOPRED server for the prediction of polypeptide disorder (Oxford Univ Press).
Wigley W C, Stidham R D, Smith N M, Hunt J F, Thomas P J (2001) Polypeptide solubility and folding monitored in vivo by structural complementation of a genetic marker polypeptide. Nat. Biotechnol 19:131-136.
Wilkinson D L, Harrison R G (1991) Predicting the solubility of recombinant polypeptides in Escherichia coli. Nature Biotechnology 9:443-448.
Wu X, Jörnvall H, Berndt K D, Oppermann U (2004) Codon optimization reveals critical factors for high level expression of two rare codon genes in Escherichia coli: RNA stability and secondary structure but not tRNA abundance. Biochemical and Biophysical Research Communications 313:89-96.
Yadava A, Ockenhouse C F (2003) Effect of Codon Optimization on Expression Levels of a Functionally Folded Malaria Vaccine Candidate in Prokaryotic and Eukaryotic Expression Systems Editor: W A Petri, Jr. Infection and immunity 71:4961-4969.

### Example 2

### Codon Replacement for Improving Protein Expression Levels and Toxicity Thereof

Proteins are made up of amino acids, which are each coded for by a sequence of three DNA bases. This triplet of DNA bases is called a codon, and each amino acid has more than one codon. However, some codons naturally translate less efficiently than other, yielding proteins with low expression levels. This is disadvantageous when attempting to over-express proteins in the laboratory for experimental studies. Therefore, codon usage is very important during protein expression.

The data presented in Example 1 demonstrated that previously published metrics for codon-translation efficiency do not match statistical trends observed in several thousand protein expression experiments conducted using standard methods with T7-polymerase-based pET vectors in E. coli strain BL21λ(DE3). These trends have been revalidated via analysis of several sub-divisions of a substantially expanded experimental dataset. These analyses demonstrate that overexpression of a specific set of “rare” tRNAs does not improve the deleterious effects on expression of the corresponding codons. The statistical trends from the large-scale protein expression dataset were used to determine a new metric for codon-translation efficiency, which is distinct from prior metrics. The metric described herein, the Columbia Metric, is uncorrelated with codon frequency or tRNA frequency, the dominant factors used to construct prior metrics.

We have now tested the use of the Columbia Metric to identify proteins whose expression is limited by poor codon usage and to improve their expression via codon optimization. Furthermore, a systematic method used to evaluate and predict the likely efficacy of codon replacement for improving the net expression of proteins that originally have low expression levels by monitoring the toxicity caused by expression is described. We obtained improved expression of five out of five target proteins selected based on having a high content of inefficiently translated codons according to the Columbia Metric. This success rate exceeds that demonstrated in previous studies of codon optimization. Furthermore, we present evidence that toxicity of the original gene (i.e., reduction in cell growth rate upon induction of its expression) can be used to further refine the prediction of the efficacy of codon optimization. Proteins showing high toxicity upon induction give erratic results, due to genetic selection for expression and toxicity reducing mutations during growth. However, proteins showing moderate toxicity tend to show reduced toxicity and moderate to high increases in expression level upon codon optimization. The single non-toxic protein examined in our set of five also shows substantial enhancement in its expression level upon codon optimization.

The experimental methods and results discussed herein validate the methods described in Example 1, and establish new, easy, and inexpensive growth assays that are useful to refine prediction of which proteins can be enhanced in their expression level by optimization of codon usage. This has not been previously shown in prior studies of codon optimization.

### Methods of the Example

Proteins were over-expressed using the pET system created by Novagen. A gene construct for the protein of interest was subcloned into an ampicillin resistant modified pET21 vector (pET21 NESG) and transformed into E. coli BL21 pMgK cells (a codon enhanced strain supplementing tRNA levels for AGA, AGG and ATT codons).

In one embodiment, two individual colonies of each construct were grown overnight at 37° C. in 5 mL cultures of Luria Broth supplemented with kanamycin and ampicillin. 40 μL of the overnight pre-culture was then used to inoculate 2 mL of MJ9 minimal media, which was grown over a second night at 37° C. The following morning, 240 μL of the overnight MJ9 culture was used to inoculate 6 mL of MJ9 media so that the OD₆₀₀ of the larger culture measured 0.2. This culture was incubated at 37° C. until the OD₆₀₀ measured 0.6, at which point protein expression was induced with IPTG (1 mM final) and the temperature lowered to 17° C. One reference culture for each protein construct was not induced by IPTG. During protein expression, the OD₆₀₀ of all the cultures was monitored every 30 minutes to assess the toxicity of the expressed protein to the host cell. At 16 h post-induction, the cells were harvested by centrifugation, washed with PBS buffer (50 mM NaH₂PO₄, pH 8, 300 mM NaCl), and resuspended in 0.6 mL of lysis buffer (50 mM NaH₂PO₄, pH 8, 300 mM NaCl, 10 mM β-mercaptoethanol), then lysed by sonciation (three 30 s pulses at 10 W).

In another embodiment, small cultures (0.5 mL) of Luria Broth supplemented with ampicillin and kanamycin were inoculated with a single colony (two isolates of each construct are assayed) and grown at 37° C. for 6 hours. 10 μL of this preculture was then used to inoculate 0.5 mL of MJ9 minimal media, which was grown over night at 37° C. The following morning, 200 μL of the overnight MJ9 culture was used to inoculate 2 mL of MJ9 media so that the OD₆₀₀ of the larger culture measured 0.2. This culture was incubated at 37° C. until the OD₆₀₀ measured 0.6, at which point protein expression was induced with IPTG (1 mM final) and the temperature lowered to 17° C. One reference culture for each protein construct was not induced by IPTG. During protein expression, the OD₆₀₀ of all the cultures were monitored every 30 minutes to assess the toxicity of the expressed protein to the host cell. At 16 h post-induction, the cells were harvested by centrifugation and resuspended in lysis buffer (200 μL) and lysed by sonciation (30 S bursts at 18 W followed by 30 S cooling periods over a 12 min cycle time).

The total amount of protein was determined by the Bradford Assay. In the experiments presented here, an equal amount of cell lysate was evaluated by SDS-PAGE, because this normalization reflects the net gain in economic and process efficiency during protein expression.

Results:

Toxicity to the host cell upon protein induction can lead to different scenarios after codon optimization. If the protein itself is highly toxic, more efficient protein expression can actually further impede cell growth, making improved expression unlikely due to both the reduction in growth-rate and genetic selection for expression-reducing mutations. Without being bound by theory, complete cessation of cell growth after induction of the unmodified gene is correlated with this mechanistic scenario. We have observed that moderate toxicity after induction (i.e., reduction in growth-rate but not complete cessation in growth) can be relieved by codon optimization. Thus, net protein expression per volume of cell culture is increased by enabling cells to grow to higher density. In addition, in this situation and for proteins not showing any toxicity upon induction, codon optimization can lead to enhanced expression in each cell due to more efficient translation.

The expression of a highly toxic protein (XR47) yielded erratic results, showing substantially improved expression in some clones but not others. In this case, codon optimization did not relieve toxicity, and the variability in the results is likely attributable to differences in selection of toxicity-reducing mutations during cell growth after induction. Without being by theory, high toxicity of this kind is an indicator that investment in codon optimization is not likely to be worthwhile.

As discussed herein, the induction of expression of the original gene is either non-toxic or only moderately toxic, and at least moderately improved expression is observed for all four target proteins.

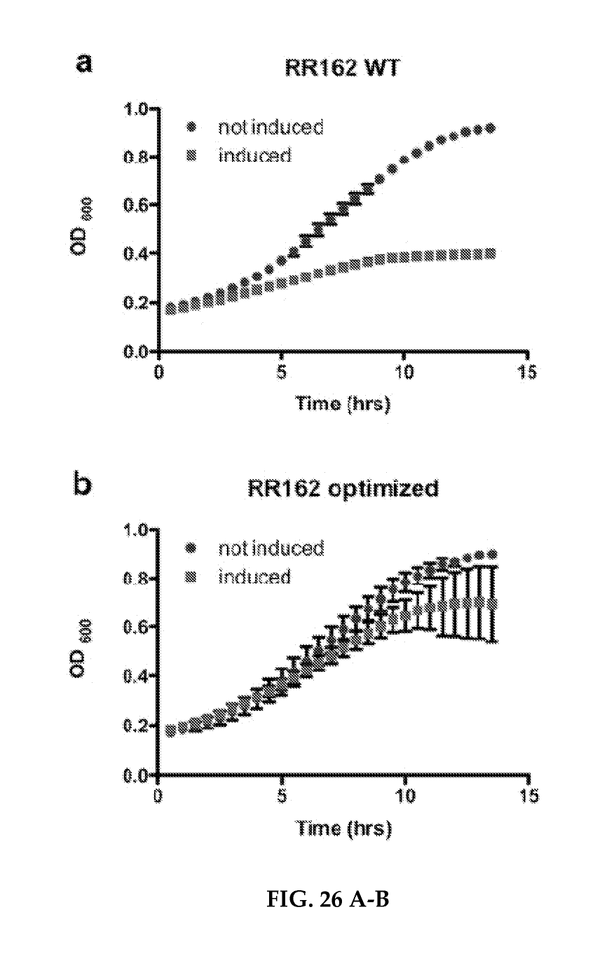

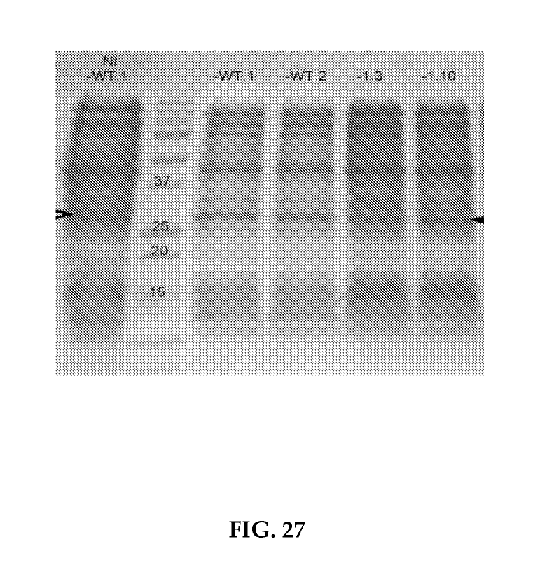

RR162 is a case where codon optimization decreases moderate toxicity upon induction and thereby increases protein expression per liter of culture, even though it does not increase the level of protein expression compared to other proteins in the cell. Prior to codon optimization, cells expressing the protein do not grow as well as cells that were left not-induced (FIG. 26A), indicating that protein expression causes toxicity. Two codon optimized clones were evaluated (RR162-1.3 and RR162-1.10) and both greatly reduced the toxicity upon induction of mRNA/protein expression (FIG. 26B). Although expression of the target protein is not consistently increased compared to other cellular proteins, SDS-PAGE analysis shows that the increased cell growth produced a net increase in expression of the target protein normalized to culture volume (FIG. 27).

SrR141 and XR92 are two examples of how codon optimization improved both toxicity and protein expression.

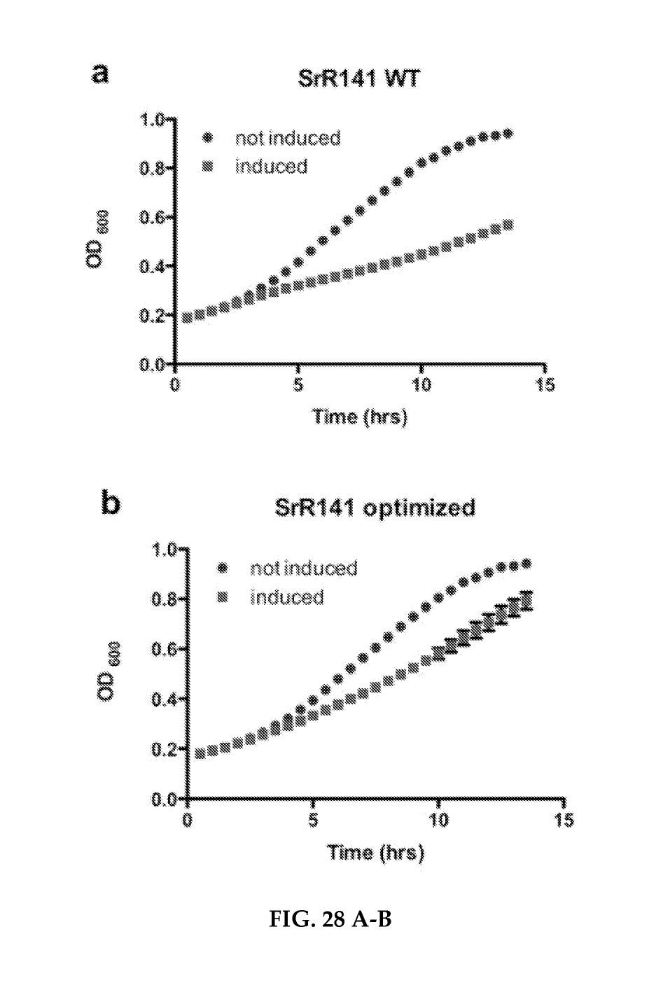

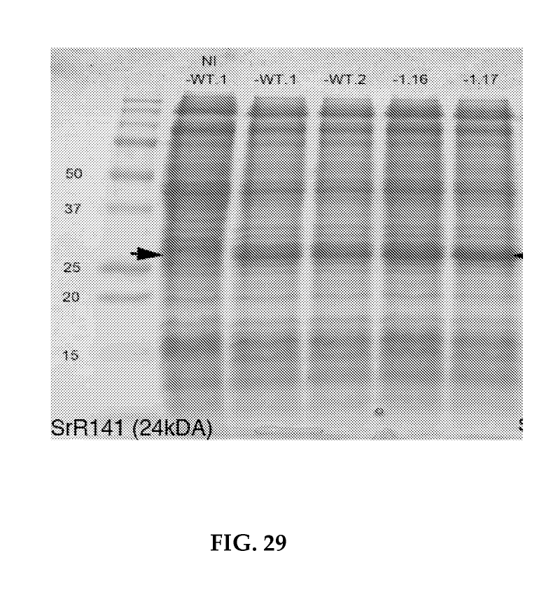

Codon optimization of SrR141 relieved cell toxicity and moderately increased protein expression level relative to other cellular proteins. Without being bound by theory, the variability in the gain in expression may be attributable to plasmid sequence variations during molecular biological manipulations, which are common, or to genetic selection during induction. Additional experiments will be carried out to determine between these possibilities. As with RR162, expression of SrR141 has a negative impact on cell growth (FIG. 28A). Codon optimization reduces cell toxicity and improves cell growth (FIG. 28B). However, the protein expression levels of codon optimized constructs (1.16 and 1.17) were only marginally higher than the wild-type gene construct (FIG. 29).

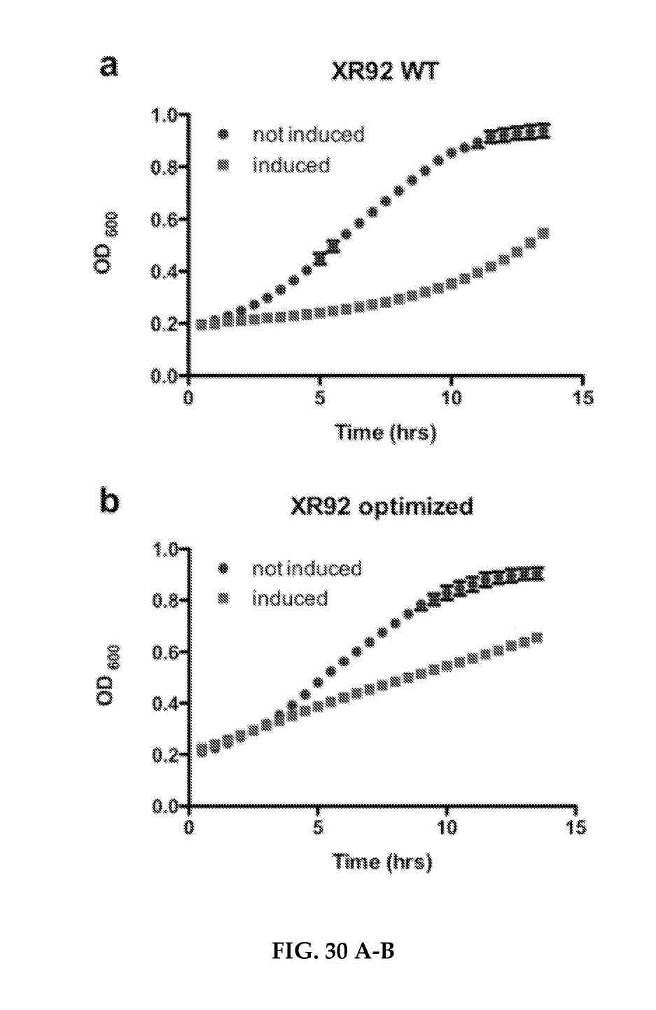

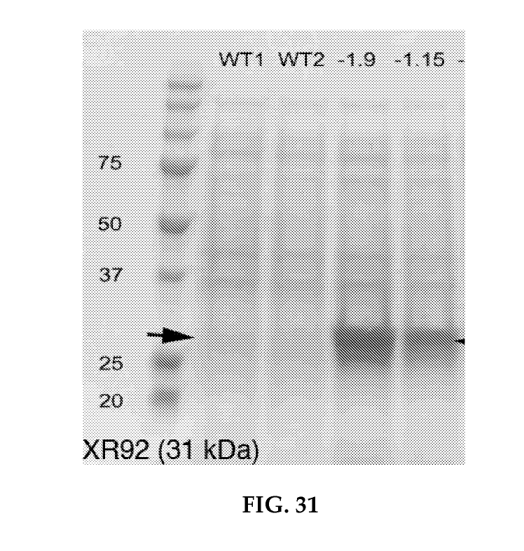

Codon optimization of XR92 resulted in a great improvement of protein expression, but had less of an effect on the toxicity to the cells. FIG. 30 shows cell growth monitored by cell density (OD₆₀₀, y-axis) over time (x-axis). Expression of the wild-type gene construct impaired cell growth (FIG. 30A). Codon optimization reduced cell toxicity and improved cell growth (FIG. 30B), albeit not as much as was observed for SrR141 (FIG. 28B). However, the improvement of protein expression of the codon optimized constructs (1.9 and 1.15) was enormous (FIG. 31). No expression was observed in cells expressing the wild-type construct (WT1, WT2).

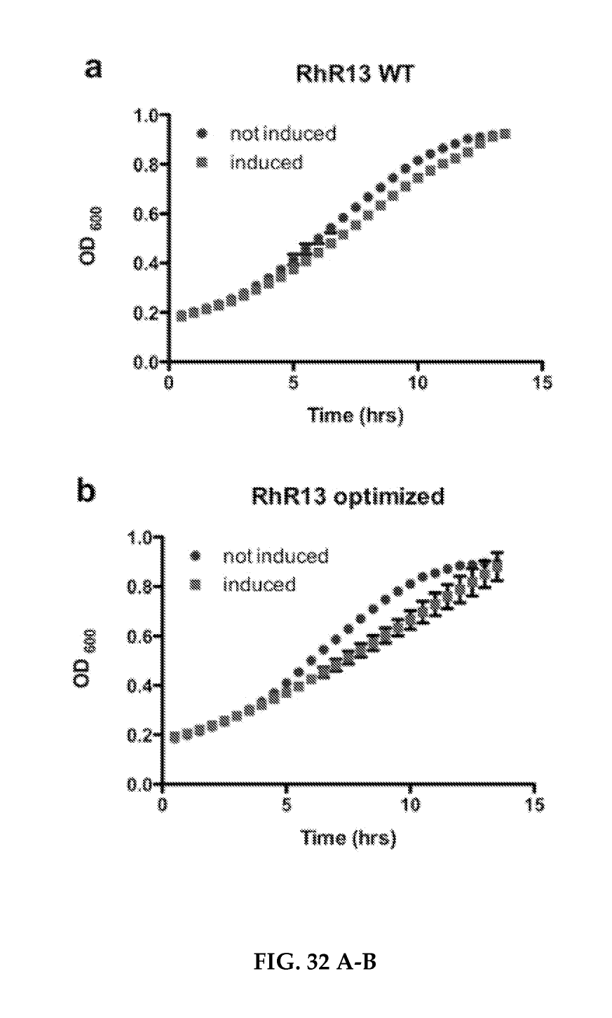

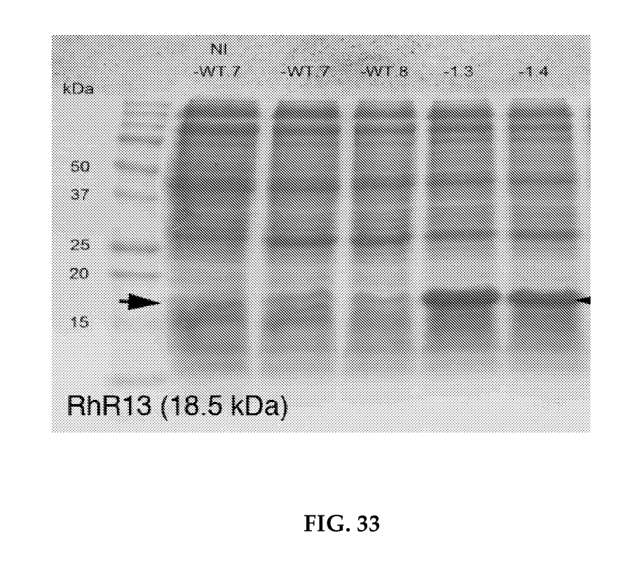

RhR13. Proteins that are not toxic to the host cell when expressed will make good candidates for codon optimization. For example, expression of the wild-type RhR13 gene construct (blue diamonds) did not affect cell growth as observed from cell density (OD₆₀₀, y-axis) measurements over time (x-axis) when compared to the non-induced culture (NI, red squares) (See FIG. 32). Codon optimization greatly improved protein expression in two constructs which had complete optimization (1.3 and 1.4; FIG. 33), while two that were only partially optimized (2.5 and 2.6, in which only a single codon was optimized) did not exhibit improved protein expression.

Conclusion:

Toxicity is a commonly observed problem during recombinant protein expression. This Example has shown that, in some cases, codon optimization can reduce the toxicity towards the host cell. Without being bound by theory, the relief of toxicity is unclear; but, codon optimization may reduce stress on the translational machinery in the cell. Checking for relief of toxicity after codon optimization is a good indicator that protein expression will also have increased. In addition to alleviating toxicity, proteins not toxic to cell growth are good candidates for codon optimization, and our data show dramatic improvement of protein yield during over-expression in this situation. The toxicity of the overexpressed protein on cell growth must be accounted for in any assessment of the effects of codon optimization on protein expression. This toxicity effect has largely been ignored by other groups when studying the effects of codon optimization on protein production.

It is noted that Kudla et al. (Science 10 Apr. 2009: Vol. 324 no. 5924 pp. 255-258) report that the secondary structure in the first 15 codons of a GFP protein affects it solubility in that the inefficiently translated message can impede cell growth. It is also noted that Wagner et al. (PNAS Sep. 23, 2008 vol. 105 no. 38 14371-14376) report that lowering message expression levels can improve the yield of toxic proteins; however, the increased expression more severely impedes growth thereby lowering net expression, thus showing that increasing the expression of toxic proteins is complex and unpredictable.

### Example 3

### Nucleic Acid Sequences Encoding Proteins from Example 2 and Amino Acid Sequences of Same

The nucleic acid sequence encoding the protein SrR141-1 (SEQ ID NO: 1)—

ATGGCCGCCATGCCCAAGCCCGCTGCGTTCTGGAACGACCGCTTTGCCAA
CGAAGAATACGTGTACGGCGAAGCCCCCAACCGTTTCGTCGCGAGCGCCG
CCCGTACGTGGCTGCCGGAAGCCGGTGAAGTTCTCCTGCTCGGGGCGGGC
GAAGGGCGTAACGCCGTGCATCTGGCCCGTGAAGGCCATACGGTCACCGC
GGTCGATTACGCCGTGGAAGGGCTCCGTAAGACGGAACGTCTCGCGACGG
AAGCCGGGGTGGAAGTCGAAGCGATTCAAGCCGATGTGCGTGAATGGAAG
CCCGCCCGTGCGTGGGATGCGGTCGTCGTCACGTTTCTCCATCTTCCCGC
CGATGAACGTCCGGGCCTGTACCGTCTCGTTCAACGTTGTTTGCGTCCCG
GGGGGCGTCTCGTGGCGGAATGGTTTCGTCCGGAACAACGTACGGATGGC
TACACGAGCGGCGGCCCGCCCGATCCTGCCATGATGGTCACCGCCGATGA
ACTCCGTGGGCATTTCGCCGAAGCGGGCATTGATCATCTCGAAGCGGCCG
AACCGACCCTCGATGAAGGCATGCATCGTGGCCCCGCGGCGACGGTTCGT
CTCGTGTGGTGCCGTCCGTCCACCTCG

The nucleic acid sequence encoding the protein SrR141-2 (SEQ ID NO: 2)—

ATGGCCGCCATGCCCAAGCCCGCTGCGTTCTGGAACGACCGCTTTGCCAA
CGAAGAATACGTGTACGGCGAAGCCCCCAACCGCTTCGTCGCGAGCGCCG
CCCGGACGTGGCTGCCGGAAGCCGGTGAAGTTCTCCTGCTCGGGGCGGGC
GAAGGGCGCAACGCCGTGCACCTGGCCCGGGAAGGCCATACGGTCACCGC
GGTCGACTACGCCGTGGAAGGGCTCCGCAAGACGGAACGCCTCGCGACGG
AAGCCGGGGTGGAAGTCGAAGCGATCCAGGCCGATGTGCGCGAATGGAAG
CCCGCCCGGGCGTGGGACGCGGTCGTCGTCACGTTTCTCCACCTTCCCGC
CGACGAACGACCGGGCCTGTACCGCCTCGTTCAGCGCTGTTTGCGGCCCG
GGGGGCGCCTCGTGGCGGAATGGTTTCGCCCGGAACAGCGCACGGACGGC
TACACGAGCGGCGGCCCGCCCGATCCTGCCATGATGGTCACCGCCGACGA
ACTCCGCGGGCACTTCGCCGAAGCGGGCATCGACCATCTCGAAGCGGCCG
AACCGACCCTCGACGAAGGCATGCACCGGGGCCCCGCGGCGACGGTTCGT
CTCGTGTGGTGCCGGCCGTCCACCTCG

The amino acid sequence of SrR141 (SEQ ID NO: 9)—

MAAMPKPAAFWNDRFANEEYVYGEAPNRFVASAARTWLPEAGEVLLLGAG
EGRNAVHLAREGHTVTAVDYAVEGLRKTERLATEAGVEVEAIQADVREWK
PARAWDAVVVTFLHLPADERPGLYRLVQRCLRPGGRLVAEWFRPEQRTDG
YTSGGPPDPAMMVTADELRGHFAEAGIDHLEAAEPTLDEGMHRGPAATVR
LVWCRPSTSLEHHHHHH

The nucleic acid sequence encoding the protein RhR13-1 (SEQ ID NO: 3)—

ATGGCGCGTTCGATCGATTACGGCAACCTCATGCACCGCGCGATGCGTGG
CCTGATTCAAAGCGTGCTCGAAGATGTGGCCGAACATGGGCTGCCCGGCG
CGCATCATTTCTTCATTACCTTCGATACGACCCATCCCGATGTGGCCATG
GCCGATTGGCTCCGTGCGCGTTATCCGCAAGAAATGACGGTCGTGATTCA
ACATTGGTACGAAAACCTCTCCGCCGATGATCATGGCTTCTCGGTCACGC
TGAACTTCGGCAACCAACCCGAACCGCTGGTCATTCCCTTCGATGCCGTG
CGTACCTTCGTCGATCCGTCCGTGGAATTCGGCCTCCGTTTCGAAACCCA
TGAAGAAGATGAAGAAGAAGAAACGGGCGGCGATGAAGATCCCGATGGCG
ATGATGAACCGCCGCGTCATGATGCGCAAGTCGTGAGCCTCGATAAGTTC
CGTAAG

The nucleic acid sequence encoding the protein RhR13-2 (SEQ ID NO: 4)—

ATGGCGCGTTCGATCGATTACGGCAACCTCATGCACCGCGCGATGCGGGG
CCTGATCCAGAGCGTGCTCGAGGATGTGGCCGAGCATGGGCTGCCCGGCG
CGCATCATTTCTTCATCACCTTCGACACGACCCATCCCGATGTGGCCATG
GCCGACTGGCTCCGCGCGCGCTATCCGCAGGAGATGACGGTCGTGATCCA
GCATTGGTACGAGAACCTCTCCGCCGACGACCATGGCTTCTCGGTCACGC
TGAACTTCGGCAACCAGCCCGAGCCGCTGGTCATCCCCTTCGATGCCGTG
CGCACCTTCGTCGACCCGTCCGTGGAATTCGGCCTCCGGTTCGAGACCCA
TGAGGAGGACGAGGAGGAGGAGACGGGCGGCGACGAGGATCCCGACGGCG
ACGACGAGCCGCCGCGCCATGACGCGCAGGTCGTGAGCCTCGACAAGTTC
CGCAAG

The amino acid sequence of RhR13 (SEQ ID NO: 10)—

MARSIDYGNLMHRAMRGLIQSVLEDVAEHGLPGAHHFFITFDTTHPDVAM
ADWLRARYPQEMTVVIQHWYENLSADDHGFSVTLNFGNQPEPLVIPFDAV
RTFVDPSVEFGLRFETHEEDEEEETGGDEDPDGDDEPPRHDAQVVSLDKF
RKAAALEHHHHHH

The nucleic acid sequence encoding the protein RR162-1 (SEQ ID NO: 5)—

ATGAGCACGCGGACGAGGACGACGGAAGAACGCCGGCACGAGATTGTGCG
TGTCGCCCGTGCCACCGGCTCGGTCGATGTCACCGCGCTCGCCGCCGAAC
TGGGCGTCGCCAAGGAAACCGTACGTCGTGATCTGCGTGCCCTGGAAGAT
CATGGCCTGGTCCGTCGTACCCATGGCGGCGCCTACCCGGTGGAAAGCGC
CGGTTTCGAAACCACGCTCGCCTTCCGTGCCACCAGCCATGTGCCCGAAA
AGCGTCGTATTGCGTCCGCCGCCGTCGAACTGCTCGGCGATGCGGAAACG
GTCTTCGTCGATGAAGGCTTCACCCCCCAACTCATTGCCGAAGCCCTGCC
CCGTGATCGTCCGCTGACCGTGGTCACCGCGTCCCTGCCGGTGGCGGGCG
CGCTGGCCGAAGCGGGCGATACGTCCGTCCTGCTGCTCGGCGGCCGTGTC
CGTTCGGGCACCCTGGCCACCGTCGATCATTGGACCACGAAGATGCTGGC
CGGCTTCGTCATTGATCTGGCGTACATTGGCGCCAACGGCATTTCCCGTG
AACATGGTCTCACCACACCCGATCCCGCGGTCAGCGAAGTCAAGGCGCAA
GCCGTCCGTGCCGCCCGTCGTACGGTGTTCGCCGGCGCGCATACCAAGTT
CGGGGCGGTGAGCTTCTGCCGTTTCGCGGAAGTCGGCGCCCTGGAAGCCA
TTGTCACCAGCACGCTGCTGCCCTCGGCCGAAGCCCATCGTTACTCCCTC
CTCGGCCCCCAAATTATTCGTGTC

The nucleic acid sequence encoding the protein RR162-2 (SEQ ID NO: 6)—

ATGAGCACGCGGACGAGGACGACGGAAGAACGCCGGCACGAGATCGTGCG
GGTCGCCCGCGCCACCGGCTCGGTCGACGTCACCGCGCTCGCCGCCGAAC
TGGGCGTCGCCAAGGAGACCGTACGACGCGACCTGCGCGCCCTGGAGGAC
CATGGCCTGGTCCGCCGCACCCATGGCGGCGCCTACCCGGTGGAGAGCGC
CGGTTTCGAGACCACGCTCGCCTTCCGCGCCACCAGCCATGTGCCCGAGA
AGCGCCGGATCGCGTCCGCCGCCGTCGAACTGCTCGGCGACGCGGAGACG
GTCTTCGTCGACGAGGGCTTCACCCCCCAGCTCATCGCCGAGGCCCTGCC
CCGGGACCGGCCGCTGACCGTGGTCACCGCGTCCCTGCCGGTGGCGGGCG
CGCTGGCCGAGGCGGGCGACACGTCCGTCCTGCTGCTCGGCGGCCGGGTC
CGCTCGGGCACCCTGGCCACCGTCGACCATTGGACCACGAAGATGCTGGC
CGGCTTCGTCATCGACCTGGCGTACATCGGCGCCAACGGCATCTCCCGGG
AGCATGGTCTCACCACACCCGACCCCGCGGTCAGCGAGGTCAAGGCGCAG
GCCGTCCGGGCCGCCCGCCGCACGGTGTTCGCCGGCGCGCATACCAAGTT
CGGGGCGGTGAGCTTCTGCCGGTTCGCGGAGGTCGGCGCCCTGGAGGCCA
TCGTCACCAGCACGCTGCTGCCCTCGGCCGAGGCCCATCGCTACTCCCTC
CTCGGCCCCCAGATCATCCGCGTC

The amino acid sequence of RR162 (SEQ ID NO: 11)—

MSTRTRTTEERRHEIVRVARATGSVDVTALAAELGVAKETVRRDLRALED
HGLVRRTHGGAYPVESAGFETTLAFRATSHVPEKRRIASAAVELLGDAET
VFVDEGFTPQLIAEALPRDRPLTVVTASLPVAGALAEAGDTSVLLLGGRV
RSGTLATVDHWTTKMLAGFVIDLAYIGANGISREHGLTTPDPAVSEVKAQ
AVRAARRTVFAGAHTKFGAVSFCRFAEVGALEAIVTSTLLPSAEAHRYSL
LGPQIIRVLEHHHHHH

The nucleic acid sequence encoding the protein XR92-1 (SEQ ID NO: 7)—

ATGAAGACAATTCAGGAGCAGCAGATGAAGATAGTTAGGAATATGCGTCG
TATTCGTTACAAGATTGCTGTTATTAGCACGAAAGGAGGTGTGGGGAAAA
GCTTTGTTACCGCTAGCCTCGCGGCAGCCCTCGCTGCGGAAGGGCGTCGT
GTTGGAGTTTTTGATGCAGATATTAGCGGTCCTAGCGTTCATAAAATGCT
CGGCCTCCAAACGGGCATGGGTATGCCCTCGCAACTCGATGGCACTGTAA
AGCCCGTGGAAGTTCCTCCGGGAATTAAAGTAGCTAGCATTGGGCTGTTG
CTGCCCATGGATGAAGTGCCCCTAATTTGGCGTGGGGCCATTAAGACGAG
TGCCATTCGTGAACTGCTTGCATACGTCGATTGGGGAGAACTCGATTATC
TCCTCATTGATCTACCTCCGGGAACAGGTGATGAAGTCCTCACGATTACC
CAAATTATTCCCAACATTACGGGCTTCCTGGTAGTCACGATTCCCAGCGA
AATTGCTAAGTCTGTCGTTAAGAAGGCTGTCAGCTTTGCCAAGCGTATTG
AAGCCCCTGTGATTGGAATTGTCGAAAACATGAGCTACTTTCGTTGTAGC
GATGGATCCATTCATTATATTTTCGGCCGTGGCGCGGCTGAAGAAATTGC
GTCACAATATGGTATTGAACTCCTCGGCAAAATTCCCATTGATCCTGCGA
TTCGTGAATCGAACGATAAAGGCAAAATTTTCTTCCTAGAAAATCCAGAA
AGCGAAGCTTCGCGTGAATTCCTTAAGATTGCCCGTCGTATTATTGAAAT
TGTTGAAAAGCTAGGCCCAAAGCCTCCTGCGTGGGGTCCCCAAATGGAA

The nucleic acid sequence encoding the protein XR92-2 (SEQ ID NO: 8)—

ATGAAGACAATTCAGGAGCAGCAGATGAAGATAGTTAGGAATATGAGGAG
GATTAGGTACAAGATTGCTGTTATTAGCACGAAAGGAGGTGTGGGGAAAA
GCTTTGTTACCGCTAGCCTCGCGGCAGCCCTCGCTGCGGAGGGGCGAAGG
GTTGGAGTTTTTGACGCAGATATTAGCGGTCCTAGCGTTCATAAAATGCT
CGGCCTCCAGACGGGCATGGGTATGCCCTCGCAGCTCGACGGCACTGTAA
AGCCCGTGGAAGTTCCTCCGGGAATTAAAGTAGCTAGCATTGGGCTGTTG
CTGCCCATGGATGAGGTGCCCCTAATTTGGAGAGGGGCCATTAAGACGAG
TGCCATTAGAGAGCTGCTTGCATACGTCGACTGGGGAGAACTCGACTATC
TCCTCATTGACCTACCTCCGGGAACAGGTGATGAGGTCCTCACGATTACC
CAGATTATTCCCAACATTACGGGCTTCCTGGTAGTCACGATTCCCAGCGA
GATTGCTAAGTCTGTCGTTAAGAAGGCTGTCAGCTTTGCCAAGAGGATTG
AAGCCCCTGTGATTGGAATTGTCGAGAACATGAGCTACTTTAGGTGTAGC
GACGGATCCATTCACTATATTTTCGGCCGCGGCGCGGCTGAGGAGATTGC
GTCACAGTATGGTATTGAACTCCTCGGCAAAATTCCCATTGACCCTGCGA
TTAGAGAGTCGAACGATAAAGGCAAAATTTTCTTCCTAGAGAATCCAGAG
AGCGAAGCTTCGAGAGAGTTCCTTAAGATTGCCCGCAGGATTATTGAGAT
TGTTGAGAAGCTAGGCCCAAAGCCTCCTGCGTGGGGTCCCCAGATGGAG

The amino acid sequence of XR92 (SEQ ID NO: 12)—

MKTIQEQQMKIVRNMRRIRYKIAVISTKGGVGKSFVTASLAAALAAEGRR
VGVFDADISGPSVHKMLGLQTGMGMPSQLDGTVKPVEVPPGIKVASIGLL
LPMDEVPLIWRGAIKTSAIRELLAYVDWGELDYLLIDLPPGTGDEVLTIT
QIIPNITGFLVVTIPSEIAKSVVKKAVSFAKRIEAPVIGIVENMSYFRCS
DGSIHYIFGRGAAEEIASQYGIELLGKIPIDPAIRESNDKGKIFFLENPE
SEASREFLKIARRIIEIVEKLGPKPPAWGPQMELEHHHHHH

### Example 4

### Codon Mutation Targets

TABLE 13
Targets

| | | | | | Gene 1 | Gene 2 |
|---|---|---|---|---|---|---|
| | | | | Original | (All | (relevant |
| ID | EXP | SOL | Length | Sequence | Changed) | codon only) |
| HIS | | | | | | |
| RHR13 | 2 | 3 | 152 | ATGGCGCGTTCGA | ATGGCGCGTTCGA | ATGGCGCGTTCGAT |
| | | | | TCGATTACGGCAAC | TCGATTACGGCAA | CGATTACGGCAACC |
| | | | | CTCATGCACCGCG | CCTCATGCACCGC | TCATGCACCGCGC |
| | | | | CGATGCGGGGCCT | GCGATGCGTGGCC | GATGCGGGGCCTG |
| | | | | GATCCAGAGCGTG | TGATTCAAAGCGT | ATCCAGAGCGTGCT |
| | | | | CTCGAGGATGTGG | GCTCGAAGATGTG | CGAGGATGTGGCC |
| | | | | CCGAGCACGGGCT | GCCGAACATGGGC | GAGCATGGGCTGC |
| | | | | GCCCGGCGCGCAC | TGCCCGGCGCGCA | CCGGCGCGCATCA |
| | | | | CATTTCTTCATCAC | TCATTTCTTCATTA | TTTCTTCATCACCTT |
| | | | | CTTCGACACGACC | CCTTCGATACGAC | CGACACGACCCATC |
| | | | | CATCCCGATGTGG | CCATCCCGATGTG | CCGATGTGGCCAT |
| | | | | CCATGGCCGACTG | GCCATGGCCGATT | GGCCGACTGGCTC |
| | | | | GCTCCGCGCGCGC | GGCTCCGTGCGCG | CGCGCGCGCTATC |
| | | | | TATCCGCAGGAGAT | TTATCCGCAAGAAA | CGCAGGAGATGAC |
| | | | | GACGGTCGTGATC | TGACGGTCGTGAT | GGTCGTGATCCAG |
| | | | | CAGCACTGGTACG | TCAACATTGGTAC | CATTGGTACGAGAA |
| | | | | AGAACCTCTCCGC | GAAAACCTCTCCG | CCTCTCCGCCGAC |
| | | | | CGACGACCACGGC | CCGATGATCATGG | GACCATGGCTTCTC |
| | | | | TTCTCGGTCACGCT | CTTCTCGGTCACG | GGTCACGCTGAACT |
| | | | | GAACTTCGGCAAC | CTGAACTTCGGCA | TCGGCAACCAGCC |
| | | | | CAGCCCGAGCCGC | ACCAACCCGAACC | CGAGCCGCTGGTC |
| | | | | TGGTCATCCCCTTC | GCTGGTCATTCCC | ATCCCCTTCGATGC |
| | | | | GATGCCGTGCGCA | TTCGATGCCGTGC | CGTGCGCACCTTC |
| | | | | CCTTCGTCGACCC | GTACCTTCGTCGA | GTCGACCCGTCCG |
| | | | | GTCCGTGGAATTC | TCCGTCCGTGGAA | TGGAATTCGGCCTC |
| | | | | GGCCTCCGGTTCG | TTCGGCCTCCGTT | CGGTTCGAGACCC |
| | | | | AGACCCACGAGGA | TCGAAACCCATGA | ATGAGGAGGACGA |
| | | | | GGACGAGGAGGAG | AGAAGATGAAGAA | GGAGGAGGAGACG |
| | | | | GAGACGGGCGGCG | GAAGAAACGGGCG | GGCGGCGACGAGG |
| | | | | ACGAGGATCCCGA | GCGATGAAGATCC | ATCCCGACGGCGA |
| | | | | CGGCGACGACGAG | CGATGGCGATGAT | CGACGAGCCGCCG |
| | | | | CCGCCGCGCCACG | GAACCGCCGCGTC | CGCCATGACGCGC |
| | | | | ACGCGCAGGTCGT | ATGATGCGCAAGT | AGGTCGTGAGCCT |
| | | | | GAGCCTCGACAAG | CGTGAGCCTCGAT | CGACAAGTTCCGCA |
| | | | | TTCCGCAAGTAG | AAGTTCCGTAAGTA | AGTAG |
| | | | | (SEQ ID NO: 13) | G | (SEQ ID NO: 15) |
| | | | | | (SEQ ID NO: 14) | |
| RR162 | 2 | 2 | 258 | ATGAGCACGCGGA | ATGAGCACGCGGA | ATGAGCACGCGGA |
| | | | | CGAGGACGACGGA | CGAGGACGACGGA | CGAGGACGACGGA |
| | | | | AGAACGCCGGCAC | AGAACGCCGGCAC | AGAACGCCGGCAC |
| | | | | GAGATCGTGCGGG | GAGATTGTGCGTG | GAGATCGTGCGGG |
| | | | | TCGCCCGCGCCAC | TCGCCCGTGCCAC | TCGCCCGCGCCAC |
| | | | | CGGCTCGGTCGAC | CGGCTCGGTCGAT | CGGCTCGGTCGAC |
| | | | | GTCACCGCGCTCG | GTCACCGCGCTCG | GTCACCGCGCTCG |
| | | | | CCGCCGAACTGGG | CCGCCGAACTGGG | CCGCCGAACTGGG |
| | | | | CGTCGCCAAGGAG | CGTCGCCAAGGAA | CGTCGCCAAGGAG |
| | | | | ACCGTACGACGCG | ACCGTACGTCGTG | ACCGTACGACGCG |
| | | | | ACCTGCGCGCCCT | ATCTGCGTGCCCT | ACCTGCGCGCCCT |
| | | | | GGAGGACCACGGC | GGAAGATCATGGC | GGAGGACCATGGC |
| | | | | CTGGTCCGCCGCA | CTGGTCCGTCGTA | CTGGTCCGCCGCA |
| | | | | CCCACGGCGGCGC | CCCATGGCGGCGC | CCCATGGCGGCGC |
| | | | | CTACCCGGTGGAG | CTACCCGGTGGAA | CTACCCGGTGGAG |
| | | | | AGCGCCGGTTTCG | AGCGCCGGTTTCG | AGCGCCGGTTTCG |
| | | | | AGACCACGCTCGC | AAACCACGCTCGC | AGACCACGCTCGC |
| | | | | CTTCCGCGCCACC | CTTCCGTGCCACC | CTTCCGCGCCACCA |
| | | | | AGCCACGTGCCCG | AGCCATGTGCCCG | GCCATGTGCCCGA |
| | | | | AGAAGCGCCGGAT | AAAAGCGTCGTATT | GAAGCGCCGGATC |
| | | | | CGCGTCCGCCGCC | GCGTCCGCCGCCG | GCGTCCGCCGCCG |
| | | | | GTCGAACTGCTCG | TCGAACTGCTCGG | TCGAACTGCTCGGC |
| | | | | GCGACGCGGAGAC | CGATGCGGAAACG | GACGCGGAGACGG |
| | | | | GGTCTTCGTCGAC | GTCTTCGTCGATG | TCTTCGTCGACGAG |
| | | | | GAGGGCTTCACCC | AAGGCTTCACCCC | GGCTTCACCCCCCA |
| | | | | CCCAGCTCATCGC | CCAACTCATTGCC | GCTCATCGCCGAG |
| | | | | CGAGGCCCTGCCC | GAAGCCCTGCCCC | GCCCTGCCCCGGG |
| | | | | CGGGACCGGCCGC | GTGATCGTCCGCT | ACCGGCCGCTGAC |
| | | | | TGACCGTGGTCAC | GACCGTGGTCACC | CGTGGTCACCGCG |
| | | | | CGCGTCCCTGCCG | GCGTCCCTGCCGG | TCCCTGCCGGTGG |
| | | | | GTGGCGGGCGCGC | TGGCGGGCGCGCT | CGGGCGCGCTGGC |
| | | | | TGGCCGAGGCGGG | GGCCGAAGCGGG | CGAGGCGGGCGAC |
| | | | | CGACACGTCCGTC | CGATACGTCCGTC | ACGTCCGTCCTGCT |
| | | | | CTGCTGCTCGGCG | CTGCTGCTCGGCG | GCTCGGCGGCCGG |
| | | | | GCCGGGTCCGCTC | GCCGTGTCCGTTC | GTCCGCTCGGGCA |
| | | | | GGGCACCCTGGCC | GGGCACCCTGGCC | CCCTGGCCACCGT |
| | | | | ACCGTCGACCACT | ACCGTCGATCATT | CGACCATTGGACCA |
| | | | | GGACCACGAAGAT | GGACCACGAAGAT | CGAAGATGCTGGC |
| | | | | GCTGGCCGGCTTC | GCTGGCCGGCTTC | CGGCTTCGTCATCG |
| | | | | GTCATCGACCTGG | GTCATTGATCTGG | ACCTGGCGTACATC |
| | | | | CGTACATCGGCGC | CGTACATTGGCGC | GGCGCCAACGGCA |
| | | | | CAACGGCATCTCC | CAACGGCATTTCC | TCTCCCGGGAGCAT |
| | | | | CGGGAGCACGGTC | CGTGAACATGGTC | GGTCTCACCACACC |
| | | | | TCACCACACCCGA | TCACCACACCCGA | CGACCCCGCGGTC |
| | | | | CCCCGCGGTCAGC | TCCCGCGGTCAGC | AGCGAGGTCAAGG |
| | | | | GAGGTCAAGGCGC | GAAGTCAAGGCGC | CGCAGGCCGTCCG |
| | | | | AGGCCGTCCGGGC | AAGCCGTCCGTGC | GGCCGCCCGCCGC |
| | | | | CGCCCGCCGCACG | CGCCCGTCGTACG | ACGGTGTTCGCCG |
| | | | | GTGTTCGCCGGCG | GTGTTCGCCGGCG | GCGCGCATACCAA |
| | | | | CGCACACCAAGTTC | CGCATACCAAGTT | GTTCGGGGCGGTG |
| | | | | GGGGCGGTGAGCT | CGGGGCGGTGAG | AGCTTCTGCCGGTT |
| | | | | TCTGCCGGTTCGC | CTTCTGCCGTTTC | CGCGGAGGTCGGC |
| | | | | GGAGGTCGGCGCC | GCGGAAGTCGGCG | GCCCTGGAGGCCA |
| | | | | CTGGAGGCCATCG | CCCTGGAAGCCAT | TCGTCACCAGCACG |
| | | | | TCACCAGCACGCT | TGTCACCAGCACG | CTGCTGCCCTCGG |
| | | | | GCTGCCCTCGGCC | CTGCTGCCCTCGG | CCGAGGCCCATCG |
| | | | | GAGGCCCACCGCT | CCGAAGCCCATCG | CTACTCCCTCCTCG |
| | | | | ACTCCCTCCTCGG | TTACTCCCTCCTCG | GCCCCCAGATCATC |
| | | | | CCCCCAGATCATCC | GCCCCCAAATTATT | CGCGTCTGA |
| | | | | GCGTCTGA | CGTGTCTGA | (SEQ ID NO: 18) |
| | | | | (SEQ ID NO: 16) | (SEQ ID NO: 17) | |
| SHR52 | 4 | 4 | 213 | ATGGATGTAACACG | ATGGATGTAACAC | ATGGATGTAACACG |
| | | | | ACAAATAGAATTAG | GACAAATAGAATTA | ACAAATAGAATTAG |
| | | | | CGCATCGATATATG | GCGCATCGATATA | CGCATCGATATATG |
| | | | | AAAGATTTTCATAA | TGAAAGACTTTCAC | AAAGATTTTCACAA |
| | | | | AAGTGATTATTCTG | AAAAGTGACTATTC | AAGTGATTATTCTG |
| | | | | GTCATGATGTTGCA | TGGTCACGACGTT | GTCACGATGTTGCA |
| | | | | CATGTAGAACGTGT | GCACACGTAGAGC | CACGTAGAACGTGT |
| | | | | AACGTCACTAGCTC | GCGTAACGTCACT | AACGTCACTAGCTC |
| | | | | AAACAATCTCTAAA | AGCTCAGACAATC | AAACAATCTCTAAA |
| | | | | TGCGAGCAACAAG | TCTAAATGCGAGC | TGCGAGCAACAAG |
| | | | | GAGAATATTTAATT | AGCAGGGAGAGTA | GAGAATATTTAATTA |
| | | | | ATCACATTATCTGC | TTTAATCATCACAT | TCACATTATCTGCA |
| | | | | ATTACTTCATGATG | TATCTGCATTACTT | TTACTTCACGATGT |
| | | | | TCATTGATGATAAG | CACGACGTCATCG | CATTGATGATAAGT |
| | | | | TTAACAAATAAAGC | ACGACAAGTTAAC | TAACAAATAAAGCC |
| | | | | CAATGCTTTAGATC | AAATAAAGCCAATG | AATGCTTTAGATCG |
| | | | | GTTTAAAAACATTTT | CTTTAGACCGCTTA | TTTAAAAACATTTTT |
| | | | | TAAAGAACATTCGC | AAAACATTTTTAAA | AAAGAACATTCGCG |
| | | | | GTATCTTCTGATCA | GAACATCCGCGTA | TATCTTCTGATCAA |
| | | | | ACAACAAAAGATTA | TCTTCTGACCAGC | CAACAAAAGATTAT |
| | | | | TTTACATCATTCAA | AGCAGAAGATCAT | TTACATCATTCAAC |
| | | | | CATTTAAGTTATAG | CTACATCATCCAG | ACTTAAGTTATAGA |
| | | | | AAATGGACAAAATA | CACTTAAGTTATAG | AATGGACAAAATAA |
| | | | | ATCATGTAGACCTT | AAATGGACAGAAT | TCACGTAGACCTTC |
| | | | | CCAATTGAAGGACA | AATCACGTAGACC | CAATTGAAGGACAA |
| | | | | AATTGTTAGAGATG | TTCCAATCGAGGG | ATTGTTAGAGATGC |
| | | | | CAGATCGACTAGAT | ACAGATCGTTAGA | AGATCGACTAGATG |
| | | | | GCGATTGGTGCTAT | GACGCAGACCGAC | CGATTGGTGCTATT |
| | | | | TGGTATTGCTAGAG | TAGACGCGATCGG | GGTATTGCTAGAGC |
| | | | | CATTTCAATTTTCA | TGCTATCGGTATC | ATTTCAATTTTCAG |
| | | | | GGCCATTTTAATGA | GCTAGAGCATTTC | GCCACTTTAATGAG |
| | | | | GCCAATGTGGACA | AGTTTTCAGGCCA | CCAATGTGGACAGA |
| | | | | GAATCACCACATAG | CTTTAATGAGCCAA | ATCACCACACAGTG |
| | | | | TGACATACCTAATA | TGTGGACAGAGTC | ACATACCTAATATT |
| | | | | TTGAAACGATTACT | ACCACACAGTGAC | GAAACGATTACTAA |
| | | | | AATTTAGAACCTTC | ATACCTAATATCGA | TTTAGAACCTTCCG |
| | | | | CGCTATACGTCACT | GACGATCACTAATT | CTATACGTCACTTT |
| | | | | TTTATGATAAATTAT | TAGAGCCTTCCGC | TATGATAAATTATTA |
| | | | | TAAAATTAAAAGAT | TATACGCCACTTTT | AAATTAAAAGATTTA |
| | | | | TTAATGCATACTGA | ATGACAAATTATTA | ATGCACACTGAAAC |
| | | | | AACTGGTCGAAAAT | AAATTAAAAGACTT | TGGTCGAAAATTAG |
| | | | | TAGCTAGAGAAAGA | AATGCACACTGAG | CTAGAGAAAGACAC |
| | | | | CATGCGTTTATGGA | ACTGGTCGAAAATT | GCGTTTATGGAACA |
| | | | | ACAGTTTTTAAATC | AGCTAGAGAGAGA | GTTTTTAAATCAATT |
| | | | | AATTTTATAAAGAAT | CACGCGTTTATGG | TTATAAAGAATGGC |
| | | | | GGCATATATAA | AGCAGTTTTTAAAT | ACATATAA |
| | | | | (SEQ ID NO: 19) | CAGTTTTATAAAGA | (SEQ ID NO: 21) |
| | | | | | GTGGCACATATAA | |
| | | | | | (SEQ ID NO: 20) | |
| SYR92 | 4 | 4 | 218 | | ATGAAACTCATTCA | |
| | | | | | AATGTCAGACCATA | |
| | | | | ATGAAACTCATTCA | TTTATAAATTAAAT | ATGAAACTCATTCA |
| | | | | AATGTCAGACCATA | ATACAGACAACAG | AATGTCAGACCATA |
| | | | | TTTATAAATTAAATA | TTGGTATCCCGATA | TTTATAAATTAAATA |
| | | | | TACAGACAACAGTT | CAGATAAACACTTG | TACAGACAACAGTT |
| | | | | GGTATCCCGATACA | GTTTATCGTGAATG | GGTATCCCGATACA |
| | | | | AATAAACACTTGGT | ACAACGACGTTTAT | AATAAACACTTGGT |
| | | | | TTATTGTGAATGAT | ATCATAGACACAG | TTATTGTGAATGAT |
| | | | | AACGACGTTTATAT | GTATGGACGACTA | AACGACGTTTATAT |
| | | | | CATAGACACAGGTA | TGCTGAGCTACAG | CATAGACACAGGTA |
| | | | | TGGATGATTATGCT | ATCACGATCGCTA | TGGATGATTATGCT |
| | | | | GAGCTACAAATCAC | AATCGCTCGGTAA | GAGCTACAAATCAC |
| | | | | GATTGCTAAATCGC | TCCTAAAGGCATCT | GATTGCTAAATCGC |
| | | | | TCGGTAATCCTAAA | TTTTAACGCACGG | TCGGTAATCCTAAA |
| | | | | GGCATTTTTTTAAC | ACACCTAGACCAC | GGCATTTTTTTAAC |
| | | | | GCATGGACATCTAG | ATCAATGGCGCAA | GCACGGACACCTA |
| | | | | ATCATATCAATGGC | AACGCATCTCTGA | GATCACATCAATGG |
| | | | | GCAAAACGTATTTC | GGCTTTGAAAATAC | CGCAAAACGTATTT |
| | | | | TGAAGCTTTGAAAA | CTATCTTTACATAT | CTGAAGCTTTGAAA |
| | | | | TACCTATCTTTACA | AAAAATGAGCTCC | ATACCTATCTTTACA |
| | | | | TATAAAAATGAACT | CTTATATCAATGGT | TATAAAAATGAACT |
| | | | | CCCTTATATCAATG | GAGCTGCCTTATC | CCCTTATATCAATG |
| | | | | GTGAGCTGCCTTAT | CAAATAAAACGCA | GTGAGCTGCCTTAT |
| | | | | CCAAATAAAACGCA | CACCGAGAATACA | CCAAATAAAACGCA |
| | | | | TACCGAAAATACAG | GGTGTTCAGTACA | CACCGAAAATACAG |
| | | | | GTGTTCAATACATT | TCGTTAAACCTCTA | GTGTTCAATACATT |
| | | | | GTTAAACCTCTAGA | GAGACTAATACAAA | GTTAAACCTCTAGA |
| | | | | AACTAATACAAATC | TCTGCCCTTCAATT | AACTAATACAAATC |
| | | | | TGCCCTTCAATTAT | ATTACTTAACTCCT | TGCCCTTCAATTAT |
| | | | | TACTTAACTCCTGG | GGTCACGCACCAG | TACTTAACTCCTGG |
| | | | | TCATGCACCAGGTC | GTCACGTCATCTAT | TCACGCACCAGGTC |
| | | | | ATGTCATCTATTTT | TTTCACAATCAGGA | ACGTCATCTATTTT |
| | | | | CATAATCAAGATAA | CAAAATCTTAATAT | CACAATCAAGATAA |
| | | | | AATTTTAATATGCG | GCGGAGACTTATT | AATTTTAATATGCG |
| | | | | GAGATTTATTTATTT | TATCTCAGACGCG | GAGATTTATTTATTT |
| | | | | CAGATGCGCAACAT | CAGCACCTGCACA | CAGATGCGCAACAC |
| | | | | CTGCATATTCCTAT | TCCCTATCAAAAAA | CTGCACATTCCTAT |
| | | | | CAAAAAATTCACTT | TTCACTTATAACAT | CAAAAAATTCACTT |
| | | | | ATAACATGACTGAA | GACTGAGAATATC | ATAACATGACTGAA |
| | | | | AATATCAAAAGCGG | AAAAGCGGTCAGA | AATATCAAAAGCGG |
| | | | | TCAAATCATAGATA | TCATAGACAATCTT | TCAAATCATAGATA |
| | | | | ATCTTTGTCCCAAA | TGTCCCAAATTAAT | ATCTTTGTCCCAAA |
| | | | | TTAATTACAACTTC | CACAACTTCACAC | TTAATTACAACTTCA |
| | | | | ACATGGCGATGATC | GGCGACGACCTAT | CACGGCGATGATCT |
| | | | | TATATTATTCAGAT | ATTATTCAGACGAC | ATATTATTCAGATG |
| | | | | GACATTTATTCAAT | ATCTATTCAATCTA | ACATTTATTCAATTT |
| | | | | TTATAAATTTAAGTA | TAAATTTAAGTACG | ATAAATTTAAGTAC |
| | | | | CGAGGAGTAA | AGGAGTAA | GAGGAGTAA |
| | | | | (SEQ ID NO: 22) | (SEQ ID NO: 23) | (SEQ ID NO: 24) |
| GLU | | | | | | |
| XR47 | 1 | 2 | 266 | GTGAGGCGGAGGG | GTGAGGCGGAGG | GTGAGGCGGAGGG |
| | | | | CTAGATGGCTGAG | GCTAGATGGCTGA | CTAGATGGCTGAG |
| | | | | GAGGGAGAGGGAG | GGAGGGAGAGGG | GAGGGAGAGGGAG |
| | | | | GAGGAAGAGAGGG | AGGAGGAAGAACG | GAGGAAGAAAGGG |
| | | | | TTAAGGACCGGGA | TGTTAAGGATCGT | TTAAGGACCGGGA |
| | | | | CATGTTTAAGATTG | GATATGTTTAAGAT | CATGTTTAAGATTG |
| | | | | TGGACGAGGTTTTC | TGTGGATGAAGTTT | TGGACGAAGTTTTC |
| | | | | GACTCCATAACCCT | TCGATTCCATTACC | GACTCCATAACCCT |
| | | | | CTCCCACCTCTACA | CTCTCCCATCTCTA | CTCCCACCTCTACA |
| | | | | GGCTCTACTCGCG | CCGTCTCTACTCG | GGCTCTACTCGCG |
| | | | | CAAGGTCCTCAGG | CGTAAGGTCCTCC | CAAGGTCCTCAGG |
| | | | | GAGCTCAAGGGCT | GTGAACTCAAGGG | GAACTCAAGGGCTC |
| | | | | CTATAAGCAGCGGT | CTCTATTAGCAGC | TATAAGCAGCGGTA |
| | | | | AAGGAGTCTAAGGT | GGTAAGGAATCTA | AGGAATCTAAGGTC |
| | | | | CTACTGGGGCGTC | AGGTCTACTGGGG | TACTGGGGCGTCG |
| | | | | GCGTGGGATAGGA | CGTCGCGTGGGAT | CGTGGGATAGGAG |
| | | | | GCGACGTCGCCGT | CGTAGCGATGTCG | CGACGTCGCCGTTA |
| | | | | TAAGATATACCTCT | CCGTTAAGATTTAC | AGATATACCTCTCG |
| | | | | CGTTCACTTCCGAC | CTCTCGTTCACTTC | TTCACTTCCGACTT |
| | | | | TTCAGGAAGAGCAT | CGATTTCCGTAAG | CAGGAAGAGCATTA |
| | | | | TAGAAAATATATTG | AGCATTCGTAAATA | GAAAATATATTGTC |
| | | | | TCGGGGACCCCAG | TATTGTCGGGGAT | GGGGACCCCAGGT |
| | | | | GTTCGAGGACATC | CCCCGTTTCGAAG | TCGAAGACATCCCC |
| | | | | CCCGCAGGCAACA | ATATTCCCGCAGG | GCAGGCAACATAAG |
| | | | | TAAGGAGGCTGATA | CAACATTCGTCGT | GAGGCTGATATACG |
| | | | | TACGAGTGGGCTA | CTGATTTACGAATG | AATGGGCTAGGAAA |
| | | | | GGAAAGAGTACAG | GGCTCGTAAAGAA | GAATACAGGAACCT |
| | | | | GAACCTCAGGAGG | TACCGTAACCTCC | CAGGAGGATGCGC |
| | | | | ATGCGCGAGTCGG | GTCGTATGCGTGA | GAATCGGGGGTCA |
| | | | | GGGTCAGGGTTCC | ATCGGGGGTCCGT | GGGTTCCCAGGCC |
| | | | | CAGGCCCGTGGCC | GTTCCCCGTCCCG | CGTGGCCGTCGAA |
| | | | | GTCGAGGCAAACA | TGGCCGTCGAAGC | GCAAACATTATAGT |
| | | | | TTATAGTTATGGAG | AAACATTATTGTTA | TATGGAATTCCTGG |
| | | | | TTCCTGGGCGAGA | TGGAATTCCTGGG | GCGAAAAGGGGTA |
| | | | | AGGGGTACAGGGC | CGAAAAGGGGTAC | CAGGGCCCCTACC |
| | | | | CCCTACCCTGGCT | CGTGCCCCTACCC | CTGGCTGAAGCTGT |
| | | | | GAGGCTGTCGAGG | TGGCTGAAGCTGT | CGAAGAACTTGATA |
| | | | | AGCTTGATAGGGG | CGAAGAACTTGAT | GGGGGGAAGCGGA |
| | | | | GGAGGCGGAGGCT | CGTGGGGAAGCG | AGCTATAGCGGCC |
| | | | | ATAGCGGCCGAGG | GAAGCTATTGCGG | GAAGTCCTCCGCCA |
| | | | | TCCTCCGCCAGGC | CCGAAGTCCTCCG | GGCGGAAGCTATA |
| | | | | GGAGGCTATAGTAT | TCAAGCGGAAGCT | GTATGTAGGGCCA |
| | | | | GTAGGGCCAGGCT | ATTGTATGTCGTGC | GGCTCGTGCACGC |
| | | | | CGTGCACGCCGAC | CCGTCTCGTGCAT | CGACCTCAGCGAAT |
| | | | | CTCAGCGAGTACAA | GCCGATCTCAGCG | ACAACATACTAGTC |
| | | | | CATACTAGTCTGGA | AATACAACATTCTA | TGGAGGGGGGAAC |
| | | | | GGGGGGAGCCCTG | GTCTGGCGTGGGG | CCTGGATAATAGAC |
| | | | | GATAATAGACGTCT | AACCCTGGATTATT | GTCTCCCAGGCGG |
| | | | | CCCAGGCGGTGCC | GATGTCTCCCAAG | TGCCCCACAGCCA |
| | | | | CCACAGCCACCCG | CGGTGCCCCATAG | CCCGAACGCTGAA |
| | | | | AACGCTGAGGAGT | CCATCCGAACGCT | GAATTTCTAGAAAG |
| | | | | TTCTAGAGAGGGA | GAAGAATTTCTAGA | GGACGTGGAAAAC |
| | | | | CGTGGAGAACCTC | ACGTGATGTGGAA | CTCCACAGGTTCTT |
| | | | | CACAGGTTCTTGAC | AACCTCCATCGTTT | GACAGGTAAGATG |
| | | | | AGGTAAGATGGGG | CTTGACAGGTAAG | GGGTTCGAATTCGA |
| | | | | TTCGAGTTCGACTT | ATGGGGTTCGAAT | CTTTGACGCTTATC |
| | | | | TGACGCTTATCTCT | TCGATTTTGATGCT | TCTCTAGGCTAAAA |
| | | | | CTAGGCTAAAAAGC | TATCTCTCTCGTCT | AGCTGTATCCACCG |
| | | | | TGTATCCACCGGG | AAAAAGCTGTATTC | GGGTGCTAGGGGT |
| | | | | GTGCTAGGGGTTG | ATCGTGGTGCTCG | TGA |
| | | | | A | TGGTTGA | (SEQ ID NO: 27) |
| | | | | (SEQ ID NO: 25) | (SEQ ID NO: 26) | |
| SRR141 | 2 | 2 | 209 | ATGGCCGCCATGC | ATGGCCGCCATGC | |
| | | | | CCAAGCCCGCTGC | CCAAGCCCGCTGC | ATGGCCGCCATGC |
| | | | | GTTCTGGAACGAC | GTTCTGGAACGAC | CCAAGCCCGCTGC |
| | | | | CGCTTTGCCAACGA | CGCTTTGCCAACG | GTTCTGGAACGACC |
| | | | | GGAGTACGTGTAC | AAGAATACGTGTA | GCTTTGCCAACGAA |
| | | | | GGCGAGGCCCCCA | CGGCGAAGCCCCC | GAATACGTGTACGG |
| | | | | ACCGCTTCGTCGC | AACCGTTTCGTCG | CGAAGCCCCCAAC |
| | | | | GAGCGCCGCCCGG | CGAGCGCCGCCC | CGCTTCGTCGCGA |
| | | | | ACGTGGCTGCCGG | GTACGTGGCTGCC | GCGCCGCCCGGAC |
| | | | | AGGCCGGTGAGGT | GGAAGCCGGTGAA | GTGGCTGCCGGAA |
| | | | | TCTCCTGCTCGGG | GTTCTCCTGCTCG | GCCGGTGAAGTTCT |
| | | | | GCGGGCGAGGGG | GGGCGGGCGAAG | CCTGCTCGGGGCG |
| | | | | CGCAACGCCGTGC | GGCGTAACGCCGT | GGCGAAGGGCGCA |
| | | | | ACCTGGCCCGGGA | GCATCTGGCCCGT | ACGCCGTGCACCT |
| | | | | GGGCCATACGGTC | GAAGGCCATACGG | GGCCCGGGAAGGC |
| | | | | ACCGCGGTCGACT | TCACCGCGGTCGA | CATACGGTCACCGC |
| | | | | ACGCCGTGGAGGG | TTACGCCGTGGAA | GGTCGACTACGCC |
| | | | | GCTCCGCAAGACG | GGGCTCCGTAAGA | GTGGAAGGGCTCC |
| | | | | GAACGCCTCGCGA | CGGAACGTCTCGC | GCAAGACGGAACG |
| | | | | CGGAGGCCGGGGT | GACGGAAGCCGG | CCTCGCGACGGAA |
| | | | | GGAGGTCGAGGCG | GGTGGAAGTCGAA | GCCGGGGTGGAAG |
| | | | | ATCCAGGCCGATG | GCGATTCAAGCCG | TCGAAGCGATCCAG |
| | | | | TGCGCGAGTGGAA | ATGTGCGTGAATG | GCCGATGTGCGCG |
| | | | | GCCCGCCCGGGCG | GAAGCCCGCCCGT | AATGGAAGCCCGC |
| | | | | TGGGACGCGGTCG | GCGTGGGATGCGG | CCGGGCGTGGGAC |
| | | | | TCGTCACGTTTCTC | TCGTCGTCACGTTT | GCGGTCGTCGTCA |
| | | | | CACCTTCCCGCCG | CTCCATCTTCCCG | CGTTTCTCCACCTT |
| | | | | ACGAGCGACCGGG | CCGATGAACGTCC | CCCGCCGACGAAC |
| | | | | CCTGTACCGCCTC | GGGCCTGTACCGT | GACCGGGCCTGTA |
| | | | | GTTCAGCGCTGTTT | CTCGTTCAACGTT | CCGCCTCGTTCAGC |
| | | | | GCGGCCCGGGGG | GTTTGCGTCCCGG | GCTGTTTGCGGCC |
| | | | | GCGCCTCGTGGCG | GGGGCGTCTCGTG | CGGGGGGCGCCTC |
| | | | | GAGTGGTTTCGCC | GCGGAATGGTTTC | GTGGCGGAATGGT |
| | | | | CGGAGCAGCGCAC | GTCCGGAACAACG | TTCGCCCGGAACA |
| | | | | GGACGGCTACACG | TACGGATGGCTAC | GCGCACGGACGGC |
| | | | | AGCGGCGGCCCGC | ACGAGCGGCGGC | TACACGAGCGGCG |
| | | | | CCGATCCTGCCAT | CCGCCCGATCCTG | GCCCGCCCGATCC |
| | | | | GATGGTCACCGCC | CCATGATGGTCAC | TGCCATGATGGTCA |
| | | | | GACGAGCTCCGCG | CGCCGATGAACTC | CCGCCGACGAACT |
| | | | | GGCACTTCGCCGA | CGTGGGCATTTCG | CCGCGGGCACTTC |
| | | | | GGCGGGCATCGAC | CCGAAGCGGGCAT | GCCGAAGCGGGCA |
| | | | | CATCTCGAAGCGG | TGATCATCTCGAA | TCGACCATCTCGAA |
| | | | | CCGAGCCGACCCT | GCGGCCGAACCGA | GCGGCCGAACCGA |
| | | | | CGACGAGGGCATG | CCCTCGATGAAGG | CCCTCGACGAAGG |
| | | | | CACCGGGGCCCCG | CATGCATCGTGGC | CATGCACCGGGGC |
| | | | | CGGCGACGGTTCG | CCCGCGGCGACG | CCCGCGGCGACGG |
| | | | | TCTCGTGTGGTGC | GTTCGTCTCGTGT | TTCGTCTCGTGTGG |
| | | | | CGGCCGTCCACCT | GGTGCCGTCCGTC | TGCCGGCCGTCCA |
| | | | | CGTAG | CACCTCGTAG | CCTCGTAG |
| | | | | (SEQ ID NO: 28) | (SEQ ID NO: 29) | (SEQ ID NO: 30) |
| EFR117 | 4 | 3 | 316 | ATGAAATACCAAGT | ATGAAATACCAAGT | ATGAAATACCAAGT |
| | | | | ATTACTTTATTACAA | ATTACTTTATTACA | ATTACTTTATTACAA |
| | | | | ATATACAACAATTG | AATATACAACAATT | ATATACAACAATTG |
| | | | | AAGATCCAGAAGCT | GAGGACCCAGAGG | AGGATCCAGAGGC |
| | | | | TTTGCGAAAGAGCA | CTTTTGCGAAAGA | TTTTGCGAAAGAGC |
| | | | | TCTAGCTTTTTGCA | GCACCTAGCTTTTT | ATCTAGCTTTTTGC |
| | | | | AATCATTAAACTTA | GCAAATCATTAAAC | AAATCATTAAACTTA |
| | | | | AAAGGCCGTATTTT | TTAAAAGGCCGCA | AAAGGCCGTATTTT |
| | | | | AGTAGCGACAGAA | TCTTAGTAGCGAC | AGTAGCGACAGAG |
| | | | | GGGATTAACGGAA | AGAGGGGATCAAC | GGGATTAACGGAAC |
| | | | | CGTTATCTGGTACT | GGAACGTTATCTG | GTTATCTGGTACTG |
| | | | | GTCGAAGAAACAG | GTACTGTCGAGGA | TCGAGGAGACAGA |
| | | | | AAAAGTATATGGAA | GACAGAGAAGTAT | GAAGTATATGGAGG |
| | | | | GCAATGCAAGCAG | ATGGAGGCAATGC | CAATGCAAGCAGAT |
| | | | | ATGAGCGCTTTAAG | AGGCAGACGAGCG | GAGCGCTTTAAGGA |
| | | | | GATACATTCTTTAA | CTTTAAGGACACAT | TACATTCTTTAAAAT |
| | | | | AATTGATCCAGCAG | TCTTTAAAATCGAC | TGATCCAGCAGAG |
| | | | | AAGAAATGGCCTTC | CCAGCAGAGGAGA | GAGATGGCCTTCC |
| | | | | CGCAAAATGTTTGT | TGGCCTTCCGCAA | GCAAAATGTTTGTT |
| | | | | TCGCCCACGTTCTG | AATGTTTGTTCGCC | CGCCCACGTTCTGA |
| | | | | AATTAGTGGCGTTG | CACGCTCTGAGTT | GTTAGTGGCGTTGA |
| | | | | AACTTAGAAGAAGA | AGTGGCGTTGAAC | ACTTAGAGGAGGAC |
| | | | | CGTTGATCCATTAG | TTAGAGGAGGACG | GTTGATCCATTAGA |
| | | | | AAACGACGGGGAA | TTGACCCATTAGA | GACGACGGGGAAA |
| | | | | ATATTTGGAACCTG | GACGACGGGGAAA | TATTTGGAGCCTGC |
| | | | | CAGAATTTAAAGAA | TATTTGGAGCCTG | AGAGTTTAAAGAGG |
| | | | | GCCTTATTAGACGA | CAGAGTTTAAAGA | CCTTATTAGACGAG |
| | | | | AGACACTGTTGTAA | GGCCTTATTAGAC | GACACTGTTGTAAT |
| | | | | TCGATGCTCGTAAC | GAGGACACTGTTG | CGATGCTCGTAACG |
| | | | | GATTATGAATATGA | TAATCGACGCTCG | ATTATGAGTATGAT |
| | | | | TTTAGGTCATTTCC | CAACGACTATGAG | TTAGGTCATTTCCG |
| | | | | GTGGTGCCGTGCG | TATGACTTAGGTCA | TGGTGCCGTGCGC |
| | | | | CCCAGATATCCGTA | CTTCCGCGGTGCC | CCAGATATCCGTAG |
| | | | | GCTTCCGTGAATTA | GTGCGCCCAGACA | CTTCCGTGAGTTAC |
| | | | | CCACAATGGATTCG | TCCGCAGCTTCCG | CACAATGGATTCGC |
| | | | | CGAGAACAAAGAA | CGAGTTACCACAG | GAGAACAAAGAGAA |
| | | | | AAATTTATGGATAA | TGGATCCGCGAGA | ATTTATGGATAAAA |
| | | | | AAAAATTGTTACCT | ACAAAGAGAAATTT | AAATTGTTACCTATT |
| | | | | ATTGTACTGGCGG | ATGGACAAAAAAAT | GTACTGGCGGGATT |
| | | | | GATTCGCTGTGAAA | CGTTACCTATTGTA | CGCTGTGAGAAATT |
| | | | | AATTTTCTGGCTGG | CTGGCGGGATCCG | TTCTGGCTGGTTAT |
| | | | | TTATTAAAAGAAGG | CTGTGAGAAATTTT | TAAAAGAGGGATTT |
| | | | | ATTTGAAGATGTTG | CTGGCTGGTTATTA | GAGGATGTTGCTCA |
| | | | | CTCAATTGCATGGT | AAAGAGGGATTTG | ATTGCATGGTGGTA |
| | | | | GGTATCGCCAACTA | AGGACGTTGCTCA | TCGCCAACTATGGA |
| | | | | TGGAAAAAATCCAG | GTTGCACGGTGGT | AAAAATCCAGAGAC |
| | | | | AAACACGTGGCGA | ATCGCCAACTATG | ACGTGGCGAGCTTT |
| | | | | ACTTTGGGACGGC | GAAAAAATCCAGA | GGGACGGCAAAAT |
| | | | | AAAATGTATGTCTT | GACACGCGGCGAG | GTATGTCTTTGATG |
| | | | | TGATGACCGAATCA | CTTTGGGACGGCA | ACCGAATCAGTGTC |
| | | | | GTGTCGAAATTAAT | AAATGTATGTCTTT | GAGATTAATCATGT |
| | | | | CATGTTGATAAAAA | GACGACCGAATCA | TGATAAAAAAGTTA |
| | | | | AGTTATTGGGAAAG | GTGTCGAGATCAA | TTGGGAAAGACTGG |
| | | | | ACTGGTTTGATGGG | TCACGTTGACAAAA | TTTGATGGGACACC |
| | | | | ACACCTTGCGAAC | AAGTTATCGGGAA | TTGCGAGCGCTACA |
| | | | | GCTACATTAACTGT | AGACTGGTTTGAC | TTAACTGTGCAAAC |
| | | | | GCAAACCCAGAAT | GGGACACCTTGCG | CCAGAGTGTAATCG |
| | | | | GTAATCGTCAAATC | AGCGCTACATCAA | TCAAATCTTAACTTC |
| | | | | TTAACTTCAGAAGA | CTGTGCAAACCCA | AGAGGAGAATGAG |
| | | | | AAATGAACATAAAC | GAGTGTAATCGCC | CATAAACATTTAGG |
| | | | | ATTTAGGTGGCTGC | AGATCTTAACTTCA | TGGCTGCTCATTAG |
| | | | | TCATTAGAATGTAG | GAGGAGAATGAGC | AGTGTAGCCAGCAT |
| | | | | CCAGCATCCTGCC | ACAAACACTTAGGT | CCTGCCAACCGTTA |
| | | | | AACCGTTATGTAAA | GGCTGCTCATTAG | TGTAAAAAAACATA |
| | | | | AAAACATAATTTAA | AGTGTAGCCAGCA | ATTTAACAGAGGCA |
| | | | | CAGAAGCAGAAGTT | CCCTGCCAACCGC | GAGGTTGCTGAGC |
| | | | | GCTGAACGTTTAGC | TATGTAAAAAAACA | GTTTAGCTTTGTTA |
| | | | | TTTGTTAGAAGCGG | CAATTTAACAGAG | GAGGCGGTTGAGG |
| | | | | TTGAAGTATAA | GCAGAGGTTGCTG | TATAA |
| | | | | (SEQ ID NO: 31) | AGCGCTTAGCTTT | (SEQ ID NO: 33) |
| | | | | | GTTAGAGGCGGTT | |
| | | | | | GAGGTATAA | |
| | | | | | (SEQ ID NO: 32) | |
| BTR251 | 4 | 3 | 184 | | ATGATATACAGATT | |
| | | | | | TACTATCATATCTG | |
| | | | | | ATGAAGTTGACGA | |
| | | | | ATGATATACAGATT | TTTTGTCAGAGAGA | ATGATATACAGATT |
| | | | | TACTATCATATCTG | TACAGATCGACCC | TACTATCATATCTG |
| | | | | ATGAAGTTGACGAT | GGAGGCTACATTT | ATGAAGTTGACGAT |
| | | | | TTTGTCAGAGAAAT | CTTGACTTCCACG | TTTGTCAGAGAGAT |
| | | | | ACAAATTGATCCGG | AGGCAATACTGAA | ACAAATTGATCCGG |
| | | | | AAGCTACATTTCTT | ATCAGTAGGGTAC | AGGCTACATTTCTT |
| | | | | GACTTCCATGAAGC | ACAAACGACCAGA | GACTTCCATGAGGC |
| | | | | AATACTGAAATCAG | TGACCTCCTTCTTT | AATACTGAAATCAG |
| | | | | TAGGGTACACAAAC | ATCTGCGACGACG | TAGGGTACACAAAC |
| | | | | GACCAGATGACCT | ACTGGGAGAAAGA | GACCAGATGACCTC |
| | | | | CCTTCTTTATCTGC | GAAAGAGGTCACT | CTTCTTTATCTGCG |
| | | | | GATGATGATTGGGA | TTGGAGGAGATGG | ATGATGATTGGGAG |
| | | | | AAAAGAAAAAGAAG | ACGACAATCCGGA | AAAGAGAAAGAGGT |
| | | | | TCACTTTGGAAGAA | GATGGACAGTTGG | CACTTTGGAGGAGA |
| | | | | ATGGACGACAATCC | ATAATGAAAGAGA | TGGACGACAATCCG |
| | | | | GGAAATGGATAGTT | CTACTATCAGCGA | GAGATGGATAGTTG |
| | | | | GGATAATGAAAGAG | GCTGGTAGAGGAC | GATAATGAAAGAGA |
| | | | | ACTACTATCAGCGA | GAGAAGCAGAAAT | CTACTATCAGCGAG |
| | | | | ACTGGTAGAAGATG | TGTTGTATGTATTC | CTGGTAGAGGATGA |
| | | | | AAAAGCAAAAATTG | GACTACATGACAG | GAAGCAAAAATTGT |
| | | | | TTGTATGTATTCGA | AGCGCTGCTTCTT | TGTATGTATTCGAC |
| | | | | CTACATGACAGAGC | CATCGAGTTGTCT | TACATGACAGAGCG |
| | | | | GTTGCTTCTTCATC | GAGATCATCACCG | TTGCTTCTTCATCG |
| | | | | GAATTGTCTGAAAT | GAAAAGACATGAA | AGTTGTCTGAGATC |
| | | | | CATCACCGGAAAA | TGGTGCCAAATGT | ATCACCGGAAAAGA |
| | | | | GATATGAATGGTGC | ACCAAGAAATCGG | TATGAATGGTGCCA |
| | | | | CAAATGTACCAAGA | GTGACGCTCCGCC | AATGTACCAAGAAA |
| | | | | AATCGGGTGATGCT | ACAGACTGTAGAC | TCGGGTGATGCTCC |
| | | | | CCGCCACAAACTGT | TTTGAGGAGATGG | GCCACAAACTGTAG |
| | | | | AGATTTTGAAGAAA | CTGCTGCAAGCGG | ATTTTGAGGAGATG |
| | | | | TGGCTGCTGCAAG | TTCACTCGACCTG | GCTGCTGCAAGCG |
| | | | | CGGTTCACTCGAC | GACGAGAATTTCTA | GTTCACTCGACCTG |
| | | | | CTGGACGAAAATTT | TGGTGACCAGGAC | GACGAGAATTTCTA |
| | | | | CTATGGTGATCAGG | TTTGACATGGAGG | TGGTGATCAGGACT |
| | | | | ACTTTGATATGGAA | ACTTTGACCAGGA | TTGATATGGAGGAT |
| | | | | GATTTTGATCAGGA | GGGCTTCGACATA | TTTGATCAGGAGGG |
| | | | | AGGCTTCGACATAG | GGTGGTAACGCGG | CTTCGACATAGGTG |
| | | | | GTGGTAACGCGGG | GTGGCTCTTATGA | GTAACGCGGGTGG |
| | | | | TGGCTCTTATGAAG | GGAGGAGAAGTTT | CTCTTATGAGGAGG |
| | | | | AAGAGAAGTTTTAA | TAA | AGAAGTTTTAA |
| | | | | (SEQ ID NO: 34) | (SEQ ID NO: 35) | (SEQ ID NO: 36) |
| ILE | | | | | | |
| XR92 | 1 | 5 | 283 | ATGAAGACAATTCA | ATGAAGACAATTCA | ATGAAGACAATTCA |
| | | | | GGAGCAGCAGATG | GGAGCAGCAGATG | GGAGCAGCAGATG |
| | | | | AAGATAGTTAGGAA | AAGATAGTTAGGA | AAGATAGTTAGGAA |
| | | | | TATGAGGAGGATTA | ATATGCGTCGTATT | TATGAGGAGGATTA |
| | | | | GGTACAAGATAGCT | CGTTACAAGATTG | GGTACAAGATTGCT |
| | | | | GTTATAAGCACGAA | CTGTTATTAGCACG | GTTATTAGCACGAA |
| | | | | AGGAGGTGTGGGG | AAAGGAGGTGTGG | AGGAGGTGTGGGG |
| | | | | AAAAGCTTTGTTAC | GGAAAAGCTTTGTT | AAAAGCTTTGTTAC |
| | | | | CGCTAGCCTCGCG | ACCGCTAGCCTCG | CGCTAGCCTCGCG |
| | | | | GCAGCCCTCGCTG | CGGCAGCCCTCGC | GCAGCCCTCGCTG |
| | | | | CGGAGGGGCGAAG | TGCGGAAGGGCGT | CGGAGGGGCGAAG |
| | | | | GGTTGGAGTTTTTG | CGTGTTGGAGTTTT | GGTTGGAGTTTTTG |
| | | | | ACGCAGATATAAGC | TGATGCAGATATTA | ACGCAGATATTAGC |
| | | | | GGTCCTAGCGTTCA | GCGGTCCTAGCGT | GGTCCTAGCGTTCA |
| | | | | TAAAATGCTCGGCC | TCATAAAATGCTCG | TAAAATGCTCGGCC |
| | | | | TCCAGACGGGCAT | GCCTCCAAACGGG | TCCAGACGGGCAT |
| | | | | GGGTATGCCCTCG | CATGGGTATGCCC | GGGTATGCCCTCG |
| | | | | CAGCTCGACGGCA | TCGCAACTCGATG | CAGCTCGACGGCA |
| | | | | CTGTAAAGCCCGT | GCACTGTAAAGCC | CTGTAAAGCCCGTG |
| | | | | GGAAGTTCCTCCG | CGTGGAAGTTCCT | GAAGTTCCTCCGG |
| | | | | GGAATTAAAGTAGC | CCGGGAATTAAAG | GAATTAAAGTAGCT |
| | | | | TAGCATAGGGCTGT | TAGCTAGCATTGG | AGCATTGGGCTGTT |
| | | | | TGCTGCCCATGGAT | GCTGTTGCTGCCC | GCTGCCCATGGAT |
| | | | | GAGGTGCCCCTAA | ATGGATGAAGTGC | GAGGTGCCCCTAAT |
| | | | | TCTGGAGAGGGGC | CCCTAATTTGGCG | TTGGAGAGGGGCC |
| | | | | CATAAAGACGAGTG | TGGGGCCATTAAG | ATTAAGACGAGTGC |
| | | | | CCATCAGAGAGCT | ACGAGTGCCATTC | CATTAGAGAGCTGC |
| | | | | GCTTGCATACGTCG | GTGAACTGCTTGC | TTGCATACGTCGAC |
| | | | | ACTGGGGAGAACT | ATACGTCGATTGG | TGGGGAGAACTCG |
| | | | | CGACTATCTCCTCA | GGAGAACTCGATT | ACTATCTCCTCATT |
| | | | | TAGACCTACCTCCG | ATCTCCTCATTGAT | GACCTACCTCCGG |
| | | | | GGAACAGGTGATG | CTACCTCCGGGAA | GAACAGGTGATGA |
| | | | | AGGTCCTCACGATA | CAGGTGATGAAGT | GGTCCTCACGATTA |
| | | | | ACCCAGATAATACC | CCTCACGATTACC | CCCAGATTATTCCC |
| | | | | CAACATAACGGGCT | CAAATTATTCCCAA | AACATTACGGGCTT |
| | | | | TCCTGGTAGTCACG | CATTACGGGCTTC | CCTGGTAGTCACGA |
| | | | | ATACCCAGCGAGAT | CTGGTAGTCACGA | TTCCCAGCGAGATT |
| | | | | AGCTAAGTCTGTCG | TTCCCAGCGAAATT | GCTAAGTCTGTCGT |
| | | | | TTAAGAAGGCTGTC | GCTAAGTCTGTCG | TAAGAAGGCTGTCA |
| | | | | AGCTTTGCCAAGAG | TTAAGAAGGCTGT | GCTTTGCCAAGAGG |
| | | | | GATAGAAGCCCCT | CAGCTTTGCCAAG | ATTGAAGCCCCTGT |
| | | | | GTGATAGGAATAGT | CGTATTGAAGCCC | GATTGGAATTGTCG |
| | | | | CGAGAACATGAGC | CTGTGATTGGAATT | AGAACATGAGCTAC |
| | | | | TACTTTAGGTGTAG | GTCGAAAACATGA | TTTAGGTGTAGCGA |
| | | | | CGACGGATCCATA | GCTACTTTCGTTGT | CGGATCCATTCACT |
| | | | | CACTATATCTTCGG | AGCGATGGATCCA | ATATTTTCGGCCGC |
| | | | | CCGCGGCGCGGCT | TTCATTATATTTTC | GGCGCGGCTGAGG |
| | | | | GAGGAGATCGCGT | GGCCGTGGCGCG | AGATTGCGTCACAG |
| | | | | CACAGTATGGTATA | GCTGAAGAAATTG | TATGGTATTGAACT |
| | | | | GAACTCCTCGGCA | CGTCACAATATGG | CCTCGGCAAAATTC |
| | | | | AAATACCCATAGAC | TATTGAACTCCTCG | CCATTGACCCTGCG |
| | | | | CCTGCGATAAGAG | GCAAAATTCCCATT | ATTAGAGAGTCGAA |
| | | | | AGTCGAACGATAAA | GATCCTGCGATTC | CGATAAAGGCAAAA |
| | | | | GGCAAAATATTCTT | GTGAATCGAACGA | TTTTCTTCCTAGAG |
| | | | | CCTAGAGAATCCAG | TAAAGGCAAAATTT | AATCCAGAGAGCGA |
| | | | | AGAGCGAAGCTTC | TCTTCCTAGAAAAT | AGCTTCGAGAGAGT |
| | | | | GAGAGAGTTCCTTA | CCAGAAAGCGAAG | TCCTTAAGATTGCC |
| | | | | AGATAGCCCGCAG | CTTCGCGTGAATT | CGCAGGATTATTGA |
| | | | | GATAATAGAGATAG | CCTTAAGATTGCC | GATTGTTGAGAAGC |
| | | | | TTGAGAAGCTAGG | CGTCGTATTATTGA | TAGGCCCAAAGCCT |
| | | | | CCCAAAGCCTCCT | AATTGTTGAAAAGC | CCTGCGTGGGGTC |
| | | | | GCGTGGGGTCCCC | TAGGCCCAAAGCC | CCCAGATGGAGTA |
| | | | | AGATGGAGTAG | TCCTGCGTGGGGT | G |
| | | | | (SEQ ID NO: 37) | CCCCAAATGGAAT | (SEQ ID NO: 39) |
| | | | | | AG | |
| | | | | | (SEQ ID NO: 38) | |
| XR49 | 2 | 5 | 188 | | ATGGGTAGTATAG | |
| | | | | ATGGGTAGTATAGA | AGGAGGTGCTTTT | ATGGGTAGTATAGA |
| | | | | GGAGGTGCTTTTG | GGAGGAGAGGCTC | GGAGGTGCTTTTGG |
| | | | | GAGGAGAGGCTCA | ATAGGATATCTAGA | AGGAGAGGCTCATA |
| | | | | TAGGATATCTAGAC | TCCCGGAGCCGAA | GGATATCTAGACCC |
| | | | | CCCGGAGCCGAGA | AAAGTTTTAGCGC | CGGAGCCGAGAAA |
| | | | | AAGTTTTAGCGAGG | GTATTAACCGTCCT | GTTTTAGCGAGGAT |
| | | | | ATAAACAGGCCTTC | TCAAAAATTGTGTC | TAACAGGCCTTCAA |
| | | | | AAAAATAGTGTCTA | TACAAGCAGTTGTA | AAATTGTGTCTACA |
| | | | | CAAGCAGTTGTACA | CAGGGCGTATTAC | AGCAGTTGTACAGG |
| | | | | GGGAGGATAACAC | ACTGATTGAAGGC | GAGGATTACACTGA |
| | | | | TGATCGAGGGCGA | GAAGCTCATTGGC | TTGAGGGCGAGGC |
| | | | | GGCTCACTGGCTC | TCCGTAACGGGGC | TCACTGGCTCAGGA |
| | | | | AGGAACGGGGCAA | ACGTGTAGCGTAC | ACGGGGCAAGAGT |
| | | | | GAGTAGCGTACAA | AAGACCCATCATC | AGCGTACAAGACCC |
| | | | | GACCCATCACCCC | CCATTTCCCGTAGT | ATCACCCCATTTCC |
| | | | | ATATCCCGGAGTGA | GAAGTTGAACGTG | CGGAGTGAGGTTG |
| | | | | GGTTGAAAGGGTT | TTCTACGTCGTGG | AAAGGGTTCTAAGG |
| | | | | CTAAGGAGGGGCT | CTTCACAAACCTTT | AGGGGCTTCACAAA |
| | | | | TCACAAACCTTTGG | GGCTCAAGGTGAC | CCTTTGGCTCAAGG |
| | | | | CTCAAGGTGACCG | CGGCCCTATTCTA | TGACCGGCCCTATT |
| | | | | GCCCTATACTACAT | CATCTCCGTGTTG | CTACATCTCAGGGT |
| | | | | CTCAGGGTTGAGG | AAGGGTGGCAATG | TGAGGGGTGGCAG |
| | | | | GGTGGCAGTGTGC | TGCAAAGTCCCTT | TGTGCAAAGTCCCT |
| | | | | AAAGTCCCTTCTCG | CTCGAAGCAGCTC | TCTCGAGGCAGCTA |
| | | | | AGGCAGCTAGGAG | GTCGTAACGGGTT | GGAGAAACGGGTT |
| | | | | AAACGGGTTCAAG | CAAGCATAGCGGA | CAAGCACAGCGGA |
| | | | | CACAGCGGAGTCA | GTCATTAGCATTGC | GTCATTAGCATTGC |
| | | | | TAAGCATAGCTGAG | TGAAGATTCACGT | TGAGGATTCAAGAC |
| | | | | GATTCAAGACTCGT | CTCGTCATTGAAAT | TCGTCATTGAAATT |
| | | | | CATAGAAATAATGA | TATGAGCAGCCAA | ATGAGCAGCCAGA |
| | | | | GCAGCCAGAGCAT | AGCATGTCAGTAC | GCATGTCAGTACCT |
| | | | | GTCAGTACCTCTAG | CTCTAGTTATGGAA | CTAGTTATGGAGGG |
| | | | | TTATGGAGGGTGCT | GGTGCTCGTATTG | TGCTAGGATTGTCG |
| | | | | AGGATAGTCGGCG | TCGGCGATGATGC | GCGACGATGCCCT |
| | | | | ACGATGCCCTAGAT | CCTAGATATGCTG | AGATATGCTGATTG |
| | | | | ATGCTGATTGAGAA | ATTGAAAAAGCAAA | AGAAAGCAAACACT |
| | | | | AGCAAACACTATAC | CACTATTCTAGTTG | ATTCTAGTTGAGTC |
| | | | | TAGTTGAGTCTAGA | AATCTCGTATTGG | TAGAATTGGGCTAG |
| | | | | ATCGGGCTAGACA | GCTAGATACGTTTT | ACACGTTTTCAAGA |
| | | | | CGTTTTCAAGAGAG | CACGTGAAGTCGA | GAGGTCGAAGAGC |
| | | | | GTCGAAGAGCTTGT | AGAACTTGTCGAAT | TTGTCGAATGCTTT |
| | | | | CGAATGCTTTTAA | GCTTTTAA | TAA |
| | | | | (SEQ ID NO: 40) | (SEQ ID NO: 41) | (SEQ ID NO: 42) |
| NSR299 | 4 | 2 | 162 | ATGACTATTGACCA | ATGACTATTGACCA | ATGACTATTGACCA |
| | | | | AATGACTATTGACC | AATGACTATTGACC | AATGACTATTGACC |
| | | | | AAATGACTAAAATT | AAATGACTAAAATT | AAATGACTAAAATTT |
| | | | | TTTCTTGCAGATAA | TTTCTTGCAGACAA | TTCTTGCAGATAAA |
| | | | | AGAGTCAACACTCA | AGAGTCAACACTC | GAGTCAACACTCAA |
| | | | | ACTTAGGTATTCTC | AACTTAGGTATCCT | CTTAGGTATCCTCT |
| | | | | TTAGGAGAAACTTT | CTTAGGAGAGACT | TAGGAGAAACTTTA |
| | | | | AACTGCTGGTAGTG | TTAACTGCTGGTA | ACTGCTGGTAGTGT |
| | | | | TGATTTTACTAGAA | GTGTGATCTTACTA | GATCTTACTAGAAG |
| | | | | GGTGATTTAGGTGC | GAGGGTGACTTAG | GTGATTTAGGTGCT |
| | | | | TGGTAAAACTACTT | GTGCTGGTAAAAC | GGTAAAACTACTTT |
| | | | | TGGTACAGGGCTT | TACTTTGGTACAG | GGTACAGGGCTTG |
| | | | | GGGTAAAGGTTTAA | GGCTTGGGTAAAG | GGTAAAGGTTTAAG |
| | | | | GTATTACTGAACCC | GTTTAAGTATCACT | TATCACTGAACCCA |
| | | | | ATTGTCAGTCCTAC | GAGCCCATCGTCA | TCGTCAGTCCTACT |
| | | | | TTTTACTCTGATTAA | GTCCTACTTTTACT | TTTACTCTGATCAAT |
| | | | | TGAGTACACAGAAG | CTGATCAATGAGTA | GAGTACACAGAAG |
| | | | | GACGTATACCCCTT | CACAGAGGGACGC | GACGTATACCCCTT |
| | | | | TACCATCTGGATTT | ATACCCCTTTACCA | TACCATCTGGATTT |
| | | | | ATACCGCTTAGAGC | CCTGGACTTATAC | ATACCGCTTAGAGC |
| | | | | CACAAGAAGTATTA | CGCTTAGAGCCAC | CACAAGAAGTATTA |
| | | | | AGTTTAAATTTAGA | AGGAGGTATTAAG | AGTTTAAATTTAGAA |
| | | | | AATTTATTGGGAAG | TTTAAATTTAGAGA | ATCTATTGGGAAGG |
| | | | | GGATTGAGATAATT | TCTATTGGGAGGG | GATCGAGATAATCC |
| | | | | CCGGGTATTGTAG | GATCGAGATAATC | CGGGTATCGTAGC |
| | | | | CGATTGAGTGGTC | CCGGGTATCGTAG | GATCGAGTGGTCG |
| | | | | GGAACGAATGCCC | CGATCGAGTGGTC | GAACGAATGCCCTA |
| | | | | TACAAGCCAAGTAC | GGAGCGAATGCCC | CAAGCCAAGTACCT |
| | | | | CTACATTAACGTAC | TACAAGCCAAGTA | ACATCAACGTACTT |
| | | | | TTTTGACTTATGGC | CCTACATCAACGTA | TTGACTTATGGCGA |
| | | | | GATGAGGGCAGTC | CTTTTGACTTATGG | TGAGGGCAGTCGT |
| | | | | GTCAAGCCGAAATT | CGACGAGGGCAGT | CAAGCCGAAATCAC |
| | | | | ACACCATTCAATTG | CGCCAGGCCGAGA | ACCATTCAATTGCA |
| | | | | CACCATCAGCGATT | TCACACCATTCAAT | CCATCAGCGATTTA |
| | | | | TAATTGCTACCAAG | TGCACCATCAGCG | ATCGCTACCAAGTG |
| | | | | TGA | ACTTAATCGCTACC | A |
| | | | | (SEQ ID NO: 43) | AAGTGA | (SEQ ID NO: 45) |
| | | | | | (SEQ ID NO: 44) | |
| SPR66 | 4 | 5 | 182 | ATGATTAAATATAG | ATGATTAAATATAG | ATGATTAAATATAGT |
| | | | | TATCCGTGGTGAAA | TATCCGTGGTGAA | ATCCGTGGTGAAAA |
| | | | | ACCTAGAAGTAACA | AACCTAGAAGTAA | CCTAGAAGTAACAG |
| | | | | GAAGCAATTCGTGA | CAGAGGCAATCCG | AAGCAATCCGTGAT |
| | | | | TTATGTAGTTTCTA | CGACTATGTAGTTT | TATGTAGTTTCTAAA |
| | | | | AACTCGAAAAGATC | CTAAACTCGAGAA | CTCGAAAAGATCGA |
| | | | | GAAAAGTACTTCCA | GATCGAGAAGTAC | AAAGTACTTCCAAC |
| | | | | ACCAGAACAAGAGT | TTCCAGCCAGAGC | CAGAACAAGAGTTG |
| | | | | TGGATGCCCGAATT | AGGAGTTGGACGC | GATGCCCGAATCAA |
| | | | | AACTTAAAAGTTTA | CCGAATCAACTTAA | CTTAAAAGTTTATC |
| | | | | TCGTGAAAAAACGG | AAGTTTATCGCGA | GTGAAAAAACGGCT |
| | | | | CTAAAGTGGAAGTA | GAAAACGGCTAAA | AAAGTGGAAGTAAC |
| | | | | ACGATTCCGCTTGG | GTGGAGGTAACGA | GATCCCGCTTGGAT |
| | | | | ATCTATTACTCTCC | TCCCGCTTGGATC | CTATCACTCTCCGC |
| | | | | GCGCAGAAGATGT | TATCACTCTCCGC | GCAGAAGATGTATC |
| | | | | ATCTCAAGATATGT | GCAGAGGACGTAT | TCAAGATATGTATG |
| | | | | ATGGTTCAATTGAC | CTCAGGACATGTA | GTTCAATCGACCTT |
| | | | | CTTGTAACTGATAA | TGGTTCAATCGAC | GTAACTGATAAAAT |
| | | | | AATTGAACGTCAGA | CTTGTAACTGACAA | CGAACGTCAGATCC |
| | | | | TTCGTAAAAATAAA | AATCGAGCGCCAG | GTAAAAATAAAACA |
| | | | | ACAAAAATCGAGCG | ATCCGCAAAAATAA | AAAATCGAGCGTAA |
| | | | | TAAAAATAAAAATA | AACAAAAATCGAG | AAATAAAAATAAGG |
| | | | | AGGTAGCAACTGG | CGCAAAAATAAAAA | TAGCAACTGGTCAA |
| | | | | TCAATTATTTACAG | TAAGGTAGCAACT | TTATTTACAGATGC |
| | | | | ATGCTTTGGTGGAA | GGTCAGTTATTTAC | TTTGGTGGAAGATT |
| | | | | GATTCAAATATTGT | AGACGCTTTGGTG | CAAATATCGTCCAG |
| | | | | CCAGTCTAAAGTTG | GAGGACTCAAATA | TCTAAAGTTGTTCG |
| | | | | TTCGTTCAAAACAA | TCGTCCAGTCTAAA | TTCAAAACAAATCG |
| | | | | ATTGATTTAAAACC | GTTGTTCGCTCAAA | ATTTAAAACCAATG |
| | | | | AATGGATTTGGAAG | ACAGATCGACTTAA | GATTTGGAAGAAGC |
| | | | | AAGCAATTCTACAA | AACCAATGGACTT | AATCCTACAAATGG |
| | | | | ATGGATTTATTGGG | GGAGGAGGCAATC | ATTTATTGGGGCAT |
| | | | | GCATGATTTCTTTA | CTACAGATGGACT | GATTTCTTTATCTAT |
| | | | | TCTATGTGGATGTT | TATTGGGGCACGA | GTGGATGTTGAAGA |
| | | | | GAAGATCAGACAAC | CTTCTTTATCTATG | TCAGACAACCAATG |
| | | | | CAATGTGATTTATC | TGGACGTTGAGGA | TGATCTATCGTCGT |
| | | | | GTCGTGAGGATGG | CCAGACAACCAAT | GAGGATGGCGAAA |
| | | | | CGAAATTGGTTTGT | GTGATCTATCGCC | TCGGTTTGTTAGAG |
| | | | | TAGAGGTTAAAGAA | GCGAGGACGGCG | GTTAAAGAATCTTA |
| | | | | TCTTAA | AGATCGGTTTGTTA | A |
| | | | | (SEQ ID NO: 46) | GAGGTTAAAGAGT | (SEQ ID NO: 48) |
| | | | | | CTTAA | |
| | | | | | (SEQ ID NO: 47) | |
| ARG | | | | | | |
| XR47 | 1 | 2 | 266 | GTGAGGCGGAGGG | NO GENE (done | GTGAGGCGGAGGG |
| | | | | CTAGATGGCTGAG | above) | CTAGATGGCTGAG |
| | | | | GAGGGAGAGGGAG | | GAGGGAGAGGGAG |
| | | | | GAGGAAGAGAGGG | | GAGGAAGAGCGTG |
| | | | | TTAAGGACCGGGA | | TTAAGGACCGTGAC |
| | | | | CATGTTTAAGATTG | | ATGTTTAAGATTGT |
| | | | | TGGACGAGGTTTTC | | GGACGAGGTTTTCG |
| | | | | GACTCCATAACCCT | | ACTCCATAACCCTC |
| | | | | CTCCCACCTCTACA | | TCCCACCTCTACCG |
| | | | | GGCTCTACTCGCG | | TCTCTACTCGCGTA |
| | | | | CAAGGTCCTCAGG | | AGGTCCTCCGTGA |
| | | | | GAGCTCAAGGGCT | | GCTCAAGGGCTCTA |
| | | | | CTATAAGCAGCGGT | | TAAGCAGCGGTAAG |
| | | | | AAGGAGTCTAAGGT | | GAGTCTAAGGTCTA |
| | | | | CTACTGGGGCGTC | | CTGGGGCGTCGCG |
| | | | | GCGTGGGATAGGA | | TGGGATCGTAGCG |
| | | | | GCGACGTCGCCGT | | ACGTCGCCGTTAAG |
| | | | | TAAGATATACCTCT | | ATATACCTCTCGTT |
| | | | | CGTTCACTTCCGAC | | CACTTCCGACTTCC |
| | | | | TTCAGGAAGAGCAT | | GTAAGAGCATTCGT |
| | | | | TAGAAAATATATTG | | AAATATATTGTCGG |
| | | | | TCGGGGACCCCAG | | GGACCCCCGTTTC |
| | | | | GTTCGAGGACATC | | GAGGACATCCCCG |
| | | | | CCCGCAGGCAACA | | CAGGCAACATACGT |
| | | | | TAAGGAGGCTGATA | | CGTCTGATATACGA |
| | | | | TACGAGTGGGCTA | | GTGGGCTCGTAAA |
| | | | | GGAAAGAGTACAG | | GAGTACCGTAACCT |
| | | | | GAACCTCAGGAGG | | CCGTCGTATGCGTG |
| | | | | ATGCGCGAGTCGG | | AGTCGGGGGTCCG |
| | | | | GGGTCAGGGTTCC | | TGTTCCCCGTCCCG |
| | | | | CAGGCCCGTGGCC | | TGGCCGTCGAGGC |
| | | | | GTCGAGGCAAACA | | AAACATTATAGTTAT |
| | | | | TTATAGTTATGGAG | | GGAGTTCCTGGGC |
| | | | | TTCCTGGGCGAGA | | GAGAAGGGGTACC |
| | | | | AGGGGTACAGGGC | | GTGCCCCTACCCTG |
| | | | | CCCTACCCTGGCT | | GCTGAGGCTGTCG |
| | | | | GAGGCTGTCGAGG | | AGGAGCTTGATCGT |
| | | | | AGCTTGATAGGGG | | GGGGAGGCGGAGG |
| | | | | GGAGGCGGAGGCT | | CTATAGCGGCCGA |
| | | | | ATAGCGGCCGAGG | | GGTCCTCCGTCAG |
| | | | | TCCTCCGCCAGGC | | GCGGAGGCTATAG |
| | | | | GGAGGCTATAGTAT | | TATGTCGTGCCCGT |
| | | | | GTAGGGCCAGGCT | | CTCGTGCACGCCG |
| | | | | CGTGCACGCCGAC | | ACCTCAGCGAGTAC |
| | | | | CTCAGCGAGTACAA | | AACATACTAGTCTG |
| | | | | CATACTAGTCTGGA | | GCGTGGGGAGCCC |
| | | | | GGGGGGAGCCCTG | | TGGATAATAGACGT |
| | | | | GATAATAGACGTCT | | CTCCCAGGCGGTG |
| | | | | CCCAGGCGGTGCC | | CCCCACAGCCACC |
| | | | | CCACAGCCACCCG | | CGAACGCTGAGGA |
| | | | | AACGCTGAGGAGT | | GTTTCTAGAGCGTG |
| | | | | TTCTAGAGAGGGA | | ACGTGGAGAACCTC |
| | | | | CGTGGAGAACCTC | | CACCGTTTCTTGAC |
| | | | | CACAGGTTCTTGAC | | AGGTAAGATGGGG |
| | | | | AGGTAAGATGGGG | | TTCGAGTTCGACTT |
| | | | | TTCGAGTTCGACTT | | TGACGCTTATCTCT |
| | | | | TGACGCTTATCTCT | | CTCGTCTAAAAAGC |
| | | | | CTAGGCTAAAAAGC | | TGTATCCACCGTGG |
| | | | | TGTATCCACCGGG | | TGCTCGTGGTTGA |
| | | | | GTGCTAGGGGTTG | | (SEQ ID NO: 50) |
| | | | | A | | |
| | | | | (SEQ ID NO: 49) | | |
| UR51 | 1 | 1 | 170 | GTGAACCTGGACG | GTGAACCTGGACG | GTGAACCTGGACG |
| | | | | CCCCACGGGTCCT | CCCCACGGGTCCT | CCCCACGGGTCCT |
| | | | | GGTCCTCAACGCC | GGTCCTCAACGCC | GGTCCTCAACGCC |
| | | | | GCCTACGAGGTCC | GCCTACGAAGTCC | GCCTACGAGGTCCT |
| | | | | TGGGCCTGGCCAG | TGGGCCTGGCCAG | GGGCCTGGCCAGC |
| | | | | CATCAAGCGGGCC | CATTAAGCGTGCC | ATCAAGCGTGCCGT |
| | | | | GTGCTCCTCGTCCT | GTGCTCCTCGTCC | GCTCCTCGTCCTCG |
| | | | | CGGGGGCGGGGC | TCGGGGGCGGGG | GGGGCGGGGCGGA |
| | | | | GGAGATGGTCTCG | CGGAAATGGTCTC | GATGGTCTCGGAAA |
| | | | | GAAAGCGGCCTCT | GGAAAGCGGCCTC | GCGGCCTCTACCTC |
| | | | | ACCTCAACACCCCC | TACCTCAACACCC | AACACCCCCTCCAC |
| | | | | TCCACCCGGATCC | CCTCCACCCGTAT | CCGTATCCCCGTCC |
| | | | | CCGTCCCCAGCGT | TCCCGTCCCCAGC | CCAGCGTCGTCCG |
| | | | | CGTCCGCCTCAAG | GTCGTCCGTCTCA | TCTCAAGCGTATGG |
| | | | | CGCATGGTCCGCC | AGCGTATGGTCCG | TCCGTCGTCGTCCG |
| | | | | GCAGGCCGGGGCG | TCGTCGTCCGGGG | GGGCGTGTTCCCTT |
| | | | | CGTTCCCTTGAACC | CGTGTTCCCTTGA | GAACCGTCGTAACG |
| | | | | GCAGAAACGTCCT | ACCGTCGTAACGT | TCCTCCGTCGTGAC |
| | | | | CCGGCGCGACCGC | CCTCCGTCGTGAT | CGTTACACCTGCCA |
| | | | | TACACCTGCCAGTA | CGTTACACCTGCC | GTACTGCGGGCAA |
| | | | | CTGCGGGCAAAAG | AATACTGCGGGCA | AAGGGCGGGGAGC |
| | | | | GGCGGGGAGCTCA | AAAGGGCGGGGAA | TCACCGTGGACCAC |
| | | | | CCGTGGACCACGT | CTCACCGTGGATC | GTCCTCCCCAAAAG |
| | | | | CCTCCCCAAAAGC | ATGTCCTCCCCAA | CCGTGGGGGCAAG |
| | | | | CGCGGGGGCAAGA | AAGCCGTGGGGGC | AGCACCTGGGACA |
| | | | | GCACCTGGGACAA | AAGAGCACCTGGG | ACCTGGTGGCCGC |
| | | | | CCTGGTGGCCGCC | ATAACCTGGTGGC | CTGCCGTAGCTGCA |
| | | | | TGCCGCAGCTGCA | CGCCTGCCGTAGC | ACCTCCGTAAGGG |
| | | | | ACCTCAGGAAGGG | TGCAACCTCCGTA | GGACCGTACCCCC |
| | | | | GGACCGCACCCCC | AGGGGGATCGTAC | GAGGAGGCGGGGA |
| | | | | GAGGAGGCGGGGA | CCCCGAAGAAGCG | TGCGTCTCCTCCGT |
| | | | | TGCGCCTCCTCCG | GGGATGCGTCTCC | CCCCCGAAGCCCC |
| | | | | CCCCCCGAAGCCC | TCCGTCCCCCGAA | CGCGTGTGCCCCT |
| | | | | CCGAGGGTGCCCC | GCCCCCGCGTGTG | CTTCCTTTTGGACC |
| | | | | TCTTCCTTTTGGAC | CCCCTCTTCCTTTT | TCAAGGAGGTCCC |
| | | | | CTCAAGGAGGTCC | GGATCTCAAGGAA | CCCGGACTGGCGT |
| | | | | CCCCGGACTGGCG | GTCCCCCCGGATT | CCCTTCGTGGAGG |
| | | | | GCCCTTCGTGGAG | GGCGTCCCTTCGT | GCCTCCTCGGCTA |
| | | | | GGCCTCCTCGGCT | GGAAGGCCTCCTC | G |
| | | | | AG | GGCTAG | (SEQ ID NO: 53) |
| | | | | (SEQ ID NO: 51) | (SEQ ID NO: 52) | |
| SMR69 | 4 | 4 | 182 | | ATGATTAAATATAG | |
| | | | | ATGATTAAATATAG | TATTCGTGGTGAAA | ATGATTAAATATAGT |
| | | | | TATTCGTGGTGAAA | ACATCGAGGTAAC | ATTCGTGGTGAAAA |
| | | | | ACATCGAGGTAACA | AGACGCAATCCGC | CATCGAGGTAACAG |
| | | | | GATGCAATCCGTAA | AACTATGTTGAGTC | ATGCAATCCGCAAC |
| | | | | CTATGTTGAGTCTA | TAAACTCAAGAAGA | TATGTTGAGTCTAA |
| | | | | AACTCAAGAAGATT | TCGAGAAGTATTTC | ACTCAAGAAGATTG |
| | | | | GAAAAGTATTTCAA | AATGCTGAGCAGG | AAAAGTATTTCAAT |
| | | | | TGCTGAACAAGAGT | AGTTGGACGCACG | GCTGAACAAGAGTT |
| | | | | TGGATGCACGTATC | CATCAATCTGAAAG | GGATGCACGCATCA |
| | | | | AATCTGAAAGTATA | TATATCGCGAGAA | ATCTGAAAGTATAT |
| | | | | TCGTGAGAAAACAG | AACAGCTAAAGTT | CGCGAGAAAACAG |
| | | | | CTAAAGTTGAAGTC | GAGGTCACTATCC | CTAAAGTTGAAGTC |
| | | | | ACTATTCCTCTTGC | CTCTTGCTCCCGTT | ACTATTCCTCTTGC |
| | | | | TCCCGTTACTCTTC | ACTCTTCGCGCAG | TCCCGTTACTCTTC |
| | | | | GTGCAGAGGATGT | AGGACGTTTCACA | GCGCAGAGGATGT |
| | | | | TTCACAAGATATGT | GGACATGTATGGT | TTCACAAGATATGT |
| | | | | ATGGTTCTATTGAT | TCTATCGACTTAGT | ATGGTTCTATTGAT |
| | | | | TTAGTTGTTGATAA | TGTTGACAAGATC | TTAGTTGTTGATAA |
| | | | | GATTGAACGTCAGA | GAGCGCCAGATCC | GATTGAACGCCAGA |
| | | | | TTCGTAAAAATAAA | GCAAAAATAAAACT | TTCGCAAAAATAAA |
| | | | | ACTAAAATTGCTAA | AAAATCGCTAAGAA | ACTAAAATTGCTAA |
| | | | | GAAGCATCGTGAAA | GCACCGCGAGAAG | GAAGCATCGCGAAA |
| | | | | AGAAACCAGCGGC | AAACCAGCGGCAC | AGAAACCAGCGGC |
| | | | | ACATGTCTTTACAG | ACGTCTTTACAGCT | ACATGTCTTTACAG |
| | | | | CTGAATTTGAAGCA | GAGTTTGAGGCAG | CTGAATTTGAAGCA |
| | | | | GAAGAGATGGAAG | AGGAGATGGAGGA | GAAGAGATGGAAG |
| | | | | AGGCTCCAGCTATA | GGCTCCAGCTATA | AGGCTCCAGCTATA |
| | | | | AAGGTTGTCAGAAC | AAGGTTGTCAGAA | AAGGTTGTCAGAAC |
| | | | | CAAAAACATCACTT | CCAAAAACATCACT | CAAAAACATCACTT |
| | | | | TAAAACCTATGGAT | TTAAAACCTATGGA | TAAAACCTATGGAT |
| | | | | ATCGAAGAGGCTC | CATCGAGGAGGCT | ATCGAAGAGGCTC |
| | | | | GTTTACAAATGGAT | CGCTTACAGATGG | GCTTACAAATGGAT |
| | | | | CTCTTAGGTCACGA | ACCTCTTAGGTCA | CTCTTAGGTCACGA |
| | | | | TTTCTTCATCTACA | CGACTTCTTCATCT | TTTCTTCATCTACAC |
| | | | | CAGATGCTAATGAT | ACACAGACGCTAA | AGATGCTAATGATA |
| | | | | AATACAACAAATGT | TGACAATACAACAA | ATACAACAAATGTT |
| | | | | TCTCTATCGTCGTG | ATGTTCTCTATCGC | CTCTATCGCCGCGA |
| | | | | AAGATGGTAATTTG | CGCGAGGACGGTA | AGATGGTAATTTGG |
| | | | | GGTCTTATTGAAGC | ATTTGGGTCTTATC | GTCTTATTGAAGCA |
| | | | | AAAATAA | GAGGCAAAATAA | AAATAA |
| | | | | (SEQ ID NO: 54) | (SEQ ID NO: 55) | (SEQ ID NO: 56) |
| BCR108 | 4 | 4 | 220 | ATGAAACAATCTTT | ATGAAACAATCTTT | ATGAAACAATCTTT |
| | | | | ATTCGGACGTGTAC | ATTCGGACGTGTA | ATTCGGACGTGTAC |
| | | | | GCGATGCAATTTTA | CGCGATGCAATTTT | GCGATGCAATTTTA |
| | | | | GCTGATTTTCATAA | AGCTGACTTTCACA | GCTGATTTTCATAA |
| | | | | CGTGTTAGATGAGA | ACGTGTTAGACGA | CGTGTTAGATGAGA |
| | | | | AGGAAAGAAAAAAT | GAAGGAGAGAAAA | AGGAAAGAAAAAAT |
| | | | | CCAATTGCGATGTT | AATCCAATCGCGA | CCAATTGCGATGTT |
| | | | | AAACCAATATTTAC | TGTTAAACCAGTAT | AAACCAATATTTAC |
| | | | | GTGATAGTGAGCG | TTACGCGACAGTG | GCGATAGTGAGCG |
| | | | | TGAAATAACAAAAA | AGCGCGAGATAAC | CGAAATAACAAAAA |
| | | | | TTGAGAAGTTAATT | AAAAATCGAGAAG | TTGAGAAGTTAATT |
| | | | | GAGCGTCATAAAAC | TTAATCGAGCGCC | GAGCGCCATAAAAC |
| | | | | ATTAAAATCTAATTT | ACAAAACATTAAAA | ATTAAAATCTAATTT |
| | | | | TGCTCGTGAGCTTG | TCTAATTTTGCTCG | TGCTCGCGAGCTTG |
| | | | | AGCAAGCACGTTAT | CGAGCTTGAGCAG | AGCAAGCACGCTAT |
| | | | | TTCGTTAATAAAAG | GCACGCTATTTCG | TTCGTTAATAAAAG |
| | | | | ATCAAAGCAAGCTA | TTAATAAAAGATCA | ATCAAAGCAAGCTA |
| | | | | TCATTGCTCAAGAA | AAGCAGGCTATCA | TCATTGCTCAAGAA |
| | | | | GCAGACGAATTACA | TCGCTCAGGAGGC | GCAGACGAATTACA |
| | | | | ATTGCACGAACGTG | AGACGAGTTACAG | ATTGCACGAACGCG |
| | | | | CGTTAGAAGAGGTA | TTGCACGAGCGCG | CGTTAGAAGAGGTA |
| | | | | GCTTATTATGAAGG | CGTTAGAGGAGGT | GCTTATTATGAAGG |
| | | | | GCAAGTAACTCGAT | AGCTTATTATGAGG | GCAAGTAACTCGAT |
| | | | | TAGAAGAAATGTAT | GGCAGGTAACTCG | TAGAAGAAATGTAT |
| | | | | GCAGGTGTTGTAG | ATTAGAGGAGATG | GCAGGTGTTGTAGA |
| | | | | AGCAAATTGATGAG | TATGCAGGTGTTG | GCAAATTGATGAGT |
| | | | | TTAGAGCGTCGTCT | TAGAGCAGATCGA | TAGAGCGCCGCCTT |
| | | | | TTCTGAAATGAAAA | CGAGTTAGAGCGC | TCTGAAATGAAAAA |
| | | | | ATAAATTAAAAGAA | CGCCTTTCTGAGA | TAAATTAAAAGAAAT |
| | | | | ATGCACGCAAAGC | TGAAAAATAAATTA | GCACGCAAAGCGC |
| | | | | GCATGGAACTAATG | AAAGAGATGCACG | ATGGAACTAATGGC |
| | | | | GCACGTGAAAATAT | CAAAGCGCATGGA | ACGCGAAAATATGG |
| | | | | GGCACATGCAAATC | GCTAATGGCACGC | CACATGCAAATCGC |
| | | | | GTCGTATGAATACT | GAGAATATGGCAC | CGCATGAATACTGC |
| | | | | GCGATGCATAAAAT | ACGCAAATCGCCG | GATGCATAAAATGG |
| | | | | GGATGAAAATAATC | CATGAATACTGCG | ATGAAAATAATCCG |
| | | | | CGTTCTTACGATTT | ATGCACAAAATGG | TTCTTACGATTTGA |
| | | | | GAAGAGATTGAAGA | ACGAGAATAATCC | AGAGATTGAAGATC |
| | | | | TCATATTCGTGACT | GTTCTTACGATTTG | ATATTCGCGACTTA |
| | | | | TAGAAACTCGTATG | AGGAGATCGAGGA | GAAACTCGCATGAA |
| | | | | AATGAAGAGCATGA | CCACATCCGCGAC | TGAAGAGCATGAGC |
| | | | | GCGTGACACGTTT | TTAGAGACTCGCA | GCGACACGTTTGAT |
| | | | | GATATGAAAATTGC | TGAATGAGGAGCA | ATGAAAATTGCAAA |
| | | | | AAAACTTGAGCGTG | CGAGCGCGACACG | ACTTGAGCGCGAAA |
| | | | | AAATGAAAGAAAAG | TTTGACATGAAAAT | TGAAAGAAAAGAAT |
| | | | | AATGATGTATCGTT | CGCAAAACTTGAG | GATGTATCGTTAAC |
| | | | | AACGAAAGAGTTAA | CGCGAGATGAAAG | GAAAGAGTTAACAA |
| | | | | CAAAATAA | AGAAGAATGACGT | AATAA |
| | | | | (SEQ ID NO: 57) | ATCGTTAACGAAA | (SEQ ID NO: 59) |
| | | | | | GAGTTAACAAAATA | |
| | | | | | A | |
| | | | | | (SEQ ID NO: 58) | |
| GLN | | | | | | |
| DRR107 | 2 | 2 | 306 | ATGGCTGCCCCGC | ATGGCTGCCCCGC | ATGGCTGCCCCGC |
| | | | | TCATCCCCGTCCTG | TCATCCCCGTCCT | TCATCCCCGTCCTG |
| | | | | ACTGCTCCCACCG | GACTGCTCCCACC | ACTGCTCCCACCGC |
| | | | | CTGCGGGCAAAAC | GCTGCGGGCAAAA | TGCGGGCAAAACG |
| | | | | GGCGCTGGCGCTG | CGGCGCTGGCGCT | GCGCTGGCGCTGC |
| | | | | CGGCTGGCGCGGG | GCGTCTGGCGCGT | GGCTGGCGCGGGA |
| | | | | AGTACGGACTCGA | GAATACGGACTCG | GTACGGACTCGAG |
| | | | | GATCGTTGCCGCC | AAATTGTTGCCGC | ATCGTTGCCGCCGA |
| | | | | GACGCCTTCACGG | CGATGCCTTCACG | CGCCTTCACGGTGT |
| | | | | TGTACCGGGGCCT | GTGTACCGTGGCC | ACCGGGGCCTCGA |
| | | | | CGACCTCGGCACT | TCGATCTCGGCAC | CCTCGGCACTGCC |
| | | | | GCCAAGCCGACGC | TGCCAAGCCGACG | AAGCCGACGCCGC |
| | | | | CGCAGGAGCGGGC | CCGCAAGAACGTG | AAGAGCGGGCGAG |
| | | | | GAGCGTCCCCCAC | CGAGCGTCCCCCA | CGTCCCCCACCATC |
| | | | | CATCTGCTTGACGT | TCATCTGCTTGATG | TGCTTGACGTGGTC |
| | | | | GGTCGACGTGACG | TGGTCGATGTGAC | GACGTGACGCAAA |
| | | | | CAGAGCTACGACG | GCAAAGCTACGAT | GCTACGACGTGGC |
| | | | | TGGCGCAGTACGC | GTGGCGCAATACG | GCAATACGCGGCG |
| | | | | GGCGCAGGCCGAG | CGGCGCAAGCCGA | CAAGCCGAGGCCG |
| | | | | GCCGCCATCGTGG | AGCCGCCATTGTG | CCATCGTGGACATC |
| | | | | ACATCCTGGCGCG | GATATTCTGGCGC | CTGGCGCGGGGGC |
| | | | | GGGGCGGCTGCCG | GTGGGCGTCTGCC | GGCTGCCGCTGGT |
| | | | | CTGGTCGTGGGCG | GCTGGTCGTGGGC | CGTGGGCGGCACC |
| | | | | GCACCGGCTTTTAC | GGCACCGGCTTTT | GGCTTTTACCTCAG |
| | | | | CTCAGTGCGCTCA | ACCTCAGTGCGCT | TGCGCTCAGCCGG |
| | | | | GCCGGGGGCTGCC | CAGCCGTGGGCTG | GGGCTGCCGCTCA |
| | | | | GCTCACGCCGCCG | CCGCTCACGCCGC | CGCCGCCGAGTGA |
| | | | | AGTGACCCGAAGA | CGAGTGATCCGAA | CCCGAAGATGCGC |
| | | | | TGCGCGCCGCCCT | GATGCGTGCCGCC | GCCGCCCTCGAAG |
| | | | | CGAAGCCGAGTTA | CTCGAAGCCGAAT | CCGAGTTACAAGAA |
| | | | | CAGGAACGCGGGC | TACAAGAACGTGG | CGCGGGCTGGACG |
| | | | | TGGACGCGCTGCT | GCTGGATGCGCTG | CGCTGCTCGCCGA |
| | | | | CGCCGAAATCGAG | CTCGCCGAAATTG | AATCGAGCAAGCCA |
| | | | | CAGGCCAATCCTG | AACAAGCCAATCC | ATCCTGCCGAGGC |
| | | | | CCGAGGCCGCCCG | TGCCGAAGCCGCC | CGCCCGCATGGAG |
| | | | | CATGGAGCGCAAC | CGTATGGAACGTA | CGCAACCCACGCC |
| | | | | CCACGCCGGGTGG | ACCCACGTCGTGT | GGGTGGTCCGGGC |
| | | | | TCCGGGCGCTGGA | GGTCCGTGCGCTG | GCTGGAGGTCTAC |
| | | | | GGTCTACCGCGCT | GAAGTCTACCGTG | CGCGCTGCCGGGC |
| | | | | GCCGGGCGTTTTC | CTGCCGGGCGTTT | GTTTTCCCGGTGAG |
| | | | | CCGGTGAGTTCGG | TCCCGGTGAATTC | TTCGGGTACTCGCC |
| | | | | GTACTCGCCACCC | GGGTACTCGCCAC | ACCCGCTTTCCAAT |
| | | | | GCTTTCCAGTATCA | CCGCTTTCCAATAT | ATCAAGTGTTTGCC |
| | | | | GGTGTTTGCCTTTT | CAAGTGTTTGCCTT | TTTTCGCCGCCCGC |
| | | | | CGCCGCCCGCCGC | TTCGCCGCCCGCC | CGCCGAGATGGAA |
| | | | | CGAGATGGAACAG | GCCGAAATGGAAC | CAACGGGTGCAAG |
| | | | | CGGGTGCAGGAGC | AACGTGTGCAAGA | AGCGCACCGCCGC |
| | | | | GCACCGCCGCCAT | ACGTACCGCCGCC | CATGCTGCGCGCC |
| | | | | GCTGCGCGCCGGC | ATGCTGCGTGCCG | GGCTGGCCGCAAG |
| | | | | TGGCCGCAGGAGG | GCTGGCCGCAAGA | AGGCGCAATGGCT |
| | | | | CGCAGTGGCTCGC | AGCGCAATGGCTC | CGCCGGGCAAGTG |
| | | | | CGGGCAGGTGCCG | GCCGGGCAAGTGC | CCGCCGGAGCAAG |
| | | | | CCGGAGCAGGAGC | CGCCGGAACAAGA | AGCCGCGCCCGAC |
| | | | | CGCGCCCGACGGT | ACCGCGTCCGACG | GGTGTGGCAAGCG |
| | | | | GTGGCAGGCGCTC | GTGTGGCAAGCGC | CTCGGGTACGCCG |
| | | | | GGGTACGCCGAGG | TCGGGTACGCCGA | AGGCGCTGGCGGT |
| | | | | CGCTGGCGGTGGC | AGCGCTGGCGGTG | GGCGCAAGGCCGC |
| | | | | GCAGGGCCGCCTG | GCGCAAGGCCGTC | CTGAGCCTCGCAG |
| | | | | AGCCTCGCAGGCG | TGAGCCTCGCAGG | GCGCCGAGCAAGC |
| | | | | CCGAGCAAGCCAT | CGCCGAACAAGCC | CATCGCCCTGGCG |
| | | | | CGCCCTGGCGACC | ATTGCCCTGGCGA | ACCCGGCAATACG |
| | | | | CGGCAGTACGGCA | CCCGTCAATACGG | GCAAACGGCAACTC |
| | | | | AACGGCAGCTCAC | CAAACGTCAACTC | ACCTGGATGCGCC |
| | | | | CTGGATGCGCCGT | ACCTGGATGCGTC | GTCAACTCGGGGC |
| | | | | CAGCTCGGGGCCG | GTCAACTCGGGGC | CGAGGTGCAATCG |
| | | | | AGGTGCAATCGCC | CGAAGTGCAATCG | CCGGACGCGGCAG |
| | | | | GGACGCGGCAGAG | CCGGATGCGGCAG | AGGCGCACCTGCG |
| | | | | GCGCACCTGCGGG | AAGCGCATCTGCG | GGCGTTTCTGGAG |
| | | | | CGTTTCTGGAGCGT | TGCGTTTCTGGAA | CGTTCCGGGGCGC |
| | | | | TCCGGGGCGCCGA | CGTTCCGGGGCGC | CGAGTTGA |
| | | | | GTTGA | CGAGTTGA | (SEQ ID NO: 62) |
| | | | | (SEQ ID NO: 60) | (SEQ ID NO: 61) | |
| HR2926 | 1 | 1 | 217 | ATGGAGTCCGTGG | ATGGAGTCCGTGG | ATGGAGTCCGTGG |
| | | | | CCCTGTACAGCTTT | CCCTGTACAGCTTT | CCCTGTACAGCTTT |
| | | | | CAGGCTACAGAGA | CAGGCTACAGAGA | CAGGCTACAGAGA |
| | | | | GCGACGAGCTGGC | GCGATGAACTGGC | GCGACGAGCTGGC |
| | | | | CTTCAACAAGGGA | CTTCAACAAGGGA | CTTCAACAAGGGAG |
| | | | | GACACACTCAAGAT | GATACACTCAAGAT | ACACACTCAAGATC |
| | | | | CCTGAACATGGAG | TCTGAACATGGAA | CTGAACATGGAGGA |
| | | | | GATGACCAGAACT | GATGATCAAAACT | TGACCAAAACTGGT |
| | | | | GGTACAAGGCCGA | GGTACAAGGCCGA | ACAAGGCCGAGCT |
| | | | | GCTCCGGGGTGTC | ACTCCGTGGTGTC | CCGGGGTGTCGAG |
| | | | | GAGGGATTTATTCC | GAAGGATTTATTCC | GGATTTATTCCCAA |
| | | | | CAAGAACTACATCC | CAAGAACTACATTC | GAACTACATCCGCG |
| | | | | GCGTCAAGCCCCA | GTGTCAAGCCCCA | TCAAGCCCCATCCG |
| | | | | TCCGTGGTACTCG | TCCGTGGTACTCG | TGGTACTCGGGCA |
| | | | | GGCAGGATTTCCC | GGCCGTATTTCCC | GGATTTCCCGGCAA |
| | | | | GGCAGCTGGCCGA | GTCAACTGGCCGA | CTGGCCGAAGAGA |
| | | | | AGAGATTCTGATGA | AGAAATTCTGATGA | TTCTGATGAAGCGG |
| | | | | AGCGGAACCATCT | AGCGTAACCATCT | AACCATCTGGGAGC |
| | | | | GGGAGCCTTCCTG | GGGAGCCTTCCTG | CTTCCTGATCCGGG |
| | | | | ATCCGGGAGAGTG | ATTCGTGAAAGTG | AGAGTGAGAGCTC |
| | | | | AGAGCTCCCCAGG | AAAGCTCCCCAGG | CCCAGGGGAGTTC |
| | | | | GGAGTTCTCTGTGT | GGAATTCTCTGTGT | TCTGTGTCTGTGAA |
| | | | | CTGTGAACTATGGA | CTGTGAACTATGG | CTATGGAGACCAAG |
| | | | | GACCAGGTGCAGC | AGATCAAGTGCAA | TGCAACACTTCAAG |
| | | | | ACTTCAAGGTGCTG | CATTTCAAGGTGCT | GTGCTGCGTGAGG |
| | | | | CGTGAGGCCTCGG | GCGTGAAGCCTCG | CCTCGGGGAAGTA |
| | | | | GGAAGTACTTCCTG | GGGAAGTACTTCC | CTTCCTGTGGGAG |
| | | | | TGGGAGGAGAAGT | TGTGGGAAGAAAA | GAGAAGTTCAACTC |
| | | | | TCAACTCCCTCAAC | GTTCAACTCCCTCA | CCTCAACGAGCTG |
| | | | | GAGCTGGTCGACT | ACGAACTGGTCGA | GTCGACTTCTACCG |
| | | | | TCTACCGCACCACC | TTTCTACCGTACCA | CACCACCACCATCG |
| | | | | ACCATCGCCAAGAA | CCACCATTGCCAA | CCAAGAAGCGGCA |
| | | | | GCGGCAGATCTTC | GAAGCGTCAAATTT | AATCTTCCTGCGCG |
| | | | | CTGCGCGACGAGG | TCCTGCGTGATGA | ACGAGGAGCCCTT |
| | | | | AGCCCTTGCTCAAG | AGAACCCTTGCTC | GCTCAAGTCACCTG |
| | | | | TCACCTGGGGCCT | AAGTCACCTGGGG | GGGCCTGCTTTGC |
| | | | | GCTTTGCCCAGGC | CCTGCTTTGCCCA | CCAAGCCCAATTTG |
| | | | | CCAGTTTGACTTCT | AGCCCAATTTGATT | ACTTCTCAGCCCAA |
| | | | | CAGCCCAGGACCC | TCTCAGCCCAAGA | GACCCCTCGCAACT |
| | | | | CTCGCAGCTCAGC | TCCCTCGCAACTC | CAGCTTCCGCCGT |
| | | | | TTCCGCCGTGGCG | AGCTTCCGTCGTG | GGCGACATCATTGA |
| | | | | ACATCATTGAGGTC | GCGATATTATTGAA | GGTCCTGGAGCGC |
| | | | | CTGGAGCGCCCAG | GTCCTGGAACGTC | CCAGACCCCCACT |
| | | | | ACCCCCACTGGTG | CAGATCCCCATTG | GGTGGCGGGGCCG |
| | | | | GCGGGGCCGGTCC | GTGGCGTGGCCGT | GTCCTGCGGGCGC |
| | | | | TGCGGGCGCGTTG | TCCTGCGGGCGTG | GTTGGCTTCTTCCC |
| | | | | GCTTCTTCCCACGG | TTGGCTTCTTCCCA | ACGGAGTTACGTGC |
| | | | | AGTTACGTGCAGC | CGTAGTTACGTGC | AACCCGTGCACCTG |
| | | | | CCGTGCACCTGTG | AACCCGTGCATCT | TGA |
| | | | | A | GTGA | (SEQ ID NO: 65) |
| | | | | (SEQ ID NO: 63) | (SEQ ID NO: 64) | |
| EFR59 | 4 | 4 | 169 | ATGCGAACCTATGA | ATGCGAACCTATG | ATGCGAACCTATGA |
| | | | | ATCAAAAGAAGCCT | AATCAAAAGAAGC | ATCAAAAGAAGCCT |
| | | | | TGATTGAGGCCATT | CTTGATTGAGGCC | TGATTGAGGCCATT |
| | | | | CAAATAGCTTCACA | ATTCAGATAGCTTC | CAGATAGCTTCACA |
| | | | | AAAATATTTAGCTG | ACAGAAATATTTAG | GAAATATTTAGCTG |
| | | | | AATTTGCAGAAATT | CTGAGTTTGCAGA | AATTTGCAGAAATT |
| | | | | CCTGAAACACTTAA | GATCCCTGAGACA | CCTGAAACACTTAA |
| | | | | AGATCACCGAATTG | CTTAAAGACCACC | AGATCACCGAATTG |
| | | | | AAACAGTAGCTAAA | GAATCGAGACAGT | AAACAGTAGCTAAA |
| | | | | ACACCTTCAGAGAA | AGCTAAAACACCTT | ACACCTTCAGAGAA |
| | | | | CTTAGCCTATCAAT | CAGAGAACTTAGC | CTTAGCCTATCAGT |
| | | | | TAGGTTGGCTCAAC | CTATCAGTTAGGTT | TAGGTTGGCTCAAC |
| | | | | TTGCTGCTTTCTTG | GGCTCAACTTGCT | TTGCTGCTTTCTTG |
| | | | | GGAAGAACAAGAA | GCTTTCTTGGGAG | GGAAGAACAGGAA |
| | | | | CAACGTGGTCTGA | GAGCAGGAGCAGC | CAGCGTGGTCTGA |
| | | | | CCGTTCAAACGCCA | GCGGTCTGACCGT | CCGTTCAGACGCCA |
| | | | | GCTGAAGGCTATAA | TCAGACGCCAGCT | GCTGAAGGCTATAA |
| | | | | ATGGAATCAACTGG | GAGGGCTATAAAT | ATGGAATCAGCTGG |
| | | | | GCGCGCTCTATCAA | GGAATCAGCTGGG | GCGCGCTCTATCAG |
| | | | | TCATTTTATCAAAC | CGCGCTCTATCAG | TCATTTTATCAGAC |
| | | | | CTATGGACAAATGA | TCATTTTATCAGAC | CTATGGACAGATGA |
| | | | | GTTTAGAAAGTCAG | CTATGGACAGATG | GTTTAGAAAGTCAG |
| | | | | CTGATTGCGTTGCA | AGTTTAGAGAGTC | CTGATTGCGTTGCA |
| | | | | AGACACCTTAGAAA | AGCTGATCGCGTT | GGACACCTTAGAAA |
| | | | | AATTACTTCATTGG | GCAGGACACCTTA | AATTACTTCATTGG |
| | | | | ATTGACTCGCTTTC | GAGAAATTACTTCA | ATTGACTCGCTTTC |
| | | | | CGAAGACGAATTAT | CTGGATCGACTCG | CGAAGACGAATTAT |
| | | | | TTTTACCTCAACAA | CTTTCCGAGGACG | TTTTACCTCAGCAG |
| | | | | CGGGCTTGGGCGA | AGTTATTTTTACCT | CGGGCTTGGGCGA |
| | | | | CCACCAAAGCACAA | CAGCAGCGGGCTT | CCACCAAAGCACAG |
| | | | | TGGCCTCTTTGGAA | GGGCGACCACCAA | TGGCCTCTTTGGAA |
| | | | | ATGGATTCACATTA | AGCACAGTGGCCT | ATGGATTCACATTA |
| | | | | ATAGCGTTGCCCCT | CTTTGGAAATGGAT | ATAGCGTTGCCCCT |
| | | | | TTTACTAGTTTCCG | CCACATCAATAGC | TTTACTAGTTTCCG |
| | | | | AACGCAAATTCGCA | GTTGCCCCTTTTAC | AACGCAGATTCGCA |
| | | | | AATGGAAAAAAGCT | TAGTTTCCGAACG | AATGGAAAAAAGCT |
| | | | | TGTCTTTAA | CAGATCCGCAAAT | TGTCTTTAA |
| | | | | (SEQ ID NO: 66) | GGAAAAAAGCTTG | (SEQ ID NO: 68) |
| | | | | | TCTTTAA | |
| | | | | | (SEQ ID NO: 67) | |
| BHR192 | 4 | 4 | 164 | ATGGATGTGAAACA | ATGGATGTGAAAC | ATGGATGTGAAACA |
| | | | | AACTTTGGAGAAGG | AAACTTTGGAGAA | AACTTTGGAGAAGG |
| | | | | CGATTGCCCTTCGC | GGCGATTGCCCTT | CGATTGCCCTTCGC |
| | | | | CAAAATAAGCGCTA | CGCCAGAATAAGC | CAGAATAAGCGCTA |
| | | | | TCAAGAGTCGAATG | GCTATCAGGAGTC | TCAGGAGTCGAATG |
| | | | | CCATCCTTGTCACA | GAATGCCATCCTT | CCATCCTTGTCACA |
| | | | | CTCTGTAAGGAGCA | GTCACACTCTGTAA | CTCTGTAAGGAGCA |
| | | | | TGCTCACGATCCAC | GGAGCACGCTCAC | TGCTCACGATCCAC |
| | | | | AAATTCTTTATCAAT | GACCCACAGATCC | AGATTCTTTATCAG |
| | | | | GTGGCTGGAGCTT | TTTATCAGTGTGGC | TGTGGCTGGAGCTT |
| | | | | TGATGTACTAGGAT | TGGAGCTTTGACG | TGATGTACTAGGAT |
| | | | | TGGAAGCTCAAGCT | TACTAGGATTGGA | TGGAAGCTCAGGCT |
| | | | | GTTCCTTATTATGA | GGCTCAGGCTGTT | GTTCCTTATTATGA |
| | | | | AAAGGCGATCGCA | CCTTATTATGAGAA | AAAGGCGATCGCAT |
| | | | | TCGGGTCTTCAAG | GGCGATCGCATCG | CGGGTCTTCAGGG |
| | | | | GAAAGGACTTGGC | GGTCTTCAGGGAA | AAAGGACTTGGCG |
| | | | | GGAGTGTTATCTCG | AGGACTTGGCGGA | GAGTGTTATCTCGG |
| | | | | GGCTAGGTAGCAC | GTGTTATCTCGGG | GCTAGGTAGCACAT |
| | | | | ATTTCGAACGCTAG | CTAGGTAGCACAT | TTCGAACGCTAGGG |
| | | | | GGGAGTATAGGAA | TTCGAACGCTAGG | GAGTATAGGAAAGC |
| | | | | AGCAGAAGCCGTT | GGAGTATAGGAAA | AGAAGCCGTTCTCG |
| | | | | CTCGCAAACGGCG | GCAGAGGCCGTTC | CAAACGGCGTGAA |
| | | | | TGAAGCAATTTCCT | TCGCAAACGGCGT | GCAGTTTCCTAACC |
| | | | | AACCATCAGGCGC | GAAGCAGTTTCCT | ATCAGGCGCTCCGT |
| | | | | TCCGTGTTTTCTAC | AACCACCAGGCGC | GTTTTCTACGCAAT |
| | | | | GCAATGGTCCTCTA | TCCGCGTTTTCTAC | GGTCCTCTACAACC |
| | | | | CAACCTTGGTCGCT | GCAATGGTCCTCT | TTGGTCGCTATGAG |
| | | | | ATGAGCAAGGGGT | ACAACCTTGGTCG | CAGGGGGTAGAATT |
| | | | | AGAATTATTGCTAA | CTATGAGCAGGGG | ATTGCTAAAAATAAT |
| | | | | AAATAATCGCTGAA | GTAGAGTTATTGCT | CGCTGAAACGAGC |
| | | | | ACGAGCGATGATG | AAAAATAATCGCTG | GATGATGAGACGAT |
| | | | | AGACGATACAATCT | AGACGAGCGACGA | ACAGTCTTACAAGC |
| | | | | TACAAGCAAGCGAT | CGAGACGATACAG | AGGCGATTCTCTTT |
| | | | | TCTCTTTTATGCAG | TCTTACAAGCAGG | TATGCAGATAAGCT |
| | | | | ATAAGCTAGATGAA | CGATCCTCTTTTAT | AGATGAAACGTGGA |
| | | | | ACGTGGAAAGCATA | GCAGACAAGCTAG | AAGCATAA |
| | | | | A | ACGAGACGTGGAA | (SEQ ID NO: 71) |
| | | | | (SEQ ID NO: 69) | AGCATAA | |
| | | | | | (SEQ ID NO: 70) | |
| ASP | | | | | | |
| HSR26 | 2 | 2 | 235 | ATGACGGACAAATA | ATGACGGACAAAT | ATGACGGACAAATA |
| | | | | CCGCCTCCGAGAG | ACCGCCTCCGAGA | CCGCCTCCGAGAG |
| | | | | CGCGTCTGGGACG | GCGCGTCTGGGAC | CGCGTCTGGGACG |
| | | | | ACCTCGAAGACAG | GACCTCGAAGATA | ACCTCGAAGATAGC |
| | | | | CGGCGTGGCGCGG | GCGGCGTGGCGC | GGCGTGGCGCGGT |
| | | | | TTCCCGTTCCCGCC | GTTTCCCGTTCCC | TCCCGTTCCCGCCA |
| | | | | ACACGGCCGCATC | GCCACATGGCCGT | CACGGCCGCATCC |
| | | | | CCGAACTACGCCG | ATTCCGAACTACG | CGAACTACGCCGG |
| | | | | GTGCCGATGAGGC | CCGGTGCCGATGA | TGCCGATGAGGCC |
| | | | | CGCCGCCCGCCTC | AGCCGCCGCCCGT | GCCGCCCGCCTCA |
| | | | | ACCGAAACGGACG | CTCACCGAAACGG | CCGAAACGGATGT |
| | | | | TGTGGCAGCGCGC | ATGTGTGGCAACG | GTGGCAGCGCGCT |
| | | | | TGAGACCGTGAAG | TGCTGAAACCGTG | GAGACCGTGAAGG |
| | | | | GCGAACCCCGACG | AAGGCGAACCCCG | CGAACCCCGATGC |
| | | | | CCCCCCAGCTGCC | ATGCCCCCCAACT | CCCCCAGCTGCCG |
| | | | | GGTGCGGCGGGCG | GCCGGTGCGTCGT | GTGCGGCGGGCGG |
| | | | | GCGCTGCGCGCGG | GCGGCGCTGCGTG | CGCTGCGCGCGGG |
| | | | | GGAAGACACTGTA | CGGGGAAGACACT | GAAGACACTGTACG |
| | | | | CGCGGCGGTGCCG | GTACGCGGCGGTG | CGGCGGTGCCGCG |
| | | | | CGGCTGCGCGACG | CCGCGTCTGCGTG | GCTGCGCGATGAG |
| | | | | AGGAGTGTTTCCTG | ATGAAGAATGTTTC | GAGTGTTTCCTGCG |
| | | | | CGCCTCGACCCAA | CTGCGTCTCGATC | CCTCGATCCAACGA |
| | | | | CGACCATCGACGA | CAACGACCATTGA | CCATCGATGATATC |
| | | | | CATCGACGCCGCC | TGATATTGATGCC | GATGCCGCCACGA |
| | | | | ACGACGGTGTCGG | GCCACGACGGTGT | CGGTGTCGGGGAT |
| | | | | GGATCGAGGAGTA | CGGGGATTGAAGA | CGAGGAGTACGGC |
| | | | | CGGCGACCCGGTC | ATACGGCGATCCG | GATCCGGTCGGTC |
| | | | | GGTCCCGGGGACG | GTCGGTCCCGGGG | CCGGGGATGTCGA |
| | | | | TCGATCCCATCGAC | ATGTCGATCCCATT | TCCCATCGATCTCA |
| | | | | CTCATCGTGTCGG | GATCTCATTGTGTC | TCGTGTCGGGGAG |
| | | | | GGAGCGTCGCGGT | GGGGAGCGTCGC | CGTCGCGGTCACC |
| | | | | CACCGACCGCGGC | GGTCACCGATCGT | GATCGCGGCGAGC |
| | | | | GAGCGCGTCGGGA | GGCGAACGTGTCG | GCGTCGGGAAAGG |
| | | | | AAGGGGAGGGGTA | GGAAAGGGGAAGG | GGAGGGGTACAGC |
| | | | | CAGCGACCTGGAG | GTACAGCGATCTG | GATCTGGAGTTCGC |
| | | | | TTCGCGCTGCTGC | GAATTCGCGCTGC | GCTGCTGCGGGCG |
| | | | | GGGCGTTCGGGCG | TGCGTGCGTTCGG | TTCGGGCGCGTCG |
| | | | | CGTCGACGACGAC | GCGTGTCGATGAT | ATGATGATACCGCG |
| | | | | ACCGCGACTGTGA | GATACCGCGACTG | ACTGTGACGACCGT |
| | | | | CGACCGTCCACGA | TGACGACCGTCCA | CCACGAGCGCCAG |
| | | | | GCGCCAGGTCGTC | TGAACGTCAAGTC | GTCGTCGATGATGC |
| | | | | GACGACGCTGTGC | GTCGATGATGCTG | TGTGCCGACCGCC |
| | | | | CGACCGCCGCCCA | TGCCGACCGCCGC | GCCCACGATGTGC |
| | | | | CGACGTGCCGATG | CCATGATGTGCCG | CGATGGAGTACGT |
| | | | | GAGTACGTGGTCA | ATGGAATACGTGG | GGTCACGCCGGAT |
| | | | | CGCCGGACCGAAC | TCACGCCGGATCG | CGAACGATCACCAC |
| | | | | GATCACCACCACC | TACGATTACCACCA | CACCCACGAGGAT |
| | | | | CACGAGGATGACA | CCCATGAAGATGA | GATACGCCCAGTG |
| | | | | CGCCCAGTGGCAT | TACGCCCAGTGGC | GCATCGATTGGGAT |
| | | | | CGACTGGGACGCA | ATTGATTGGGATG | GCACTGGATGAGC |
| | | | | CTGGACGAGCAGC | CACTGGATGAACA | AGCGCCTGGCGGA |
| | | | | GCCTGGCGGAGAT | ACGTCTGGCGGAA | GATCCCGGTGTTG |
| | | | | CCCGGTGTTGGAC | ATTCCGGTGTTGG | GATCGTCGCTCGC |
| | | | | CGTCGCTCGCCGT | ATCGTCGTTCGCC | CGTAG |
| | | | | AG | GTAG | (SEQ ID NO: 74) |
| | | | | (SEQ ID NO: 72) | (SEQ ID NO: 73) | |
| HSR56 | 2 | 2 | 247 | ATGAACGCTCGATC | ATGAACGCTCGAT | ATGAACGCTCGATC |
| | | | | CACGCTCAGTGTGT | CCACGCTCAGTGT | CACGCTCAGTGTGT |
| | | | | GTGCCGTCGCCGC | GTGTGCCGTCGCC | GTGCCGTCGCCGC |
| | | | | CGTCCTCGTTGTCG | GCCGTCCTCGTTG | CGTCCTCGTTGTCG |
| | | | | CCGGGATCGCGGG | TCGCCGGGATTGC | CCGGGATCGCGGG |
| | | | | CGCGACCGCCCTC | GGGCGCGACCGC | CGCGACCGCCCTC |
| | | | | GGCATGGGGCCGG | CCTCGGCATGGGG | GGCATGGGGCCGG |
| | | | | CGTCGGCCGACAC | CCGGCGTCGGCC | CGTCGGCCGATAC |
| | | | | CCACACCACCGAC | GATACCCATACCA | CCACACCACCGATT |
| | | | | TCGAAAGCCATCAC | CCGATTCGAAAGC | CGAAAGCCATCACG |
| | | | | GGTGTCGGCCGCC | CATTACGGTGTCG | GTGTCGGCCGCCG |
| | | | | GGCACCGTCGACG | GCCGCCGGCACC | GCACCGTCGATGC |
| | | | | CAACCGCCAACCA | GTCGATGCAACCG | AACCGCCAACCAG |
| | | | | GGCGGTCATCGAC | CCAACCAAGCGGT | GCGGTCATCGATGT |
| | | | | GTCGCCGTGACCG | CATTGATGTCGCC | CGCCGTGACCGCC |
| | | | | CCAGCGGGAACGA | GTGACCGCCAGCG | AGCGGGAACGATT |
| | | | | CTCCACCGCAGTC | GGAACGATTCCAC | CCACCGCAGTCCG |
| | | | | CGGGAGTCGTTGG | CGCAGTCCGTGAA | GGAGTCGTTGGCG |
| | | | | CGGCCGACGTGCA | TCGTTGGCGGCCG | GCCGATGTGCAGT |
| | | | | GTCCGTCCGCGAC | ATGTGCAATCCGT | CCGTCCGCGATGC |
| | | | | GCCCTCGCCGACG | CCGTGATGCCCTC | CCTCGCCGATGATG |
| | | | | ACGGCGTCCCCGC | GCCGATGATGGCG | GCGTCCCCGCCAA |
| | | | | CAACACCGTCCGC | TCCCCGCCAACAC | CACCGTCCGCACC |
| | | | | ACCACGAACTTCGA | CGTCCGTACCACG | ACGAACTTCGATAT |
| | | | | CATCCGACAGCAA | AACTTCGATATTCG | CCGACAGCAACGC |
| | | | | CGCGACCGCACCC | TCAACAACGTGAT | GATCGCACCCCGA |
| | | | | CGAACGGCGTCGA | CGTACCCCGAACG | ACGGCGTCGAATAC |
| | | | | ATACAGCGGCTAC | GCGTCGAATACAG | AGCGGCTACCGCG |
| | | | | CGCGGCGTCCACG | CGGCTACCGTGGC | GCGTCCACGATCTC |
| | | | | ACCTCGAAATCACG | GTCCATGATCTCG | GAAATCACGACCAA |
| | | | | ACCAACGACACGT | AAATTACGACCAAC | CGATACGTCCGCG |
| | | | | CCGCGGCGGGCGA | GATACGTCCGCGG | GCGGGCGAACTCA |
| | | | | ACTCATCGACGTCG | CGGGCGAACTCAT | TCGATGTCGCCGTC |
| | | | | CCGTCACCAACGG | TGATGTCGCCGTC | ACCAACGGCGCGG |
| | | | | CGCGGACACCATC | ACCAACGGCGCGG | ATACCATCGATGGC |
| | | | | GACGGCACGTCGT | ATACCATTGATGG | ACGTCGTTCACGCT |
| | | | | TCACGCTCTCCGAC | CACGTCGTTCACG | CTCCGATGCCAAAC |
| | | | | GCCAAACGGGACC | CTCTCCGATGCCA | GGGATCGCCTCCA |
| | | | | GCCTCCACAACGA | AACGTGATCGTCT | CAACGATGCGCTGA |
| | | | | CGCGCTGAACACC | CCATAACGATGCG | ACACCGCGATGGC |
| | | | | GCGATGGCCAACG | CTGAACACCGCGA | CAACGCCAGACAG |
| | | | | CCAGACAGCGCGC | TGGCCAACGCCCG | CGCGCCGATACCC |
| | | | | CGACACCCTCGCG | TCAACGTGCCGAT | TCGCGTCCGCCGG |
| | | | | TCCGCCGGCGGGC | ACCCTCGCGTCCG | CGGGCTCGGCGTC |
| | | | | TCGGCGTCGCCGG | CCGGCGGGCTCG | GCCGGCGTCCACG |
| | | | | CGTCCACGCCATC | GCGTCGCCGGCGT | CCATCGATTCCGCG |
| | | | | GACTCCGCGGACA | CCATGCCATTGATT | GATACGACCGCCC |
| | | | | CGACCGCCCATCC | CCGCGGATACGAC | ATCCTCGCGCCGA |
| | | | | TCGCGCCGAGGCC | CGCCCATCCTCGT | GGCCGGCGGGATG |
| | | | | GGCGGGATGGTCC | GCCGAAGCCGGC | GTCCCCCAGAGCA |
| | | | | CCCAGAGCACCAC | GGGATGGTCCCCC | CCACCGCCACCAC |
| | | | | CGCCACCACCATC | AAAGCACCACCGC | CATCGATTCCGGCC |
| | | | | GACTCCGGCCCGG | CACCACCATTGATT | CGGTCACCGTCAC |
| | | | | TCACCGTCACGGC | CCGGCCCGGTCAC | GGCCTCCGTCCAG |
| | | | | CTCCGTCCAGGTG | CGTCACGGCCTCC | GTGACGTACAACGC |
| | | | | ACGTACAACGCGA | GTCCAAGTGACGT | GACGGCGTAG |
| | | | | CGGCGTAG | ACAACGCGACGGC | (SEQ ID NO: 77) |
| | | | | (SEQ ID NO: 75) | GTAG | |
| | | | | | (SEQ ID NO: 76) | |
| EFR62 | 4 | 4 | 192 | | ATGGAAAACAAAA | |
| | | | | ATGGAAAACAAAAC | CAAATAATACAAAA | ATGGAAAACAAAAC |
| | | | | AAATAATACAAAAA | ACAGAGATCAAAA | AAATAATACAAAAA |
| | | | | CAGAGATCAAAAAA | AAAAGGACATGTC | CAGAGATCAAAAAA |
| | | | | AAGGACATGTCAAA | AAAAACTTTTGAGA | AAGGACATGTCAAA |
| | | | | AACTTTTGAGACTA | CTATCAAAGGAGA | AACTTTTGAGACTA |
| | | | | TTAAAGGAGAACTA | GCTATTTTTTGAGG | TTAAAGGAGAACTA |
| | | | | TTTTTTGAAGATAA | ACAAAGTAATCCA | TTTTTTGAAGACAA |
| | | | | AGTAATTCAAAAAA | GAAAATAATCGGTA | AGTAATTCAAAAAA |
| | | | | TAATTGGTATTGCA | TCGCATTAGACGA | TAATTGGTATTGCA |
| | | | | TTAGATGAGATTGA | GATCGACGGTCTT | TTAGACGAGATTGA |
| | | | | TGGTCTTCTAACGA | CTAACGATCGACG | CGGTCTTCTAACGA |
| | | | | TTGATGGAGGCTTC | GAGGCTTCTTCTC | TTGACGGAGGCTTC |
| | | | | TTCTCAAATATAGC | AAATATAGCTGGAA | TTCTCAAATATAGC |
| | | | | TGGAAAACTAGTAA | AACTAGTAAATACG | TGGAAAACTAGTAA |
| | | | | ATACGGATAACACA | GACAACACAACTT | ATACGGACAACACA |
| | | | | ACTTCTGGAGTGGA | CTGGAGTGGACGT | ACTTCTGGAGTGGA |
| | | | | TGTTGAAGTAGGAA | TGAGGTAGGAAAA | CGTTGAAGTAGGAA |
| | | | | AAAAACAAGTCGCA | AAACAGGTCGCAG | AAAAACAAGTCGCA |
| | | | | GTAGATCTTTCAAT | TAGACCTTTCAATA | GTAGACCTTTCAAT |
| | | | | AGTGGCTGAATATG | GTGGCTGAGTATG | AGTGGCTGAATATG |
| | | | | GTAAAGATGTAACT | GTAAAGACGTAAC | GTAAAGACGTAACT |
| | | | | ACAATTTATGATAA | TACAATCTATGACA | ACAATTTATGACAA |
| | | | | AATGAAGCAAGTTA | AAATGAAGCAGGT | AATGAAGCAAGTTA |
| | | | | TTTCAAATGAAGTT | TATCTCAAATGAGG | TTTCAAATGAAGTT |
| | | | | AAGAAAATGACTGG | TTAAGAAAATGACT | AAGAAAATGACTGG |
| | | | | CCTAGATGTAATTG | GGCCTAGACGTAA | CCTAGACGTAATTG |
| | | | | AGATTAATGTAAAC | TCGAGATCAATGTA | AGATTAATGTAAAC |
| | | | | GTCGTAGATGTAAA | AACGTCGTAGACG | GTCGTAGACGTAAA |
| | | | | AACGAAAGAACAAC | TAAAAACGAAAGA | AACGAAAGAACAAC |
| | | | | ATGAAAATGATTCA | GCAGCACGAGAAT | ATGAAAATGACTCA |
| | | | | GTTACTCTACAAGA | GACTCAGTTACTCT | GTTACTCTACAAGA |
| | | | | TCATCTTTCCGATG | ACAGGACCACCTT | CCATCTTTCCGACG |
| | | | | CAGCTTCTGCTACT | TCCGACGCAGCTT | CAGCTTCTGCTACT |
| | | | | GGAGAATTTGCTTC | CTGCTACTGGAGA | GGAGAATTTGCTTC |
| | | | | AAAACAATTTGAAA | GTTTGCTTCAAAAC | AAAACAATTTGAAA |
| | | | | AATCAAAAGAAGCT | AGTTTGAGAAATCA | AATCAAAAGAAGCT |
| | | | | TTAGGCGTAGCAA | AAAGAGGCTTTAG | TTAGGCGTAGCAAG |
| | | | | GTGAAAAAGTAAGT | GCGTAGCAAGTGA | TGAAAAAGTAAGTG |
| | | | | GATGGTGTACAAAA | GAAAGTAAGTGAC | ACGGTGTACAAAAC |
| | | | | CGTAAAAGAAGAAA | GGTGTACAGAACG | GTAAAAGAAGAAAC |
| | | | | CTGAACCTCGCGTA | TAAAAGAGGAGAC | TGAACCTCGCGTAA |
| | | | | AAATAA | TGAGCCTCGCGTA | AATAA |
| | | | | (SEQ ID NO: 78) | AAATAA | (SEQ ID NO: 80) |
| | | | | | (SEQ ID NO: 79) | |
| SR562 | 4 | 4 | 194 | | ATGAGCCAATCGA | |
| | | | | | GCGATGCGTCAGA | |
| | | | | ATGAGCCAATCGA | GAAGGAAAAACCG | ATGAGCCAATCGAG |
| | | | | GCGATGCGTCAGA | AAAGAGAAAAAATC | CGATGCGTCAGAG |
| | | | | GAAGGAAAAACCG | GCAGGAGGAGCTT | AAGGAAAAACCGAA |
| | | | | AAAGAGAAAAAATC | GAGAAGGAGCTTG | AGAGAAAAAATCGC |
| | | | | GCAAGAAGAGCTT | ACAAGGAGTTGAA | AAGAAGAGCTTGAA |
| | | | | GAAAAGGAACTTGA | AAAAGGCGGTGAG | AAGGAACTTGACAA |
| | | | | TAAGGAATTGAAAA | CCGAAGACCAAAA | GGAATTGAAAAAAG |
| | | | | AAGGCGGTGAGCC | AAGACGACCAGAT | GCGGTGAGCCGAA |
| | | | | GAAGACCAAAAAA | ACACAAAATAGGA | GACCAAAAAAGACG |
| | | | | GATGATCAAATACA | GAGACATTTAAAG | ACCAAATACATAAA |
| | | | | TAAAATAGGAGAAA | CAGGACACACGAA | ATAGGAGAAACATT |
| | | | | CATTTAAAGCAGGA | TTTTACAGTGAATA | TAAAGCAGGACATA |
| | | | | CATACGAATTTTAC | AAGTTGACAGAGT | CGAATTTTACAGTG |
| | | | | AGTGAATAAAGTTG | GCAGAAAGGTGAG | AATAAAGTTGACAG |
| | | | | ATAGAGTGCAAAAA | TATATGAATGTTGG | AGTGCAAAAAGGTG |
| | | | | GGTGAATATATGAA | CGGAGCTGTAAAT | AATATATGAATGTT |
| | | | | TGTTGGCGGAGCT | GAGGAGACAAAAA | GGCGGAGCTGTAA |
| | | | | GTAAATGAGGAGA | CAATAAAAGACGA | ATGAGGAGACAAAA |
| | | | | CAAAAACAATAAAA | CGAGGAGCGGCTT | ACAATAAAAGACGA |
| | | | | GATGATGAGGAAC | ATCATAGAGGTTAC | CGAGGAACGGCTT |
| | | | | GGCTTATTATAGAA | GATGGAGAATATA | ATTATAGAAGTTAC |
| | | | | GTTACGATGGAAAA | GGGGAGGACTCAA | GATGGAAAATATAG |
| | | | | TATAGGGGAAGATT | TAAGCTACAATTTT | GGGAAGACTCAATA |
| | | | | CAATAAGCTACAAT | ATCGGGTTTGACTT | AGCTACAATTTTAT |
| | | | | TTTATCGGGTTTGA | AAGAGACAAGAAT | CGGGTTTGACTTAA |
| | | | | TTTAAGAGATAAGA | GACCAGTCAGTGC | GAGACAAGAATGAC |
| | | | | ATGATCAATCAGTG | GGCCTGTTTTTTCT | CAATCAGTGCGGC |
| | | | | CGGCCTGTTTTTTC | ATAGAGGAGAAGG | CTGTTTTTTCTATAG |
| | | | | TATAGAAGAGAAGG | GCAGAATCCTTAT | AAGAGAAGGGCAG |
| | | | | GCAGAATCCTTATG | GGGAGGAACACTA | AATCCTTATGGGAG |
| | | | | GGAGGAACACTAG | GTATCGGGGAAAA | GAACACTAGTATCG |
| | | | | TATCGGGGAAAAA | AGGTTACAGGTGT | GGGAAAAAGGTTAC |
| | | | | GGTTACAGGTGTAC | ACTCAGTTATGTCA | AGGTGTACTCAGTT |
| | | | | TCAGTTATGTCATC | TCCCTAAAGGAGA | ATGTCATCCCTAAA |
| | | | | CCTAAAGGAGAACA | GCAGAAACACTAC | GGAGAACAGAAACA |
| | | | | GAAACATTACACAC | ACACTGGTATATAA | TTACACACTGGTAT |
| | | | | TGGTATATAATCCG | TCCGTTTTTAGCTG | ATAATCCGTTTTTA |
| | | | | TTTTTAGCTGATAC | ACACAAATAGCAG | GCTGACACAAATAG |
| | | | | AAATAGCAGTAATA | TAATACAGAGGAG | CAGTAATACAGAAG |
| | | | | CAGAAGAGAGAGT | AGAGTAAAGGACG | AGAGAGTAAAGGAC |
| | | | | AAAGGACGATATTG | ACATCGACTACTTG | GACATTGACTACTT |
| | | | | ATTACTTGGTGAAG | GTGAAGTTAGACT | GGTGAAGTTAGACT |
| | | | | TTAGATTAG | AG | AG |
| | | | | (SEQ ID NO: 81) | (SEQ ID NO: 82) | (SEQ ID NO: 83) |

### Example 5

### Additional Useful Nucleic Acid Sequences

TABLE 14
Additional Useful Nucleic Acid Sequences

| | SEQ | |
|---|---|---|
| | ID | |
| Target | NO: | Sequence |
| RHR13-1 | 1. | ATGGCGCGTTCGATCGATTACGGCAACCTCATGCACCGCGCGATGCGTGGCCTGATT |
| | | CAAAGCGTGCTCGAAGATGTGGCCGAACATGGGCTGCCCGGCGCGCATCATTTCTTC |
| | | ATTACCTTCGATACGACCCATCCCGATGTGGCCATGGCCGATTGGCTCCGTGCGCGT |
| | | TATCCGCAAGAAATGACGGTCGTGATTCAACATTGGTACGAAAACCTCTCCGCCGAT |
| | | GATCATGGCTTCTCGGTCACGCTGAACTTCGGCAACCAACCCGAACCGCTGGTCATT |
| | | CCCTTCGATGCCGTGCGTACCTTCGTCGATCCGTCCGTGGAATTCGGCCTCCGTTTC |
| | | GAAACCCATGAAGAAGATGAAGAAGAAGAAACGGGCGGCGATGAAGATCCCGATGGC |
| | | GATGATGAACCGCCGCGTCATGATGCGCAAGTCGTGAGCCTCGATAAGTTCCGTAAG |
| RHR13-2 | 2. | ATGGCGCGTTCGATCGATTACGGCAACCTCATGCACCGCGCGATGCGGGGCCTGATC |
| | | CAGAGCGTGCTCGAGGATGTGGCCGAGCATGGGCTGCCCGGCGCGCATCATTTCTTC |
| | | ATCACCTTCGACACGACCCATCCCGATGTGGCCATGGCCGACTGGCTCCGCGCGCGC |
| | | TATCCGCAGGAGATGACGGTCGTGATCCAGCATTGGTACGAGAACCTCTCCGCCGAC |
| | | GACCATGGCTTCTCGGTCACGCTGAACTTCGGCAACCAGCCCGAGCCGCTGGTCATC |
| | | CCCTTCGATGCCGTGCGCACCTTCGTCGACCCGTCCGTGGAATTCGGCCTCCGGTTC |
| | | GAGACCCATGAGGAGGACGAGGAGGAGGAGACGGGCGGCGACGAGGATCCCGACGGC |
| | | GACGACGAGCCGCCGCGCCATGACGCGCAGGTCGTGAGCCTCGACAAGTTCCGCAAG |
| RR162-1 | 3. | ATGAGCACGCGGACGAGGACGACGGAAGAACGCCGGCACGAGATTGTGCGTGTCGCC |
| | | CGTGCCACCGGCTCGGTCGATGTCACCGCGCTCGCCGCCGAACTGGGCGTCGCCAAG |
| | | GAAACCGTACGTCGTGATCTGCGTGCCCTGGAAGATCATGGCCTGGTCCGTCGTACC |
| | | CATGGCGGCGCCTACCCGGTGGAAAGCGCCGGTTTCGAAACCACGCTCGCCTTCCGT |
| | | GCCACCAGCCATGTGCCCGAAAAGCGTCGTATTGCGTCCGCCGCCGTCGAACTGCTC |
| | | GGCGATGCGGAAACGGTCTTCGTCGATGAAGGCTTCACCCCCCAACTCATTGCCGAA |
| | | GCCCTGCCCCGTGATCGTCCGCTGACCGTGGTCACCGCGTCCCTGCCGGTGGCGGGC |
| | | GCGCTGGCCGAAGCGGGCGATACGTCCGTCCTGCTGCTCGGCGGCCGTGTCCGTTCG |
| | | GGCACCCTGGCCACCGTCGATCATTGGACCACGAAGATGCTGGCCGGCTTCGTCATT |
| | | GATCTGGCGTACATTGGCGCCAACGGCATTTCCCGTGAACATGGTCTCACCACACCC |
| | | GATCCCGCGGTCAGCGAAGTCAAGGCGCAAGCCGTCCGTGCCGCCCGTCGTACGGTG |
| | | TTCGCCGGCGCGCATACCAAGTTCGGGGCGGTGAGCTTCTGCCGTTTCGCGGAAGTC |
| | | GGCGCCCTGGAAGCCATTGTCACCAGCACGCTGCTGCCCTCGGCCGAAGCCCATCGT |
| | | TACTCCCTCCTCGGCCCCCAAATTATTCGTGTC |
| RR162-2 | 4. | ATGAGCACGCGGACGAGGACGACGGAAGAACGCCGGCACGAGATCGTGCGGGTCGCC |
| | | CGCGCCACCGGCTCGGTCGACGTCACCGCGCTCGCCGCCGAACTGGGCGTCGCCAAG |
| | | GAGACCGTACGACGCGACCTGCGCGCCCTGGAGGACCATGGCCTGGTCCGCCGCACC |
| | | CATGGCGGCGCCTACCCGGTGGAGAGCGCCGGTTTCGAGACCACGCTCGCCTTCCGC |
| | | GCCACCAGCCATGTGCCCGAGAAGCGCCGGATCGCGTCCGCCGCCGTCGAACTGCTC |
| | | GGCGACGCGGAGACGGTCTTCGTCGACGAGGGCTTCACCCCCCAGCTCATCGCCGAG |
| | | GCCCTGCCCCGGGACCGGCCGCTGACCGTGGTCACCGCGTCCCTGCCGGTGGCGGGC |
| | | GCGCTGGCCGAGGCGGGCGACACGTCCGTCCTGCTGCTCGGCGGCCGGGTCCGCTCG |
| | | GGCACCCTGGCCACCGTCGACCATTGGACCACGAAGATGCTGGCCGGCTTCGTCATC |
| | | GACCTGGCGTACATCGGCGCCAACGGCATCTCCCGGGAGCATGGTCTCACCACACCC |
| | | GACCCCGCGGTCAGCGAGGTCAAGGCGCAGGCCGTCCGGGCCGCCCGCCGCACGGTG |
| | | TTCGCCGGCGCGCATACCAAGTTCGGGGCGGTGAGCTTCTGCCGGTTCGCGGAGGTC |
| | | GGCGCCCTGGAGGCCATCGTCACCAGCACGCTGCTGCCCTCGGCCGAGGCCCATCGC |
| | | TACTCCCTCCTCGGCCCCCAGATCATCCGCGTC |
| SHR52-1 | 5. | ATGGATGTAACACGACAAATAGAATTAGCGCATCGATATATGAAAGACTTTCACAAA |
| | | AGTGACTATTCTGGTCACGACGTTGCACACGTAGAGCGCGTAACGTCACTAGCTCAG |
| | | ACAATCTCTAAATGCGAGCAGCAGGGAGAGTATTTAATCATCACATTATCTGCATTA |
| | | CTTCACGACGTCATCGACGACAAGTTAACAAATAAAGCCAATGCTTTAGACCGCTTA |
| | | AAAACATTTTTAAAGAACATCCGCGTATCTTCTGACCAGCAGCAGAAGATCATCTAC |
| | | ATCATCCAGCACTTAAGTTATAGAAATGGACAGAATAATCACGTAGACCTTCCAATC |
| | | GAGGGACAGATCGTTAGAGACGCAGACCGACTAGACGCGATCGGTGCTATCGGTATC |
| | | GCTAGAGCATTTCAGTTTTCAGGCCACTTTAATGAGCCAATGTGGACAGAGTCACCA |
| | | CACAGTGACATACCTAATATCGAGACGATCACTAATTTAGAGCCTTCCGCTATACGC |
| | | CACTTTTATGACAAATTATTAAAATTAAAAGACTTAATGCACACTGAGACTGGTCGA |
| | | AAATTAGCTAGAGAGAGACACGCGTTTATGGAGCAGTTTTTAAATCAGTTTTATAAA |
| | | GAGTGGCACATA |
| SHR52-2 | 6. | ATGGATGTAACACGACAAATAGAATTAGCGCATCGATATATGAAAGATTTTCACAAA |
| | | AGTGATTATTCTGGTCACGATGTTGCACACGTAGAACGTGTAACGTCACTAGCTCAA |
| | | ACAATCTCTAAATGCGAGCAACAAGGAGAATATTTAATTATCACATTATCTGCATTA |
| | | CTTCACGATGTCATTGATGATAAGTTAACAAATAAAGCCAATGCTTTAGATCGTTTA |
| | | AAAACATTTTTAAAGAACATTCGCGTATCTTCTGATCAACAACAAAAGATTATTTAC |
| | | ATCATTCAACACTTAAGTTATAGAAATGGACAAAATAATCACGTAGACCTTCCAATT |
| | | GAAGGACAAATTGTTAGAGATGCAGATCGACTAGATGCGATTGGTGCTATTGGTATT |
| | | GCTAGAGCATTTCAATTTTCAGGCCACTTTAATGAGCCAATGTGGACAGAATCACCA |
| | | CACAGTGACATACCTAATATTGAAACGATTACTAATTTAGAACCTTCCGCTATACGT |
| | | CACTTTTATGATAAATTATTAAAATTAAAAGATTTAATGCACACTGAAACTGGTCGA |
| | | AAATTAGCTAGAGAAAGACACGCGTTTATGGAACAGTTTTTAAATCAATTTTATAAA |
| | | GAATGGCACATA |
| SyR92-1 | 7. | ATGAAACTCATTCAAATGTCAGACCATATTTATAAATTAAATATACAGACAACAGTT |
| | | GGTATCCCGATACAGATAAACACTTGGTTTATCGTGAATGACAACGACGTTTATATC |
| | | ATAGACACAGGTATGGACGACTATGCTGAGCTACAGATCACGATCGCTAAATCGCTC |
| | | GGTAATCCTAAAGGCATCTTTTTAACGCACGGACACCTAGACCACATCAATGGCGCA |
| | | AAACGCATCTCTGAGGCTTTGAAAATACCTATCTTTACATATAAAAATGAGCTCCCT |
| | | TATATCAATGGTGAGCTGCCTTATCCAAATAAAACGCACACCGAGAATACAGGTGTT |
| | | CAGTACATCGTTAAACCTCTAGAGACTAATACAAATCTGCCCTTCAATTATTACTTA |
| | | ACTCCTGGTCACGCACCAGGTCACGTCATCTATTTTCACAATCAGGACAAAATCTTA |
| | | ATATGCGGAGACTTATTTATCTCAGACGCGCAGCACCTGCACATCCCTATCAAAAAA |
| | | TTCACTTATAACATGACTGAGAATATCAAAAGCGGTCAGATCATAGACAATCTTTGT |
| | | CCCAAATTAATCACAACTTCACACGGCGACGACCTATATTATTCAGACGACATCTAT |
| | | TCAATCTATAAATTTAAGTACGAGGAG |
| SyR92-2 | 8. | ATGAAACTCATTCAAATGTCAGACCATATTTATAAATTAAATATACAGACAACAGTT |
| | | GGTATCCCGATACAAATAAACACTTGGTTTATTGTGAATGATAACGACGTTTATATC |
| | | ATAGACACAGGTATGGATGATTATGCTGAGCTACAAATCACGATTGCTAAATCGCTC |
| | | GGTAATCCTAAAGGCATTTTTTTAACGCACGGACACCTAGATCACATCAATGGCGCA |
| | | AAACGTATTTCTGAAGCTTTGAAAATACCTATCTTTACATATAAAAATGAACTCCCT |
| | | TATATCAATGGTGAGCTGCCTTATCCAAATAAAACGCACACCGAAAATACAGGTGTT |
| | | CAATACATTGTTAAACCTCTAGAAACTAATACAAATCTGCCCTTCAATTATTACTTA |
| | | ACTCCTGGTCACGCACCAGGTCACGTCATCTATTTTCACAATCAAGATAAAATTTTA |
| | | ATATGCGGAGATTTATTTATTTCAGATGCGCAACACCTGCACATTCCTATCAAAAAA |
| | | TTCACTTATAACATGACTGAAAATATCAAAAGCGGTCAAATCATAGATAATCTTTGT |
| | | CCCAAATTAATTACAACTTCACACGGCGATGATCTATATTATTCAGATGACATTTAT |
| | | TCAATTTATAAATTTAAGTACGAGGAG |
| XR47-1 | 9. | ATGAGGCGGAGGGCTAGATGGCTGAGGAGGGAGAGGGAGGAGGAAGAACGTGTTAAG |
| | | GATCGTGATATGTTTAAGATTGTGGATGAAGTTTTCGATTCCATTACCCTCTCCCAT |
| | | CTCTACCGTCTCTACTCGCGTAAGGTCCTCCGTGAACTCAAGGGCTCTATTAGCAGC |
| | | GGTAAGGAATCTAAGGTCTACTGGGGCGTCGCGTGGGATCGTAGCGATGTCGCCGTT |
| | | AAGATTTACCTCTCGTTCACTTCCGATTTCCGTAAGAGCATTCGTAAATATATTGTC |
| | | GGGGATCCCCGTTTCGAAGATATTCCCGCAGGCAACATTCGTCGTCTGATTTACGAA |
| | | TGGGCTCGTAAAGAATACCGTAACCTCCGTCGTATGCGTGAATCGGGGGTCCGTGTT |
| | | CCCCGTCCCGTGGCCGTCGAAGCAAACATTATTGTTATGGAATTCCTGGGCGAAAAG |
| | | GGGTACCGTGCCCCTACCCTGGCTGAAGCTGTCGAAGAACTTGATCGTGGGGAAGCG |
| | | GAAGCTATTGCGGCCGAAGTCCTCCGTCAAGCGGAAGCTATTGTATGTCGTGCCCGT |
| | | CTCGTGCATGCCGATCTCAGCGAATACAACATTCTAGTCTGGCGTGGGGAACCCTGG |
| | | ATTATTGATGTCTCCCAAGCGGTGCCCCATAGCCATCCGAACGCTGAAGAATTTCTA |
| | | GAACGTGATGTGGAAAACCTCCATCGTTTCTTGACAGGTAAGATGGGGTTCGAATTC |
| | | GATTTTGATGCTTATCTCTCTCGTCTAAAAAGCTGTATTCATCGTGGTGCTCGTGGT |
| XR47-2 | 10. | ATGAGGCGGAGGGCTAGATGGCTGAGGAGGGAGAGGGAGGAGGAAGAAAGGGTTAAG |
| | | GACCGGGACATGTTTAAGATTGTGGACGAAGTTTTCGACTCCATAACCCTCTCCCAC |
| | | CTCTACAGGCTCTACTCGCGCAAGGTCCTCAGGGAACTCAAGGGCTCTATAAGCAGC |
| | | GGTAAGGAATCTAAGGTCTACTGGGGCGTCGCGTGGGATAGGAGCGACGTCGCCGTT |
| | | AAGATATACCTCTCGTTCACTTCCGACTTCAGGAAGAGCATTAGAAAATATATTGTC |
| | | GGGGACCCCAGGTTCGAAGACATCCCCGCAGGCAACATAAGGAGGCTGATATACGAA |
| | | TGGGCTAGGAAAGAATACAGGAACCTCAGGAGGATGCGCGAATCGGGGGTCAGGGTT |
| | | CCCAGGCCCGTGGCCGTCGAAGCAAACATTATAGTTATGGAATTCCTGGGCGAAAAG |
| | | GGGTACAGGGCCCCTACCCTGGCTGAAGCTGTCGAAGAACTTGATAGGGGGGAAGCG |
| | | GAAGCTATAGCGGCCGAAGTCCTCCGCCAGGCGGAAGCTATAGTATGTAGGGCCAGG |
| | | CTCGTGCACGCCGACCTCAGCGAATACAACATACTAGTCTGGAGGGGGGAACCCTGG |
| | | ATAATAGACGTCTCCCAGGCGGTGCCCCACAGCCACCCGAACGCTGAAGAATTTCTA |
| | | GAAAGGGACGTGGAAAACCTCCACAGGTTCTTGACAGGTAAGATGGGGTTCGAATTC |
| | | GACTTTGACGCTTATCTCTCTAGGCTAAAAAGCTGTATCCACCGGGGTGCTAGGGGT |
| SRR141-1 | 11. | ATGGCCGCCATGCCCAAGCCCGCTGCGTTCTGGAACGACCGCTTTGCCAACGAAGAA |
| | | TACGTGTACGGCGAAGCCCCCAACCGTTTCGTCGCGAGCGCCGCCCGTACGTGGCTG |
| | | CCGGAAGCCGGTGAAGTTCTCCTGCTCGGGGCGGGCGAAGGGCGTAACGCCGTGCAT |
| | | CTGGCCCGTGAAGGCCATACGGTCACCGCGGTCGATTACGCCGTGGAAGGGCTCCGT |
| | | AAGACGGAACGTCTCGCGACGGAAGCCGGGGTGGAAGTCGAAGCGATTCAAGCCGAT |
| | | GTGCGTGAATGGAAGCCCGCCCGTGCGTGGGATGCGGTCGTCGTCACGTTTCTCCAT |
| | | CTTCCCGCCGATGAACGTCCGGGCCTGTACCGTCTCGTTCAACGTTGTTTGCGTCCC |
| | | GGGGGGCGTCTCGTGGCGGAATGGTTTCGTCCGGAACAACGTACGGATGGCTACACG |
| | | AGCGGCGGCCCGCCCGATCCTGCCATGATGGTCACCGCCGATGAACTCCGTGGGCAT |
| | | TTCGCCGAAGCGGGCATTGATCATCTCGAAGCGGCCGAACCGACCCTCGATGAAGGC |
| | | ATGCATCGTGGCCCCGCGGCGACGGTTCGTCTCGTGTGGTGCCGTCCGTCCACCTCG |
| SRR141-2 | 12. | ATGGCCGCCATGCCCAAGCCCGCTGCGTTCTGGAACGACCGCTTTGCCAACGAAGAA |
| | | TACGTGTACGGCGAAGCCCCCAACCGCTTCGTCGCGAGCGCCGCCCGGACGTGGCTG |
| | | CCGGAAGCCGGTGAAGTTCTCCTGCTCGGGGCGGGCGAAGGGCGCAACGCCGTGCAC |
| | | CTGGCCCGGGAAGGCCATACGGTCACCGCGGTCGACTACGCCGTGGAAGGGCTCCGC |
| | | AAGACGGAACGCCTCGCGACGGAAGCCGGGGTGGAAGTCGAAGCGATCCAGGCCGAT |
| | | GTGCGCGAATGGAAGCCCGCCCGGGCGTGGGACGCGGTCGTCGTCACGTTTCTCCAC |
| | | CTTCCCGCCGACGAACGACCGGGCCTGTACCGCCTCGTTCAGCGCTGTTTGCGGCCC |
| | | GGGGGGCGCCTCGTGGCGGAATGGTTTCGCCCGGAACAGCGCACGGACGGCTACACG |
| | | AGCGGCGGCCCGCCCGATCCTGCCATGATGGTCACCGCCGACGAACTCCGCGGGCAC |
| | | TTCGCCGAAGCGGGCATCGACCATCTCGAAGCGGCCGAACCGACCCTCGACGAAGGC |
| | | ATGCACCGGGGCCCCGCGGCGACGGTTCGTCTCGTGTGGTGCCGGCCGTCCACCTCG |
| EFR117-1 | 13. | ATGAAATACCAAGTATTACTTTATTACAAATATACAACAATTGAGGACCCAGAGGCT |
| | | TTTGCGAAAGAGCACCTAGCTTTTTGCAAATCATTAAACTTAAAAGGCCGCATCTTA |
| | | GTAGCGACAGAGGGGATCAACGGAACGTTATCTGGTACTGTCGAGGAGACAGAGAAG |
| | | TATATGGAGGCAATGCAGGCAGACGAGCGCTTTAAGGACACATTCTTTAAAATCGAC |
| | | CCAGCAGAGGAGATGGCCTTCCGCAAAATGTTTGTTCGCCCACGCTCTGAGTTAGTG |
| | | GCGTTGAACTTAGAGGAGGACGTTGACCCATTAGAGACGACGGGGAAATATTTGGAG |
| | | CCTGCAGAGTTTAAAGAGGCCTTATTAGACGAGGACACTGTTGTAATCGACGCTCGC |
| | | AACGACTATGAGTATGACTTAGGTCACTTCCGCGGTGCCGTGCGCCCAGACATCCGC |
| | | AGCTTCCGCGAGTTACCACAGTGGATCCGCGAGAACAAAGAGAAATTTATGGACAAA |
| | | AAAATCGTTACCTATTGTACTGGCGGGATCCGCTGTGAGAAATTTTCTGGCTGGTTA |
| | | TTAAAAGAGGGATTTGAGGACGTTGCTCAGTTGCACGGTGGTATCGCCAACTATGGA |
| | | AAAAATCCAGAGACACGCGGCGAGCTTTGGGACGGCAAAATGTATGTCTTTGACGAC |
| | | CGAATCAGTGTCGAGATCAATCACGTTGACAAAAAAGTTATCGGGAAAGACTGGTTT |
| | | GACGGGACACCTTGCGAGCGCTACATCAACTGTGCAAACCCAGAGTGTAATCGCCAG |
| | | ATCTTAACTTCAGAGGAGAATGAGCACAAACACTTAGGTGGCTGCTCATTAGAGTGT |
| | | AGCCAGCACCCTGCCAACCGCTATGTAAAAAAACACAATTTAACAGAGGCAGAGGTT |
| | | GCTGAGCGCTTAGCTTTGTTAGAGGCGGTTGAGGTA |
| EFR117-2 | 14. | ATGAAATACCAAGTATTACTTTATTACAAATATACAACAATTGAGGATCCAGAGGCT |
| | | TTTGCGAAAGAGCATCTAGCTTTTTGCAAATCATTAAACTTAAAAGGCCGTATTTTA |
| | | GTAGCGACAGAGGGGATTAACGGAACGTTATCTGGTACTGTCGAGGAGACAGAGAAG |
| | | TATATGGAGGCAATGCAAGCAGATGAGCGCTTTAAGGATACATTCTTTAAAATTGAT |
| | | CCAGCAGAGGAGATGGCCTTCCGCAAAATGTTTGTTCGCCCACGTTCTGAGTTAGTG |
| | | GCGTTGAACTTAGAGGAGGACGTTGATCCATTAGAGACGACGGGGAAATATTTGGAG |
| | | CCTGCAGAGTTTAAAGAGGCCTTATTAGACGAGGACACTGTTGTAATCGATGCTCGT |
| | | AACGATTATGAGTATGATTTAGGTCATTTCCGTGGTGCCGTGCGCCCAGATATCCGT |
| | | AGCTTCCGTGAGTTACCACAATGGATTCGCGAGAACAAAGAGAAATTTATGGATAAA |
| | | AAAATTGTTACCTATTGTACTGGCGGGATTCGCTGTGAGAAATTTTCTGGCTGGTTA |
| | | TTAAAAGAGGGATTTGAGGATGTTGCTCAATTGCATGGTGGTATCGCCAACTATGGA |
| | | AAAAATCCAGAGACACGTGGCGAGCTTTGGGACGGCAAAATGTATGTCTTTGATGAC |
| | | CGAATCAGTGTCGAGATTAATCATGTTGATAAAAAAGTTATTGGGAAAGACTGGTTT |
| | | GATGGGACACCTTGCGAGCGCTACATTAACTGTGCAAACCCAGAGTGTAATCGTCAA |
| | | ATCTTAACTTCAGAGGAGAATGAGCATAAACATTTAGGTGGCTGCTCATTAGAGTGT |
| | | AGCCAGCATCCTGCCAACCGTTATGTAAAAAAACATAATTTAACAGAGGCAGAGGTT |
| | | GCTGAGCGTTTAGCTTTGTTAGAGGCGGTTGAGGTA |
| BTR251-1 | 15. | ATGATATACAGATTTACTATCATATCTGATGAAGTTGACGATTTTGTCAGAGAGATA |
| | | CAGATCGACCCGGAGGCTACATTTCTTGACTTCCACGAGGCAATACTGAAATCAGTA |
| | | GGGTACACAAACGACCAGATGACCTCCTTCTTTATCTGCGACGACGACTGGGAGAAA |
| | | GAGAAAGAGGTCACTTTGGAGGAGATGGACGACAATCCGGAGATGGACAGTTGGATA |
| | | ATGAAAGAGACTACTATCAGCGAGCTGGTAGAGGACGAGAAGCAGAAATTGTTGTAT |
| | | GTATTCGACTACATGACAGAGCGCTGCTTCTTCATCGAGTTGTCTGAGATCATCACC |
| | | GGAAAAGACATGAATGGTGCCAAATGTACCAAGAAATCGGGTGACGCTCCGCCACAG |
| | | ACTGTAGACTTTGAGGAGATGGCTGCTGCAAGCGGTTCACTCGACCTGGACGAGAAT |
| | | TTCTATGGTGACCAGGACTTTGACATGGAGGACTTTGACCAGGAGGGCTTCGACATA |
| | | GGTGGTAACGCGGGTGGCTCTTATGAGGAGGAGAAGTTT |
| BTR251-2 | 16. | ATGATATACAGATTTACTATCATATCTGATGAAGTTGACGATTTTGTCAGAGAGATA |
| | | CAAATTGATCCGGAGGCTACATTTCTTGACTTCCATGAGGCAATACTGAAATCAGTA |
| | | GGGTACACAAACGACCAGATGACCTCCTTCTTTATCTGCGATGATGATTGGGAGAAA |
| | | GAGAAAGAGGTCACTTTGGAGGAGATGGACGACAATCCGGAGATGGATAGTTGGATA |
| | | ATGAAAGAGACTACTATCAGCGAGCTGGTAGAGGATGAGAAGCAAAAATTGTTGTAT |
| | | GTATTCGACTACATGACAGAGCGTTGCTTCTTCATCGAGTTGTCTGAGATCATCACC |
| | | GGAAAAGATATGAATGGTGCCAAATGTACCAAGAAATCGGGTGATGCTCCGCCACAA |
| | | ACTGTAGATTTTGAGGAGATGGCTGCTGCAAGCGGTTCACTCGACCTGGACGAGAAT |
| | | TTCTATGGTGATCAGGACTTTGATATGGAGGATTTTGATCAGGAGGGCTTCGACATA |
| | | GGTGGTAACGCGGGTGGCTCTTATGAGGAGGAGAAGTTT |
| XR92-1 | 17. | ATGAAGACAATTCAGGAGCAGCAGATGAAGATAGTTAGGAATATGCGTCGTATTCGT |
| | | TACAAGATTGCTGTTATTAGCACGAAAGGAGGTGTGGGGAAAAGCTTTGTTACCGCT |
| | | AGCCTCGCGGCAGCCCTCGCTGCGGAAGGGCGTCGTGTTGGAGTTTTTGATGCAGAT |
| | | ATTAGCGGTCCTAGCGTTCATAAAATGCTCGGCCTCCAAACGGGCATGGGTATGCCC |
| | | TCGCAACTCGATGGCACTGTAAAGCCCGTGGAAGTTCCTCCGGGAATTAAAGTAGCT |
| | | AGCATTGGGCTGTTGCTGCCCATGGATGAAGTGCCCCTAATTTGGCGTGGGGCCATT |
| | | AAGACGAGTGCCATTCGTGAACTGCTTGCATACGTCGATTGGGGAGAACTCGATTAT |
| | | CTCCTCATTGATCTACCTCCGGGAACAGGTGATGAAGTCCTCACGATTACCCAAATT |
| | | ATTCCCAACATTACGGGCTTCCTGGTAGTCACGATTCCCAGCGAAATTGCTAAGTCT |
| | | GTCGTTAAGAAGGCTGTCAGCTTTGCCAAGCGTATTGAAGCCCCTGTGATTGGAATT |
| | | GTCGAAAACATGAGCTACTTTCGTTGTAGCGATGGATCCATTCATTATATTTTCGGC |
| | | CGTGGCGCGGCTGAAGAAATTGCGTCACAATATGGTATTGAACTCCTCGGCAAAATT |
| | | CCCATTGATCCTGCGATTCGTGAATCGAACGATAAAGGCAAAATTTTCTTCCTAGAA |
| | | AATCCAGAAAGCGAAGCTTCGCGTGAATTCCTTAAGATTGCCCGTCGTATTATTGAA |
| | | ATTGTTGAAAAGCTAGGCCCAAAGCCTCCTGCGTGGGGTCCCCAAATGGAA |
| XR92-2 | 18. | ATGAAGACAATTCAGGAGCAGCAGATGAAGATAGTTAGGAATATGAGGAGGATTAGG |
| | | TACAAGATTGCTGTTATTAGCACGAAAGGAGGTGTGGGGAAAAGCTTTGTTACCGCT |
| | | AGCCTCGCGGCAGCCCTCGCTGCGGAGGGGCGAAGGGTTGGAGTTTTTGACGCAGAT |
| | | ATTAGCGGTCCTAGCGTTCATAAAATGCTCGGCCTCCAGACGGGCATGGGTATGCCC |
| | | TCGCAGCTCGACGGCACTGTAAAGCCCGTGGAAGTTCCTCCGGGAATTAAAGTAGCT |
| | | AGCATTGGGCTGTTGCTGCCCATGGATGAGGTGCCCCTAATTTGGAGAGGGGCCATT |
| | | AAGACGAGTGCCATTAGAGAGCTGCTTGCATACGTCGACTGGGGAGAACTCGACTAT |
| | | CTCCTCATTGACCTACCTCCGGGAACAGGTGATGAGGTCCTCACGATTACCCAGATT |
| | | ATTCCCAACATTACGGGCTTCCTGGTAGTCACGATTCCCAGCGAGATTGCTAAGTCT |
| | | GTCGTTAAGAAGGCTGTCAGCTTTGCCAAGAGGATTGAAGCCCCTGTGATTGGAATT |
| | | GTCGAGAACATGAGCTACTTTAGGTGTAGCGACGGATCCATTCACTATATTTTCGGC |
| | | CGCGGCGCGGCTGAGGAGATTGCGTCACAGTATGGTATTGAACTCCTCGGCAAAATT |
| | | CCCATTGACCCTGCGATTAGAGAGTCGAACGATAAAGGCAAAATTTTCTTCCTAGAG |
| | | AATCCAGAGAGCGAAGCTTCGAGAGAGTTCCTTAAGATTGCCCGCAGGATTATTGAG |
| | | ATTGTTGAGAAGCTAGGCCCAAAGCCTCCTGCGTGGGGTCCCCAGATGGAG |
| XR49-1 | 19. | ATGGGTAGTATAGAGGAGGTGCTTTTGGAGGAGAGGCTCATAGGATATCTAGATCCC |
| | | GGAGCCGAAAAAGTTTTAGCGCGTATTAACCGTCCTTCAAAAATTGTGTCTACAAGC |
| | | AGTTGTACAGGGCGTATTACACTGATTGAAGGCGAAGCTCATTGGCTCCGTAACGGG |
| | | GCACGTGTAGCGTACAAGACCCATCATCCCATTTCCCGTAGTGAAGTTGAACGTGTT |
| | | CTACGTCGTGGCTTCACAAACCTTTGGCTCAAGGTGACCGGCCCTATTCTACATCTC |
| | | CGTGTTGAAGGGTGGCAATGTGCAAAGTCCCTTCTCGAAGCAGCTCGTCGTAACGGG |
| | | TTCAAGCATAGCGGAGTCATTAGCATTGCTGAAGATTCACGTCTCGTCATTGAAATT |
| | | ATGAGCAGCCAAAGCATGTCAGTACCTCTAGTTATGGAAGGTGCTCGTATTGTCGGC |
| | | GATGATGCCCTAGATATGCTGATTGAAAAAGCAAACACTATTCTAGTTGAATCTCGT |
| | | ATTGGGCTAGATACGTTTTCACGTGAAGTCGAAGAACTTGTCGAATGCTTT |
| XR49-2 | 20. | ATGGGTAGTATAGAGGAGGTGCTTTTGGAGGAGAGGCTCATAGGATATCTAGACCCC |
| | | GGAGCCGAGAAAGTTTTAGCGAGGATTAACAGGCCTTCAAAAATTGTGTCTACAAGC |
| | | AGTTGTACAGGGAGGATTACACTGATTGAGGGCGAGGCTCACTGGCTCAGGAACGGG |
| | | GCAAGAGTAGCGTACAAGACCCATCACCCCATTTCCCGGAGTGAGGTTGAAAGGGTT |
| | | CTAAGGAGGGGCTTCACAAACCTTTGGCTCAAGGTGACCGGCCCTATTCTACATCTC |
| | | AGGGTTGAGGGGTGGCAGTGTGCAAAGTCCCTTCTCGAGGCAGCTAGGAGAAACGGG |
| | | TTCAAGCACAGCGGAGTCATTAGCATTGCTGAGGATTCAAGACTCGTCATTGAAATT |
| | | ATGAGCAGCCAGAGCATGTCAGTACCTCTAGTTATGGAGGGTGCTAGGATTGTCGGC |
| | | GACGATGCCCTAGATATGCTGATTGAGAAAGCAAACACTATTCTAGTTGAGTCTAGA |
| | | ATTGGGCTAGACACGTTTTCAAGAGAGGTCGAAGAGCTTGTCGAATGCTTT |
| IR165-1 | 21. | ATGAAACAATCGTTACGCCATCAAAAAATTATTAAACTGGTGGAGCAGTCTGGCTAT |
| | | TTAAGCACGGAGGAGTTGGTTGCTGCCTTAGACGTTAGCCCTCAGACGATCCGCCGC |
| | | GACTTGAATATCTTGGCGGAGTTAGACTTAATCCGCCGCCACCACGGTGGTGCGGCA |
| | | TCGCCATCTTCTGCAGAGAATTCTGACTACGTGGACCGCAAACAGTTCTTTTCATTA |
| | | CAGAAAAATAATATCGCACAGGAGGTTGCGAAGTTGATCCCTAACGGTGCATCGTTG |
| | | TTTATCGACATCGGTACGACGCCGGAGGCTGTCGCCAATGCGTTGCTTGGTCACGAG |
| | | AAACTCAGAATCGTGACGAACAATCTGAATGCCGCTCACCTTTTACGCCAGAATGAG |
| | | AGTTTTGACATCGTCATGGCGGGCGGATCATTACGAATGGACGGTGGAATCATCGGC |
| | | GAGGCTACGGTAAATTTTATCTCTCAGTTTCGCCTAGACTTCGGTATCTTAGGGATC |
| | | AGTGCGATCGACGCAGACGGTTCATTATTGGACTATGACTACCACGAGGTACAGGTA |
| | | AAACGAGCGATCATCGAGAGTTCACGCCAGACCTTATTAGTGGCCGACCACTCTAAA |
| | | TTTACTCGCCAGGCGATCGTTCGCTTGGGCGAGTTAAGTGACGTGGAGTATTTGTTT |
| | | ACAGGTGACGTTCCTGAGGGCATCGTCAATTATTTGAAAGAGCAGAAAACGAAATTG |
| | | GTTTTATGTAATGGTAAAGTGCGG |
| IR165-2 | 22. | ATGAAACAATCGTTACGCCATCAAAAAATTATTAAACTGGTGGAACAATCTGGCTAT |
| | | TTAAGCACGGAAGAATTGGTTGCTGCCTTAGATGTTAGCCCTCAAACGATCCGTCGT |
| | | GATTTGAATATCTTGGCGGAGTTAGATTTAATCCGCCGCCATCACGGTGGTGCGGCA |
| | | TCGCCATCTTCTGCAGAAAATTCTGATTACGTGGATCGTAAACAATTCTTTTCATTA |
| | | CAAAAAAATAATATCGCACAAGAAGTTGCGAAGTTGATCCCTAACGGTGCATCGTTG |
| | | TTTATCGATATCGGTACGACGCCGGAGGCTGTCGCCAATGCGTTGCTTGGTCATGAA |
| | | AAACTCAGAATCGTGACGAACAATCTGAATGCCGCTCATCTTTTACGCCAAAATGAA |
| | | AGTTTTGATATCGTCATGGCGGGCGGATCATTACGAATGGATGGTGGAATCATCGGC |
| | | GAAGCTACGGTAAATTTTATCTCTCAATTTCGCCTAGATTTCGGTATCTTAGGGATC |
| | | AGTGCGATCGATGCAGATGGTTCATTATTGGATTATGATTACCATGAAGTACAAGTA |
| | | AAACGAGCGATCATCGAAAGTTCACGTCAGACCTTATTAGTGGCCGATCACTCTAAA |
| | | TTTACTCGCCAAGCGATCGTTCGCTTGGGCGAATTAAGTGATGTGGAATATTTGTTT |
| | | ACAGGTGATGTTCCTGAGGGCATCGTCAATTATTTGAAAGAGCAGAAAACGAAATTG |
| | | GTTTTATGTAATGGTAAAGTGCGG |
| SPR66-1 | 23. | ATGATTAAATATAGTATCCGTGGTGAAAACCTAGAAGTAACAGAGGCAATCCGCGAC |
| | | TATGTAGTTTCTAAACTCGAGAAGATCGAGAAGTACTTCCAGCCAGAGCAGGAGTTG |
| | | GACGCCCGAATCAACTTAAAAGTTTATCGCGAGAAAACGGCTAAAGTGGAGGTAACG |
| | | ATCCCGCTTGGATCTATCACTCTCCGCGCAGAGGACGTATCTCAGGACATGTATGGT |
| | | TCAATCGACCTTGTAACTGACAAAATCGAGCGCCAGATCCGCAAAAATAAAACAAAA |
| | | ATCGAGCGCAAAAATAAAAATAAGGTAGCAACTGGTCAGTTATTTACAGACGCTTTG |
| | | GTGGAGGACTCAAATATCGTCCAGTCTAAAGTTGTTCGCTCAAAACAGATCGACTTA |
| | | AAACCAATGGACTTGGAGGAGGCAATCCTACAGATGGACTTATTGGGGCACGACTTC |
| | | TTTATCTATGTGGACGTTGAGGACCAGACAACCAATGTGATCTATCGCCGCGAGGAC |
| | | GGCGAGATCGGTTTGTTAGAGGTTAAAGAGTCT |
| SPR66-2 | 24. | ATGATTAAATATAGTATCCGTGGTGAAAACCTAGAAGTAACAGAAGCAATCCGTGAT |
| | | TATGTAGTTTCTAAACTCGAAAAGATCGAAAAGTACTTCCAACCAGAACAAGAGTTG |
| | | GATGCCCGAATCAACTTAAAAGTTTATCGTGAAAAAACGGCTAAAGTGGAAGTAACG |
| | | ATCCCGCTTGGATCTATCACTCTCCGCGCAGAAGATGTATCTCAAGATATGTATGGT |
| | | TCAATCGACCTTGTAACTGATAAAATCGAACGTCAGATCCGTAAAAATAAAACAAAA |
| | | ATCGAGCGTAAAAATAAAAATAAGGTAGCAACTGGTCAATTATTTACAGATGCTTTG |
| | | GTGGAAGATTCAAATATCGTCCAGTCTAAAGTTGTTCGTTCAAAACAAATCGATTTA |
| | | AAACCAATGGATTTGGAAGAAGCAATCCTACAAATGGATTTATTGGGGCATGATTTC |
| | | TTTATCTATGTGGATGTTGAAGATCAGACAACCAATGTGATCTATCGTCGTGAGGAT |
| | | GGCGAAATCGGTTTGTTAGAGGTTAAAGAATCT |

What is claimed is:

1. A method for increasing the solubility of a recombinant polypeptide produced from a nucleic acid in an expression system, the method comprising replacing one or more solubility decreasing codons in the nucleotide sequence encoding the recombinant polypeptide with a synonymous solubility increasing codon.

2. A method for decreasing the solubility of a recombinant polypeptide produced from a nucleic acid in an expression system, the method comprising replacing one or more solubility increasing codons in the nucleotide sequence encoding the recombinant polypeptide with a synonymous solubility decreasing codon.

3. A method for increasing the expression of a recombinant polypeptide produced from a nucleic acid in an expression system, the method comprising replacing one or more expression decreasing codons in the nucleotide sequence encoding the recombinant polypeptide with a synonymous expression increasing codon.

4. A method for decreasing the expression of a recombinant polypeptide produced from a nucleic acid in an expression system, the method comprising replacing one or more expression increasing codons in the nucleotide sequence encoding the recombinant polypeptide with a synonymous expression decreasing codon.

5. The method of claim 1 or 2, wherein the solubility decreasing codon is ATA (Ile) and the solubility increasing codon is ATT (Ile).

6. The method of claim 1 or 2, wherein the solubility decreasing codon is ATC (Ile) and the solubility increasing codon is ATT (Ile).

7. The method of claim 1 or 2, wherein the solubility decreasing codon is ATC (Ile) and the solubility increasing codon is ATT (Ile).

8. The method of claim 1 or 2, wherein the solubility decreasing codon is any of AGA (Arg), AGG (Arg), CGA (Arg), or CGC (Arg) and the solubility increasing codon is CTG (Arg).

9. The method of claim 1 or 2, wherein the solubility decreasing codon is GGG (Gly) and the solubility increasing codon is GGT (Gly).

10. The method of claim 1 or 2, wherein the solubility decreasing codon is GTG (Val) and the solubility increasing codon is GTT (Val).

11. The method of claim 3 or 4, wherein the expression decreasing codon is GAG (Glu) and the expression increasing codon is GAA (Glu).

12. The method of claim 3 or 4, wherein the expression decreasing codon is GAC (Asp) and the expression increasing codon is GAT (Asp).

13. The method of claim 3 or 4, wherein the expression decreasing codon is CAC (His) and the expression increasing codon is CAT (His).

14. The method of claim 3 or 4, wherein the expression decreasing codon is CAG (Gln) and the expression increasing codon is CAA (Gln).

15. The method of claim 3 or 4, wherein the expression decreasing codon is any of AGA (Asn), AGG (Asn), CGT (Asn), CGC(Asn), or CGG (Asn) and the expression increasing codon is CGA (Asn).

16. The method of claim 3 or 4, wherein the expression decreasing codon is GGG (Gly) and the expression increasing codon is GGT (Gly).

17. The method of claim 3 or 4, wherein the expression decreasing codon is TTC (Phe) and the expression increasing codon is TTT (Phe).

18. The method of claim 3 or 4, wherein the expression decreasing codon is CCC (Pro) or CCG (Pro) and the expression increasing codon is CCT (Pro).

19. The method of claim 3 or 4, wherein the expression decreasing codon is TCC (Ser) or TCG (Ser) and the expression increasing codon is AGT (Ser).

20. A method for increasing the solubility of a recombinant polypeptide produced from a nucleic acid in an expression system, the method comprising replacing one or more solubility decreasing codons in the nucleotide sequence encoding the recombinant polypeptide with a non-synonymous solubility increasing codon.

21. A method for decreasing the solubility of a recombinant polypeptide produced from a nucleic acid in an expression system, the method comprising replacing one or more solubility increasing codons in the nucleotide sequence encoding the recombinant polypeptide with a non-synonymous solubility decreasing codon.

22. A method for increasing the expression of a recombinant polypeptide produced from a nucleic acid in an expression system, the method comprising replacing one or more expression decreasing codons in the nucleotide sequence encoding the recombinant polypeptide with a non-synonymous expression increasing codon.

23. A method for decreasing the expression of a recombinant polypeptide produced from a nucleic acid in an expression system, the method comprising replacing one or more expression increasing codons in the nucleotide sequence encoding the recombinant polypeptide with a non-synonymous expression decreasing codon.

24. The method of claim 20 or 21, wherein the solubility decreasing codon is any of TTA (Leu), TTG (Leu), CTT (Leu), CTC (Leu), CTA (Leu), CTG (Leu) and the solubility increasing codon is ATT (Ile).

25. The method of claim 22 or 23, wherein the expression decreasing codon is any of TTA (Leu), TTG (Leu), CTT (Leu), CTC (Leu), CTA (Leu), CTG (Leu) and the expression increasing codon is ATT (Ile).

26. A method for increasing the solubility of a recombinant polypeptide produced in an expression system, the method comprising replacing one or more solubility decreasing amino acid residues in the recombinant polypeptide with a solubility increasing amino acid residue.

27. A method for decreasing the solubility of a recombinant polypeptide produced in an expression system, the method comprising replacing one or more solubility increasing amino acid residues in the recombinant polypeptide with a solubility decreasing amino acid residue.

28. The method of claim 26 or claim 27, wherein the solubility decreasing amino acid is arginine and the solubility increasing amino acid is lysine.

29. The method of claim 26 or claim 27, wherein the solubility decreasing amino acid is valine and the solubility increasing amino acid is isoleucine.

30. The method of claim 26 or claim 27, wherein the solubility decreasing amino acid is leucine and the solubility increasing amino acid is valine.

31. The method of claim 26 or claim 27, wherein the solubility decreasing amino acid is leucine and the solubility increasing amino acid is isoleucine.

32. The method of claim 26 or claim 27, wherein the solubility decreasing amino acid is phenylalanine and the solubility increasing amino acid is valine.

33. The method of claim 26 or claim 27, wherein the solubility decreasing amino acid is phenylalanine and the solubility increasing amino acid is isoleucine.

34. The method of claim 26 or claim 27, wherein the solubility decreasing amino acid is cysteine and the solubility increasing amino acid is phenylalanine.

35. The method of claim 26 or claim 27, wherein the solubility decreasing amino acid is cysteine and the solubility increasing amino acid is valine.

36. The method of claim 26 or claim 27, wherein the solubility decreasing amino acid is cysteine and the solubility increasing amino acid is isoleucine.

37. The method of claim 26 or claim 27, wherein the solubility decreasing amino acid is histidine and the solubility increasing amino acid is threonine.

38. The method of claim 26 or claim 27, wherein the solubility decreasing amino acid is proline and the solubility increasing amino acid is valine.

39. A method for increasing the expression of a recombinant polypeptide produced in an expression system, the method comprising replacing one or more expression decreasing amino acid residues in the recombinant polypeptide with a expression increasing amino acid residue.

40. A method for decreasing the expression of a recombinant polypeptide produced in an expression system, the method comprising replacing one or more expression increasing amino acid residues in the recombinant polypeptide with a expression decreasing amino acid residue.

41. The method of claim 39 or claim 40, wherein the expression decreasing amino acid is arginine and the expression increasing amino acid is lysine.

42. The method of claim 39 or claim 40, wherein the expression decreasing amino acid is valine and the expression increasing amino acid is isoleucine.

43. The method of claim 39 or claim 40, wherein the expression decreasing amino acid is leucine and the expression increasing amino acid is valine.

44. The method of claim 39 or claim 40, wherein the expression decreasing amino acid is leucine and the expression increasing amino acid is isoleucine.

45. The method of claim 39 or claim 40, wherein the expression decreasing amino acid is cysteine and the expression increasing amino acid is phenylalanine.

46. The method of claim 39 or claim 40, wherein the expression decreasing amino acid is alanine and the expression increasing amino acid is methionine.

47. The method of claim 39 or claim 40, wherein the expression decreasing amino acid is alanine and the expression increasing amino acid is cysteine.

48. The method of claim 39 or claim 40, wherein the expression decreasing amino acid is alanine and the expression increasing amino acid is phenylalanine.

49. The method of claim 39 or claim 40, wherein the expression decreasing amino acid is alanine and the expression increasing amino acid is leucine.

50. The method of claim 39 or claim 40, wherein the expression decreasing amino acid is alanine and the expression increasing amino acid is valine.

51. The method of claim 39 or claim 40, wherein the expression decreasing amino acid is alanine and the expression increasing amino acid is isoleucine.

52. The method of claim 39 or claim 40, wherein the expression decreasing amino acid is tryptophan and the expression increasing amino acid is methionine.

53. The method of claim 39 or claim 40, wherein the expression decreasing amino acid is arginine and the expression increasing amino acid is isoleucine.

54. The method of claim 39 or claim 40, wherein the expression decreasing amino acid is arginine and the expression increasing amino acid is glutamic acid.

55. The method of claim 39 or claim 40, wherein the expression decreasing amino acid is arginine and the expression increasing amino acid is aspartic acid.

56. The method of claim 39 or claim 40, wherein the expression decreasing amino acid is lysine and the expression increasing amino acid is glutamic acid.

57. The method of claim 39 or claim 40, wherein the expression decreasing amino acid is lysine and the expression increasing amino acid is aspartic acid.

58. A method for increasing the solubility of a recombinant polypeptide produced in an expression system, the method comprising replacing a first type of amino acid at one or more positions in the recombinant polypeptide with a second type of amino acid residue, wherein the second amino acid residue has a greater or equivalent hydrophobicity and a greater solubility predictive value as compared to the first type of amino acid.

59. A method for increasing the expression of a recombinant polypeptide produced in an expression system, the method comprising replacing a first type of amino acid at one or more positions in the recombinant polypeptide with a second type of amino acid residue, wherein the second amino acid residue has a greater expression predictive value as compared to the first amino acid.

60. A method for decreasing the solubility of a recombinant polypeptide produced in an expression system, the method comprising replacing a first type of amino acid at one or more positions in the recombinant polypeptide with a second type of amino acid residue, wherein the second amino acid residue has a greater or equivalent hydrophilicity and a lesser solubility predictive value as compared to the first amino acid.

61. A method for decreasing the expression of a recombinant polypeptide produced in an expression system, the method comprising replacing a first type of amino acid at one or more positions in the recombinant polypeptide with a second type of amino acid residue, wherein the second amino acid residue has a lesser expression predictive value as compared to the first amino acid.

62. The method of claim 59 or 61, wherein the second amino acid residue has a greater or equivalent hydrophobicity compared to the first amino acid.

63. The method of any of claim 1-4, 20-24, 26, 27, 39, 40, or 58-61, wherein the expression system in an in vitro expression system.

64. The method of claim 63, wherein the in vitro expression system is a cell-free transcription/translation system.

65. The method of any of claim 1-4, 20-24, 26, 27, 39, 40, or 58-61, wherein the expression system in an in vivo expression system.

66. The method of claim 65, wherein the in vivo expression system is a bacterial expression system or a eukaryotic expression system.

67. The method of claim 66, wherein the in vivo expression system is an E. coli cell.

68. The method of claim 66, wherein the in vivo expression system is a mammalian cell.

69. The method of any of claim 1-4, 20-24, 26, 27, 39, 40, or 58-61, wherein the recombinant polypeptide is a human polypeptide, or a fragment thereof.

70. The method of any of claim 1-4, 20-24, 26, 27, 39, 40, or 58-61, wherein the recombinant polypeptide is a viral polypeptide, or a fragment thereof.

71. The method of any of claim 1-4, 20-24, 26, 27, 39, 40, or 58-61, wherein the recombinant polypeptide is an antibody, an antibody fragment, an antibody derivative, a diabody, a tribody, a tetrabody, an antibody dimer, an antibody trimer or a minibody.

72. The method of claim 71, wherein the antibody fragment is a Fab fragment, a Fab′ fragment, a F(ab)2 fragment, a Fd fragment, a Fv fragment, or a ScFv fragment.

73. The method of any of claim 1-4, 20-24, 26, 27, 39, 40, or 58-61, wherein the recombinant polypeptide is a cytokine, an inflammatory molecule, a growth factor, a cytokine receptor, an inflammatory molecule receptor, a growth factor receptor, an oncogene product, or any fragment thereof.

74. The method of any of claim 1-4, 20-24, 26, 27, 39, 40, or 58-61, wherein the recombinant polypeptide is a fusion polypeptide.

75. A recombinant polypeptide produced by the method of any of claim 1-4, 20-24, 26, 27, 39, 40, or 58-61.

76. A pharmaceutical composition comprising the recombinant polypeptide of claim 75.

77. An immunogenic composition comprising the recombinant polypeptide of claim 76.

78. A method for predicting whether first polypeptide encoded by a first nucleic acid sequence will have greater solubility than a second polypeptide encoded by a second nucleic acid sequence when expressed in an expression system, the method comprising,
a) calculating a value for one or more sequence parameters of the first nucleic acid sequence,
b) calculating a value for one or more sequence parameters of the second nucleic acid sequence,
c) multiplying the value for each sequence parameter in step (a) by the solubility regression slope of the sequence parameter to determine a combined solubility value for the sequence parameter of the first nucleic acid sequence,
d) multiplying the value for each sequence parameter in step (b) by the solubility regression slope of the sequence parameter to determine a combined solubility value for the sequence parameter of the second nucleic acid sequence,
e) comparing the combined solubility value for the sequence parameter of the first nucleic acid sequence to the combined solubility value for the sequence parameter of the second nucleic acid sequence, wherein a greater combined solubility value for the sequence parameter of the first nucleic acid sequence as compared to the combined solubility value for the sequence parameter of the second nucleic acid sequence indicates that first polypeptide will have greater solubility than a second polypeptide when expressed in an expression system.

79. A method for predicting whether first polypeptide encoded by a first nucleic acid sequence will have greater expression than a second polypeptide encoded by a second nucleic acid sequence when expressed in an expression system, the method comprising,
a) calculating a value for one or more sequence parameters of the first nucleic acid sequence,
b) calculating a value for one or more sequence parameters of the second nucleic acid sequence,
c) multiplying the value for each sequence parameter in step (a) by the expression regression slope of the sequence parameter to determine a combined expression value for the sequence parameter of the first nucleic acid sequence,
d) multiplying the value for each sequence parameter in step (b) by the expression regression slope of the sequence parameter to determine a combined expression value for the sequence parameter of the second nucleic acid sequence,
e) comparing the combined expression value for the sequence parameter of the first nucleic acid sequence to the combined expression value for the sequence parameter of the second nucleic acid sequence, wherein a greater combined expression value for the sequence parameter of the first nucleic acid sequence as compared to the combined expression value for the sequence parameter of the second nucleic acid sequence indicates that first polypeptide will have greater expression than a second polypeptide when expressed in an expression system.

80. A method for predicting whether first polypeptide encoded by a first nucleic acid sequence will have greater usability than a second polypeptide encoded by a second nucleic acid sequence when expressed in an expression system, the method comprising,
a) calculating a value for one or more sequence parameters of the first nucleic acid sequence,
b) calculating a value for one or more sequence parameters of the second nucleic acid sequence,
c) multiplying the value for each sequence parameter in step (a) by the usability regression slope of the sequence parameter to determine a combined usability value for the sequence parameter of the first nucleic acid sequence,
d) multiplying the value for each sequence parameter in step (b) by the usability regression slope of the sequence parameter to determine a combined usability value for the sequence parameter of the second nucleic acid sequence,
e) comparing the combined usability value for the sequence parameter of the first nucleic acid sequence to the combined usability value for the sequence parameter of the second nucleic acid sequence, wherein a greater combined usability value for the sequence parameter of the first nucleic acid sequence as compared to the combined usability value for the sequence parameter of the second nucleic acid sequence indicates that first polypeptide will have greater usability than a second polypeptide when expressed in an expression system.

81. The method of any of claims 78-80, wherein the one or more sequence parameter is selected from the group comprising the fraction of amino acid residues in the polypeptide that are predicted to be disordered; the surface exposure and/or burial status of each residue in the polypeptide; the fractional content of the polypeptide made up by each amino acid; the fractional content of the polypeptide made up by each amino acid predicted to be buried or exposed; the fractional content of the polypeptide made up by each codon; the length of the polypeptide chain; the net charge of the polypeptide; the absolute value of the net charge of the polypeptide; the value for the net charge of the polypeptide divided by the length of the polypeptide; the absolute value of the net charge of the polypeptide divided by the length of the polypeptide; the isoelectric point of the polypeptide; the mean side-chain entropy of the polypeptide; the mean side-chain entropy of all residues predicted to be surface-exposed; and the mean hydrophobicity of the polypeptide.

82. The method of claim 81, wherein the one or more sequence parameter is the fractional content of the polypeptide made up by rare codons.

83. The method of claim 82, wherein the rare codons are selected from the group comprising AGG(Arg), AGA(Arg), CGG(Arg), CGA(Arg), ATA(Ile), CTA(Leu), and CCC(Pro).

84. The method of any of claims 78-80 wherein the sequence parameters in step (b) and step (c) are the same.
